(19) United States
(12) Patent Application Publication
(10) Pub. No.: US 2024/0270709 A1
(43) Pub. Date: Aug. 15, 2024

(54) NOVEL ORGANIC COMPOUND AND ORGANIC LIGHT-EMITTING DEVICE COMPRISING SAME

(71) Applicant: SFC CO., LTD., Cheongju-si (KR)

(72) Inventors: Si-In KIM, Cheongju-si (KR); Se-Jin LEE, Cheongju-si (KR); Seok-Bae PARK, Cheongju-si (KR); Hee-Dae KIM, Cheongju-si (KR); Yeong-Tae CHOI, Cheongju-si (KR); Ji-Ying KIM, Cheongju-si (KR); Kyungtae KIM, Cheongju-si (KR); Myeong-Jun KIM, Cheongju-si (KR); Kyeong-Hyeon KIM, Cheongju-si (KR); Seung-Soo LEE, Cheongju-si (KR); Tae Gyun LEE, Cheongju-si (KR); Joon-Ho KIM, Cheongju-si (KR)

(21) Appl. No.: 18/289,365

(22) PCT Filed: May 12, 2022
(86) PCT No.: PCT/KR2022/006841

§ 371 Date: Nov. 2, 2023

(30) Foreign Application Priority Data
May 14, 2021 (KR) 10-2021-0062819
Nov. 4, 2021 (KR) 10-2021-0150557

(51) Int. Cl. C07D 307/79; C07D 307/92; C07D 405/10; C07D 407/04; C07D 407/10; C07D 407/14; C07D 409/14; C07D 491/04; C07D 493/04; C09K 11/06; H10K 50/12; H10K 85/60

(52) U.S. Cl. CPC C07D 307/79; C07D 307/92; C07D 405/10; C07D 407/04; C07D 407/10; C07D 407/14; C07D 409/14; C07D 491/04; C07D 493/04; C09K 11/06; H10K 50/12; H10K 85/636; H10K 85/6574; H10K 85/6576; H10K 85/658; C09K 2211/1018

(57) ABSTRACT

The present invention relates to a novel heterocyclic compound which can be used for an organic light-emitting device, and an organic light-emitting device comprising same, wherein [Chemical Formula A] above is as set forth in the detailed description of the invention.

### TECHNICAL FIELD

The present disclosure relates to a novel compound useful for an organic light-emitting diode and, more specifically, to a novel compound that can be used as a host material in an organic light-emitting diode and allows for excellent diode characteristics including high luminous efficiency, low driving voltage, and high longevity, and an organic light-emitting diode including same.

### BACKGROUND ART

Organic light-emitting diodes (OLEDs), based on self-luminescence, enjoy the advantage of having a wide viewing angle and being able to be made thinner and lighter than liquid crystal displays (LCDs). In addition, an OLED display exhibits a very fast response time. Accordingly, OLEDs find applications in the illumination field as well as the full-color display field.

In general, the term “organic light-emitting phenomenon” refers to a phenomenon in which electrical energy is converted to light energy by means of an organic material. An OLED using the organic light-emitting phenomenon has a structure usually comprising an anode, a cathode, and an organic material layer interposed therebetween. In this regard, the organic material layer may be, for the most part, of a multilayer structure consisting of different materials, for example, a hole injection layer, a hole transport layer, a light-emitting layer, an electron transport layer, and an electron injection layer, in order to improve the efficiency and stability of the organic light-emitting diode (OLED). In the organic light-emitting diode having such a structure, when a voltage is applied between the two electrodes, a hole injected from the anode migrates to the organic layer while an electron is released from the cathode and moves toward the organic layer. In the luminescent zone, the hole and the electron recombine to produce an exciton. When the exciton returns to the ground state from the excited state, the molecule of the organic layer emits light. Such an organic light emitting diode is known to have characteristics such as self-luminescence, high luminance, high efficiency, low driving voltage, a wide viewing angle, high contrast, and high-speed response.

Materials used as organic layers in OLEDs may be divided into luminescent materials and charge transport materials, for example, a hole injection material, a hole transport material, an electron injection material, and an electron transport material. As for the luminescent materials, there are two main families of OLED: those based on small molecules and those employing polymers. The light-emitting mechanism forms the basis for classification of the luminescent materials as fluorescent or phosphorescent materials, which use excitons in singlet and triplet states, respectively.

Meanwhile, when a single material is employed as the luminescent material, intermolecular actions cause the wavelength of maximum luminescence to shift toward a longer wavelength, decreasing color purity or attenuating light with consequent reduction in the efficiency of the diode. In this regard, a host-dopant system may be used as a luminescent material so as to increase the color purity and the light emission efficiency through energy transfer.

This is based on the principle whereby, when a dopant is smaller in energy band gap than a host accounting for the light-emitting layer, the addition of a small amount of the dopant to the host generates excitons from the light emitting layer so that the excitons are transported to the dopant, emitting light at high efficiency. Here, light of desired wavelengths can be obtained depending on the kind of dopant because the wavelength of the host moves to the wavelength range of the dopant.

For use as host compounds in a light-emitting layer, heterocyclic compounds have been recently studied. With regard to related art, reference may be made to Korean Patent No. 10-2016-0089693 A (Jul. 28, 2016), which discloses a compound structured to have a dibenzofuran ring moiety bonded to an anthracene ring, and an organic light-emitting diode including same. In addition, Korean Patent No. 10-2017-0055743 A (May 22, 2017) discloses a compound in which an aryl substituent or a heteroaryl substituent is bonded to a fused fluorene ring bearing a heteroatom such as oxygen, nitrogen, sulfur, etc., and an organic light-emitting diode including same.

Despites a variety of types of compounds prepared for use in light emitting layers in organic light emitting diodes including the related arts, there is still a continuing need to develop a novel compound that allows an OLED to be stably driven at a lower voltage with high efficiency and longevity, and an OLED including same.

### DISCLOSURE

### Technical Problem

Therefore, an aspect of the present disclosure is to provide a novel organic compound which can be used as a host material in a light-emitting layer of an organic light-emitting diode

In addition, another aspect of the present disclosure is to provide an organic light-emitting diode (OLED) having the organic compound as a host material therein and exhibiting characteristics including high luminous efficiency, low voltage driving, and high longevity.

### Technical Solution

In order to accomplish the purposes, the present disclosure provides an organic compound represented by the following Chemical Formula A:

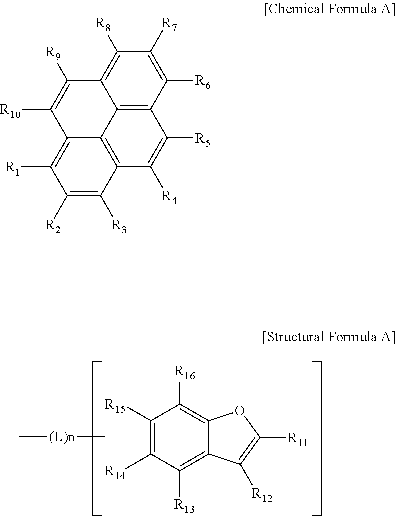

wherein,
R₁ to R₁₀, which are same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, at least one of R₁ to R₁₀ being a single bond that is linked to linker L in Structural Formula A to connect the pyrene ring moiety to the moiety represented by Structural Formula A;
if existing to connect with the pyrene ring moiety, two or more moieties of Structural Formula A are same or different;
the linker L in Structural Formula A is selected from a single bond, a substituted or unsubstituted arylene of 6 to 20 carbon atoms, and a substituted or unsubstituted heteroarylene of 2 to 20 carbon atoms;
n is an integer of 0 to 2, wherein when n is 2, the corresponding linkers L's are same or different;
R₁₁ to R₁₆, which is same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, one of R₁₁ to R₁₆ being a single bond linked to the linker L; and
R₁₃ to R₁₆ may each further form mono- or polycyclic aliphatic or aromatic ring with an adjacent radical thereto,
wherein the term ‘substituted’ in the expression “a substituted or unsubstituted” means having at least one substituent selected from the group consisting of a deuterium atom, a cyano, a halogen, a hydroxy, a nitro, an alkyl of 1 to 24 carbon atoms, a hydrogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

### Advantageous Effects

When used as a host material, the novel compound represented by Chemical Formula A according to the present disclosure allows for the provision of an organic light-emitting diode that can be driven at a lower voltage with improved luminous efficiency and longevity, compared to conventional organic light-emitting diodes.

### DESCRIPTION OF DRAWINGS

FIG. 1 is a schematic diagram of an organic light-emitting diode according to an embodiment of the present disclosure.

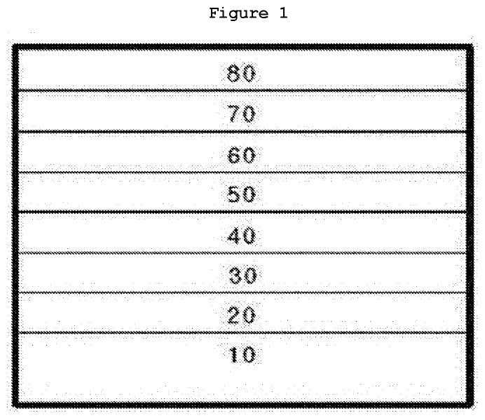

### BEST MODE FOR INVENTION

Hereinafter, exemplary embodiments which can be easily implemented by those skilled in the art will be described with reference to the accompanying drawing. In each drawing of the present disclosure, sizes or scales of components may be enlarged or reduced from their actual sizes or scales for better illustration, and known components may not be depicted therein to clearly show features of the present disclosure. Therefore, the present disclosure is not limited to the drawings. When describing the principle of the embodiments of the present disclosure in detail, details of well-known functions and features may be omitted to avoid unnecessarily obscuring the presented embodiments.

In drawings, for convenience of description, sizes of components may be exaggerated for clarity. For example, since sizes and thicknesses of components in drawings are arbitrarily shown for convenience of description, the sizes and thicknesses are not limited thereto. Furthermore, throughout the description, the terms “on” and “over” are used to refer to the relative positioning, and mean not only that one component or layer is directly disposed on another component or layer but also that one component or layer is indirectly disposed on another component or layer with a further component or layer being interposed therebetween. Also, spatially relative terms, such as “below”, “beneath”, “lower”, and “between” may be used herein for ease of description to refer to the relative positioning.

Throughout the specification, when a portion may “comprise” or “include” a certain constituent element, unless explicitly described to the contrary, it may not be construed to exclude another constituent element but may be construed to further include other constituent elements. Further, throughout the specification, the word “on” means positioning on or below the object portion, but does not essentially mean positioning on the lower side of the object portion based on a gravity direction.

The present disclosure provides an organic light-emitting compound represented by the following Chemical Formula A:

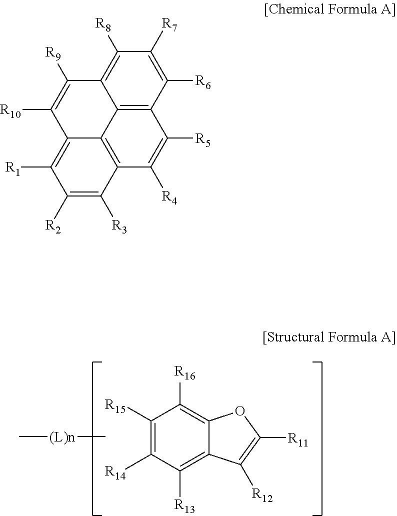

wherein,
R₁ to R₁₀, which are same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, at least one of R₁ to R₁₀ being a single bond that is linked to linker L in Structural Formula A to connect the pyrene ring moiety to the moiety represented by Structural Formula A;
if existing to connect with the pyrene ring moiety, two or more moieties of Structural Formula A are same or different;
the linker L in Structural Formula A is selected from a single bond, a substituted or unsubstituted arylene of 6 to 20 carbon atoms, and a substituted or unsubstituted heteroarylene of 2 to 20 carbon atoms;
n is an integer of 0 to 2, wherein when n is 2, the corresponding linkers L's are same or different;
R₁₁ to R₁₆, which is same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, one of R₁₁ to R₁₆ being a single bond linked to the linker L; and
R₁₃ to R₁₆ may each further form mono- or polycyclic alicyclic or aromatic ring with an adjacent radical thereto,
wherein the term ‘substituted’ in the expression “a substituted or unsubstituted” means having at least one substituent selected from the group consisting of a deuterium atom, a cyano, a halogen, a hydroxy, a nitro, an alkyl of 1 to 24 carbon atoms, a hydrogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

The expression indicating the number of carbon atoms, such as “a substituted or unsubstituted alkyl of 1 to 30 carbon atoms”, “a substituted or unsubstituted aryl of 5 to 50 carbon atoms”, etc., means the total number of carbon atoms of, for example, the alkyl or aryl radical or moiety alone, exclusive of the number of carbon atoms of substituents attached thereto. For instance, a phenyl group with a butyl at the para position falls within the scope of an aryl of 6 carbon atoms, even though it is substituted with a butyl radical of 4 carbon atoms.

As used herein, the term “aryl” means an organic radical derived from an aromatic hydrocarbon by removing one hydrogen that is bonded to the aromatic hydrocarbon. Further, the aromatic system may include a fused ring that is formed by adjacent substituents on the aryl radical.

Concrete examples of the aryl include phenyl, o-biphenyl, m-biphenyl, p-biphenyl, o-terphenyl, m-terphenyl, p-terphenyl, naphthyl, anthryl, phenanthryl, pyrenyl, indenyl, fluorenyl, tetrahydronaphthyl, perylenyl, chrysenyl, naphthacenyl, and fluoranthenyl, etc. at least one hydrogen atom of which may be substituted by a deuterium atom, a halogen atom, a hydroxy, a nitro, a cyano, a silyl, an amino (—NH₂, —NH(R), —N(R′) (R″) wherein R′ and R″ are each independently an alkyl of 1 to 10 carbon atoms, in this case, called “alkylamino”), an amidino, a hydrazine, a hydrazone, a carboxyl, a sulfonic acid, a phosphoric acid, an alkyl of 1 to 24 carbon atoms, a halogenated alkyl of 1 to 24 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 6 to 24 carbon atoms, a heteroaryl of 2 to 24 carbon atoms, or a heteroarylalkyl of 2 to 24 carbon atoms.

The substituent heteroaryl used in the compound of the present disclosure refers to a cyclic aromatic system of 2 to 24 carbon atoms bearing as ring members one to three heteroatoms selected from among N, O, P, Si, S, Ge, Se, and Te. In the aromatic system, two or more rings may be fused. One or more hydrogen atoms on the heteroaryl may be substituted by the same substituents as on the aryl.

In addition, the term “heteroaromatic ring”, as used herein, refers to an aromatic hydrocarbon ring bearing at least one hetero atom as an aromatic ring member. Preferably, the heteroaromatic ring bears as aromatic ring members one to three heteroatoms selected particularly from N, O, P, Si, S, Ge, Se, and Te.

As used herein, the term “alkyl” refers to an alkane missing one hydrogen atom and includes linear or branched structures. Examples of the alkyl substituent useful in the present disclosure include methyl, ethyl, propyl, isopropyl, isobutyl, sec-butyl, tert-butyl, pentyl, iso-amyl, and hexyl, etc. At least one hydrogen atom of the alkyl may be substituted by the same substituent as on the aryl.

The term “cyclo” as used in substituents of the present disclosure, such as cycloalkyl, refers to a structure responsible for a mono- or polycyclic ring of saturated hydrocarbons such as alkyl. Concrete examples of cycloalkyl radicals include cyclopropyl, cyclobutyl, cyclopentyl, cyclohexyl, methylcyclopentyl, methylcyclohexyl, ethylcyclopentyl, ethylcyclohexyl, adamantyl, dicyclopentadienyl, decahydronaphthyl, norbornyl, bornyl, and isobornyl, etc. One or more hydrogen atoms on the cycloalkyl may be substituted by the same substituents as on the aryl.

The term “alkoxy” as used in the compounds of the present disclosure refers to an alkyl or cycloalkyl singularly bonded to oxygen. Concrete examples of the alkoxy include methoxy, ethoxy, propoxy, isobutoxy, sec-butoxy, pentoxy, iso-amyloxy, hexyloxy, cyclobutyloxy, cyclopentyloxy, adamantyloxy, dicyclopentyloxy, bornyloxy, and isobornyloxy, etc. One or more hydrogen atoms on the alkoxy may be substituted by the same substituents as on the aryl.

Concrete examples of the arylalkyl used in the compounds of the present disclosure include phenylmethyl(benzyl), phenylethyl, phenylpropyl, naphthylmethyl, and naphthylethyl, etc. One or more hydrogen atoms on the arylalkyl may be substituted by the same substituents as on the aryl.

Concrete examples of the silyl radicals used in the compounds of the present disclosure include trimethylsilyl, triethylsilyl, triphenylsilyl, trimethoxysilyl, dimethoxyphenylsilyl, diphenylmethylsilyl, diphenylvinlysilyl, methylcyclobutylsilyl, and dimethyl furylsilyl, etc. One or more hydrogen atoms on the silyl may be substituted by the same substituents as on the aryl.

As used herein, the term “alkenyl” refers to an unsaturated hydrocarbon group that contains a carbon-carbon double bond between two carbon atoms and the team “alkynyl” refers to an unsaturated hydrocarbon group that contains a carbon-carbon triple bond between two carbon atoms.

As used herein, the term “alkylene” refers to an organic aliphatic radical regarded as derived from a linear or branched saturated hydrocarbon alkane by removal of two hydrogen atoms from different carbon atoms. Concrete examples of the alkylene include methylene, ethylene, propylene, isopropylene, isobutylene, sec-butylene, tert-butylene, pentylene, iso-amylene, hexylene, and so on. One or more hydrogen atoms on the alkylene may be substituted by the same substituents as on the aryl.

Furthermore, as used herein, the term “diarylamino” refers to an amine radical having two identical or different aryl groups bonded to the nitrogen atom thereof, the term “diheteroarylamino” refers to an amine radical having two identical or different heteroaryl groups bonded to the nitrogen atom thereof, and the term “aryl(heteroaryl)amino” refers to an amine radical having an aryl group and a heteroaryl group both bonded to the nitrogen atom thereof.

As more particular examples accounting for the term “substituted” in the expression “substituted or unsubstituted” used for compounds of Chemical Formula A, the compounds may be substituted by at least one substituents selected from the group consisting of a deuterium atom, a cyano, a halogen, a hydroxy, a nitro, an alkyl of 1 to 12 carbon atoms, a halogenated alkyl of 1 to 12 carbon atoms, an alkenyl of 2 to 12 carbon atoms, an alkynyl of 2 to 12 carbon atoms, a cycloalkyl of 3 to 12 carbon atoms, a heteroalkyl of 1 to 12 carbon atoms, an aryl of 6 to 18 carbon atoms, an arylalkyl of 7 to 20 carbon atoms, an alkylaryl of 7 to 20 carbon atoms, a heteroaryl of 2 to 18 carbon atoms, a heteroarylalkyl of 2 to 18 carbon atoms, an alkoxy of 1 to 12 carbon atoms, an alkylamino of 1 to 12 carbon atoms, a diarylamino of 12 to 18 carbon atoms, a diheteroarylamino of 2 to 18 carbon atoms, an aryl(heteroaryl)amino of 7 to 18 carbon atoms, an alkylsilyl of 1 to 12 carbon atoms, an arylsilyl of 6 to 18 carbon atoms, an aryloxy of 6 to 18 carbon atoms, and an arylthionyl of 6 to 18 carbon atoms.

In the present disclosure, the organic compound represented by Chemical Formula A is characterized by the structure in which a substituted or unsubstituted pyrene ring moiety is connected to a linker (L)n, and the linker (L)n is connected to a substituted or unsubstituted benzofuran moiety so that a substituent represented by Structural Formula A is connected to the substituted or unsubstituted pyrene ring:

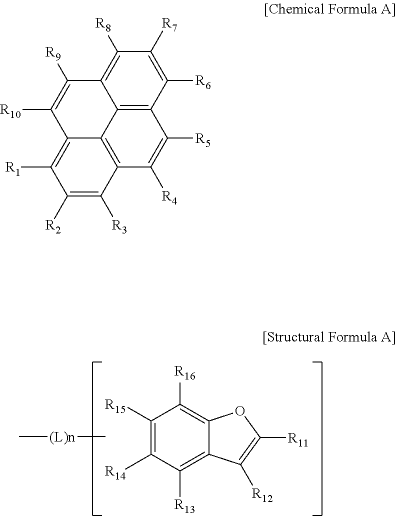

In an embodiment of the present disclosure, at least one of the substituents R₁, R₃, R₆, and R₈ on the pyrene moiety in Chemical Formula A may be a single bond linked to the linker L in structural Formula A. That is, at least one of the substituents R₁, R₃, R₆, and R₈ in the pyrene ring moiety of Chemical Formula A is a single bond connected to the moiety represented by Structural Formula A. In the organic light-emitting compound according to the present disclosure, the linker L may be bonded to the substituted or unsubstituted pyrene ring moiety at a specific position (see the following Structural Formula C):

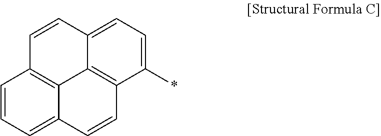 [SMILES: *c1ccc2ccc3cccc4ccc1c2c34]

*: bonding site to linker L

In an embodiment of the present disclosure, the substituent R₁ in Chemical Formula A may be a single bond linked to the linker L in Structural Formula A, and the substituent R₆ may be any one selected from a hydrogen atom, a deuterium atom, a substituted or unsubstituted aryl of 6 to 18 carbon atoms, and a substituted or unsubstituted heteroaryl of 2 to 30 carbon atoms. In the organic light-emitting compound represented by Chemical Formula A, the linker L may be bonded to the substituted or unsubstituted pyrene ring moiety at a specific position (see the following Structural Formula C-1) while a substituent selected from a hydrogen atom, a deuterium atom, a substituted or unsubstituted aryl of 6 to 18 carbon atoms, and a substituted or unsubstituted heteroaryl of 2 to 30 carbon atoms may be bonded to the pyrene ring moiety at a different specific position (see the following Structural Formula C-1):

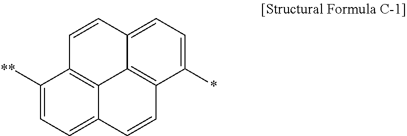

*: bonding site to linker L
**: bonding site to hydrogen, deuterium, aryl, or heteroaryl

In an embodiment of the present disclosure, substituents R₁ and R₆ in Chemical Formula A, which may each be a single bond linked to the linker L in Structural Formula A. That is, in the organic light-emitting compound represented by Chemical Formula A, each of the linkers L's in each of Structural Formula A may be bonded to the substituted or unsubstituted pyrene ring moiety at respective specific positions (see the following Structural Formula C-2) so that the organic light-emitting compound includes two substituents represented by Structural Formula A.

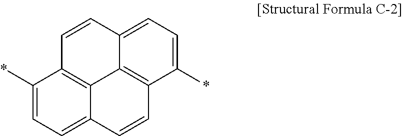 [SMILES: *c1ccc2ccc3c(*)ccc4ccc1c2c43]

*: bonding site to linker L

In an embodiment of the present disclosure, the compound represented by Chemical Formula A may bear at least one deuterium atom.

In Chemical Formula A, more specifically, at least one of R₁, R₃, R₆, and R₈ in Chemical Formula A may be a substituent bearing a deuterium atom, or at least one of R₁₁ to R₁₆ may be a substituent bearing a deuterium atom.

In addition, in an embodiment according to the present disclosure, R₁ to R₁₀, and R₁ to R₁₆, which may be same or different, are each independently a substituent selected from a hydrogen atom, a deuterium atom, a substituted or unsubstituted alkyl of 1 to 10 carbon atoms, a substituted or unsubstituted aryl of 6 to 18 carbon atoms, a substituted or unsubstituted heteroaryl of 2 to 18 carbon atoms, a substituted or unsubstituted alkylsilyl of 1 to 15 carbon atoms, a substituted or unsubstituted arylsilyl of 6 to 20 carbon atoms, a cyano, and a halogen.

In an embodiment according to the present disclosure, R₁₁ or R₁₂ in Structural Formula A may be a substituted or unsubstituted aryl of 6 to 18 carbon atoms.

Moreover, according to an embodiment of the present disclosure, one of the substituents R₁₃ to R₁₆ within Structural Formula A in Chemical Formula A may be a single bond linked to the linker L. In other words, Structural Formula A in Chemical Formula A may have the structure in which a connection is made between the linker L and the substituent R₁₃, R₁₄, R₁₅, or R₁₆.

According to an embodiment of the present disclosure, when one of the substituents R₁₃ to Ric is a single bond bonded to the linker L, the substituent R₁₁ or R₁₂ may be a substituted or unsubstituted aryl of 6 to 18 carbon atoms, or the substituents R₁₁ and R₁₂ may each be a substituted or unsubstituted aryl of 6 to 18 carbon atoms.

According to an embodiment according to the present disclosure, the substituent R₁₂ within Structural Formula A in Chemical Formula A may be a single bond linked to the linker L. In this regard, the substituent R₁₁ within Structural Formula A may be selected from a hydrogen atom, a deuterium atom, a substituted or unsubstituted aryl of 6 to 18 carbon atoms, and a substituted or unsubstituted heteroaryl of 3 to 18 carbon atoms, with preference for a substituted or unsubstituted aryl of 6 to 18 carbon atoms or substituted or unsubstituted heteroaryl of 3 to 18 carbon atoms.

In an embodiment according to the present disclosure, the linker L in Chemical Formula A may be a single bond or any one selected from the following Structural Formulas 1 to 5:

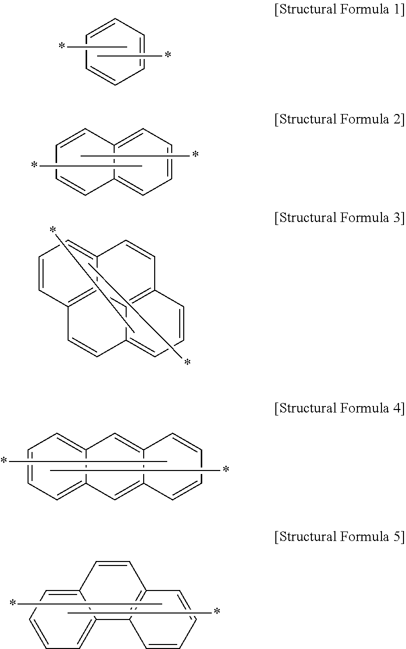

Each of the unsubstituted carbon atoms of the aromatic ring moiety in Structural Formulas 1 to 5 may be bound with a hydrogen atom or a deuterium atom.

In an embodiment according to the present disclosure, when the substituent R₁₂ within Structural Formula A in Chemical Formula A is a single bond linked to the linker L, the linker L may be a single bond or a substituted or unsubstituted aryl of 6 to 18 carbon atoms.

In an embodiment according to the present disclosure, the substituent R₁₁ within Structural Formula A in Chemical Formula A may be a single bond linked to the linker L while the linker L may be a substituted or unsubstituted aryl of 6 to 18 carbon atoms or a substituted or unsubstituted heteroaryl of 3 to 18 carbon atoms, with preference for a substituted or unsubstituted aryl of 6 to 18 carbon atoms.

In an embodiment according to the present disclosure, n in Chemical Formula A may be 1.

The organic light-emitting compound represented by Chemical Formula A according to the present disclosure may be any one selected from the following [Compound] 1 to [Compound 157], but with no limitations thereto:

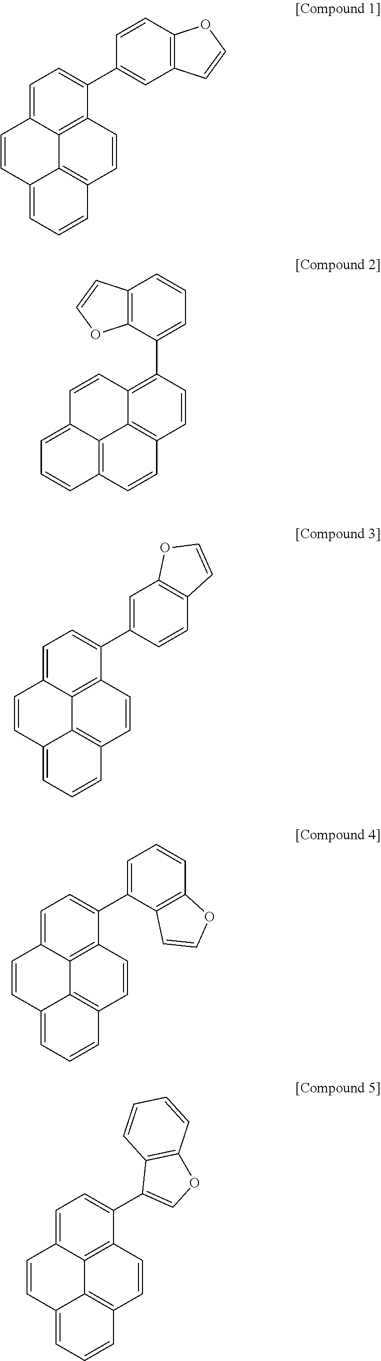 [SMILES: c1cc2ccc3ccc(-c4ccc5occc5c4)c4ccc(c1)c2c34 ; c1cc(-c2ccc3ccc4cccc5ccc2c3c45)c2occc2c1 ; c1cc2ccc3ccc(-c4ccc5ccoc5c4)c4ccc(c1)c2c34 ; c1cc2ccc3ccc(-c4cccc5occc45)c4ccc(c1)c2c34 ; c1cc2ccc3ccc(-c4coc5ccccc45)c4ccc(c1)c2c34]

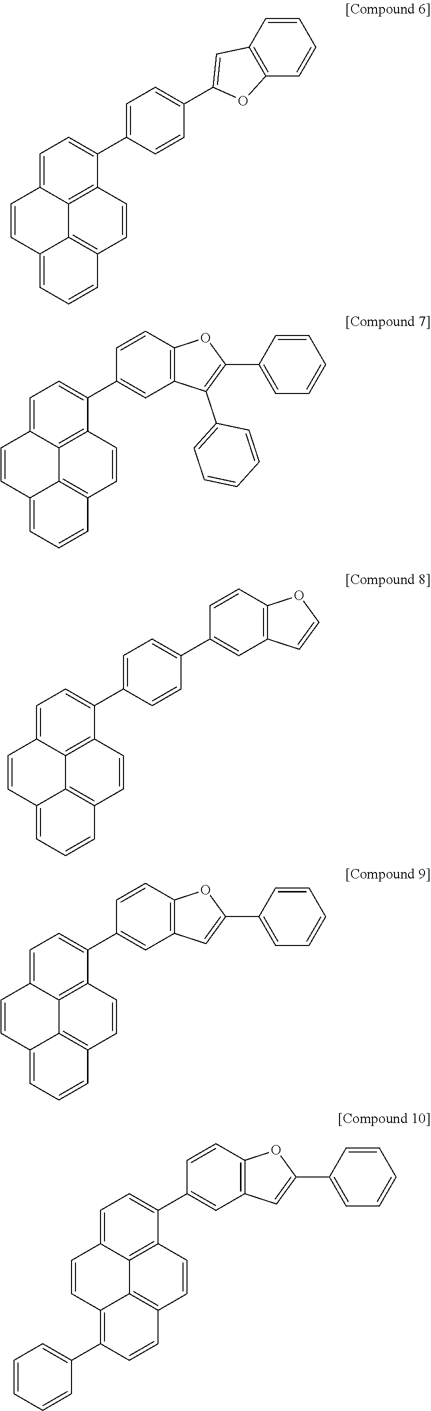 [SMILES: c1ccc2oc(-c3ccc(-c4ccc5ccc6cccc7ccc4c5c67)cc3)cc2c1 ; c1ccc(-c2oc3ccc(-c4ccc5ccc6cccc7ccc4c5c67)cc3c2-c2ccccc2)cc1 ; c1cc2ccc3ccc(-c4ccc(-c5ccc6occc6c5)cc4)c4ccc(c1)c2c34 ; c1ccc(-c2cc3cc(-c4ccc5ccc6cccc7ccc4c5c67)ccc3o2)cc1 ; c1ccc(-c2cc3cc(-c4ccc5ccc6c(-c7ccccc7)ccc7ccc4c5c76)ccc3o2)cc1]

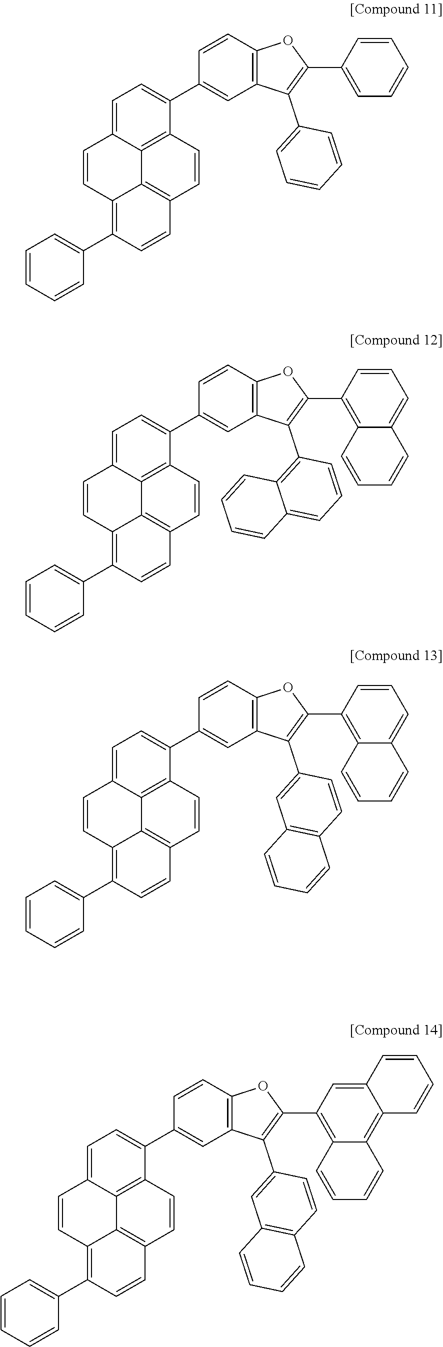 [SMILES: c1ccc(-c2oc3ccc(-c4ccc5ccc6c(-c7ccccc7)ccc7ccc4c5c76)cc3c2-c2ccccc2)cc1 ; c1ccc(-c2ccc3ccc4c(-c5ccc6oc(-c7cccc8ccccc78)c(-c7cccc8ccccc78)c6c5)ccc5ccc2c3c54)cc1 ; c1ccc(-c2ccc3ccc4c(-c5ccc6oc(-c7cccc8ccccc78)c(-c7ccc8ccccc8c7)c6c5)ccc5ccc2c3c54)cc1 ; c1ccc(-c2ccc3ccc4c(-c5ccc6oc(-c7cc8ccccc8c8ccccc78)c(-c7ccc8ccccc8c7)c6c5)ccc5ccc2c3c54)cc1]

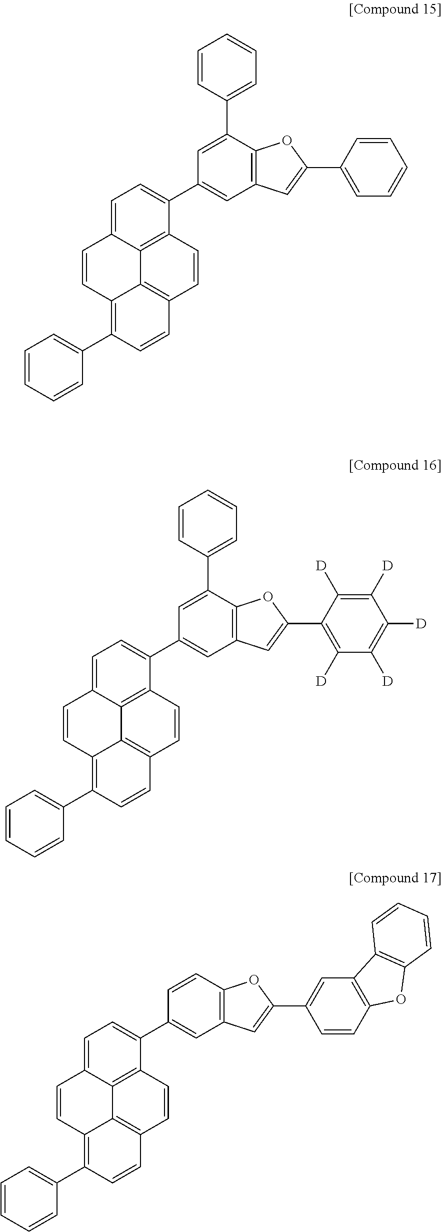 [SMILES: c1ccc(-c2cc3cc(-c4ccc5ccc6c(-c7ccccc7)ccc7ccc4c5c76)cc(-c4ccccc4)c3o2)cc1 ; [2H]c1c([2H])c([2H])c(-c2cc3cc(-c4ccc5ccc6c(-c7ccccc7)ccc7ccc4c5c76)cc(-c4ccccc4)c3o2)c([2H])c1[2H] ; c1ccc(-c2ccc3ccc4c(-c5ccc6oc(-c7ccc8oc9ccccc9c8c7)cc6c5)ccc5ccc2c3c54)cc1]

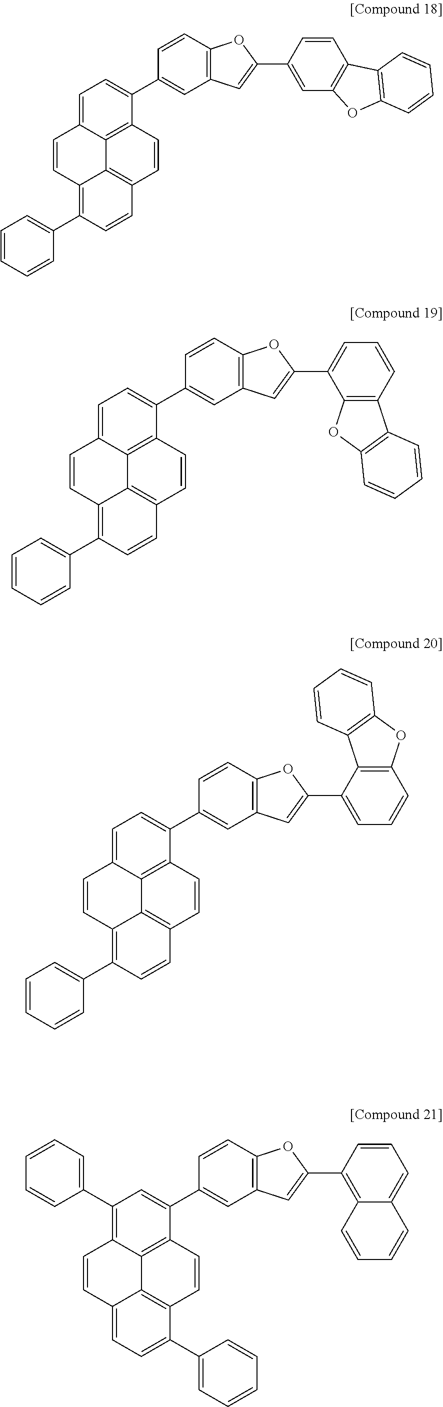 [SMILES: c1ccc(-c2ccc3ccc4c(-c5ccc6oc(-c7ccc8c(c7)oc7ccccc78)cc6c5)ccc5ccc2c3c54)cc1 ; c1ccc(-c2ccc3ccc4c(-c5ccc6oc(-c7cccc8c7oc7ccccc78)cc6c5)ccc5ccc2c3c54)cc1 ; c1ccc(-c2ccc3ccc4c(-c5ccc6oc(-c7cccc8oc9ccccc9c78)cc6c5)ccc5ccc2c3c54)cc1 ; c1ccc(-c2ccc3ccc4c(-c5ccccc5)cc(-c5ccc6oc(-c7cccc8ccccc78)cc6c5)c5ccc2c3c45)cc1]

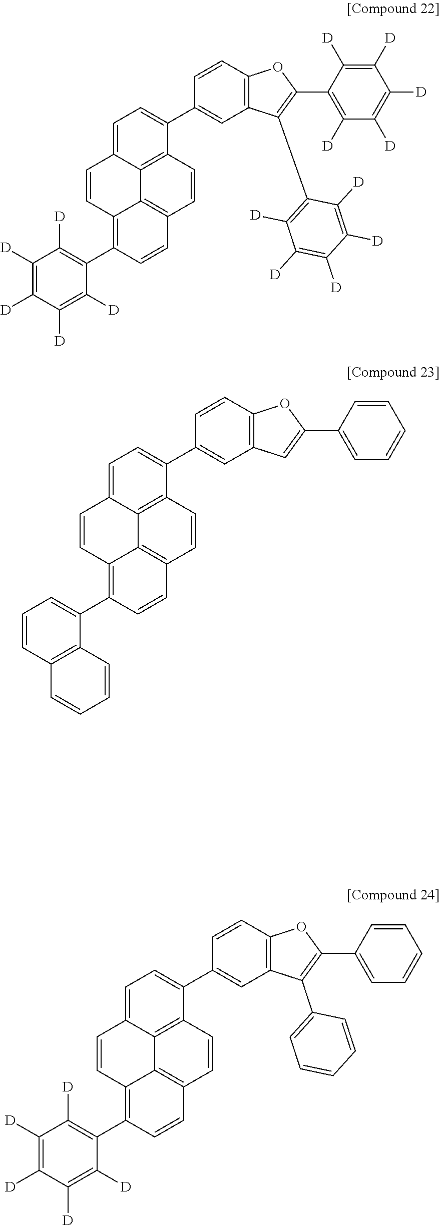 [SMILES: [2H]c1c([2H])c([2H])c(-c2oc3ccc(-c4ccc5ccc6c(-c7c([2H])c([2H])c([2H])c([2H])c7[2H])ccc7ccc4c5c76)cc3c2-c2c([2H])c([2H])c([2H])c([2H])c2[2H])c([2H])c1[2H] ; c1ccc(-c2cc3cc(-c4ccc5ccc6c(-c7cccc8ccccc78)ccc7ccc4c5c76)ccc3o2)cc1 ; [2H]c1c([2H])c([2H])c(-c2ccc3ccc4c(-c5ccc6oc(-c7ccccc7)c(-c7ccccc7)c6c5)ccc5ccc2c3c54)c([2H])c1[2H]]

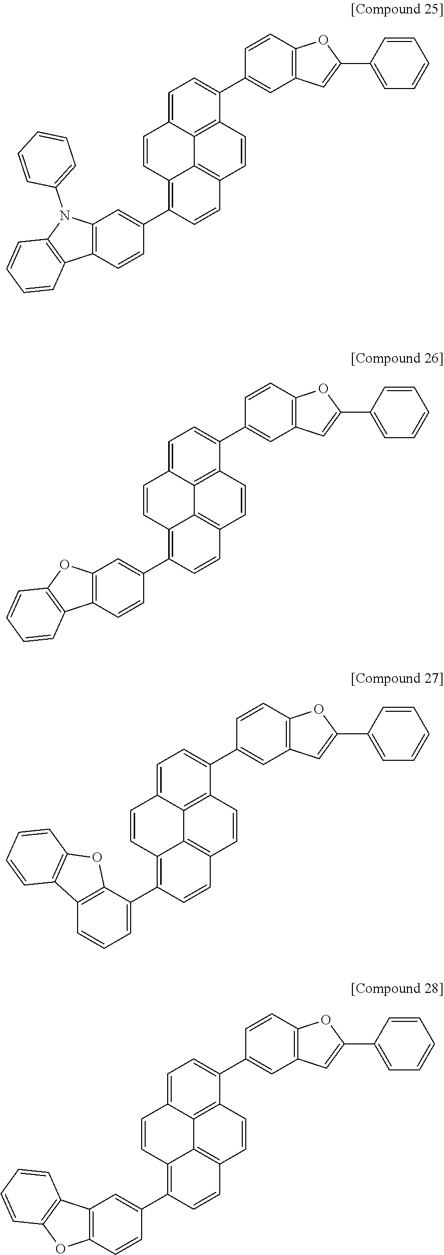 [SMILES: c1ccc(-c2cc3cc(-c4ccc5ccc6c(-c7ccc8c9ccccc9n(-c9ccccc9)c8c7)ccc7ccc4c5c76)ccc3o2)cc1 ; c1ccc(-c2cc3cc(-c4ccc5ccc6c(-c7ccc8c(c7)oc7ccccc78)ccc7ccc4c5c76)ccc3o2)cc1 ; c1ccc(-c2cc3cc(-c4ccc5ccc6c(-c7cccc8c7oc7ccccc78)ccc7ccc4c5c76)ccc3o2)cc1 ; c1ccc(-c2cc3cc(-c4ccc5ccc6c(-c7ccc8oc9ccccc9c8c7)ccc7ccc4c5c76)ccc3o2)cc1]

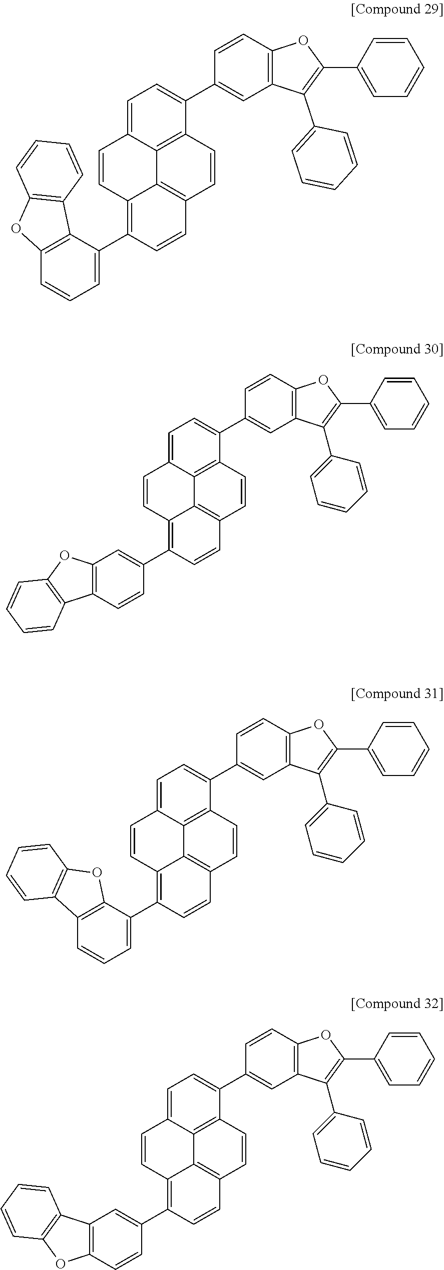 [SMILES: c1ccc(-c2oc3ccc(-c4ccc5ccc6c(-c7cccc8oc9ccccc9c78)ccc7ccc4c5c76)cc3c2-c2ccccc2)cc1 ; c1ccc(-c2oc3ccc(-c4ccc5ccc6c(-c7ccc8c(c7)oc7ccccc78)ccc7ccc4c5c76)cc3c2-c2ccccc2)cc1 ; c1ccc(-c2oc3ccc(-c4ccc5ccc6c(-c7cccc8c7oc7ccccc78)ccc7ccc4c5c76)cc3c2-c2ccccc2)cc1 ; c1ccc(-c2oc3ccc(-c4ccc5ccc6c(-c7ccc8oc9ccccc9c8c7)ccc7ccc4c5c76)cc3c2-c2ccccc2)cc1]

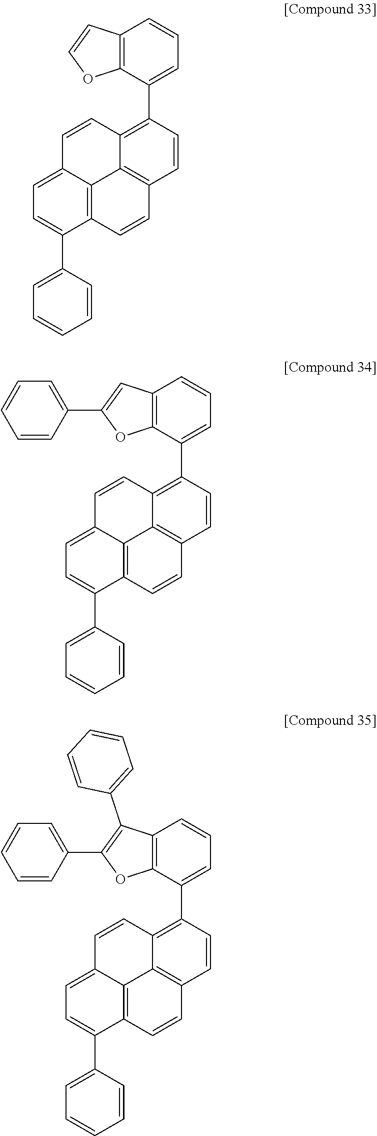

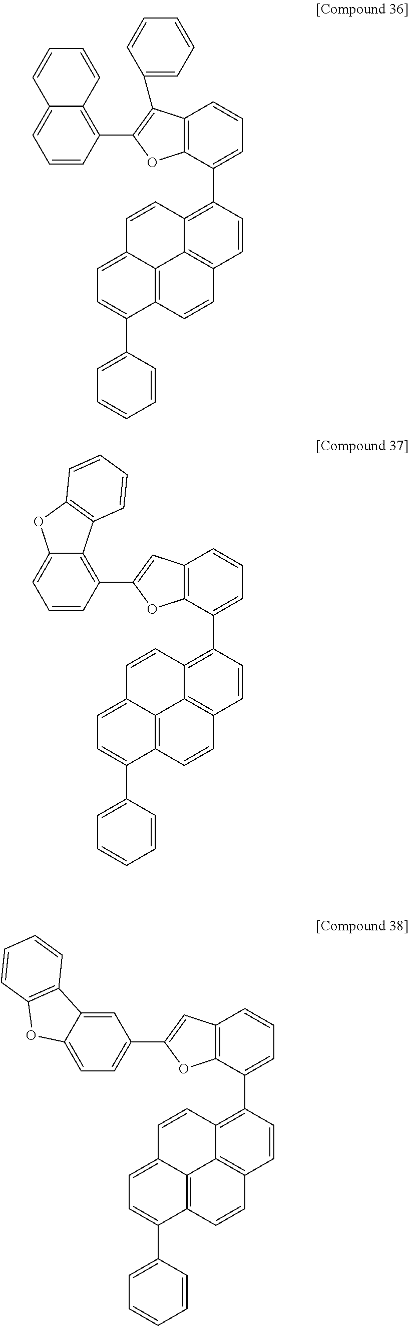

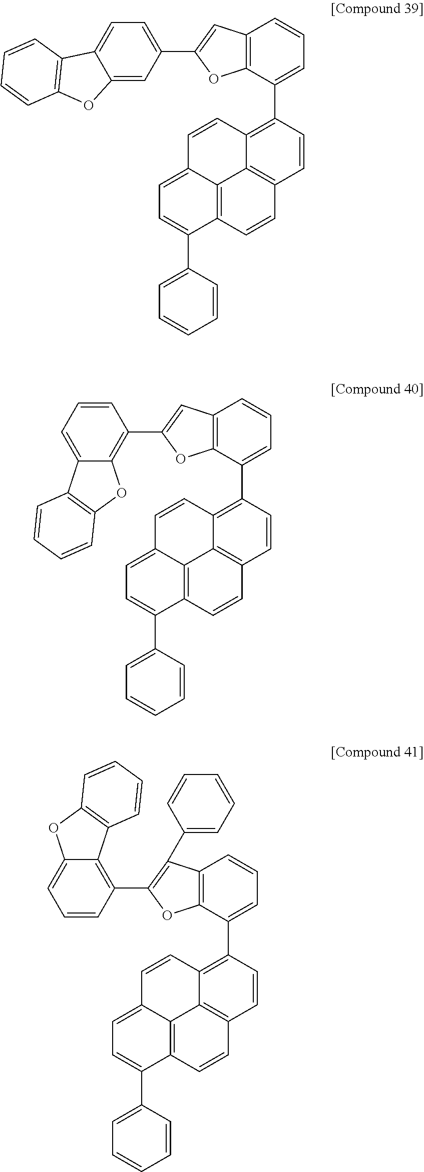 [SMILES: c1ccc(-c2ccc3ccc4c(-c5cccc6cc(-c7ccc8c(c7)oc7ccccc78)oc56)ccc5ccc2c3c54)cc1 ; c1ccc(-c2ccc3ccc4c(-c5cccc6cc(-c7cccc8c7oc7ccccc78)oc56)ccc5ccc2c3c54)cc1 ; c1ccc(-c2c(-c3cccc4oc5ccccc5c34)oc3c(-c4ccc5ccc6c(-c7ccccc7)ccc7ccc4c5c76)cccc23)cc1]

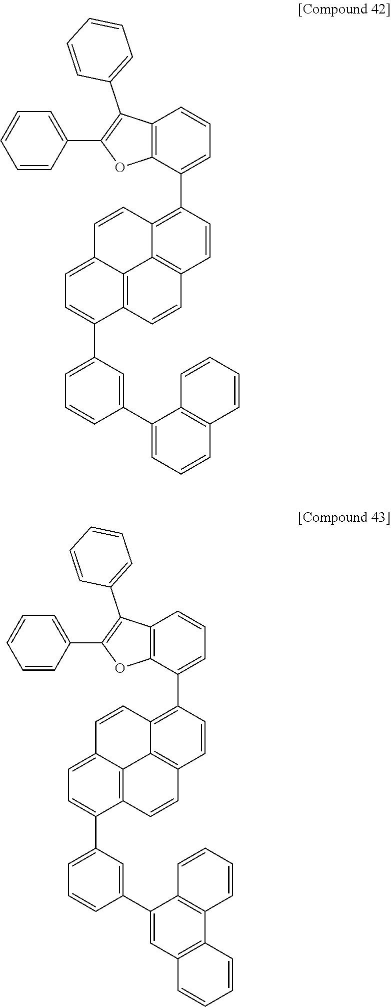 [SMILES: c1ccc(-c2oc3c(-c4ccc5ccc6c(-c7cccc(-c8cccc9ccccc89)c7)ccc7ccc4c5c76)cccc3c2-c2ccccc2)cc1 ; c1ccc(-c2oc3c(-c4ccc5ccc6c(-c7cccc(-c8cc9ccccc9c9ccccc89)c7)ccc7ccc4c5c76)cccc3c2-c2ccccc2)cc1]

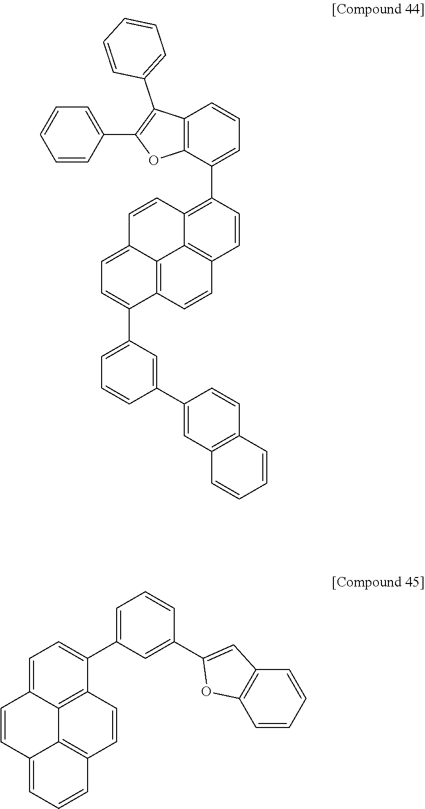 [SMILES: c1ccc(-c2oc3c(-c4ccc5ccc6c(-c7cccc(-c8ccc9ccccc9c8)c7)ccc7ccc4c5c76)cccc3c2-c2ccccc2)cc1 ; c1cc(-c2cc3ccccc3o2)cc(-c2ccc3ccc4cccc5ccc2c3c45)c1]

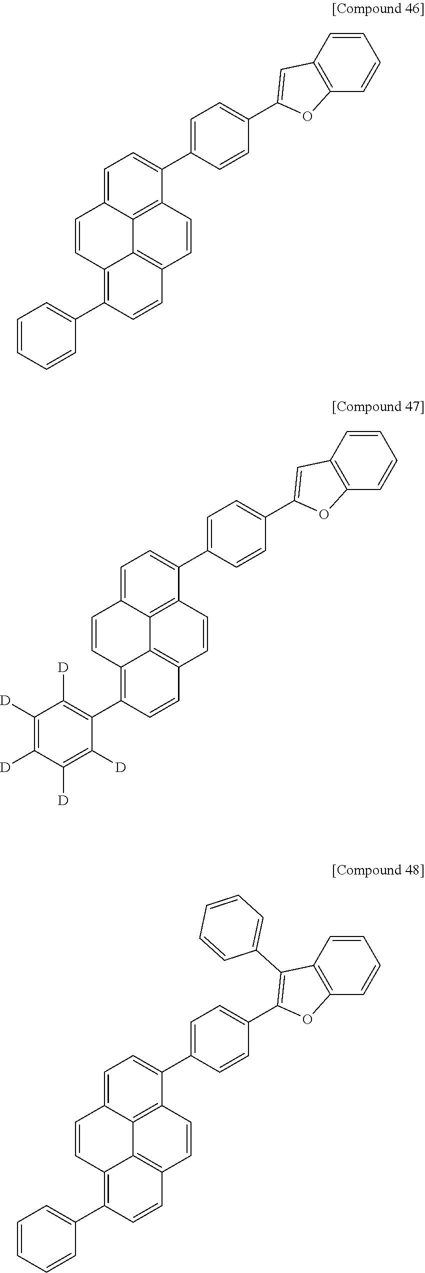 [SMILES: c1ccc(-c2ccc3ccc4c(-c5ccc(-c6cc7ccccc7o6)cc5)ccc5ccc2c3c54)cc1 ; [2H]c1c([2H])c([2H])c(-c2ccc3ccc4c(-c5ccc(-c6cc7ccccc7o6)cc5)ccc5ccc2c3c54)c([2H])c1[2H] ; c1ccc(-c2c(-c3ccc(-c4ccc5ccc6c(-c7ccccc7)ccc7ccc4c5c76)cc3)oc3ccccc23)cc1]

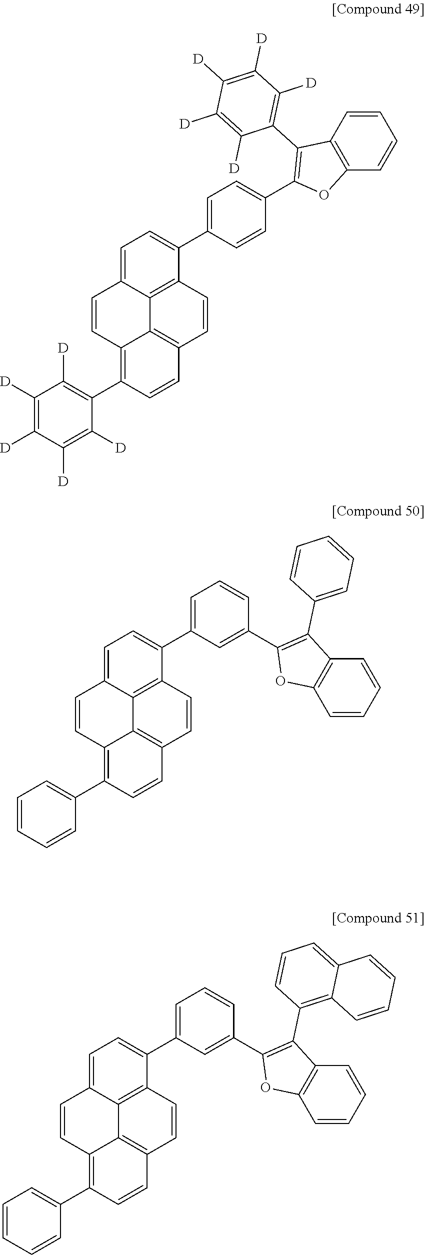 [SMILES: [2H]c1c([2H])c([2H])c(-c2c(-c3ccc(-c4ccc5ccc6c(-c7c([2H])c([2H])c([2H])c([2H])c7[2H])ccc7ccc4c5c76)cc3)oc3ccccc23)c([2H])c1[2H] ; c1ccc(-c2c(-c3cccc(-c4ccc5ccc6c(-c7ccccc7)ccc7ccc4c5c76)c3)oc3ccccc23)cc1 ; c1ccc(-c2ccc3ccc4c(-c5cccc(-c6oc7ccccc7c6-c6cccc7ccccc67)c5)ccc5ccc2c3c54)cc1]

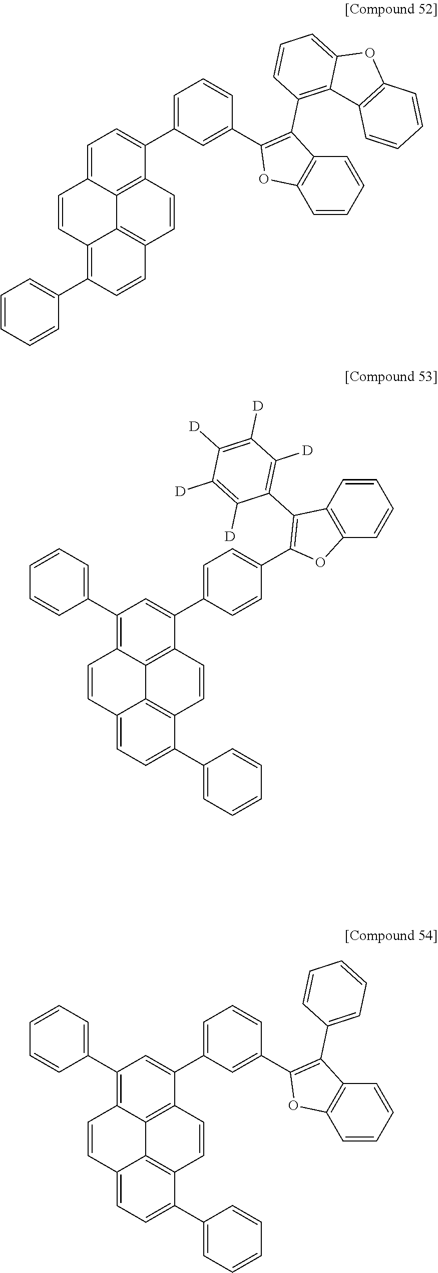 [SMILES: c1ccc(-c2ccc3ccc4c(-c5cccc(-c6oc7ccccc7c6-c6cccc7oc8ccccc8c67)c5)ccc5ccc2c3c54)cc1 ; [2H]c1c([2H])c([2H])c(-c2c(-c3ccc(-c4cc(-c5ccccc5)c5ccc6ccc(-c7ccccc7)c7ccc4c5c67)cc3)oc3ccccc23)c([2H])c1[2H] ; c1ccc(-c2c(-c3cccc(-c4cc(-c5ccccc5)c5ccc6ccc(-c7ccccc7)c7ccc4c5c67)c3)oc3ccccc23)cc1]

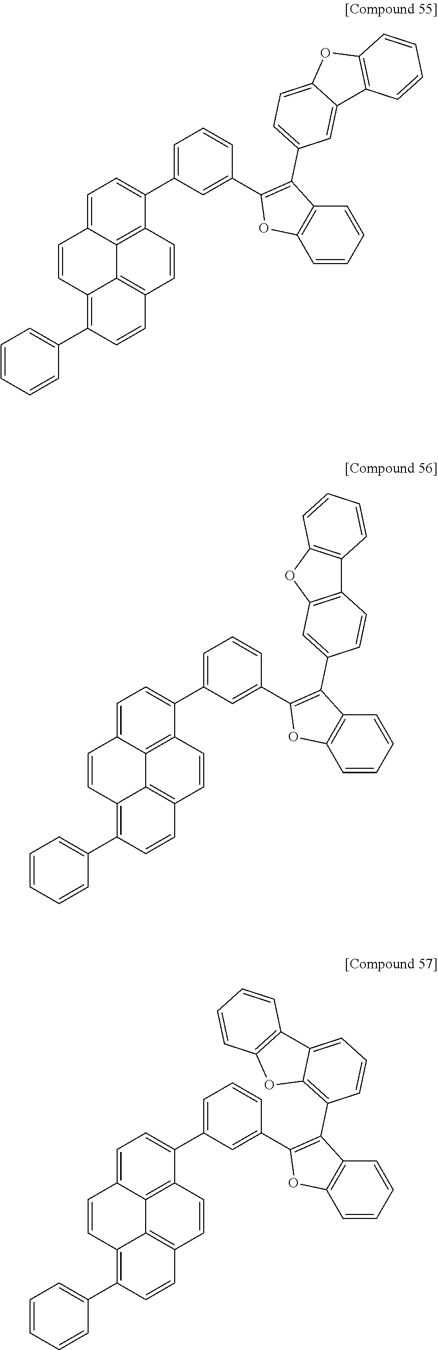 [SMILES: c1ccc(-c2ccc3ccc4c(-c5cccc(-c6oc7ccccc7c6-c6ccc7oc8ccccc8c7c6)c5)ccc5ccc2c3c54)cc1 ; c1ccc(-c2ccc3ccc4c(-c5cccc(-c6oc7ccccc7c6-c6ccc7c(c6)oc6ccccc67)c5)ccc5ccc2c3c54)cc1 ; c1ccc(-c2ccc3ccc4c(-c5cccc(-c6oc7ccccc7c6-c6cccc7c6oc6ccccc67)c5)ccc5ccc2c3c54)cc1]

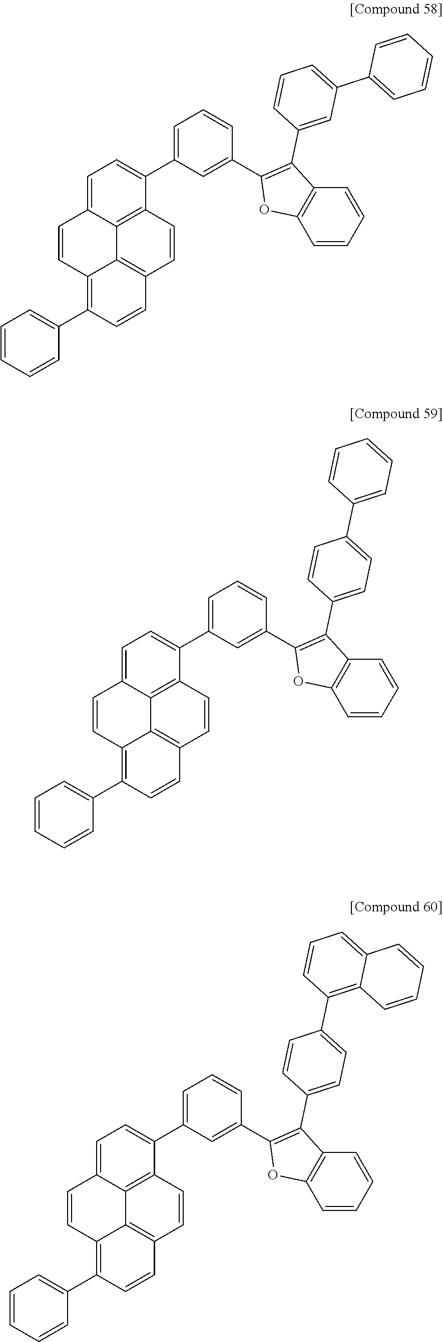 [SMILES: c1ccc(-c2cccc(-c3c(-c4cccc(-c5ccc6ccc7c(-c8ccccc8)ccc8ccc5c6c87)c4)oc4ccccc34)c2)cc1 ; c1ccc(-c2ccc(-c3c(-c4cccc(-c5ccc6ccc7c(-c8ccccc8)ccc8ccc5c6c87)c4)oc4ccccc34)cc2)cc1 ; c1ccc(-c2ccc3ccc4c(-c5cccc(-c6oc7ccccc7c6-c6ccc(-c7cccc8ccccc78)cc6)c5)ccc5ccc2c3c54)cc1]

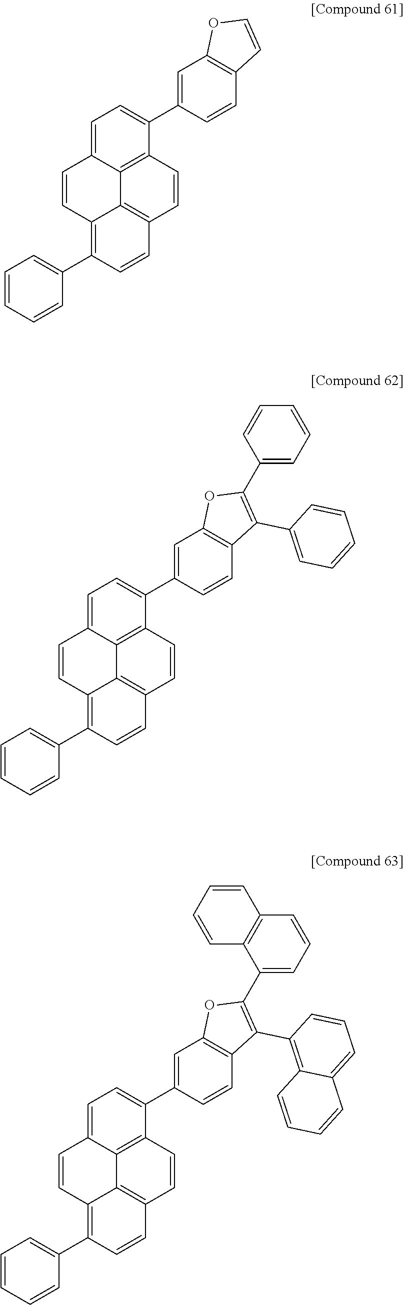 [SMILES: c1ccc(-c2ccc3ccc4c(-c5ccc6ccoc6c5)ccc5ccc2c3c54)cc1 ; c1ccc(-c2oc3cc(-c4ccc5ccc6c(-c7ccccc7)ccc7ccc4c5c76)ccc3c2-c2ccccc2)cc1 ; c1ccc(-c2ccc3ccc4c(-c5ccc6c(-c7cccc8ccccc78)c(-c7cccc8ccccc78)oc6c5)ccc5ccc2c3c54)cc1]

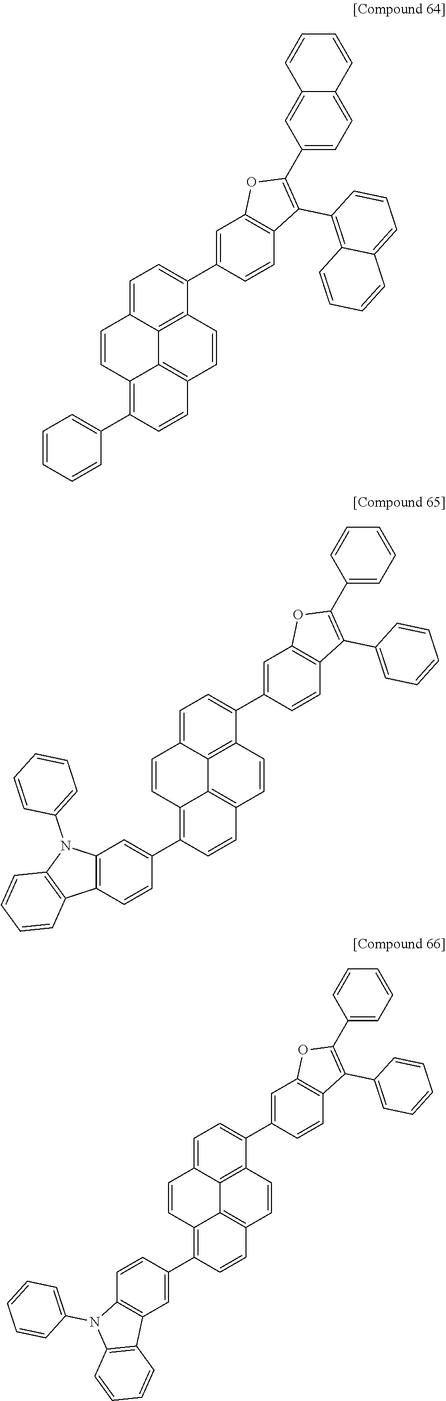 [SMILES: c1ccc(-c2ccc3ccc4c(-c5ccc6c(-c7cccc8ccccc78)c(-c7ccc8ccccc8c7)oc6c5)ccc5ccc2c3c54)cc1 ; c1ccc(-c2oc3cc(-c4ccc5ccc6c(-c7ccc8c9ccccc9n(-c9ccccc9)c8c7)ccc7ccc4c5c76)ccc3c2-c2ccccc2)cc1 ; c1ccc(-c2oc3cc(-c4ccc5ccc6c(-c7ccc8c(c7)c7ccccc7n8-c7ccccc7)ccc7ccc4c5c76)ccc3c2-c2ccccc2)cc1]

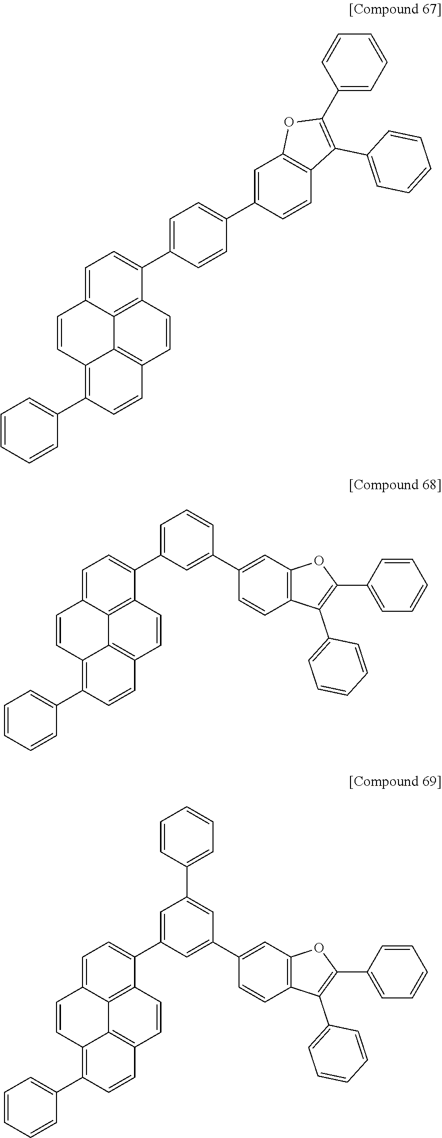 [SMILES: c1ccc(-c2oc3cc(-c4ccc(-c5ccc6ccc7c(-c8ccccc8)ccc8ccc5c6c87)cc4)ccc3c2-c2ccccc2)cc1 ; c1ccc(-c2oc3cc(-c4cccc(-c5ccc6ccc7c(-c8ccccc8)ccc8ccc5c6c87)c4)ccc3c2-c2ccccc2)cc1 ; c1ccc(-c2cc(-c3ccc4c(-c5ccccc5)c(-c5ccccc5)oc4c3)cc(-c3ccc4ccc5c(-c6ccccc6)ccc6ccc3c4c65)c2)cc1]

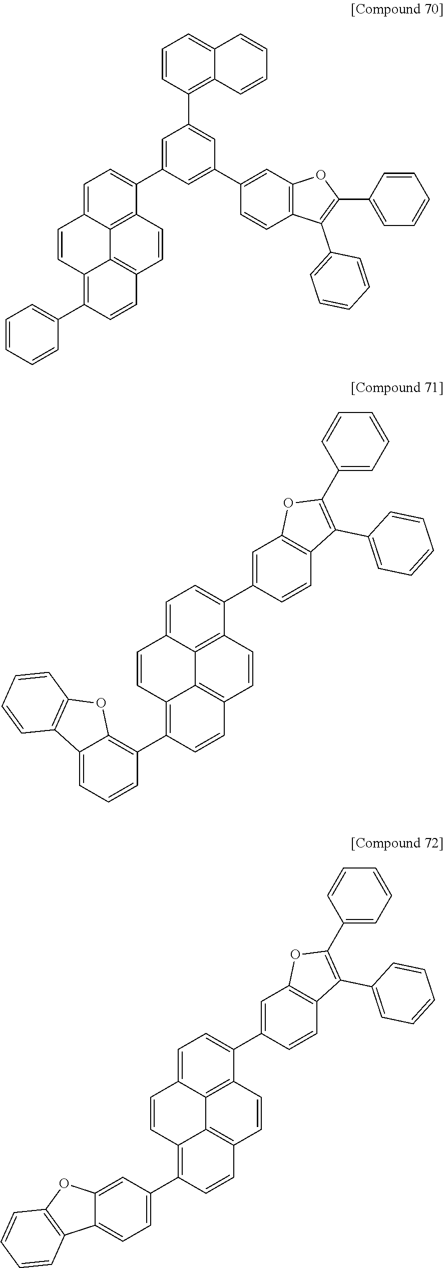 [SMILES: c1ccc(-c2oc3cc(-c4cc(-c5cccc6ccccc56)cc(-c5ccc6ccc7c(-c8ccccc8)ccc8ccc5c6c87)c4)ccc3c2-c2ccccc2)cc1 ; c1ccc(-c2oc3cc(-c4ccc5ccc6c(-c7cccc8c7oc7ccccc78)ccc7ccc4c5c76)ccc3c2-c2ccccc2)cc1 ; c1ccc(-c2oc3cc(-c4ccc5ccc6c(-c7ccc8c(c7)oc7ccccc78)ccc7ccc4c5c76)ccc3c2-c2ccccc2)cc1]

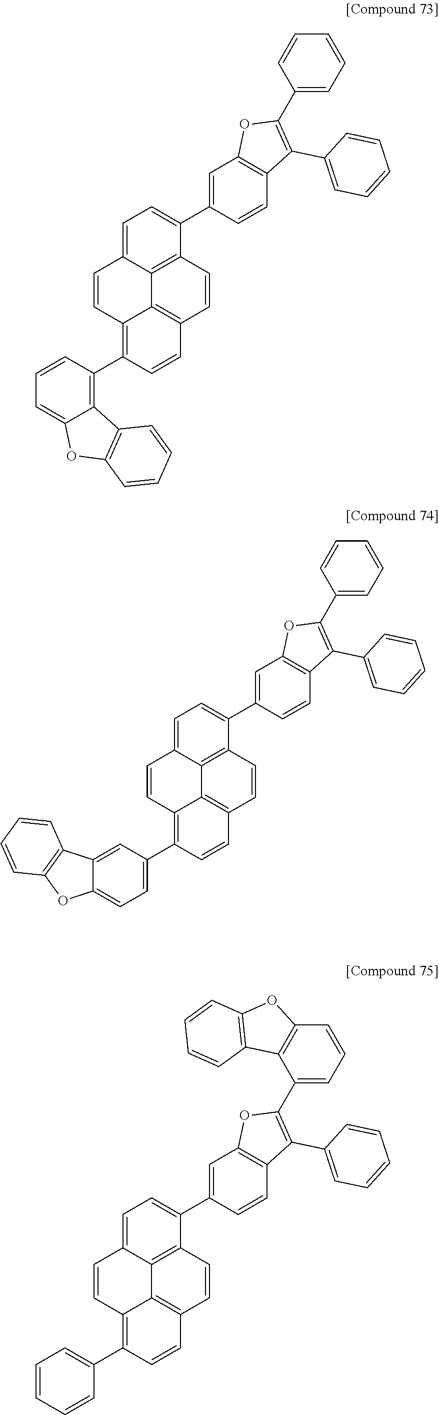 [SMILES: c1ccc(-c2oc3cc(-c4ccc5ccc6c(-c7cccc8oc9ccccc9c78)ccc7ccc4c5c76)ccc3c2-c2ccccc2)cc1 ; c1ccc(-c2oc3cc(-c4ccc5ccc6c(-c7ccc8oc9ccccc9c8c7)ccc7ccc4c5c76)ccc3c2-c2ccccc2)cc1 ; c1ccc(-c2c(-c3cccc4oc5ccccc5c34)oc3cc(-c4ccc5ccc6c(-c7ccccc7)ccc7ccc4c5c76)ccc23)cc1]

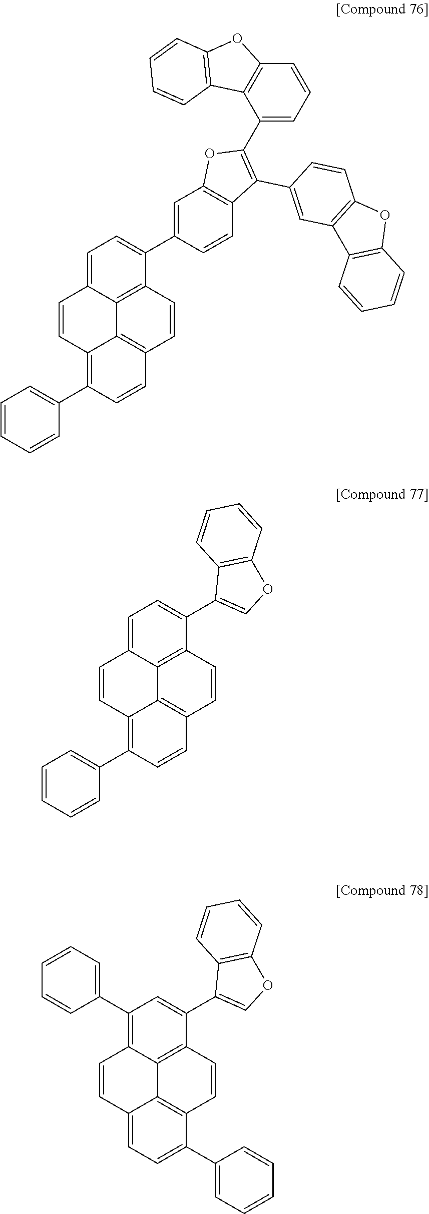 [SMILES: c1ccc(-c2ccc3ccc4c(-c5ccc6c(-c7ccc8oc9ccccc9c8c7)c(-c7cccc8oc9ccccc9c78)oc6c5)ccc5ccc2c3c54)cc1 ; c1ccc(-c2ccc3ccc4c(-c5coc6ccccc56)ccc5ccc2c3c54)cc1 ; c1ccc(-c2ccc3ccc4c(-c5ccccc5)cc(-c5coc6ccccc56)c5ccc2c3c45)cc1]

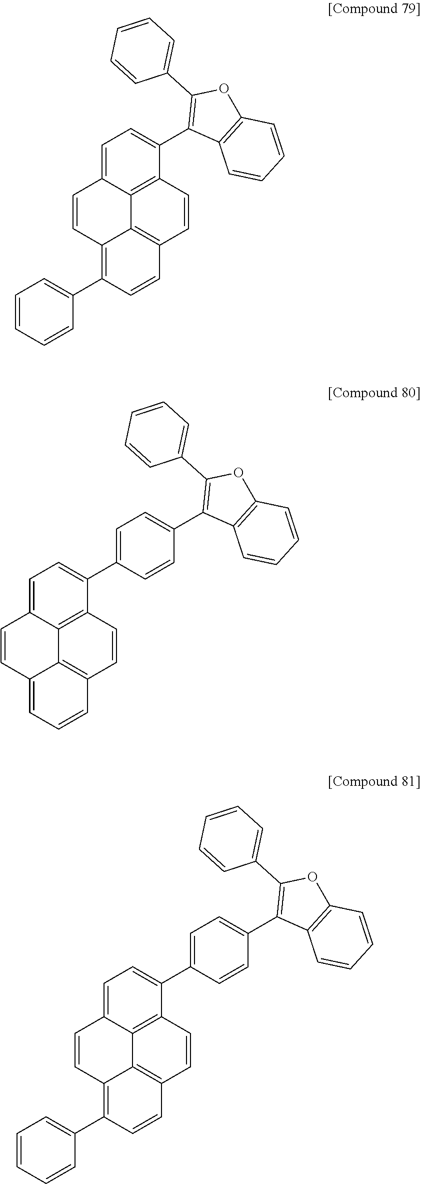 [SMILES: c1ccc(-c2oc3ccccc3c2-c2ccc3ccc4c(-c5ccccc5)ccc5ccc2c3c54)cc1 ; c1ccc(-c2oc3ccccc3c2-c2ccc(-c3ccc4ccc5cccc6ccc3c4c56)cc2)cc1 ; c1ccc(-c2oc3ccccc3c2-c2ccc(-c3ccc4ccc5c(-c6ccccc6)ccc6ccc3c4c65)cc2)cc1]

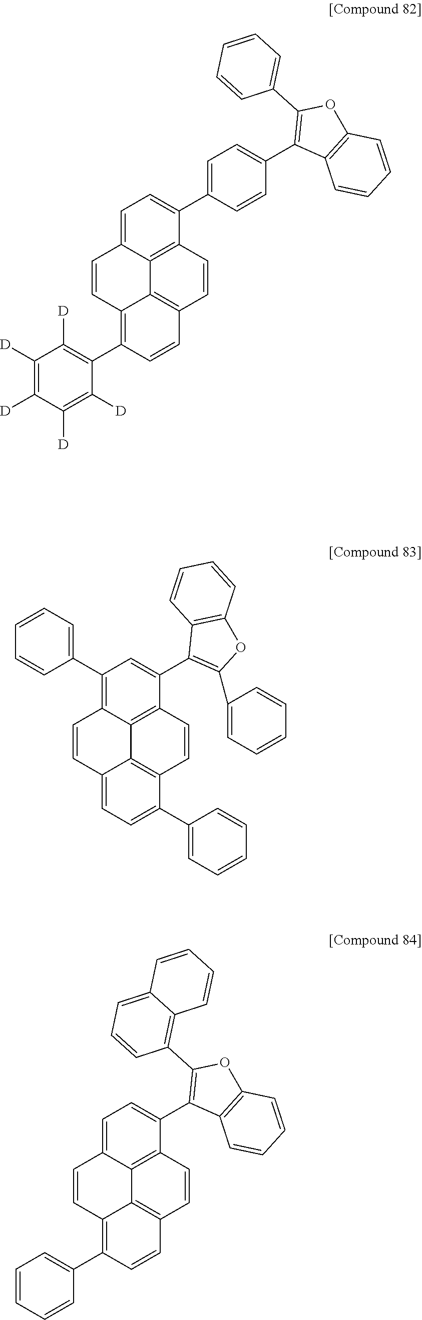 [SMILES: [2H]c1c([2H])c([2H])c(-c2ccc3ccc4c(-c5ccc(-c6c(-c7ccccc7)oc7ccccc67)cc5)ccc5ccc2c3c54)c([2H])c1[2H] ; c1ccc(-c2oc3ccccc3c2-c2cc(-c3ccccc3)c3ccc4ccc(-c5ccccc5)c5ccc2c3c45)cc1 ; c1ccc(-c2ccc3ccc4c(-c5c(-c6cccc7ccccc67)oc6ccccc56)ccc5ccc2c3c54)cc1]

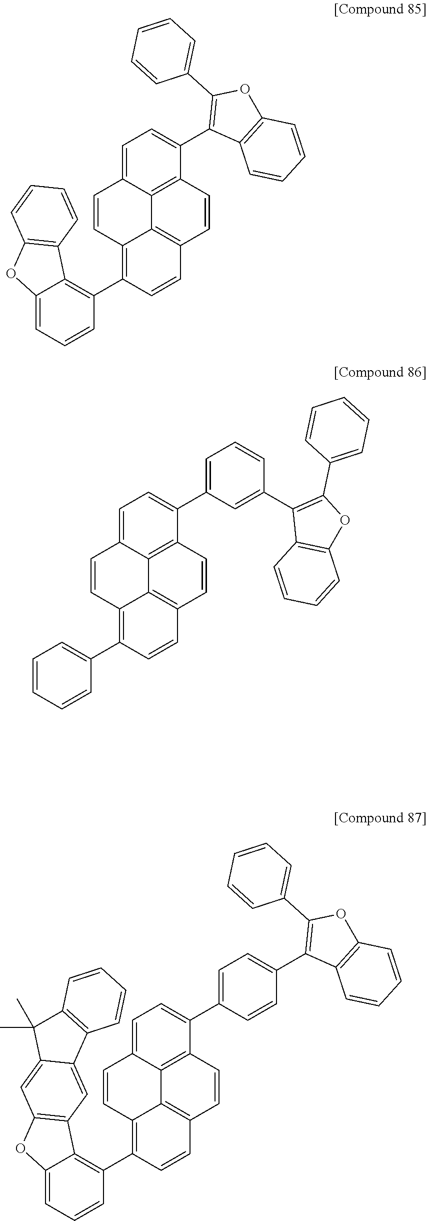 [SMILES: c1ccc(-c2oc3ccccc3c2-c2ccc3ccc4c(-c5cccc6oc7ccccc7c56)ccc5ccc2c3c54)cc1 ; c1ccc(-c2oc3ccccc3c2-c2cccc(-c3ccc4ccc5c(-c6ccccc6)ccc6ccc3c4c65)c2)cc1 ; CC1(C)c2ccccc2-c2cc3c(cc21)oc1cccc(-c2ccc4ccc5c(-c6ccc(-c7c(-c8ccccc8)oc8ccccc78)cc6)ccc6ccc2c4c65)c13]

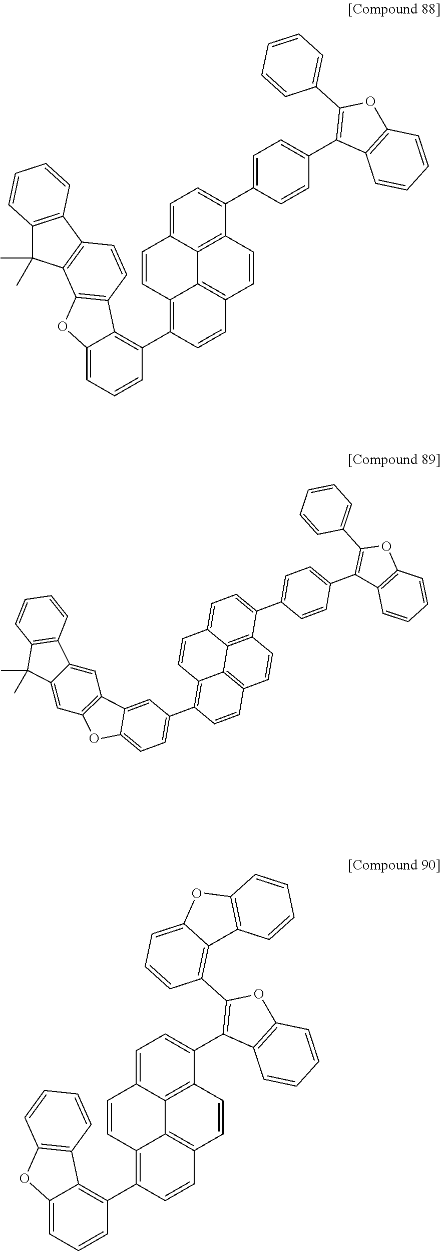 [SMILES: CC1(C)c2ccccc2-c2ccc3c(oc4cccc(-c5ccc6ccc7c(-c8ccc(-c9c(-c%10ccccc%10)oc%10ccccc9%10)cc8)ccc8ccc5c6c87)c43)c21 ; CC1(C)c2ccccc2-c2cc3c(cc21)oc1ccc(-c2ccc4ccc5c(-c6ccc(-c7c(-c8ccccc8)oc8ccccc78)cc6)ccc6ccc2c4c65)cc13 ; c1ccc2c(-c3ccc4ccc5c(-c6cccc7oc8ccccc8c67)ccc6ccc3c4c65)c(-c3cccc4oc5ccccc5c34)oc2c1]

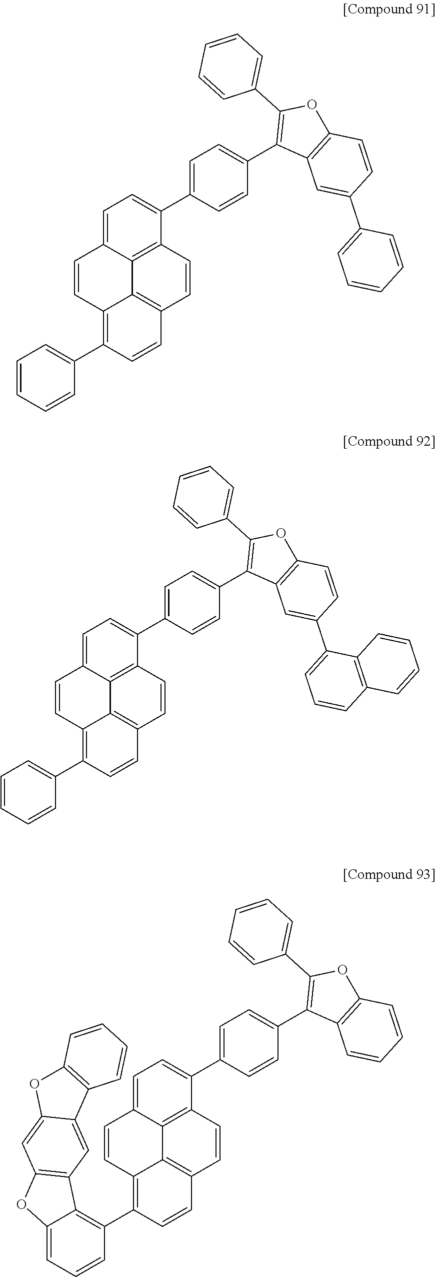 [SMILES: c1ccc(-c2ccc3oc(-c4ccccc4)c(-c4ccc(-c5ccc6ccc7c(-c8ccccc8)ccc8ccc5c6c87)cc4)c3c2)cc1 ; c1ccc(-c2oc3ccc(-c4cccc5ccccc45)cc3c2-c2ccc(-c3ccc4ccc5c(-c6ccccc6)ccc6ccc3c4c65)cc2)cc1 ; c1ccc(-c2oc3ccccc3c2-c2ccc(-c3ccc4ccc5c(-c6cccc7oc8cc9oc%10ccccc%10c9cc8c67)ccc6ccc3c4c65)cc2)cc1]

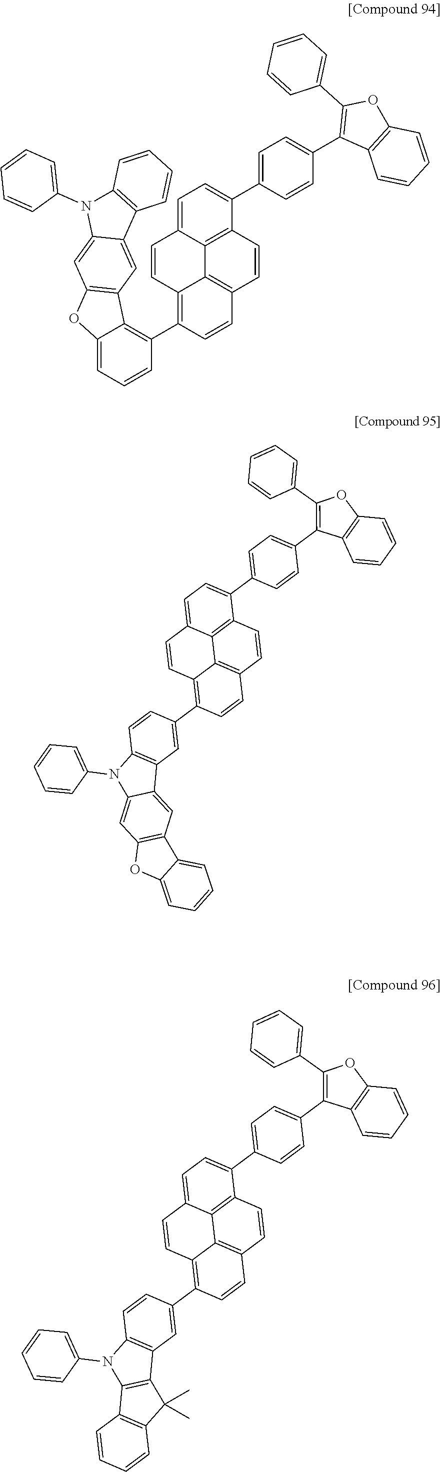 [SMILES: c1ccc(-c2oc3ccccc3c2-c2ccc(-c3ccc4ccc5c(-c6cccc7oc8cc9c(cc8c67)c6ccccc6n9-c6ccccc6)ccc6ccc3c4c65)cc2)cc1 ; c1ccc(-c2oc3ccccc3c2-c2ccc(-c3ccc4ccc5c(-c6ccc7c(c6)c6cc8c(cc6n7-c6ccccc6)oc6ccccc68)ccc6ccc3c4c65)cc2)cc1 ; CC1(C)c2ccccc2-c2c1c1cc(-c3ccc4ccc5c(-c6ccc(-c7c(-c8ccccc8)oc8ccccc78)cc6)ccc6ccc3c4c65)ccc1n2-c1ccccc1]

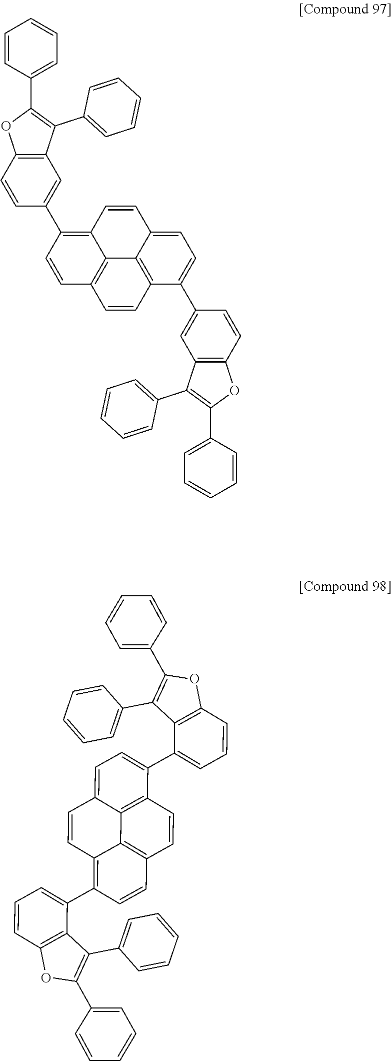 [SMILES: c1ccc(-c2oc3ccc(-c4ccc5ccc6c(-c7ccc8oc(-c9ccccc9)c(-c9ccccc9)c8c7)ccc7ccc4c5c76)cc3c2-c2ccccc2)cc1 ; c1ccc(-c2oc3cccc(-c4ccc5ccc6c(-c7cccc8oc(-c9ccccc9)c(-c9ccccc9)c78)ccc7ccc4c5c76)c3c2-c2ccccc2)cc1]

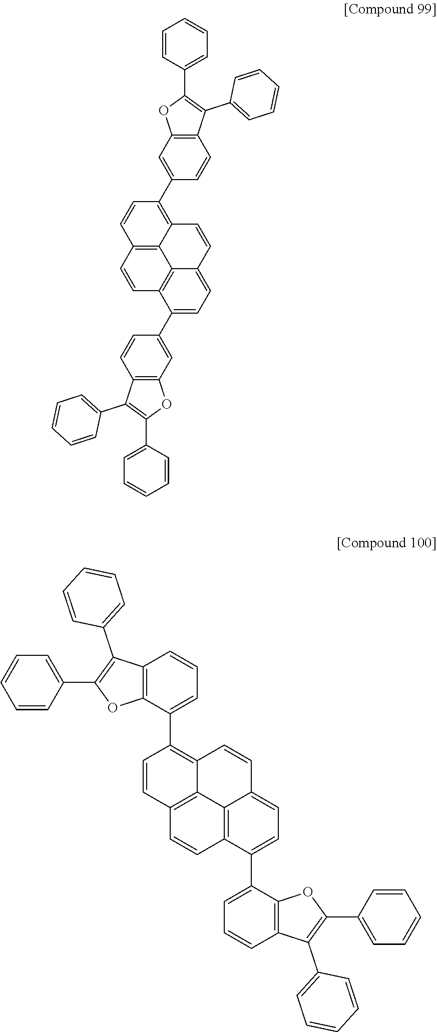 [SMILES: c1ccc(-c2oc3cc(-c4ccc5ccc6c(-c7ccc8c(-c9ccccc9)c(-c9ccccc9)oc8c7)ccc7ccc4c5c76)ccc3c2-c2ccccc2)cc1 ; c1ccc(-c2oc3c(-c4ccc5ccc6c(-c7cccc8c(-c9ccccc9)c(-c9ccccc9)oc78)ccc7ccc4c5c76)cccc3c2-c2ccccc2)cc1]

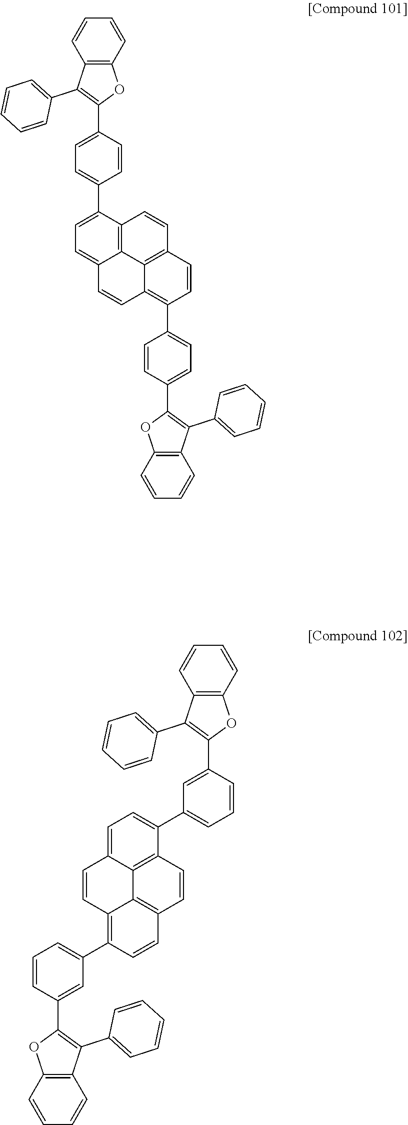 [SMILES: c1ccc(-c2c(-c3ccc(-c4ccc5ccc6c(-c7ccc(-c8oc9ccccc9c8-c8ccccc8)cc7)ccc7ccc4c5c76)cc3)oc3ccccc23)cc1 ; c1ccc(-c2c(-c3cccc(-c4ccc5ccc6c(-c7cccc(-c8oc9ccccc9c8-c8ccccc8)c7)ccc7ccc4c5c76)c3)oc3ccccc23)cc1]

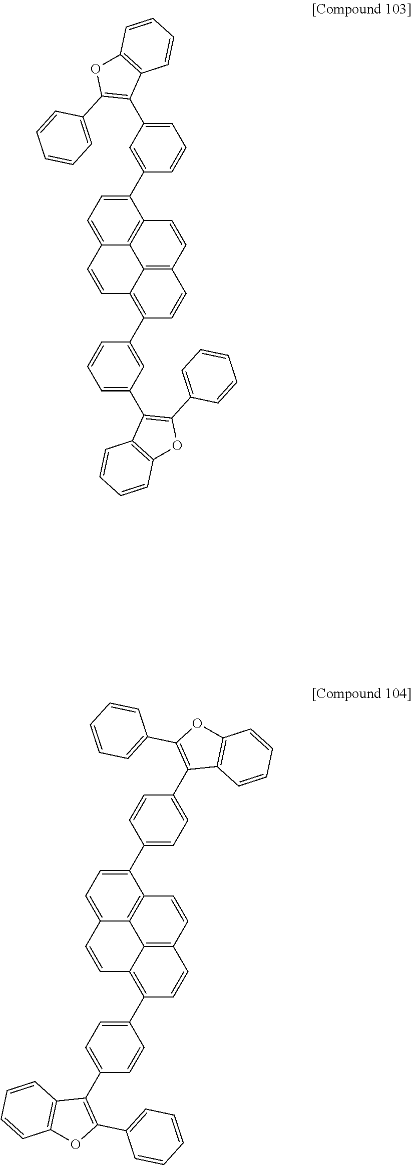 [SMILES: c1ccc(-c2oc3ccccc3c2-c2cccc(-c3ccc4ccc5c(-c6cccc(-c7c(-c8ccccc8)oc8ccccc78)c6)ccc6ccc3c4c65)c2)cc1 ; c1ccc(-c2oc3ccccc3c2-c2ccc(-c3ccc4ccc5c(-c6ccc(-c7c(-c8ccccc8)oc8ccccc78)cc6)ccc6ccc3c4c65)cc2)cc1]

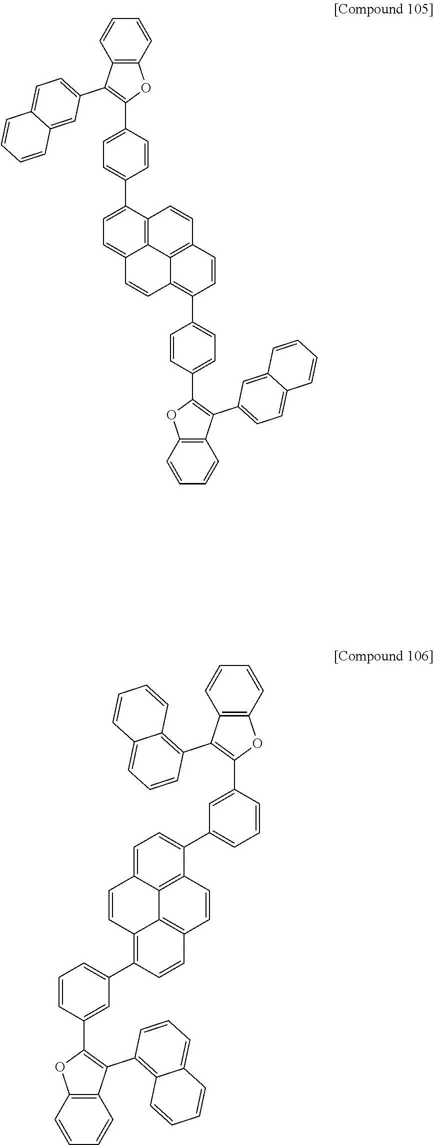 [SMILES: c1ccc2cc(-c3c(-c4ccc(-c5ccc6ccc7c(-c8ccc(-c9oc%10ccccc%10c9-c9ccc%10ccccc%10c9)cc8)ccc8ccc5c6c87)cc4)oc4ccccc34)ccc2c1 ; c1cc(-c2oc3ccccc3c2-c2cccc3ccccc23)cc(-c2ccc3ccc4c(-c5cccc(-c6oc7ccccc7c6-c6cccc7ccccc67)c5)ccc5ccc2c3c54)c1]

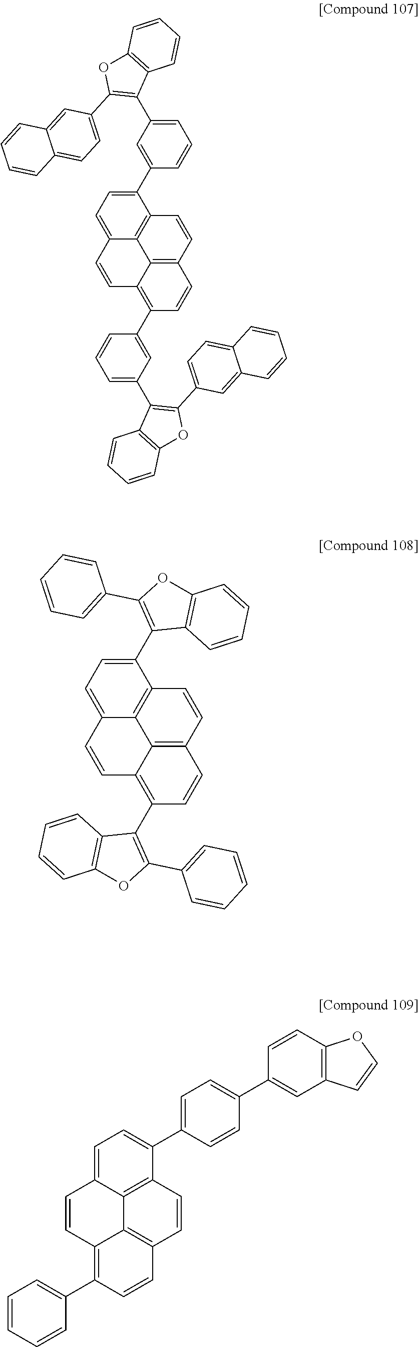 [SMILES: c1cc(-c2c(-c3ccc4ccccc4c3)oc3ccccc23)cc(-c2ccc3ccc4c(-c5cccc(-c6c(-c7ccc8ccccc8c7)oc7ccccc67)c5)ccc5ccc2c3c54)c1 ; c1ccc(-c2oc3ccccc3c2-c2ccc3ccc4c(-c5c(-c6ccccc6)oc6ccccc56)ccc5ccc2c3c54)cc1 ; c1ccc(-c2ccc3ccc4c(-c5ccc(-c6ccc7occc7c6)cc5)ccc5ccc2c3c54)cc1]

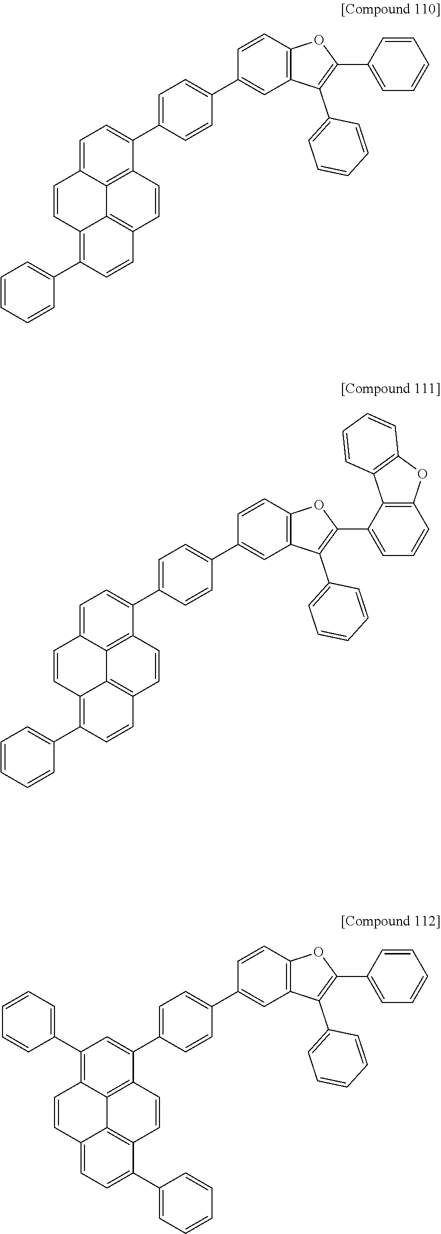 [SMILES: c1ccc(-c2oc3ccc(-c4ccc(-c5ccc6ccc7c(-c8ccccc8)ccc8ccc5c6c87)cc4)cc3c2-c2ccccc2)cc1 ; c1ccc(-c2c(-c3cccc4oc5ccccc5c34)oc3ccc(-c4ccc(-c5ccc6ccc7c(-c8ccccc8)ccc8ccc5c6c87)cc4)cc23)cc1 ; c1ccc(-c2oc3ccc(-c4ccc(-c5cc(-c6ccccc6)c6ccc7ccc(-c8ccccc8)c8ccc5c6c78)cc4)cc3c2-c2ccccc2)cc1]

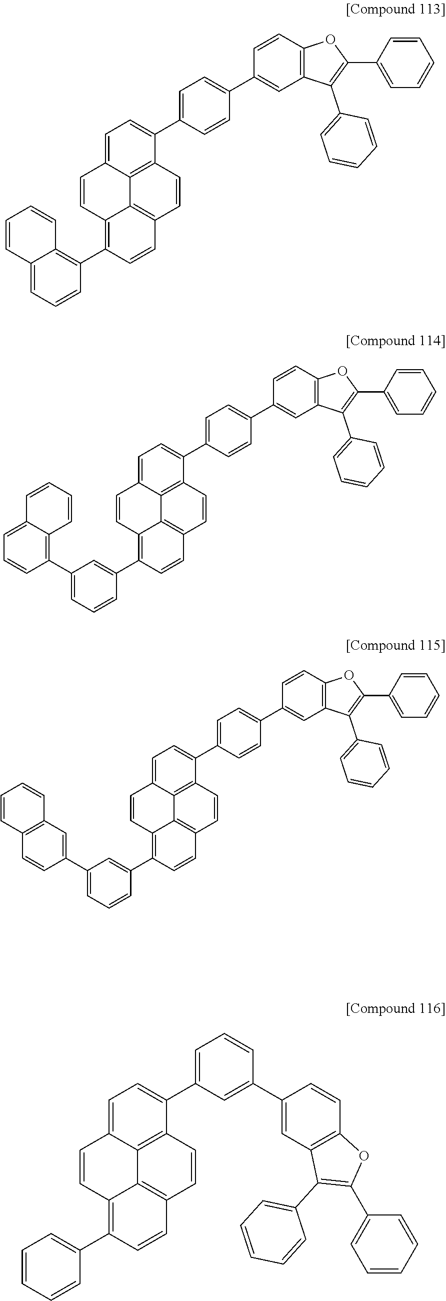 [SMILES: c1ccc(-c2oc3ccc(-c4ccc(-c5ccc6ccc7c(-c8cccc9ccccc89)ccc8ccc5c6c87)cc4)cc3c2-c2ccccc2)cc1 ; c1ccc(-c2oc3ccc(-c4ccc(-c5ccc6ccc7c(-c8cccc(-c9cccc%10ccccc9%10)c8)ccc8ccc5c6c87)cc4)cc3c2-c2ccccc2)cc1 ; c1ccc(-c2oc3ccc(-c4ccc(-c5ccc6ccc7c(-c8cccc(-c9ccc%10ccccc%10c9)c8)ccc8ccc5c6c87)cc4)cc3c2-c2ccccc2)cc1 ; c1ccc(-c2oc3ccc(-c4cccc(-c5ccc6ccc7c(-c8ccccc8)ccc8ccc5c6c87)c4)cc3c2-c2ccccc2)cc1]

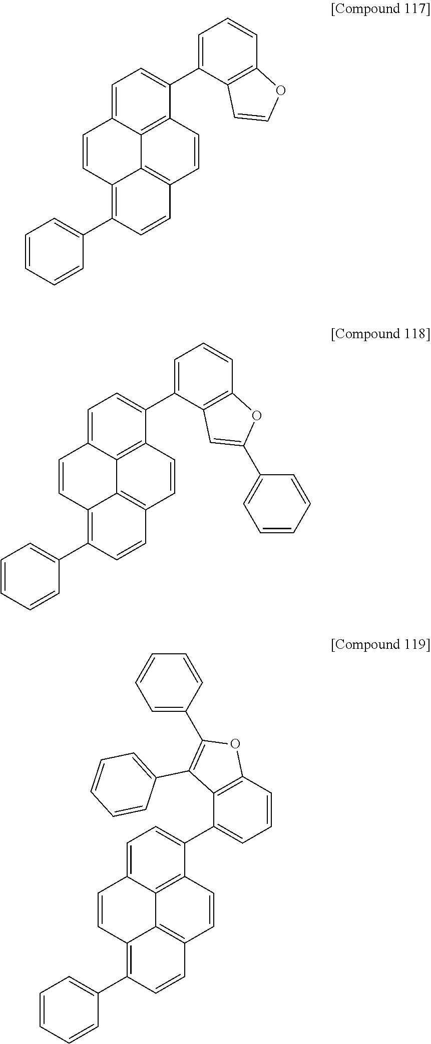 [SMILES: c1ccc(-c2ccc3ccc4c(-c5cccc6occc56)ccc5ccc2c3c54)cc1 ; c1ccc(-c2cc3c(-c4ccc5ccc6c(-c7ccccc7)ccc7ccc4c5c76)cccc3o2)cc1 ; c1ccc(-c2oc3cccc(-c4ccc5ccc6c(-c7ccccc7)ccc7ccc4c5c76)c3c2-c2ccccc2)cc1]

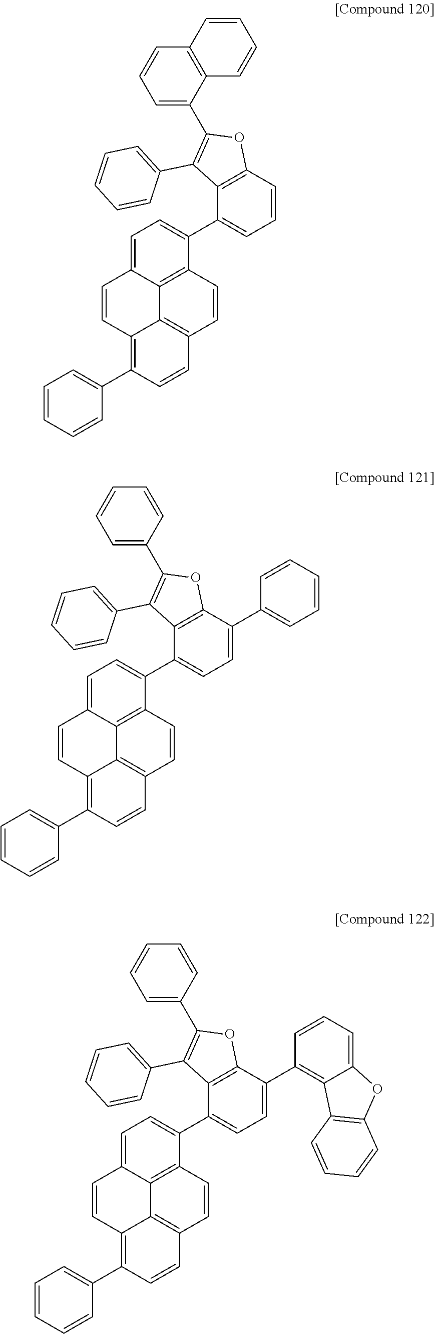 [SMILES: c1ccc(-c2ccc3ccc4c(-c5cccc6oc(-c7cccc8ccccc78)c(-c7ccccc7)c56)ccc5ccc2c3c54)cc1 ; c1ccc(-c2oc3c(-c4ccccc4)ccc(-c4ccc5ccc6c(-c7ccccc7)ccc7ccc4c5c76)c3c2-c2ccccc2)cc1 ; c1ccc(-c2oc3c(-c4cccc5oc6ccccc6c45)ccc(-c4ccc5ccc6c(-c7ccccc7)ccc7ccc4c5c76)c3c2-c2ccccc2)cc1]

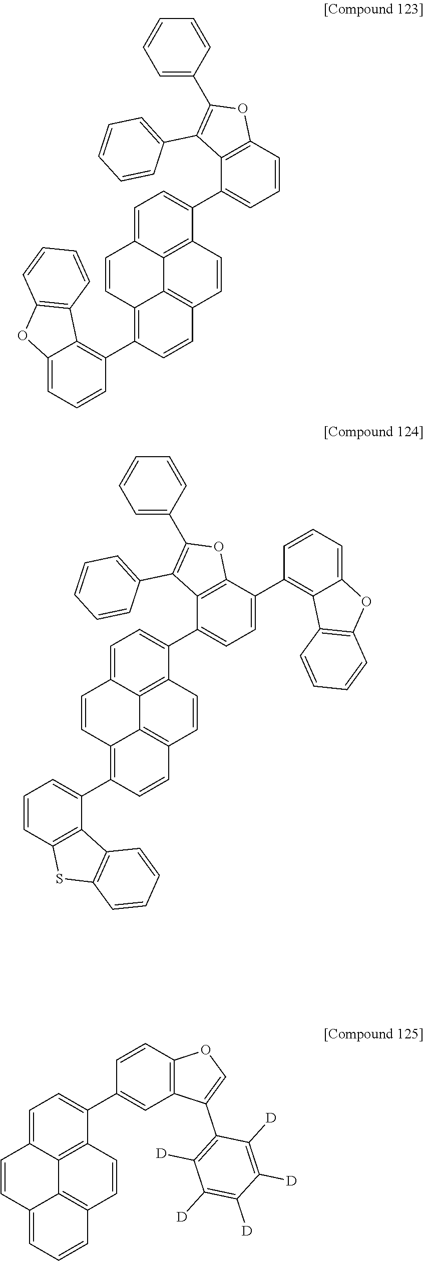 [SMILES: c1ccc(-c2oc3cccc(-c4ccc5ccc6c(-c7cccc8oc9ccccc9c78)ccc7ccc4c5c76)c3c2-c2ccccc2)cc1 ; c1ccc(-c2oc3c(-c4cccc5oc6ccccc6c45)ccc(-c4ccc5ccc6c(-c7cccc8sc9ccccc9c78)ccc7ccc4c5c76)c3c2-c2ccccc2)cc1 ; [2H]c1c([2H])c([2H])c(-c2coc3ccc(-c4ccc5ccc6cccc7ccc4c5c67)cc23)c([2H])c1[2H]]

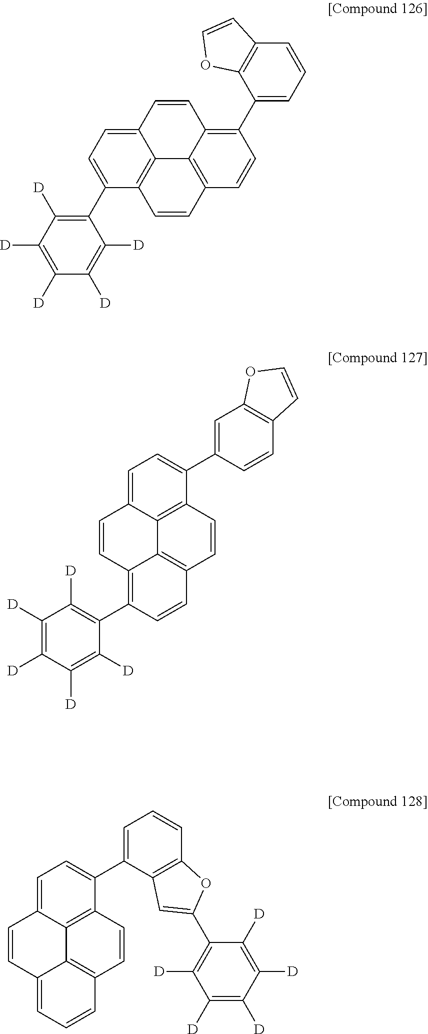 [SMILES: [2H]c1c([2H])c([2H])c(-c2ccc3ccc4c(-c5cccc6ccoc56)ccc5ccc2c3c54)c([2H])c1[2H] ; [2H]c1c([2H])c([2H])c(-c2ccc3ccc4c(-c5ccc6ccoc6c5)ccc5ccc2c3c54)c([2H])c1[2H] ; [2H]c1c([2H])c([2H])c(-c2cc3c(-c4ccc5ccc6cccc7ccc4c5c67)cccc3o2)c([2H])c1[2H]]

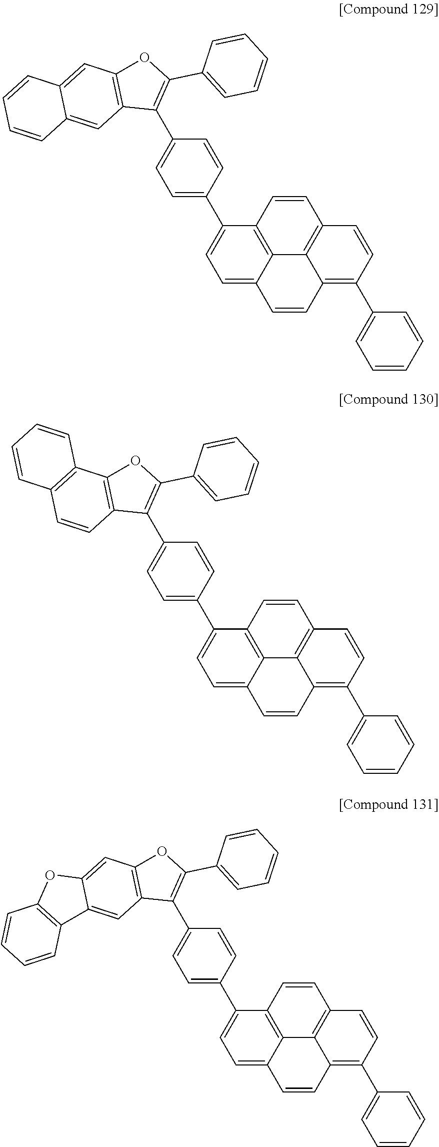 [SMILES: c1ccc(-c2oc3cc4ccccc4cc3c2-c2ccc(-c3ccc4ccc5c(-c6ccccc6)ccc6ccc3c4c65)cc2)cc1 ; c1ccc(-c2oc3c(ccc4ccccc43)c2-c2ccc(-c3ccc4ccc5c(-c6ccccc6)ccc6ccc3c4c65)cc2)cc1 ; c1ccc(-c2oc3cc4oc5ccccc5c4cc3c2-c2ccc(-c3ccc4ccc5c(-c6ccccc6)ccc6ccc3c4c65)cc2)cc1]

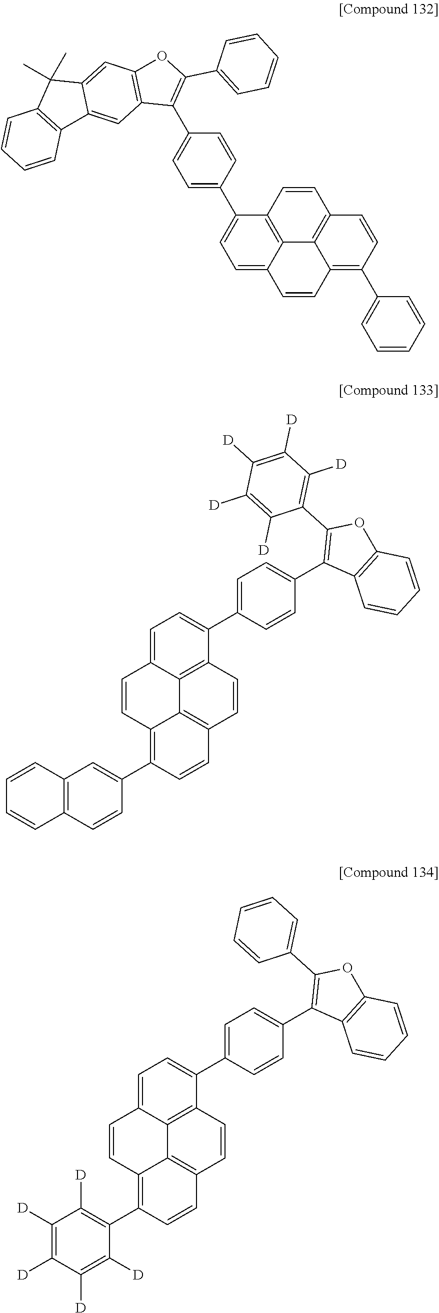 [SMILES: CC1(C)c2ccccc2-c2cc3c(-c4ccc(-c5ccc6ccc7c(-c8ccccc8)ccc8ccc5c6c87)cc4)c(-c4ccccc4)oc3cc21 ; [2H]c1c([2H])c([2H])c(-c2oc3ccccc3c2-c2ccc(-c3ccc4ccc5c(-c6ccc7ccccc7c6)ccc6ccc3c4c65)cc2)c([2H])c1[2H] ; [2H]c1c([2H])c([2H])c(-c2ccc3ccc4c(-c5ccc(-c6c(-c7ccccc7)oc7ccccc67)cc5)ccc5ccc2c3c54)c([2H])c1[2H]]

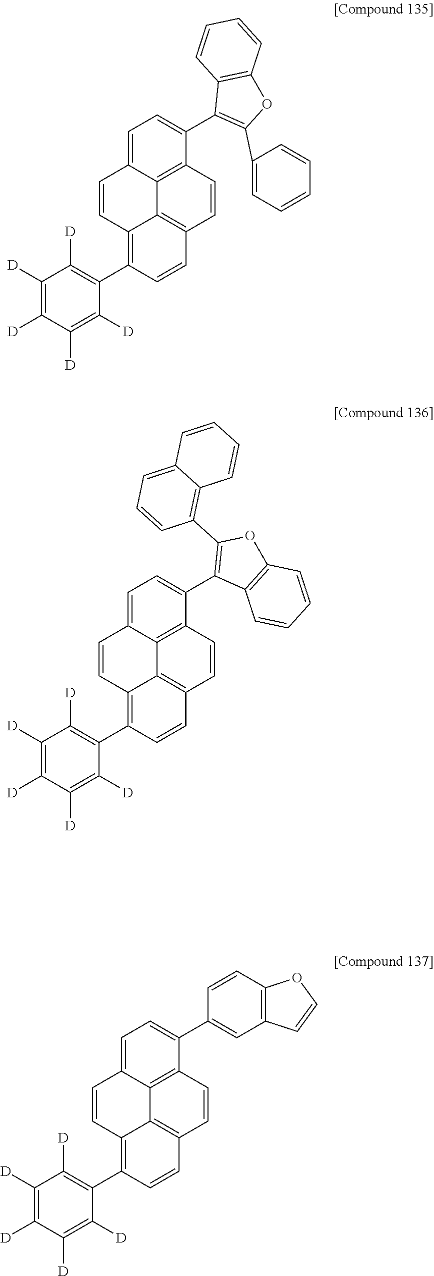 [SMILES: [2H]c1c([2H])c([2H])c(-c2ccc3ccc4c(-c5c(-c6ccccc6)oc6ccccc56)ccc5ccc2c3c54)c([2H])c1[2H] ; [2H]c1c([2H])c([2H])c(-c2ccc3ccc4c(-c5c(-c6cccc7ccccc67)oc6ccccc56)ccc5ccc2c3c54)c([2H])c1[2H] ; [2H]c1c([2H])c([2H])c(-c2ccc3ccc4c(-c5ccc6occc6c5)ccc5ccc2c3c54)c([2H])c1[2H]]

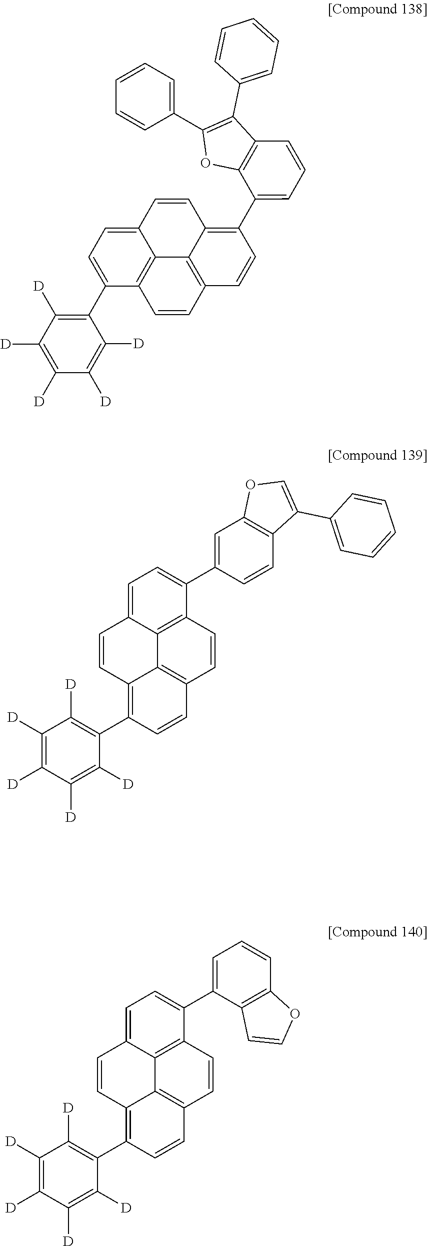 [SMILES: [2H]c1c([2H])c([2H])c(-c2ccc3ccc4c(-c5cccc6c(-c7ccccc7)c(-c7ccccc7)oc56)ccc5ccc2c3c54)c([2H])c1[2H] ; [2H]c1c([2H])c([2H])c(-c2ccc3ccc4c(-c5ccc6c(-c7ccccc7)coc6c5)ccc5ccc2c3c54)c([2H])c1[2H] ; [2H]c1c([2H])c([2H])c(-c2ccc3ccc4c(-c5cccc6occc56)ccc5ccc2c3c54)c([2H])c1[2H]]

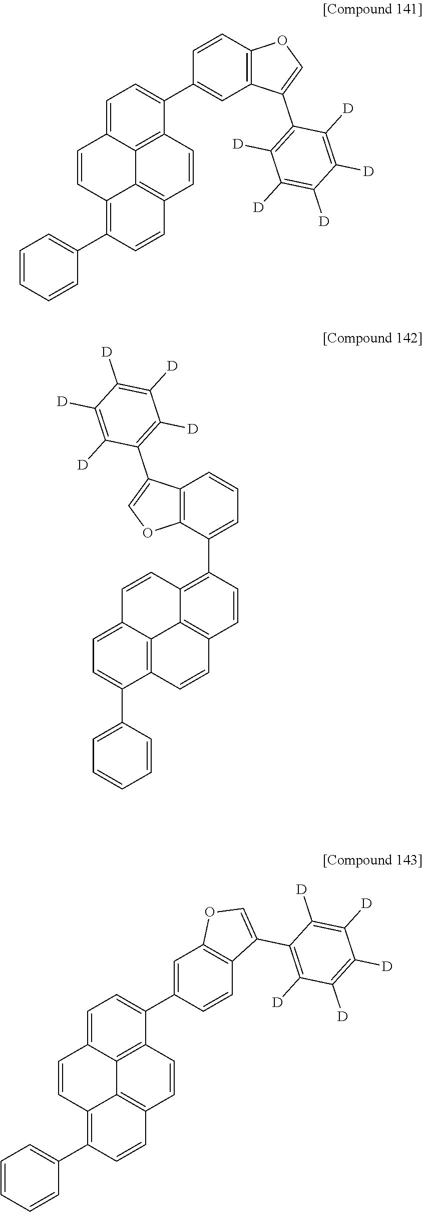 [SMILES: [2H]c1c([2H])c([2H])c(-c2coc3ccc(-c4ccc5ccc6c(-c7ccccc7)ccc7ccc4c5c76)cc23)c([2H])c1[2H] ; [2H]c1c([2H])c([2H])c(-c2coc3c(-c4ccc5ccc6c(-c7ccccc7)ccc7ccc4c5c76)cccc23)c([2H])c1[2H] ; [2H]c1c([2H])c([2H])c(-c2coc3cc(-c4ccc5ccc6c(-c7ccccc7)ccc7ccc4c5c76)ccc23)c([2H])c1[2H]]

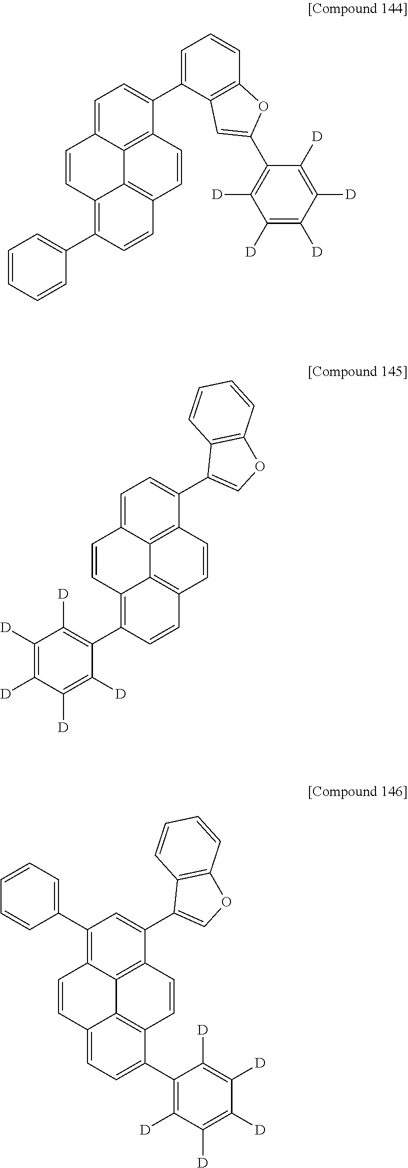 [SMILES: [2H]c1c([2H])c([2H])c(-c2cc3c(-c4ccc5ccc6c(-c7ccccc7)ccc7ccc4c5c76)cccc3o2)c([2H])c1[2H] ; [2H]c1c([2H])c([2H])c(-c2ccc3ccc4c(-c5coc6ccccc56)ccc5ccc2c3c54)c([2H])c1[2H] ; [2H]c1c([2H])c([2H])c(-c2ccc3ccc4c(-c5ccccc5)cc(-c5coc6ccccc56)c5ccc2c3c45)c([2H])c1[2H]]

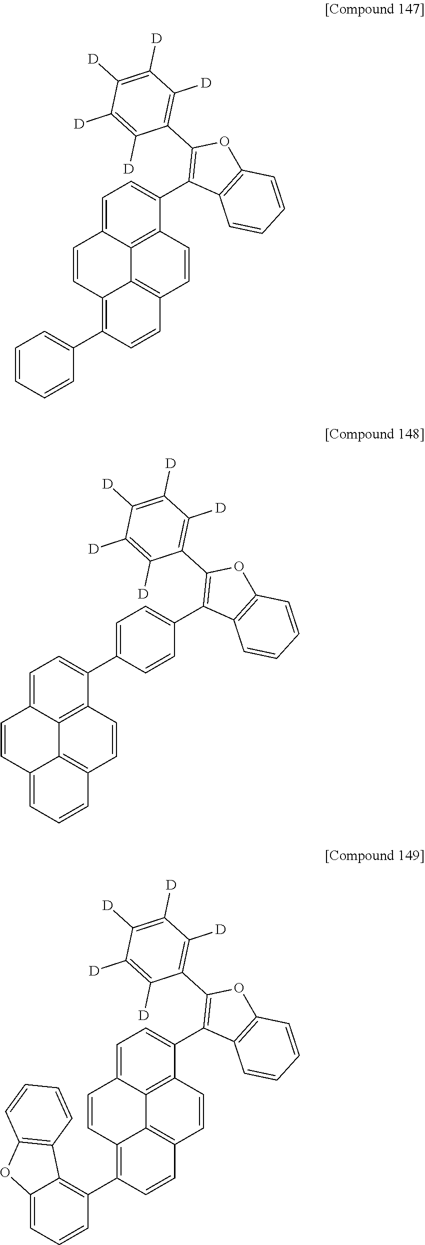 [SMILES: [2H]c1c([2H])c([2H])c(-c2oc3ccccc3c2-c2ccc3ccc4c(-c5ccccc5)ccc5ccc2c3c54)c([2H])c1[2H] ; [2H]c1c([2H])c([2H])c(-c2oc3ccccc3c2-c2ccc(-c3ccc4ccc5cccc6ccc3c4c56)cc2)c([2H])c1[2H] ; [2H]c1c([2H])c([2H])c(-c2oc3ccccc3c2-c2ccc3ccc4c(-c5cccc6oc7ccccc7c56)ccc5ccc2c3c54)c([2H])c1[2H]]

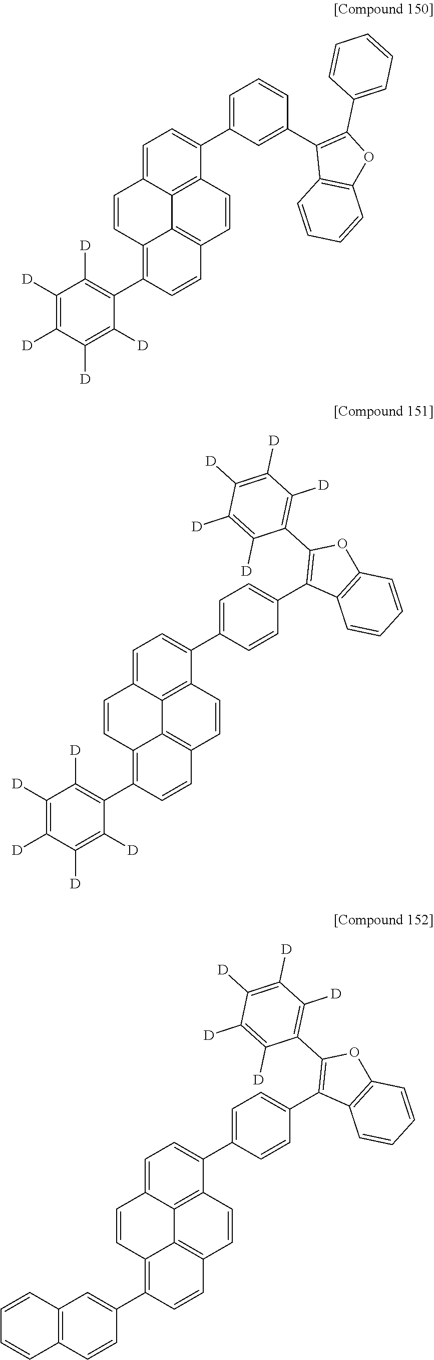 [SMILES: [2H]c1c([2H])c([2H])c(-c2ccc3ccc4c(-c5cccc(-c6c(-c7ccccc7)oc7ccccc67)c5)ccc5ccc2c3c54)c([2H])c1[2H] ; [2H]c1c([2H])c([2H])c(-c2oc3ccccc3c2-c2ccc(-c3ccc4ccc5c(-c6c([2H])c([2H])c([2H])c([2H])c6[2H])ccc6ccc3c4c65)cc2)c([2H])c1[2H] ; [2H]c1c([2H])c([2H])c(-c2oc3ccccc3c2-c2ccc(-c3ccc4ccc5c(-c6ccc7ccccc7c6)ccc6ccc3c4c65)cc2)c([2H])c1[2H]]

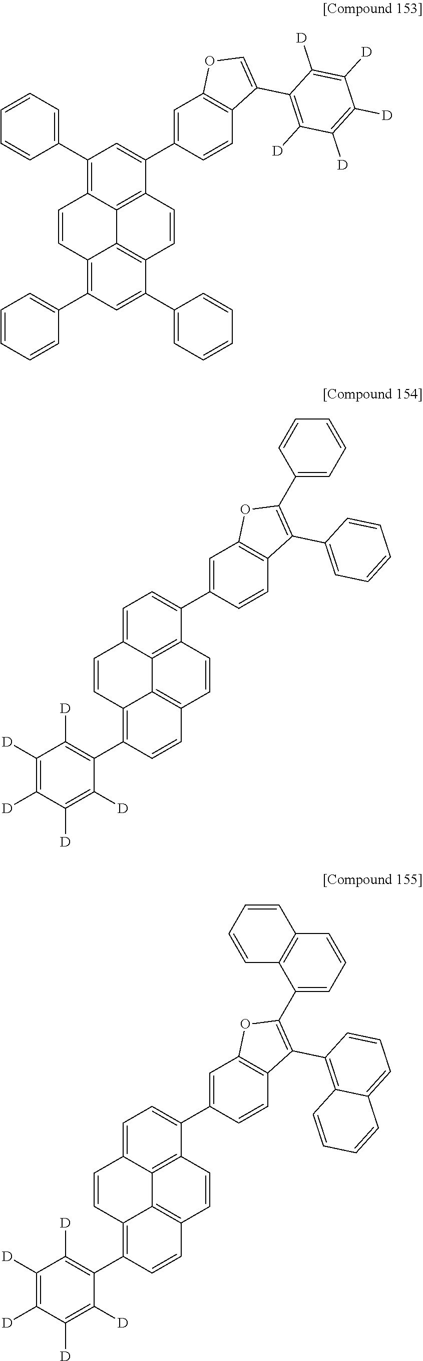 [SMILES: [2H]c1c([2H])c([2H])c(-c2coc3cc(-c4cc(-c5ccccc5)c5ccc6c(-c7ccccc7)cc(-c7ccccc7)c7ccc4c5c67)ccc23)c([2H])c1[2H] ; [2H]c1c([2H])c([2H])c(-c2ccc3ccc4c(-c5ccc6c(-c7ccccc7)c(-c7ccccc7)oc6c5)ccc5ccc2c3c54)c([2H])c1[2H] ; [2H]c1c([2H])c([2H])c(-c2ccc3ccc4c(-c5ccc6c(-c7cccc8ccccc78)c(-c7cccc8ccccc78)oc6c5)ccc5ccc2c3c54)c([2H])c1[2H]]

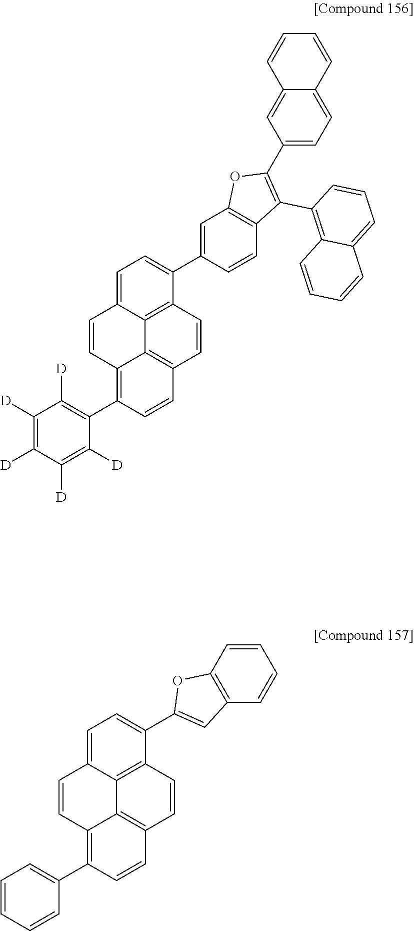 [SMILES: [2H]c1c([2H])c([2H])c(-c2ccc3ccc4c(-c5ccc6c(-c7cccc8ccccc78)c(-c7ccc8ccccc8c7)oc6c5)ccc5ccc2c3c54)c([2H])c1[2H] ; c1ccc(-c2ccc3ccc4c(-c5cc6ccccc6o5)ccc5ccc2c3c54)cc1]

In addition, the present disclosure provides an organic light-emitting diode including: a first electrode: a second electrode facing the first electrode; and a light-emitting layer disposed between the first electrode and the second electrode, wherein the light-emitting layer includes at least one of the compounds represented by Chemical Formula A.

Throughout the description of the present disclosure, the phrase “(an organic layer) includes (comprises) at least one organic compound” may be construed to mean that “(an organic layer) may include (comprise) a single organic compound species or two or more different species of organic compounds falling within the scope of the present disclosure”.

In this regard, the organic layer in the organic light-emitting diode according to the present disclosure may include at least one of a hole injection layer, a hole transport layer, a functional layer capable of both hole injection and hole transport, a light-emitting layer, an electron transport layer, and an electron injection layer.

In more particular embodiments of the present disclosure, the organic layer disposed between the first electrode and the second electrode includes a light-emitting layer composed of a host and a dopant, wherein the compound represented by Chemical Formula A serves as a host in the light-emitting layer.

In an embodiment, the organic light-emitting diode according to the present disclosure may employ at least one of the compounds represented by the following Chemical Formulas D1 to Chemical Formula D10 as a dopant compound in the light-emitting layer:

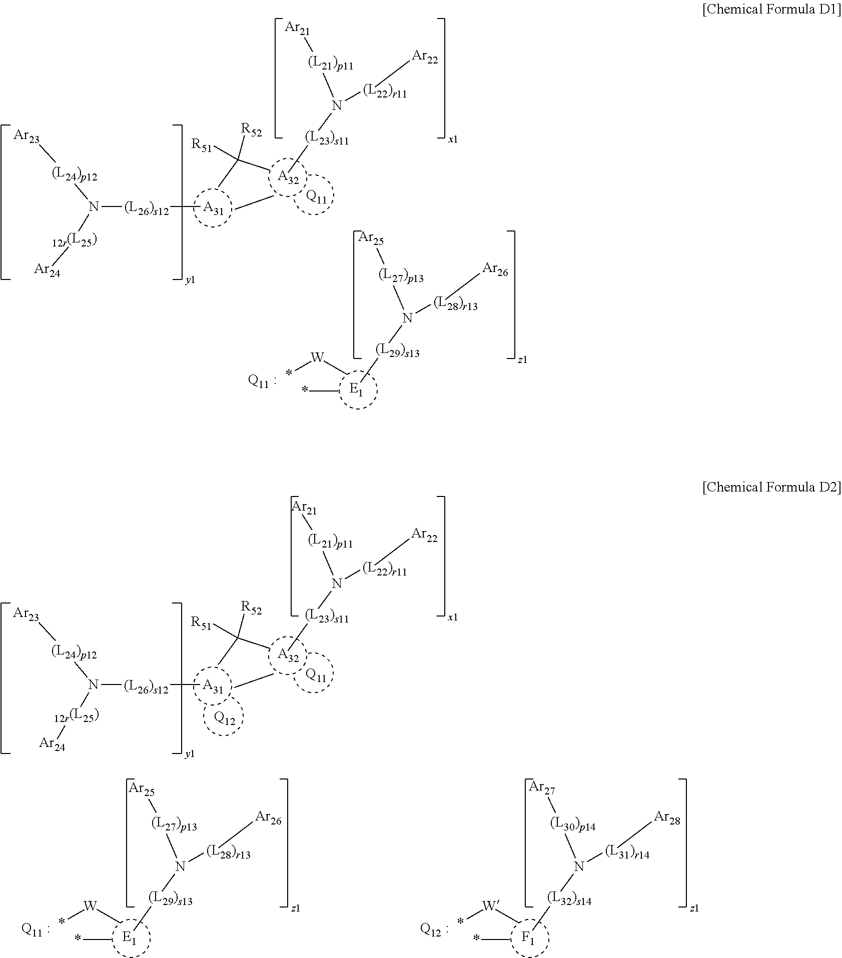

wherein, A₃₁, A₃₂, E₁, and F₁, which are same or different, are each independently a substituted or unsubstituted aromatic hydrocarbon ring of 6 to 50 carbon atoms, or a substituted or unsubstituted heteroaromatic ring of 2 to 40 carbon atoms;
two adjacent carbon atoms of the aromatic ring A₃₁ and two adjacent carbon atoms of the aromatic ring A₃₂ form a 5-membered fused ring together with a carbon atom to which substituents R₅₁ and R₅₂ are bonded;
linkers L₂₁ to L₃₂, which are same or different, are each independently selected from among a single bond, a substituted or unsubstituted alkylene of 1 to 60 carbon atoms, a substituted or unsubstituted alkenylene of 2 to 60 carbon atoms, a substituted or unsubstituted alkynylene of 2 to 60 carbon atoms, a substituted or unsubstituted cycloalkylene of 3 to 60 carbon atoms, a substituted or unsubstituted heterocycloalkylene of 2 to 60 carbon atoms, a substituted or unsubstituted arylene of 6 to 60 carbon atoms, and a substituted or unsubstituted heteroarylene of 2 to 60 carbon atoms,
W and W′, which are same or different, are each independently any one selected from among N—R₅₃, CR₅₄R₅₅, SiR₅₆R₅₇, GeR₅₈R₅₉, O, S, and Se;
R₅₁ to R₅₉ and Ar₂₁ to Ar₂₈, which are same or different, are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a substituted or unsubstituted alkylgermyl of 1 to 30 carbon atoms, a substituted or unsubstituted arylgermyl of 1 to 30 carbon atoms, a cyano, a nitro, and a halogen,
wherein R₅₁ and R₅₂ together may form a mono- or polycyclic aliphatic or aromatic ring that may be a heterocyclic ring bearing a heteroatom selected from among N, O, P, Si, S, Ge, Se, and Te as a ring member;
p11 to p14, r11 to r14, and s11 to s14 are each independently an integer of 1 to 3, wherein when any of them is 2 or greater, the corresponding linkers L₂₁ to L₃₂ may be same or different,
x1 is 1, and y1, z1, and z2, which are same or different, are each independently an integer of 0 to 1; and
Ar₂₁ may form a ring with Ar₂₂, Ar₂₃ may form a ring with Ar₂₄, Ar₂₅ may form a ring with Ar₂₆, and Ar₂₇ may form a ring with Ar₂₈;
two adjacent carbon atoms of the A₃₂ ring moiety of Chemical Formula D1 may occupy respective positions * of Structural Formula Q₁₁ to form a fused ring, and
two adjacent carbon atoms of the A₃₁ ring moiety of Chemical Formula D2 may occupy respective positions * of Structural Formula Q₁₂ to form a fused ring, and two adjacent carbon atoms of the A₃₂ ring moiety of Chemical Formula D2 may occupy respective positions * of Structural Formula Q₁₁ to form a fused ring,

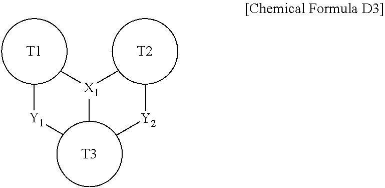

wherein,
X₁ is any one selected from among B, P, and P═O;
T1 to T3, which are same or different, are each independently a substituted or unsubstituted aromatic hydrocarbon ring of 6 to 50 carbon atoms, or a substituted or unsubstituted heteroaromatic ring of 2 to 40 carbon atoms;
Y₁ is any one selected from among N—R₆₁, CR₆₂R₆₃, O, S, and SiR₆₄R₆₅; and
Y₂ is any one selected from among N—R₆₆, CR₆₇R₆₈, O, S, and SiR₆₉R₇₀;
wherein R₆₁ to R₇₀, which are same or different, are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen, and wherein at least one of R₆₁ to R₇₀ may be connected to at least one of T1 to T3 to form an additional mono- or polycyclic aliphatic or aromatic ring.

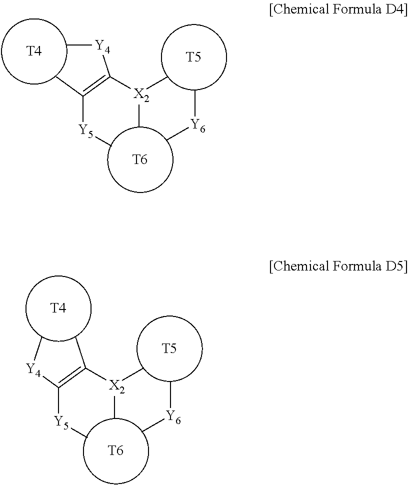

wherein,
X₂ is any one selected from among B, P, and P═O;
T4 to T6 are as defined for T1 to T3 in Chemical Formula D3;
Y₄ is any one selected from among N—R₆₁, CR₆₂R₆₃, O, S, and SiR₆₄R₆₅;
Y₅ is any one selected from among N—R₆₆, CR₆₇R₆₈, O, S, and SiR₆₉R₇₀; and
Y₆ is any one selected from among N—R₇₁, CR₇₂R₇₃, O, S, and SiR₇₄R₇₅,
R₆₁ to R₇₅ being as defined for R₆₁ to R₇₀ in Chemical Formula D3,

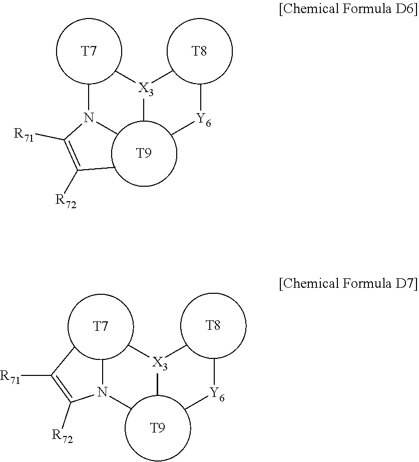

wherein, X₃ is any one selected from among B, P, and P═O;
T₇ to T₉ are defined as for T₁ to T₃ in Chemical Formula D3; and
Y₆ is any one selected from among N—R₆₁, CR₆₂R₆₃, O, S, and SiR₆₄R₆₅/R₆₁
R₆₁ to R₆₅ and R₇₁ to R₇₂ being each as defined R₆₁ to R₇₀ in Chemical Formula D3,
wherein R₇₁ and R₇₂ may be connected to each other to form an additional mono- or polycyclic aliphatic or aromatic ring, or may be connected to the T7 ring moiety or T9 ring moiety to form an additional mono- or polycyclic aliphatic or aromatic ring,

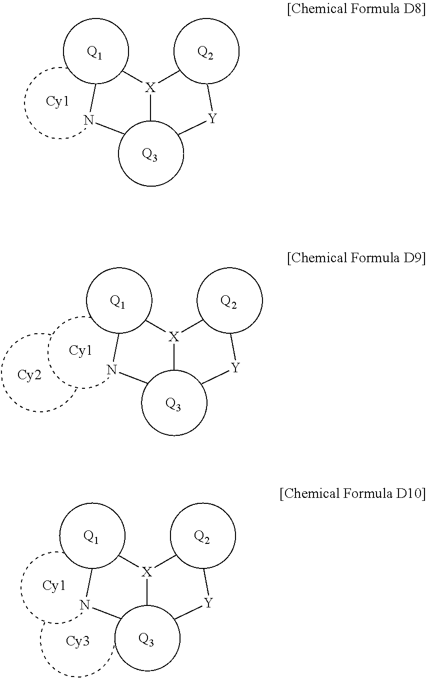

wherein,
X is any one selected from among B, P, and P═O; Q₁ to Q₃ are each as defined for T1 to T3 in Chemical Formula D3;
Y is any one selected from among N—R₃, CR₄R₅, O, S, and Se,
R₃ to R₅ being each as defined for R₆₁ to R₇₀ in Chemical Formula D3,
wherein R₃ to R₅ may each be connected to the Q₂ or Q₃ ring moiety to form an additional mono- or polycyclic aliphatic or aromatic ring, and
R₄ and R₅ may be connected to each other to form an additional mono- or polycyclic aliphatic or aromatic ring;
the ring formed by Cy1 is a substituted or unsubstituted alkylene of 1 to 10 carbon atoms, except for the nitrogen (N) atom, the aromatic carbon atom of @1 to which the nitrogen (N) atom is connected, and the aromatic carbon atom of @ to which Cy1 is to bond;
“Cy2” in Chemical Formula D9 may form a saturated hydrocarbon ring added to Cy1 wherein the ring formed by Cy2 is a substituted or unsubstituted alkylene of 1 to 10 carbon atoms, except for the carbon atoms included in Cy1; and
the ring formed by Cy3 in Chemical Formula D10 is a substituted or unsubstituted alkylene of 1 to 10 carbon atoms, except for the aromatic carbon atom of Q₃ to which Cy3 is to bond, the aromatic carbon atom of Q₃ to which the nitrogen (N) atom is connected, the nitrogen (N) atom, and the carbon atom of Cy1 to which the nitrogen (N) atom is connected,
wherein the term “substituted” in the expression “substituted or unsubstituted” used for compounds of Chemical Formulas D1 to D10 means having at least one substituent selected from the group consisting of a deuterium atom, a cyano, a halogen, a hydroxy, a nitro, an alkyl of 1 to 24 carbon atoms, a hydrogenated alkyl of 1 to 24 carbon atoms, an alkenyl of 2 to 24 carbon atoms, an alkynyl of 2 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms, and more particularly, having at least one substituent selected from the group consisting of a deuterium atom, a cyano, a halogen, a hydroxy, a nitro, an alkyl of 1 to 12 carbon atoms, a halogenated alkyl of 1 to 12 carbon atoms, an alkenyl of 2 to 12 carbon atoms, an alkynyl of 2 to 12 carbon atoms, a cycloalkyl of 3 to 12 carbon atoms, a heteroalkyl of 1 to 12 carbon atoms, an aryl of 6 to 18 carbon atoms, an arylalkyl of 7 to 20 carbon atoms, an alkylaryl of 7 to 20 carbon atoms, a heteroaryl of 2 to 18 carbon atoms, a heteroarylalkyl of 2 to 18 carbon atoms, an alkoxy of 1 to 12 carbon atoms, an alkylamino of 1 to 12 carbon atoms, a diarylamino of 12 to 18 carbon atoms, a diheteroarylamino of 2 to 18 carbon atoms, an aryl(heteroaryl)amino of 7 to 18 carbon atoms, an alkylsilyl of 1 to 12 carbon atoms, an arylsilyl of 6 to 18 carbon atoms, an aryloxy of 6 to 18 carbon atoms, and an arylthionyl of 6 to 18 carbon atoms.

Among the dopant compounds according to the present disclosure, the boron compounds represented by Chemical Formulas D3 to D10 may have, on the aromatic hydrocarbon rings or heteroaromatic rings of T1 to T9 or on the aromatic hydrocarbon rings or heteroaromatic rings of Q₁ to Q₃, a substituent selected from a deuterium atom, an alkyl of 1 to 24 carbon atoms, an aryl of 6 to 24 carbon atoms, an alkylamino of 1 to 24 carbon atoms, and an arylamino of 6 to 24 carbon atoms, wherein the alkyl radicals or the aryl radicals in the alkylamino of 1 to 24 carbon atoms and the arylamino of 6 to 24 carbon atoms on the rings may be linked to each other, and particularly a substituent selected from an alkyl of 1 to 12 carbon atoms, an aryl of 6 to 18 carbon atoms, an alkylamino of 1 to 12 carbon atoms, and an arylamino of 6 to 18 carbon atoms wherein the alkyl radicals or aryl radicals in the alkylamino of 1 to 12 carbon atoms and the arylamino of 6 to 18 carbon atoms on the rings may be linked to each other.

Concrete examples of the dopant compounds of Chemical Formulas D1 and D2 used in the light-emitting layer include the compounds of the following Chemical Formulas <d1> to <d239>:

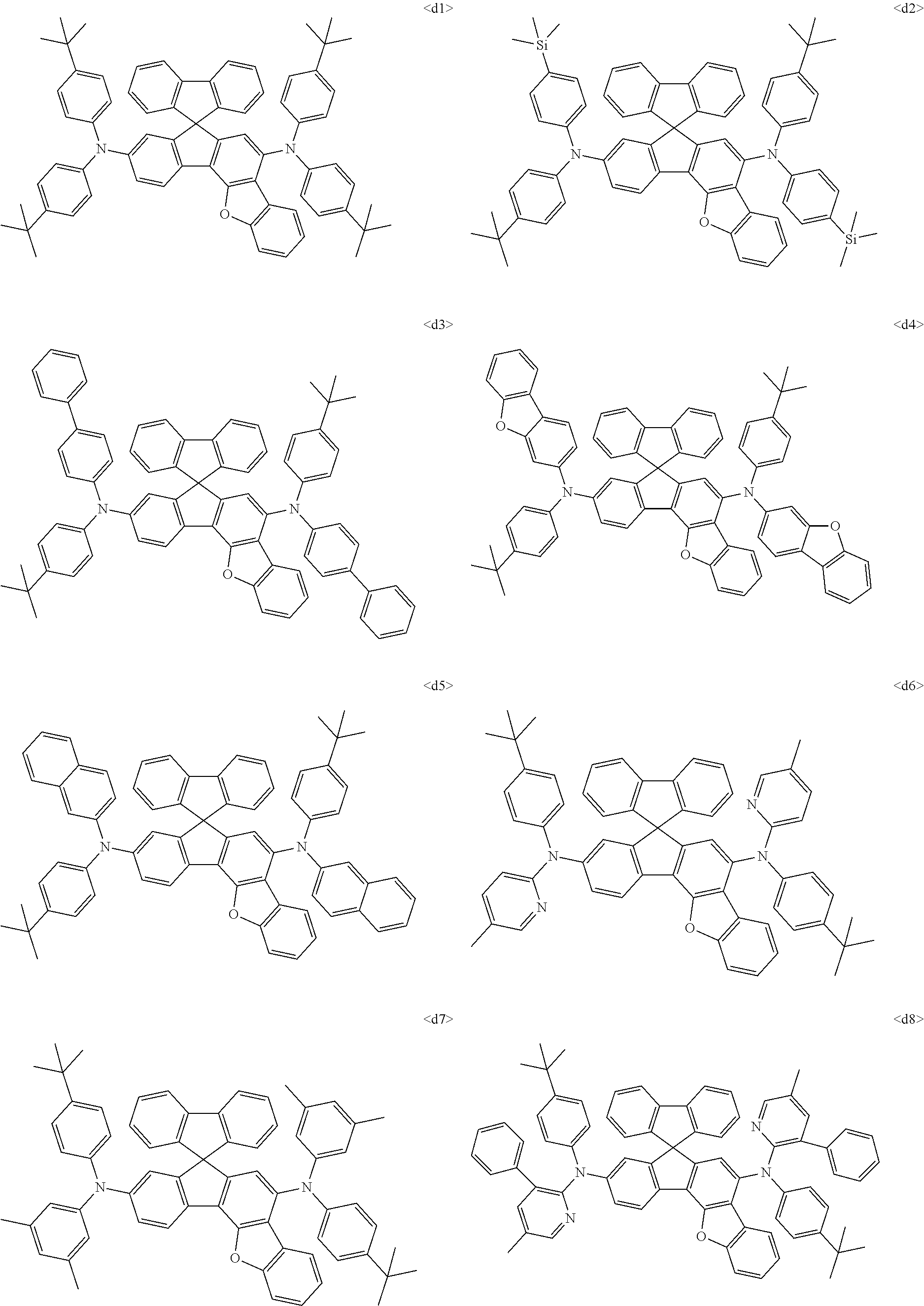

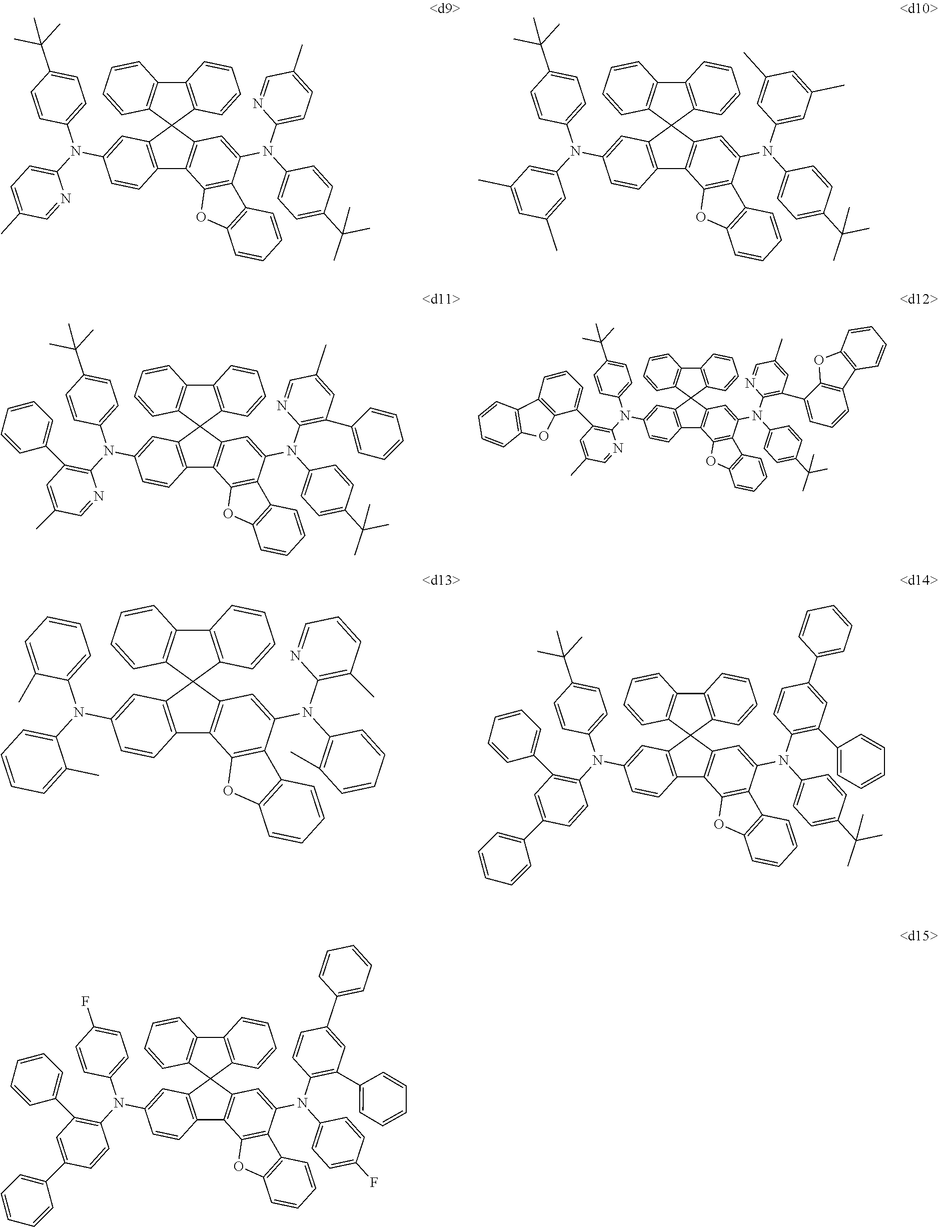

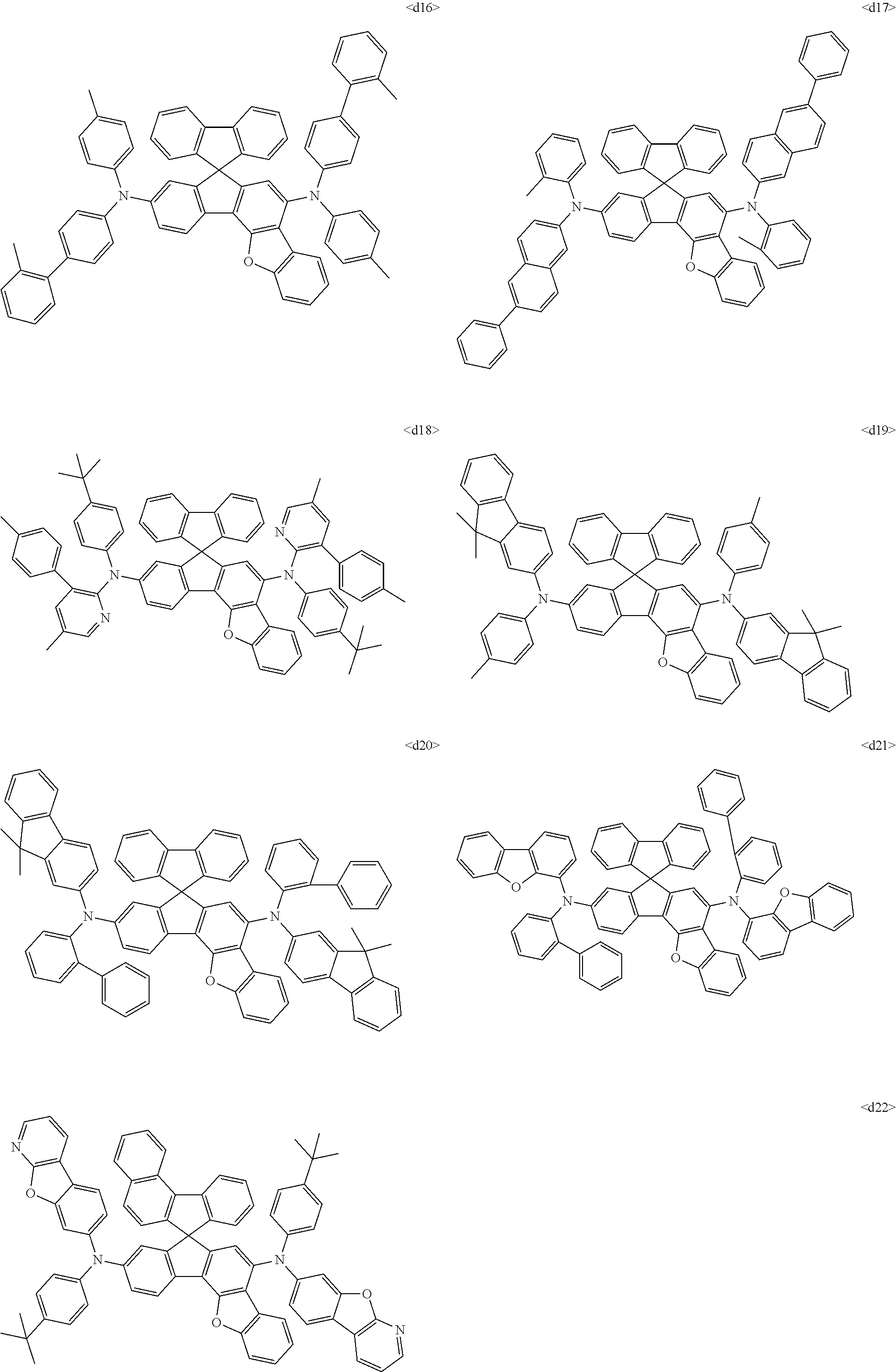

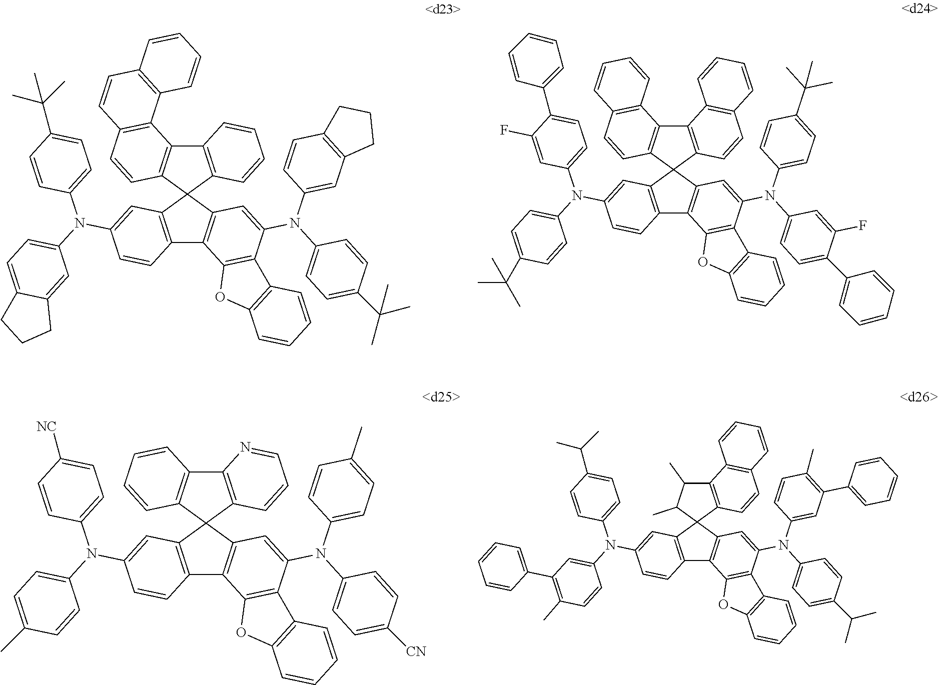

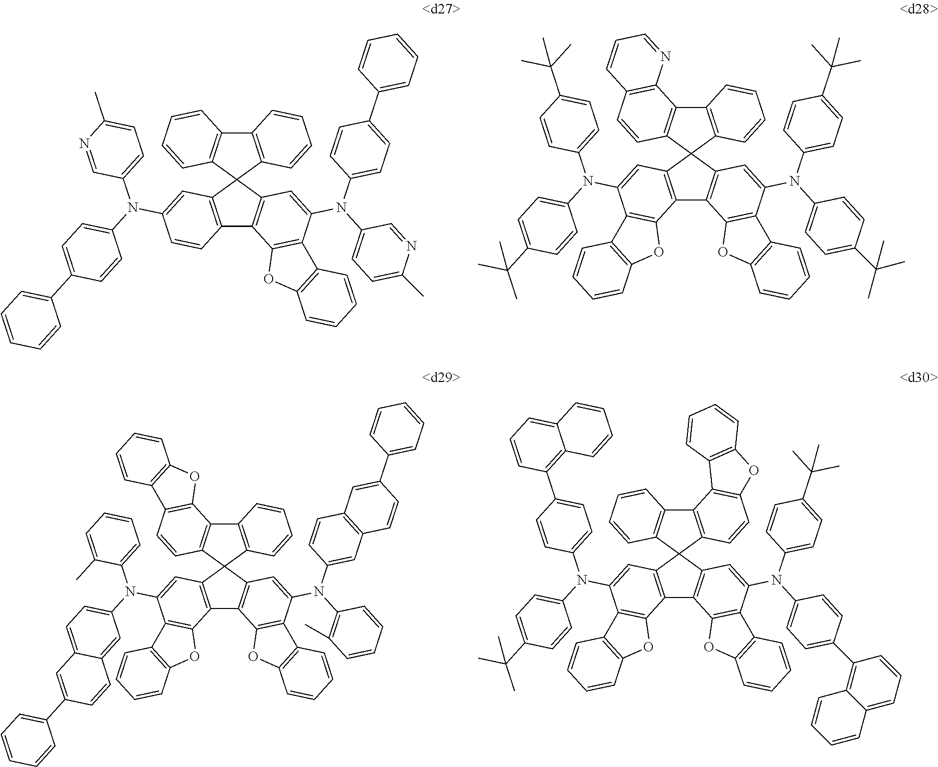

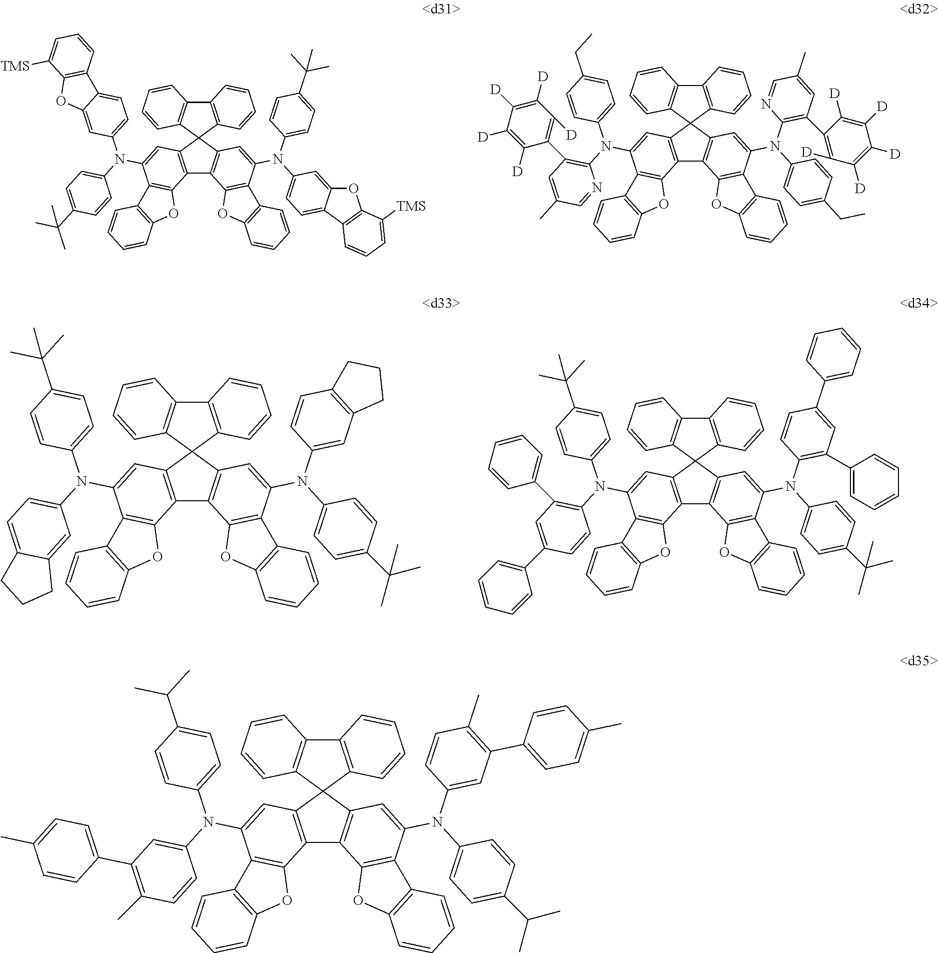

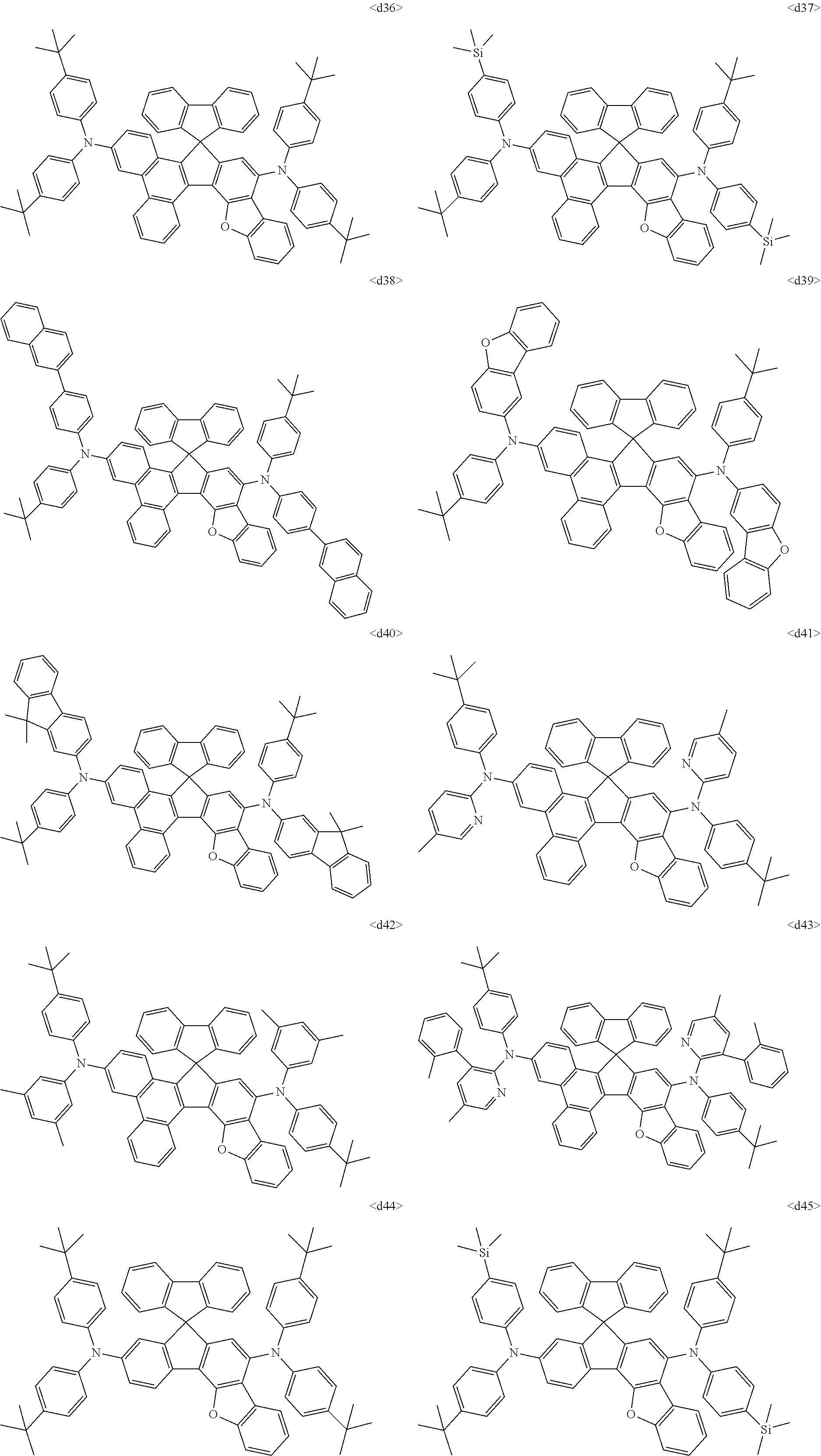

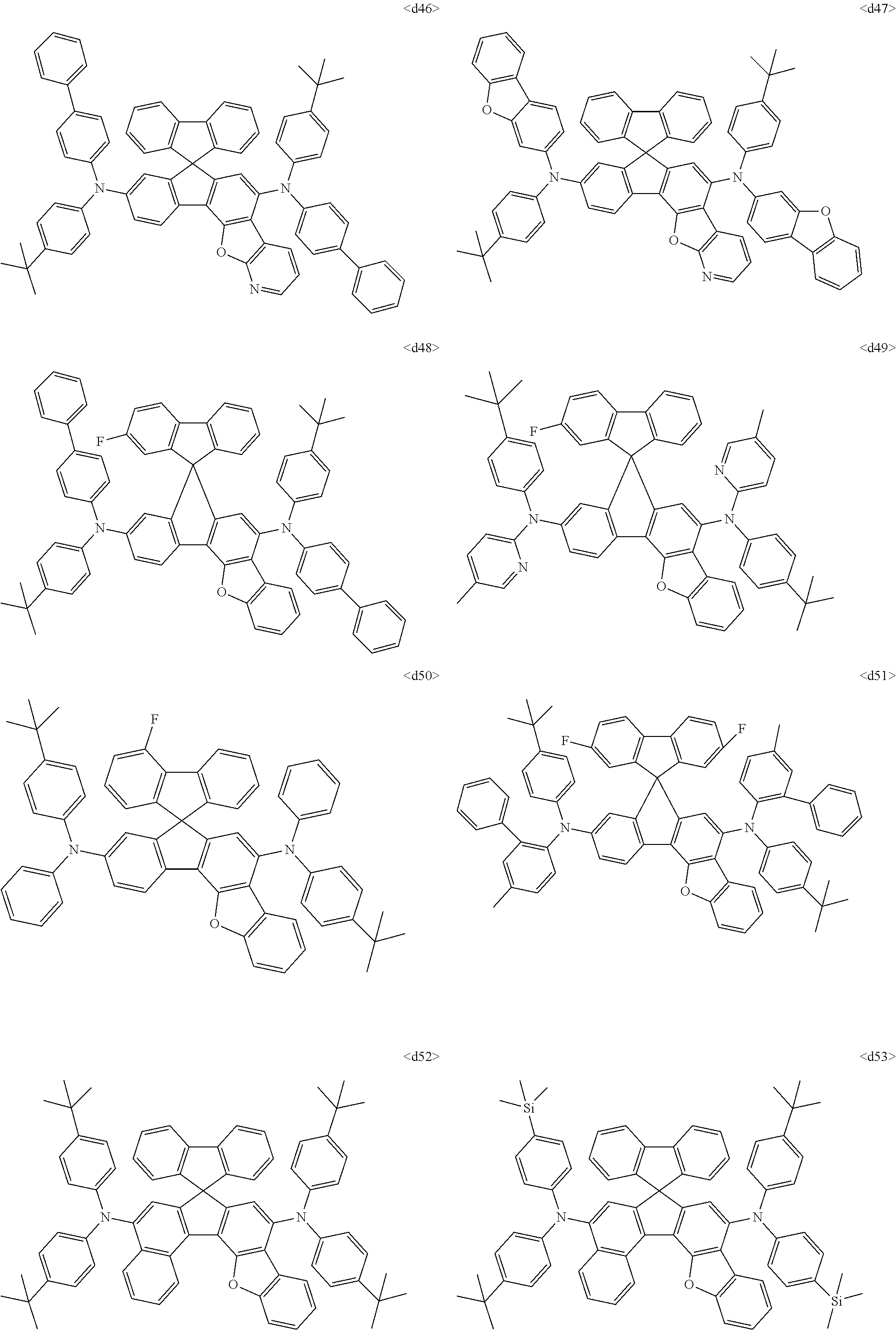

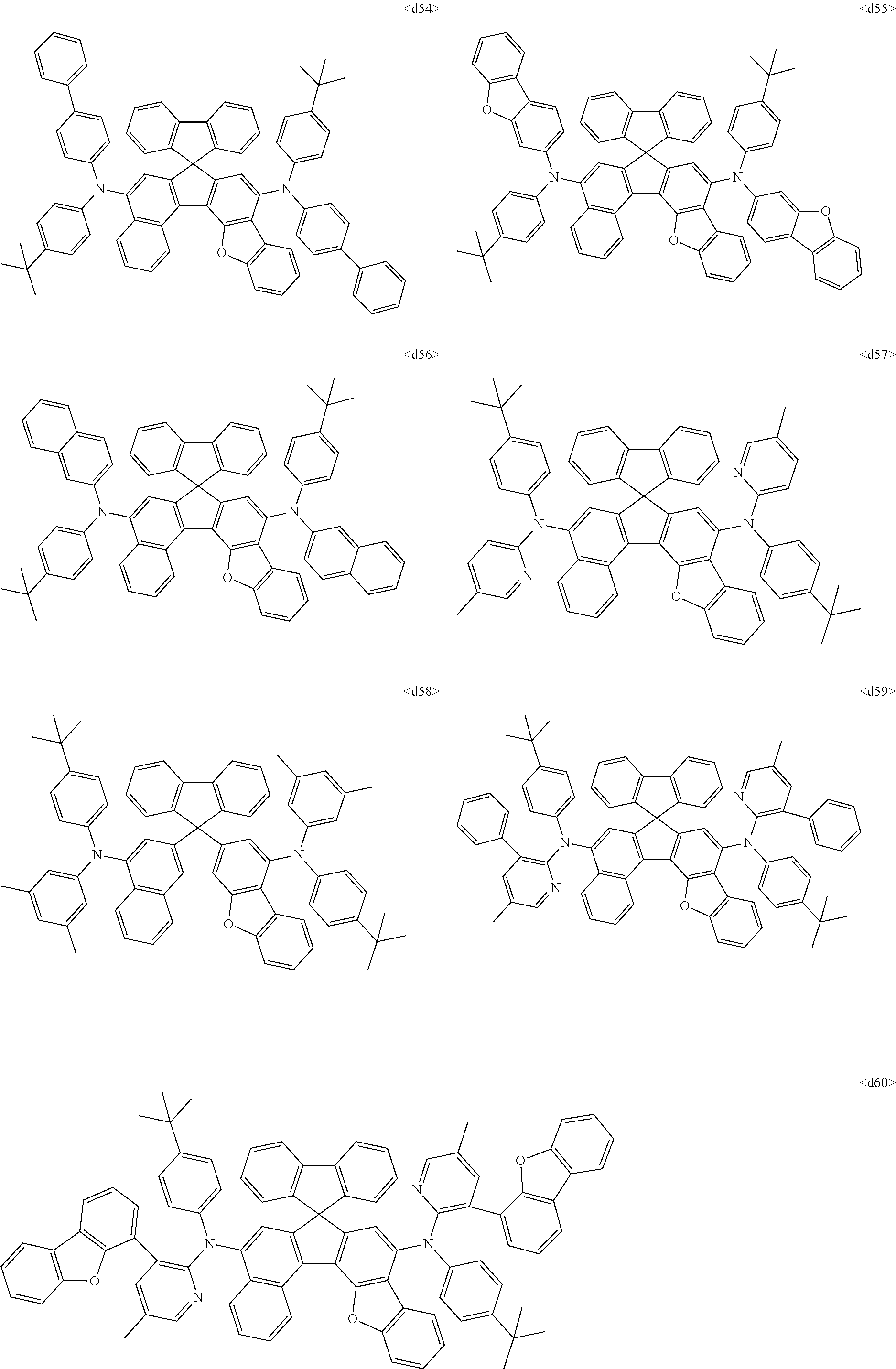

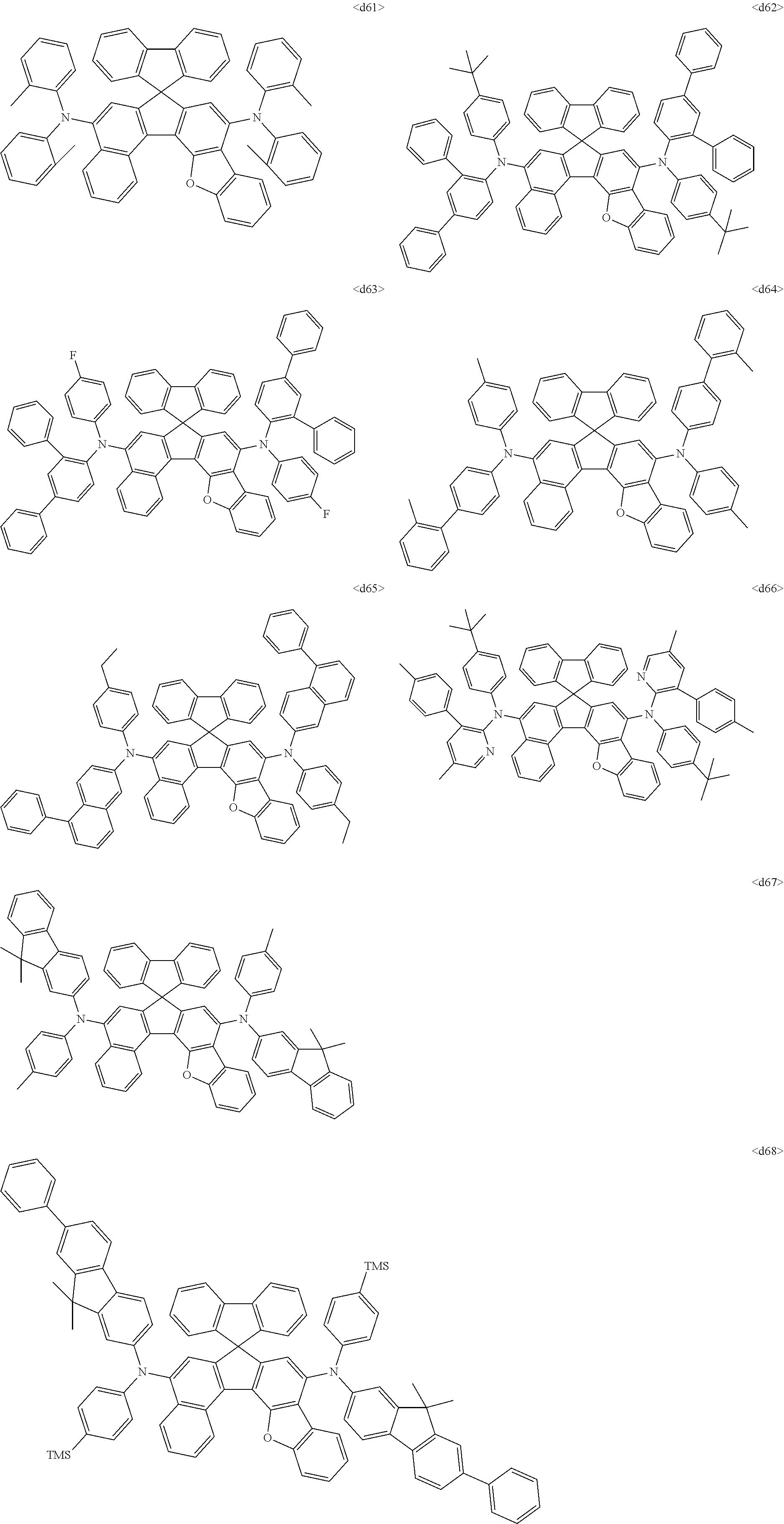

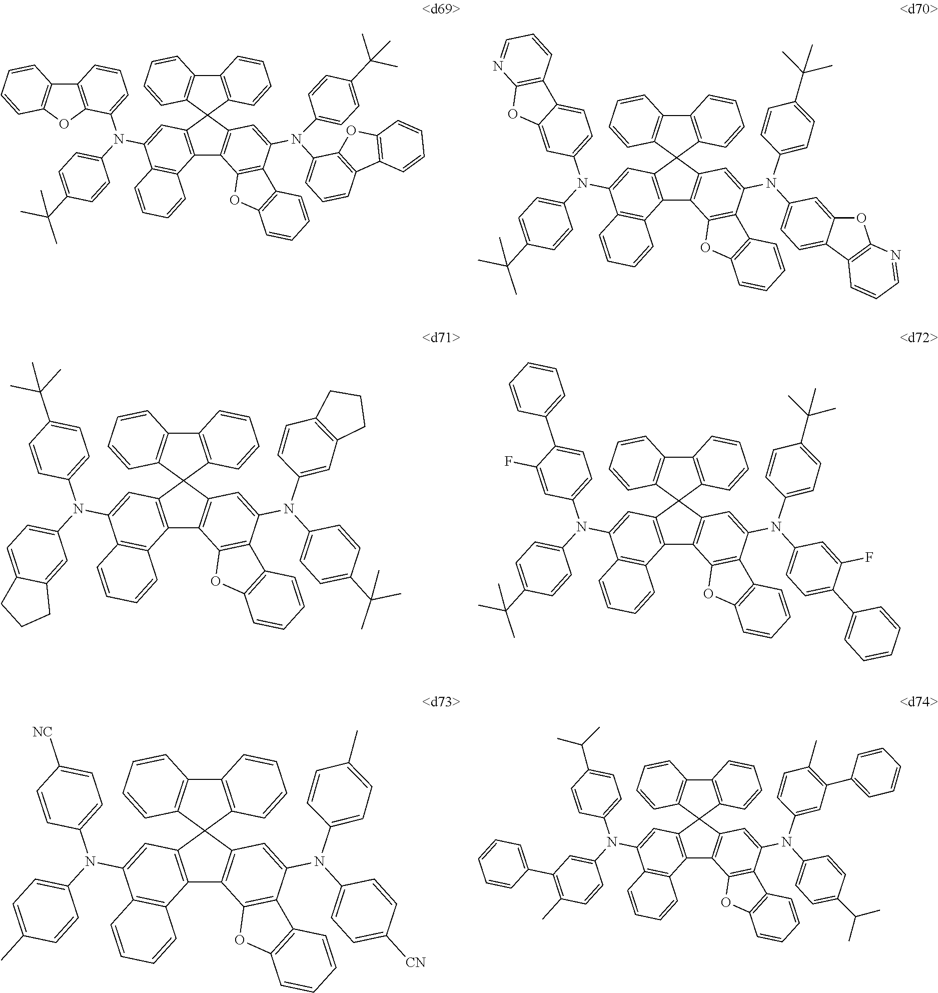

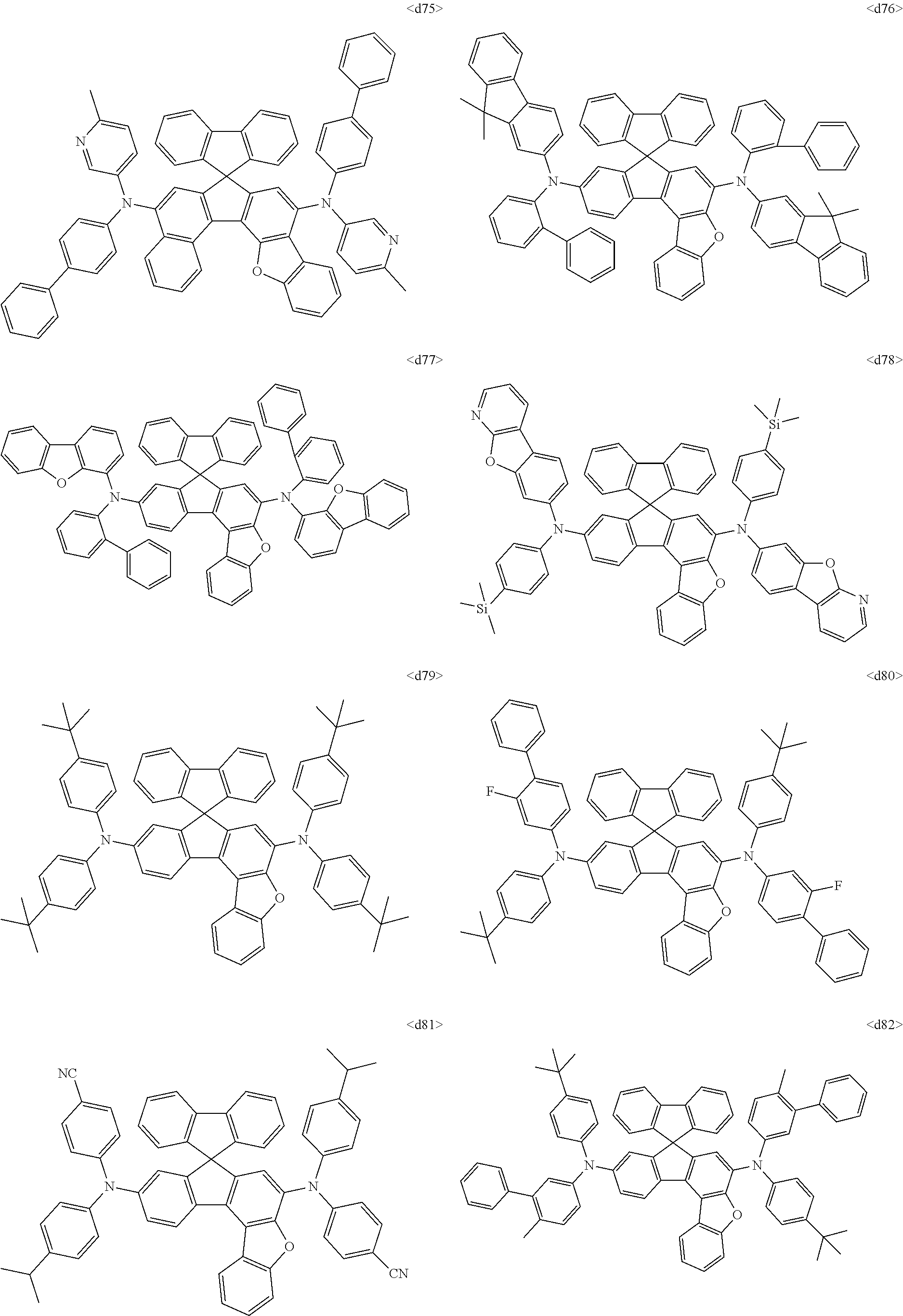

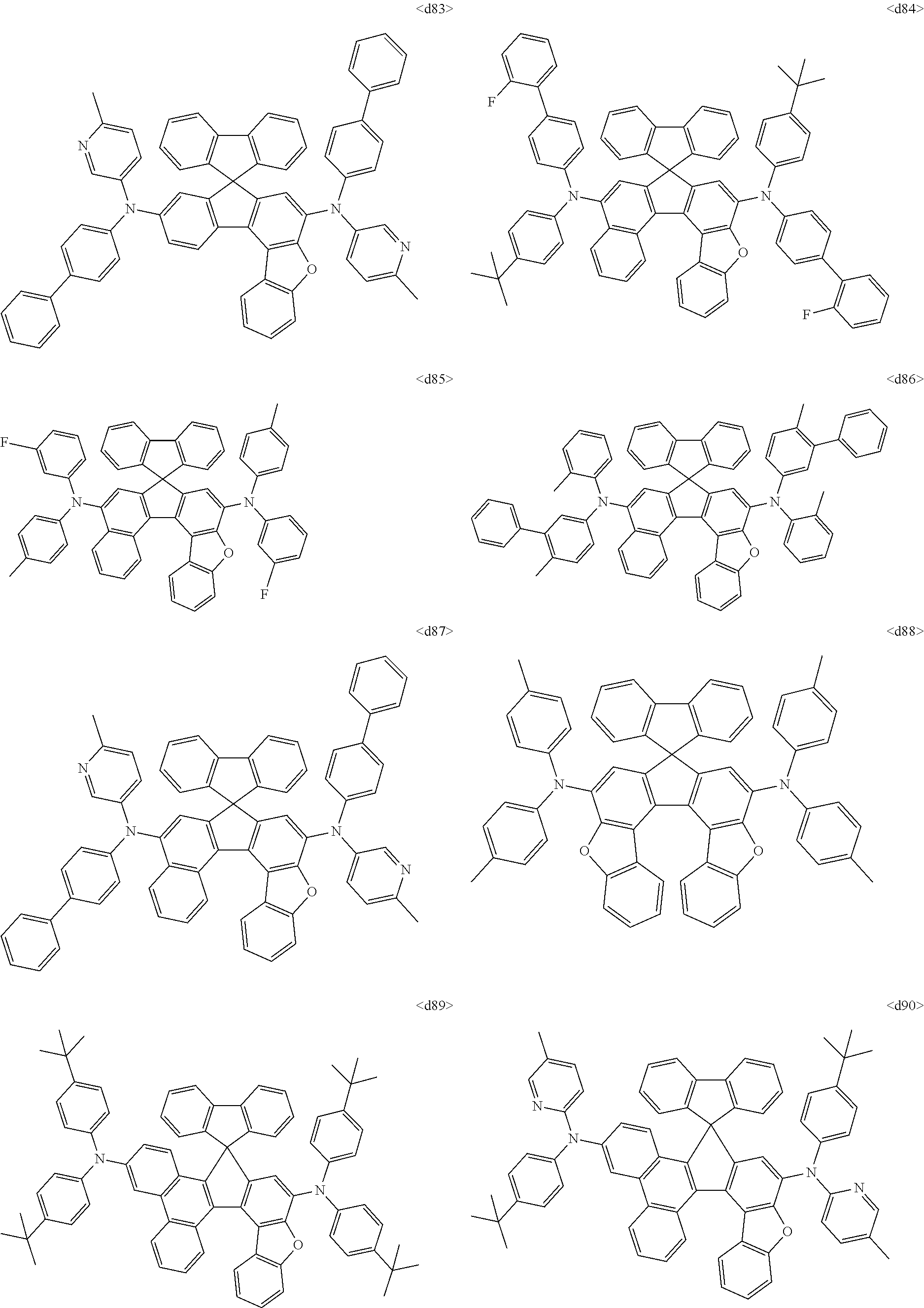

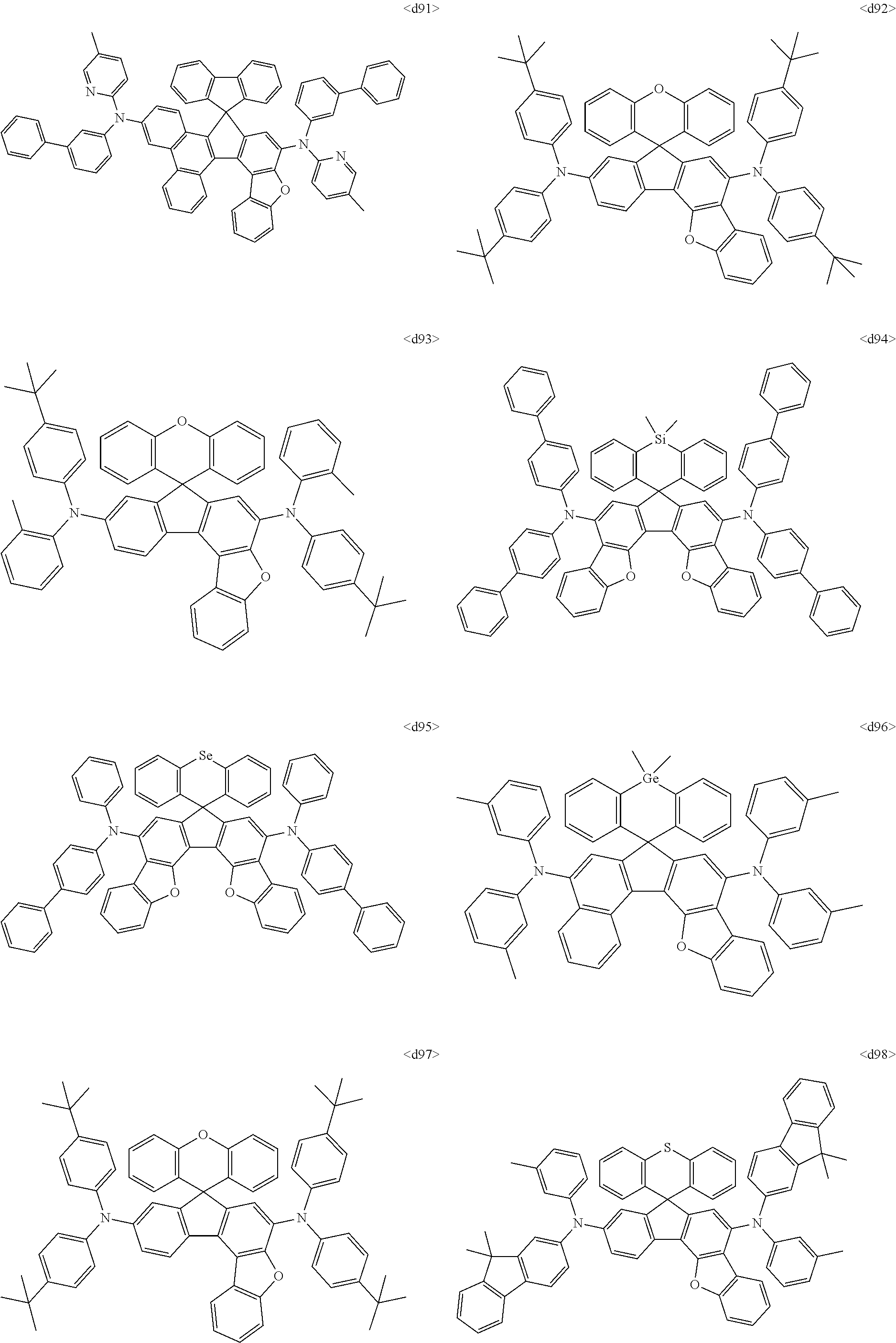

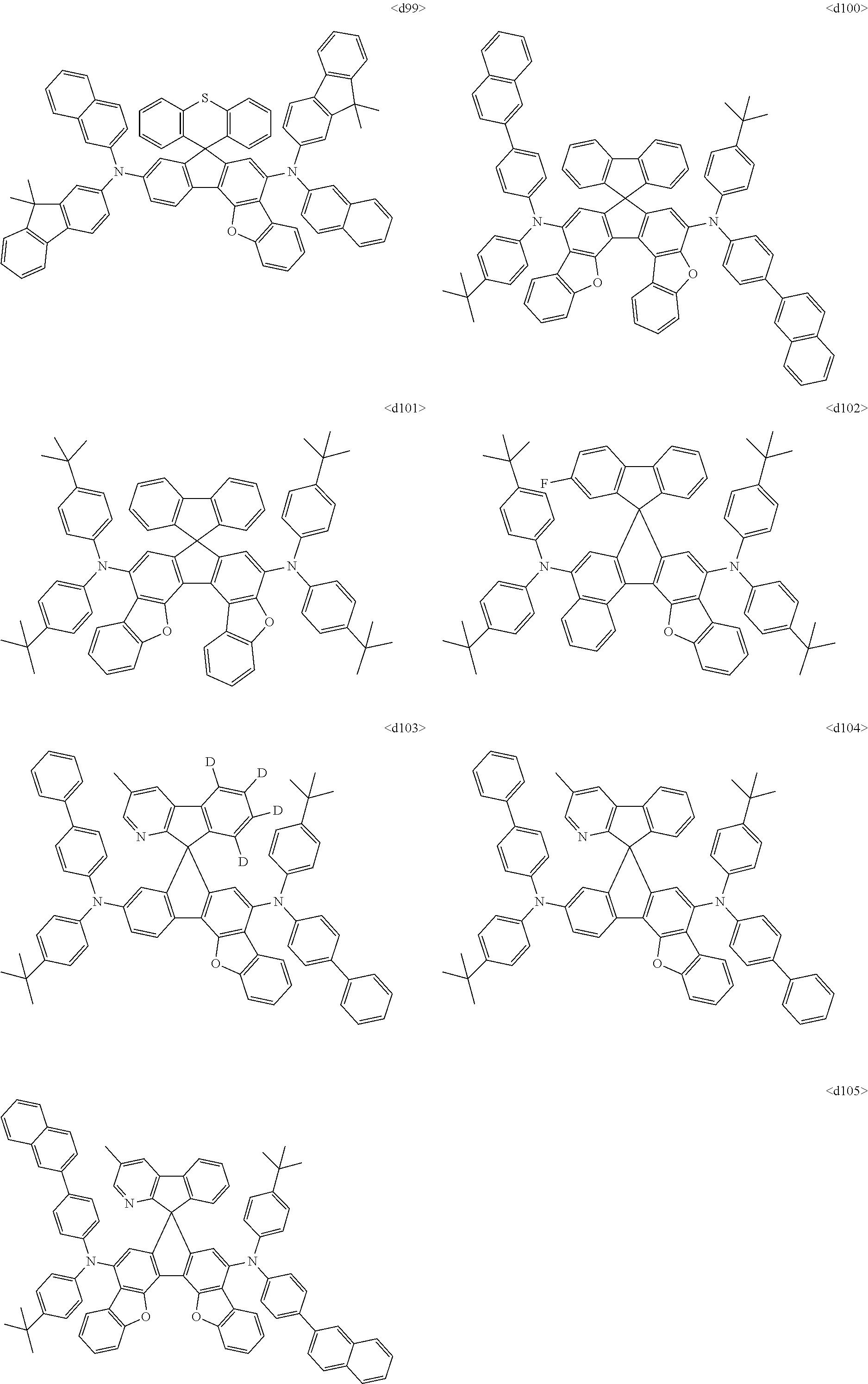

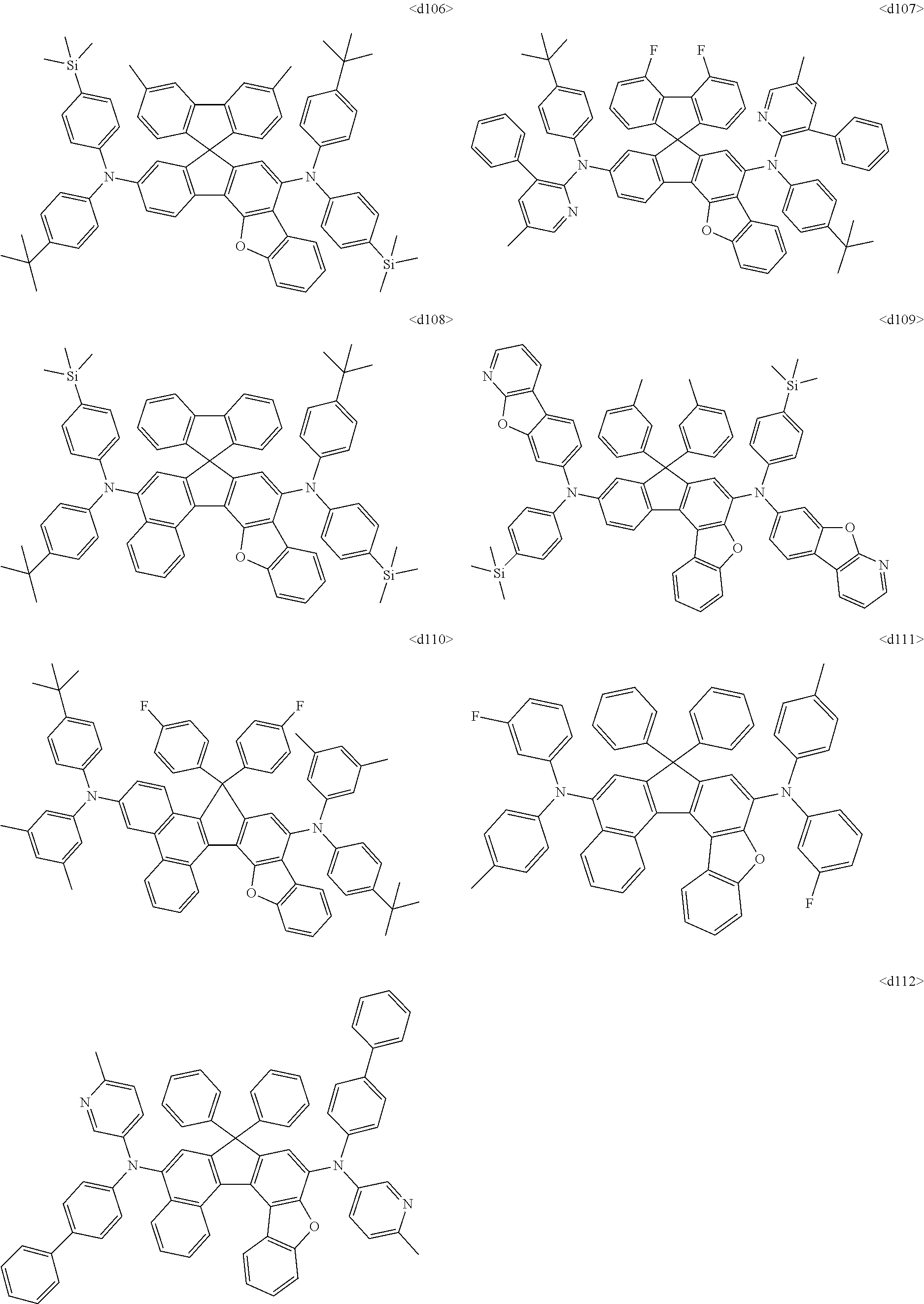

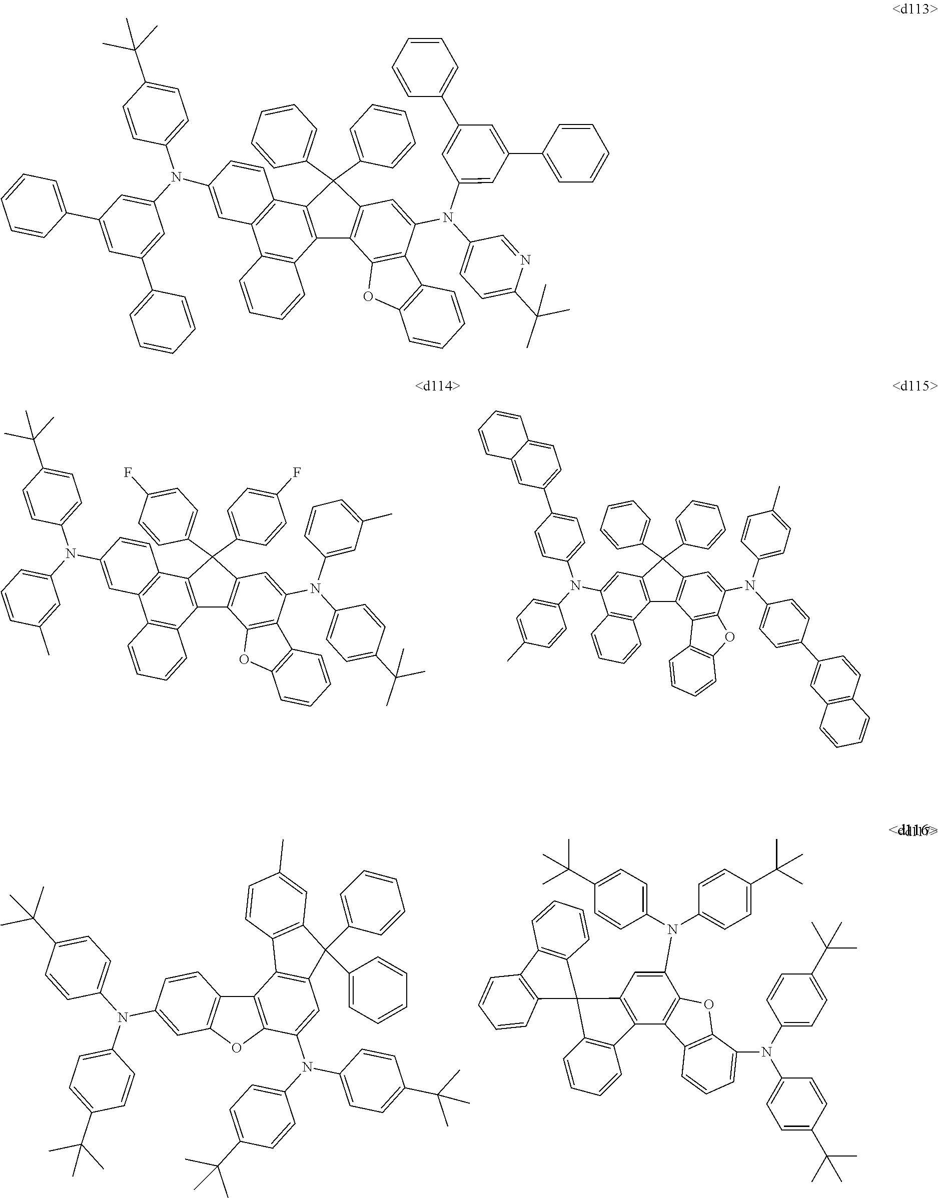

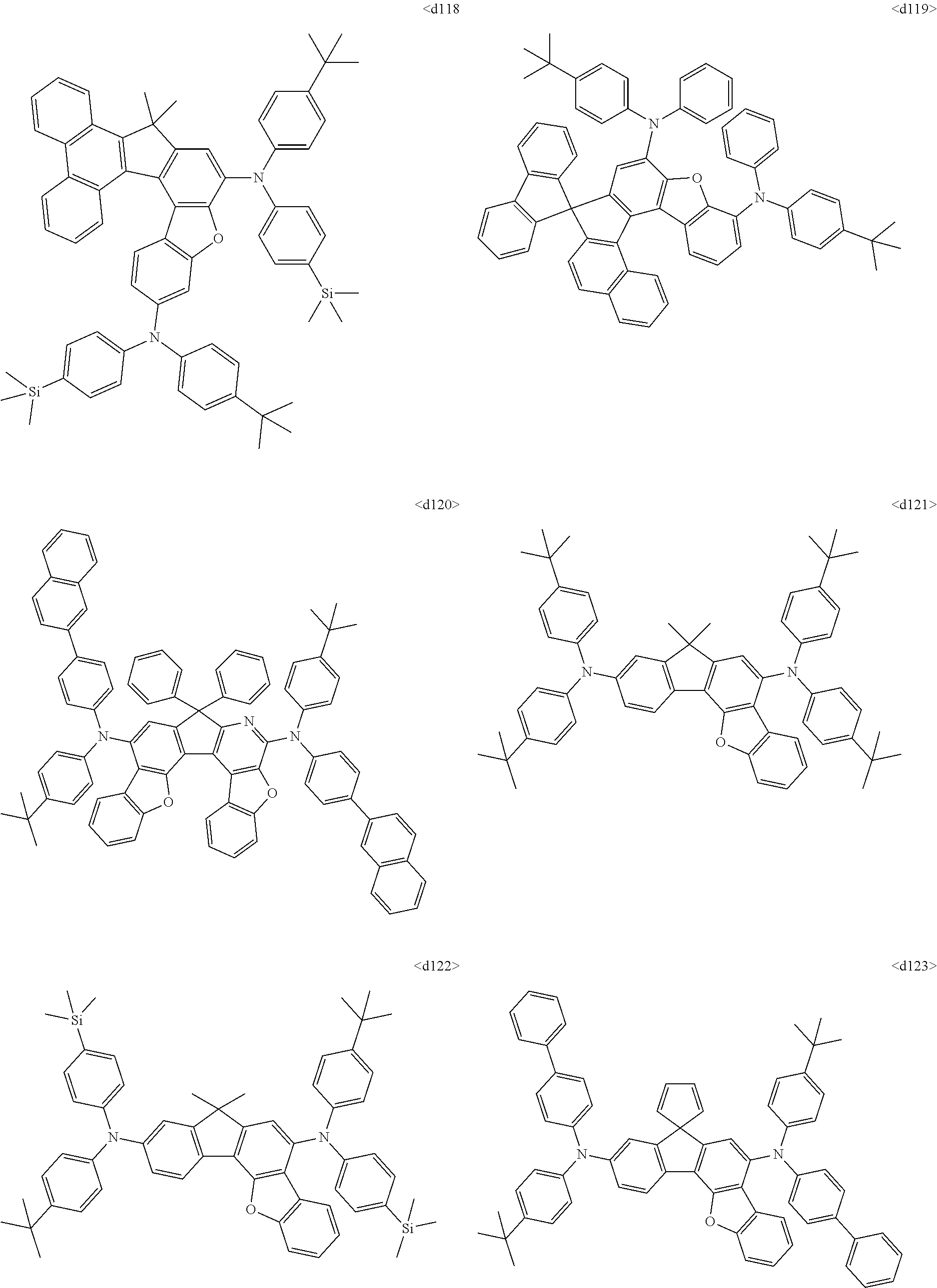

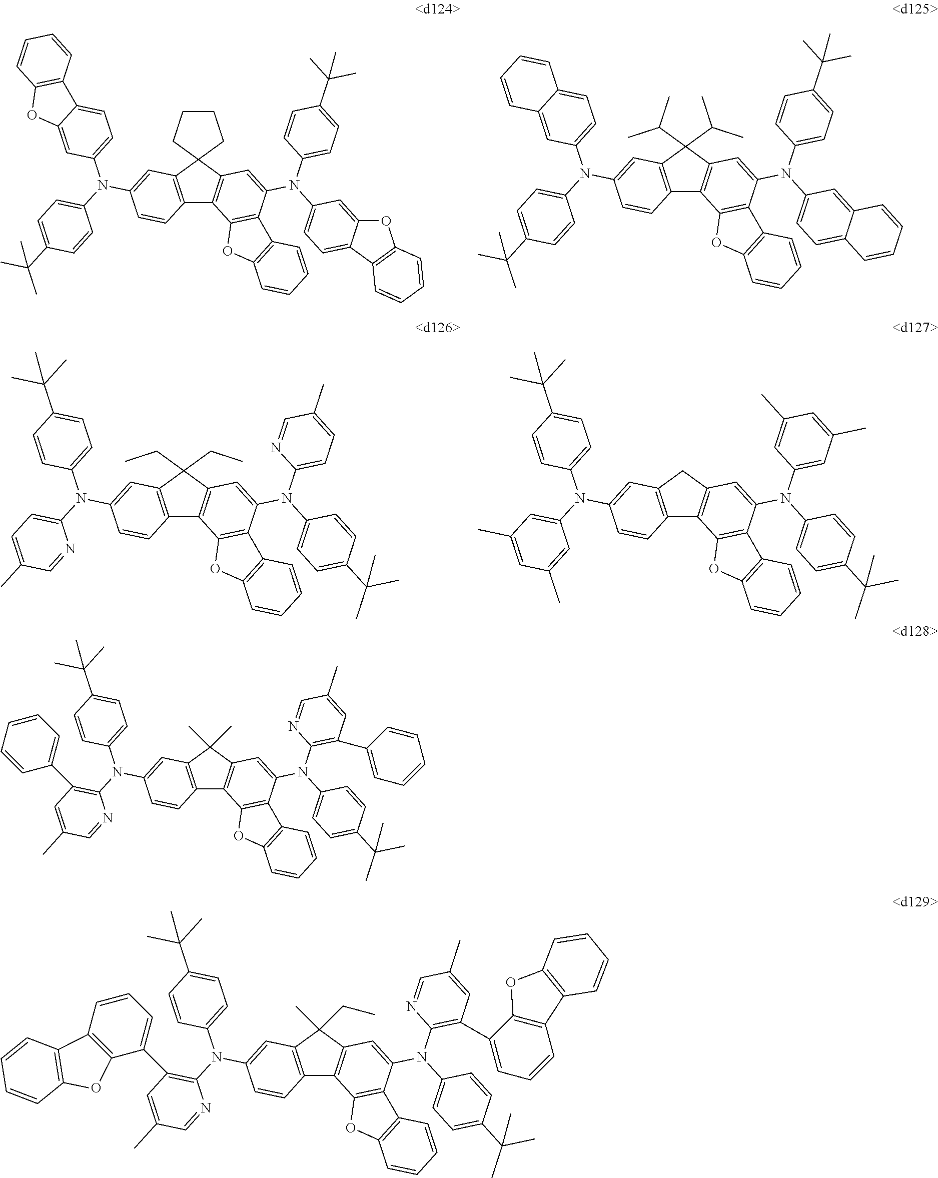

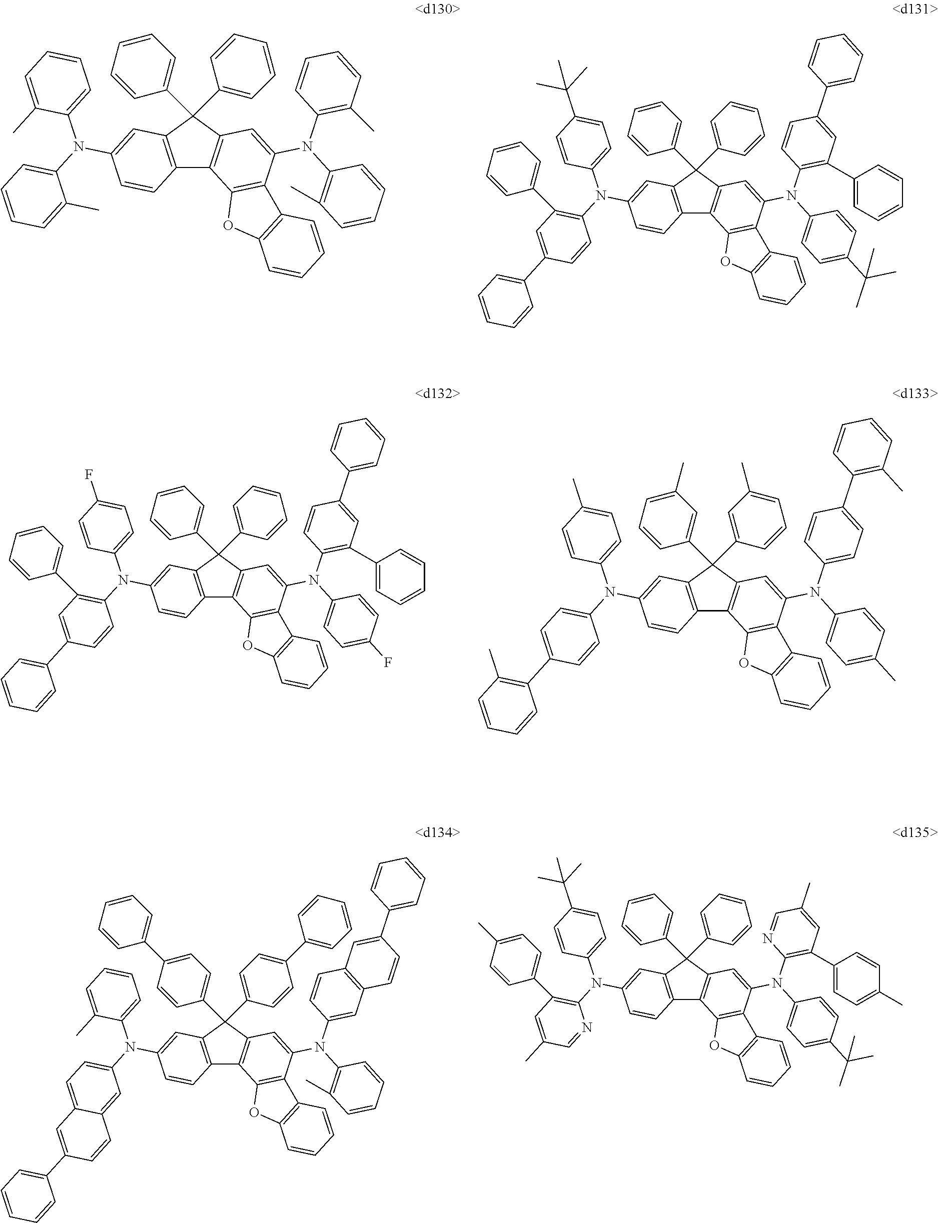

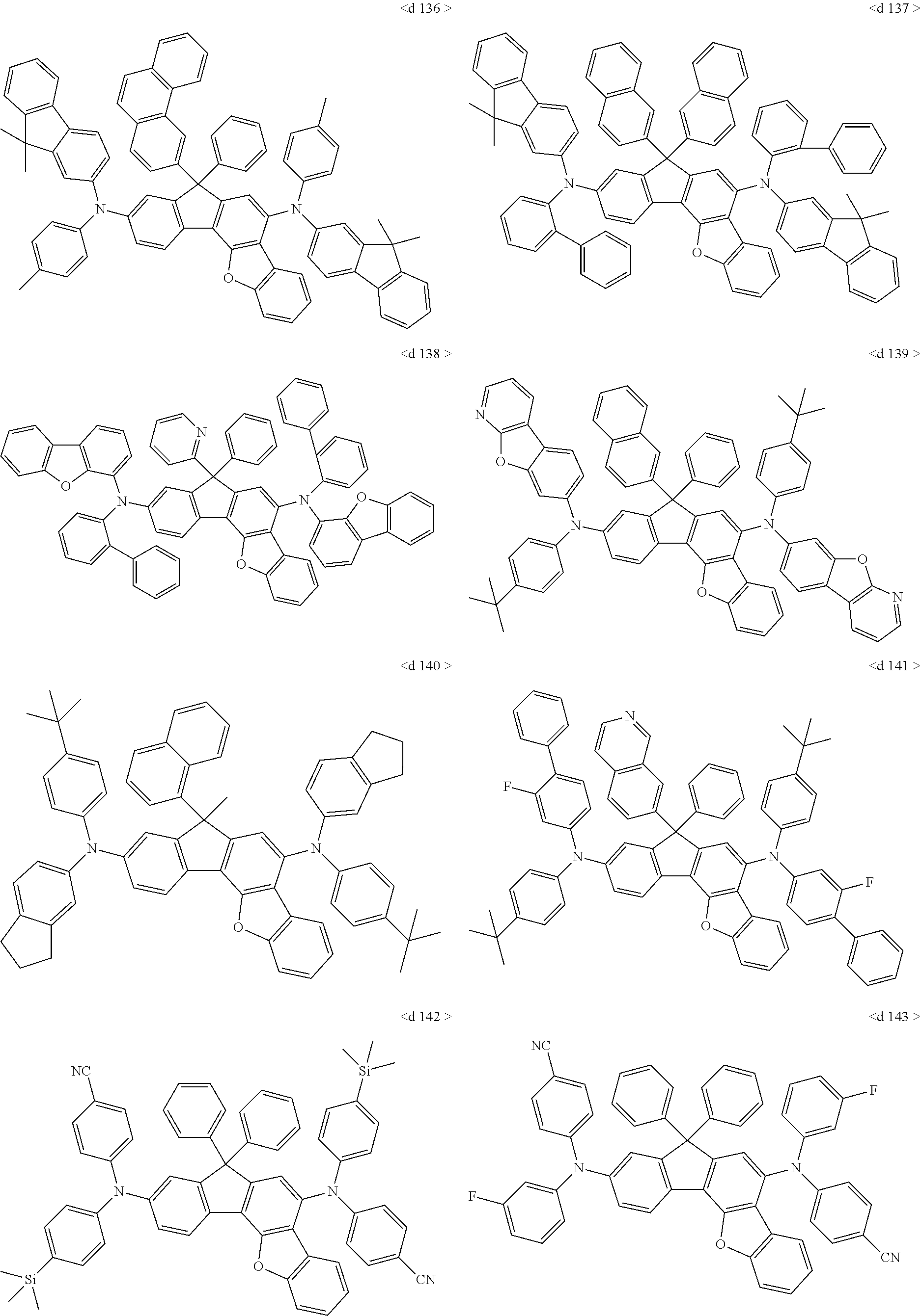

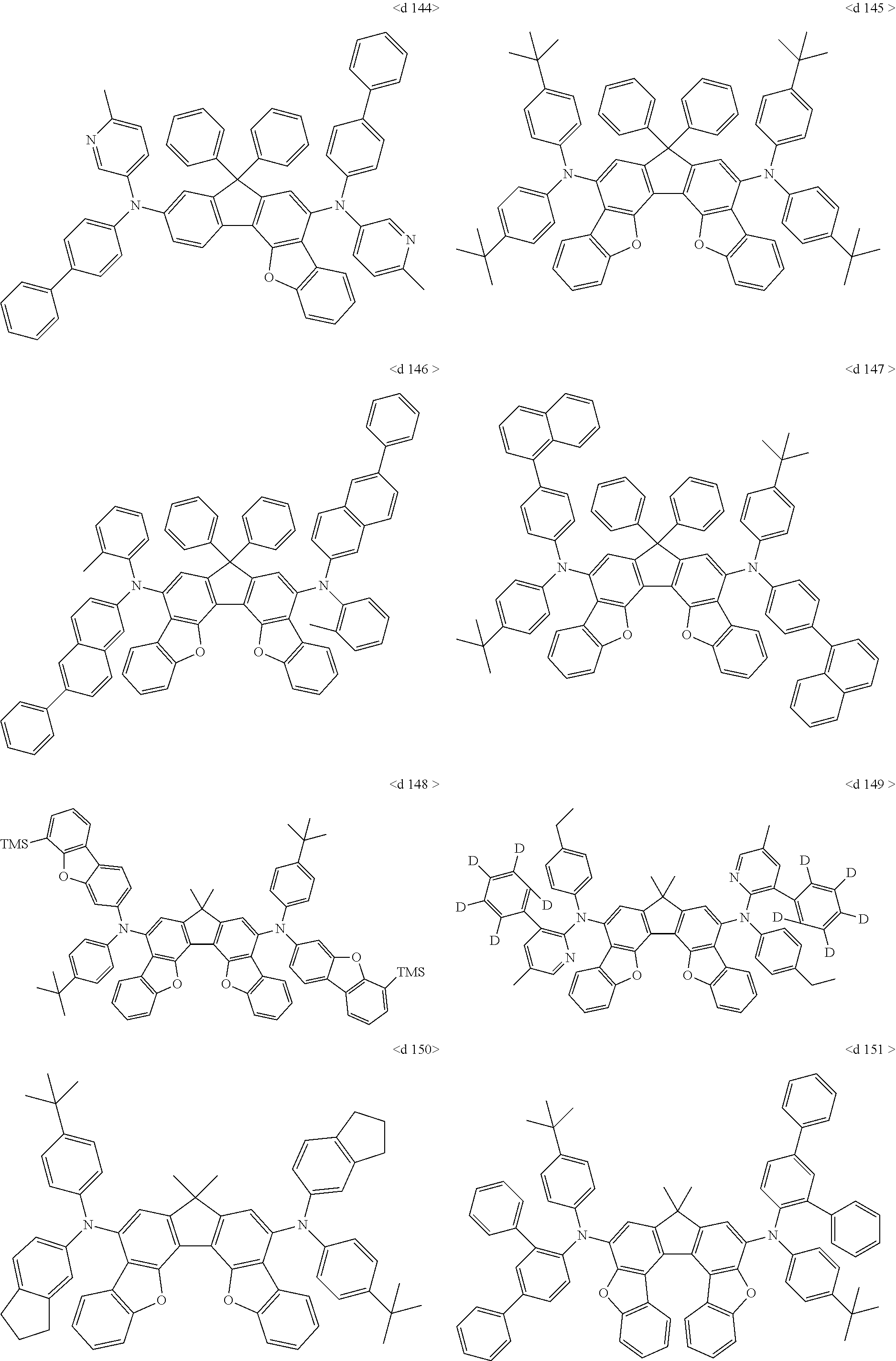

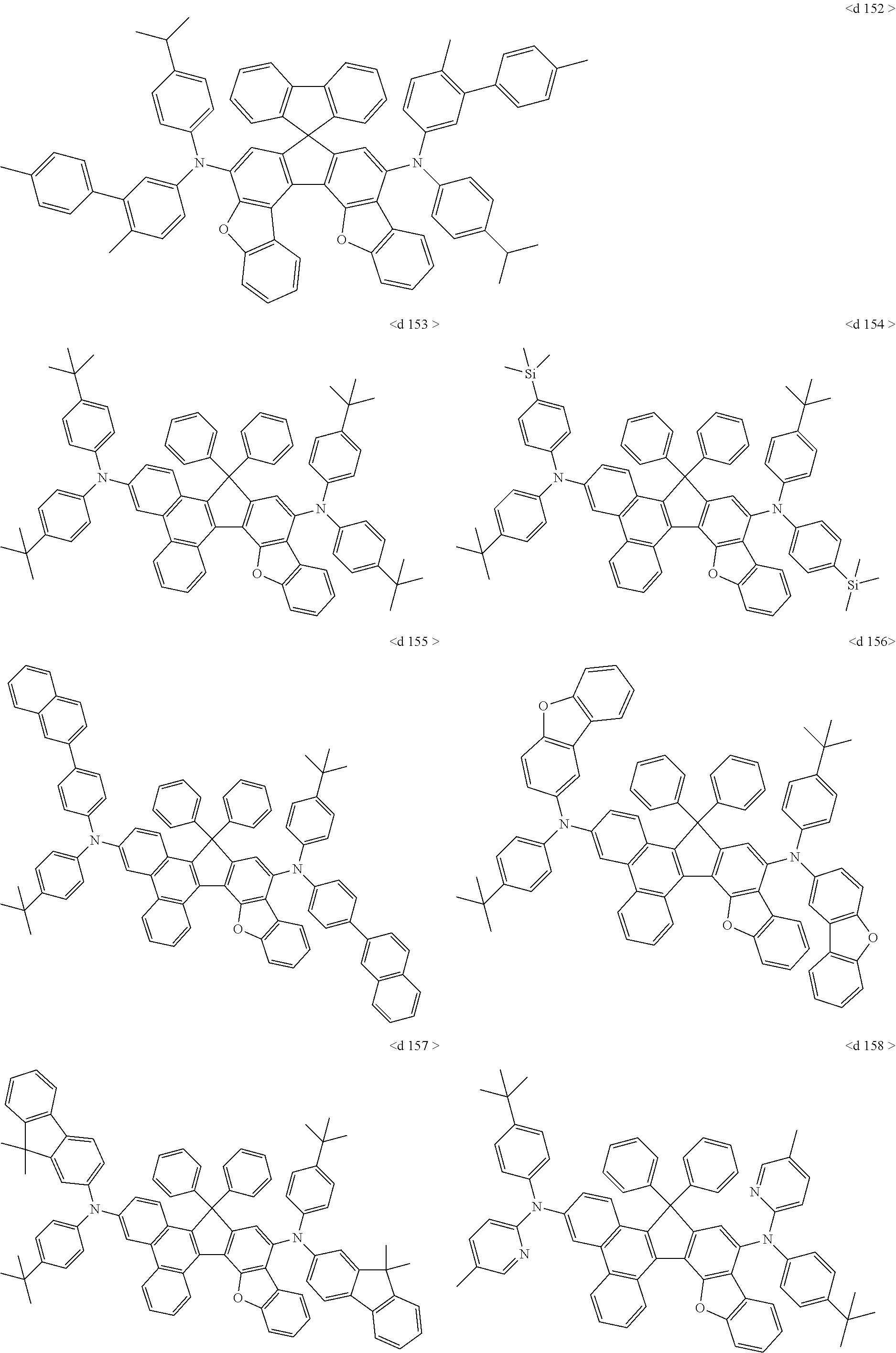

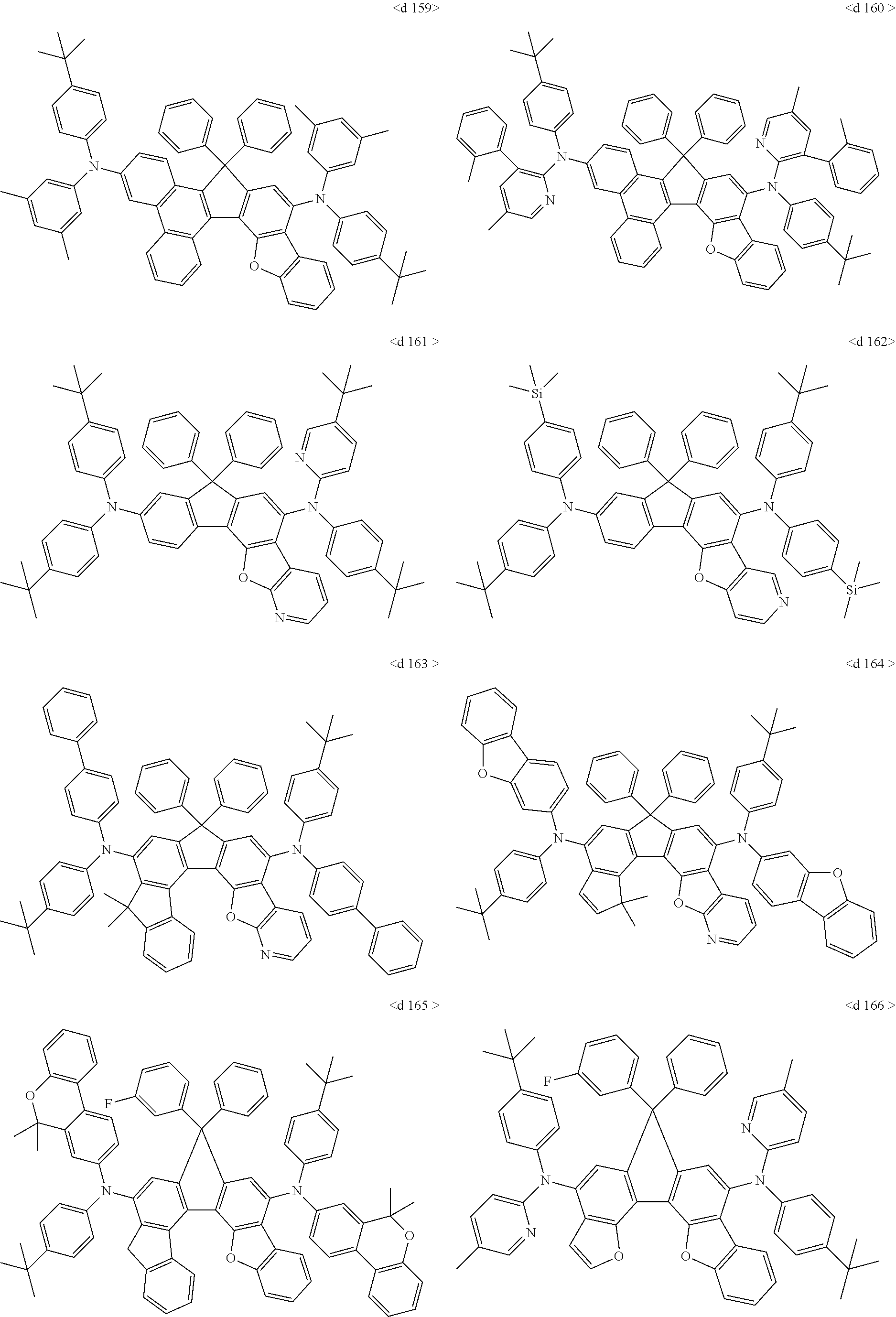

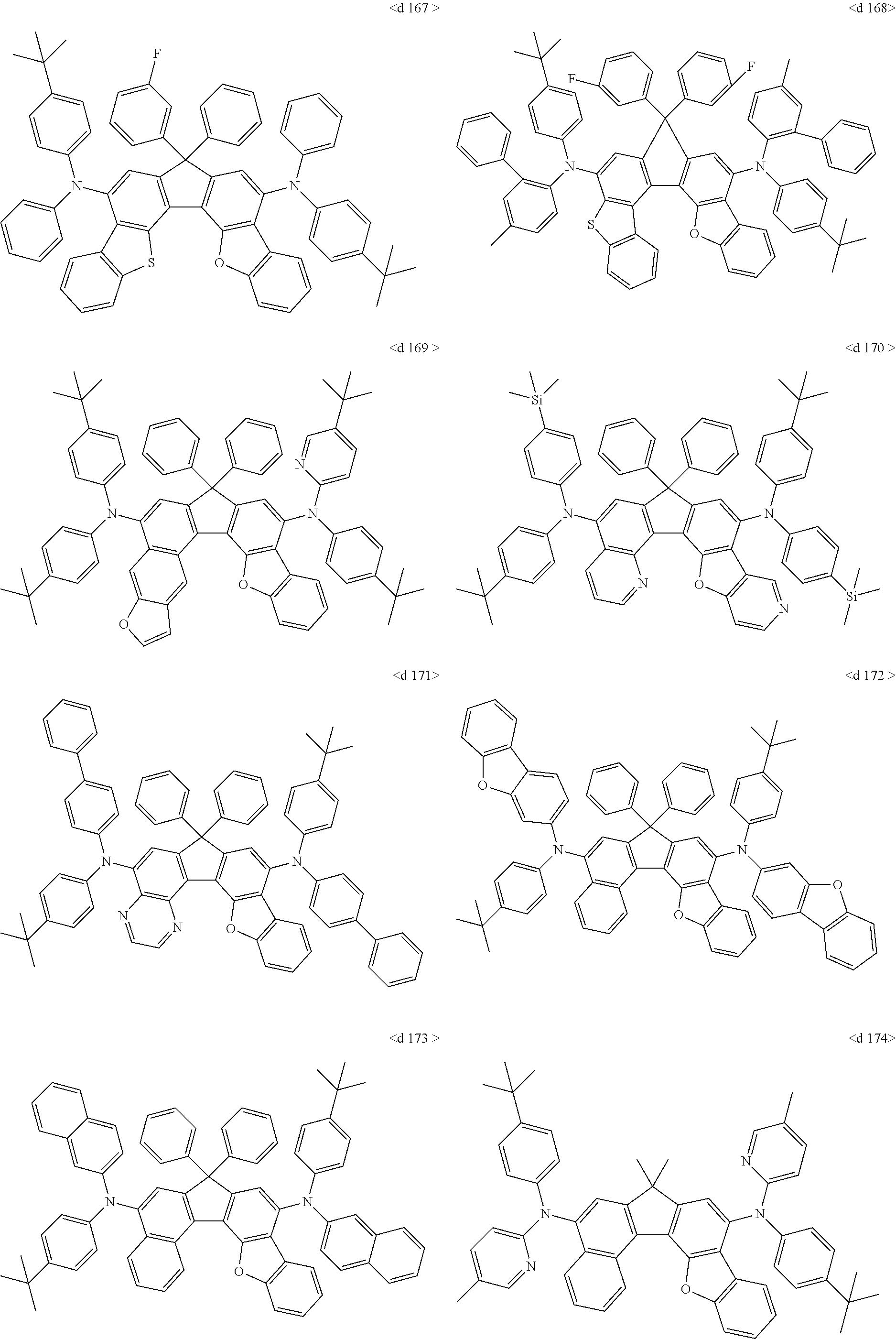

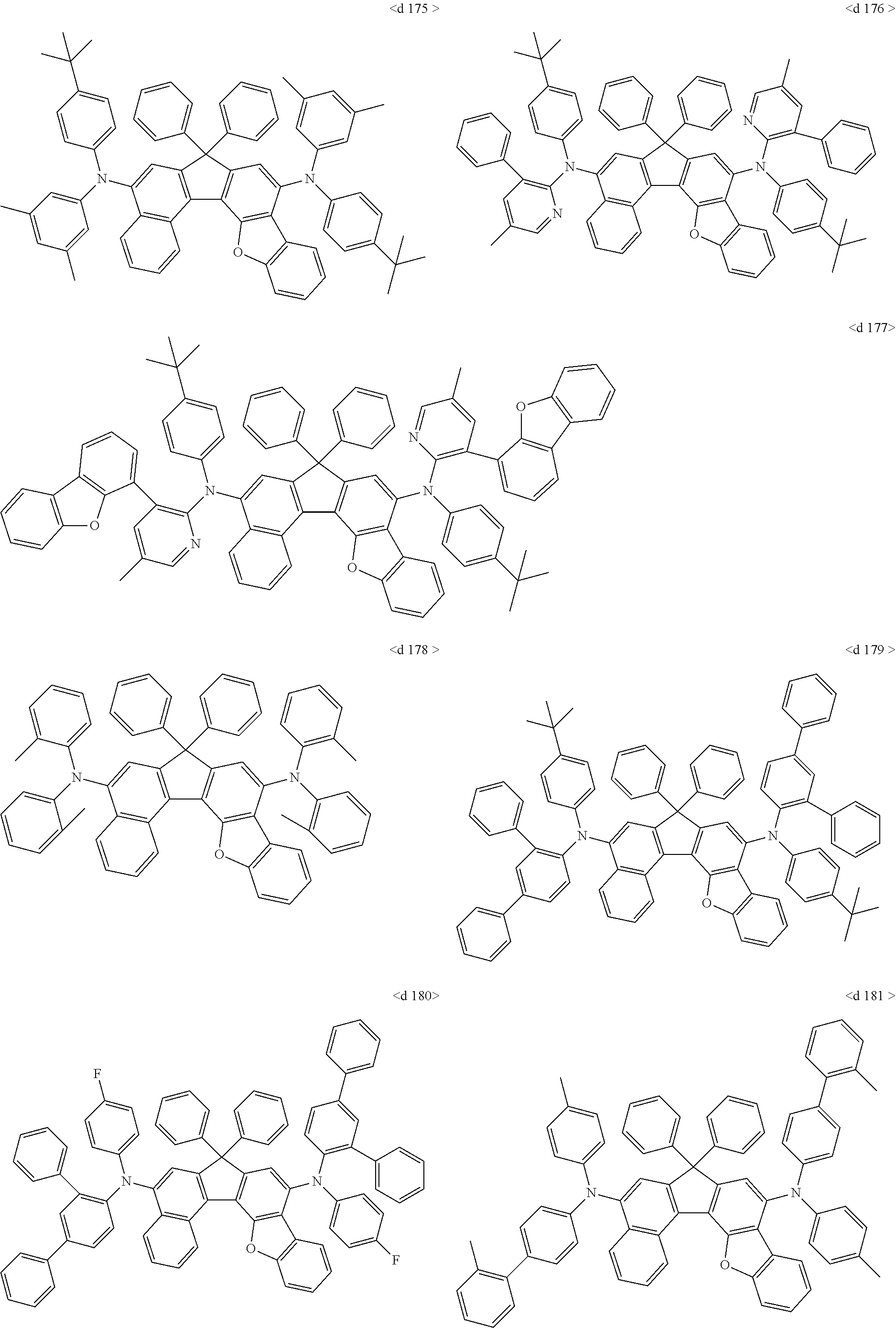

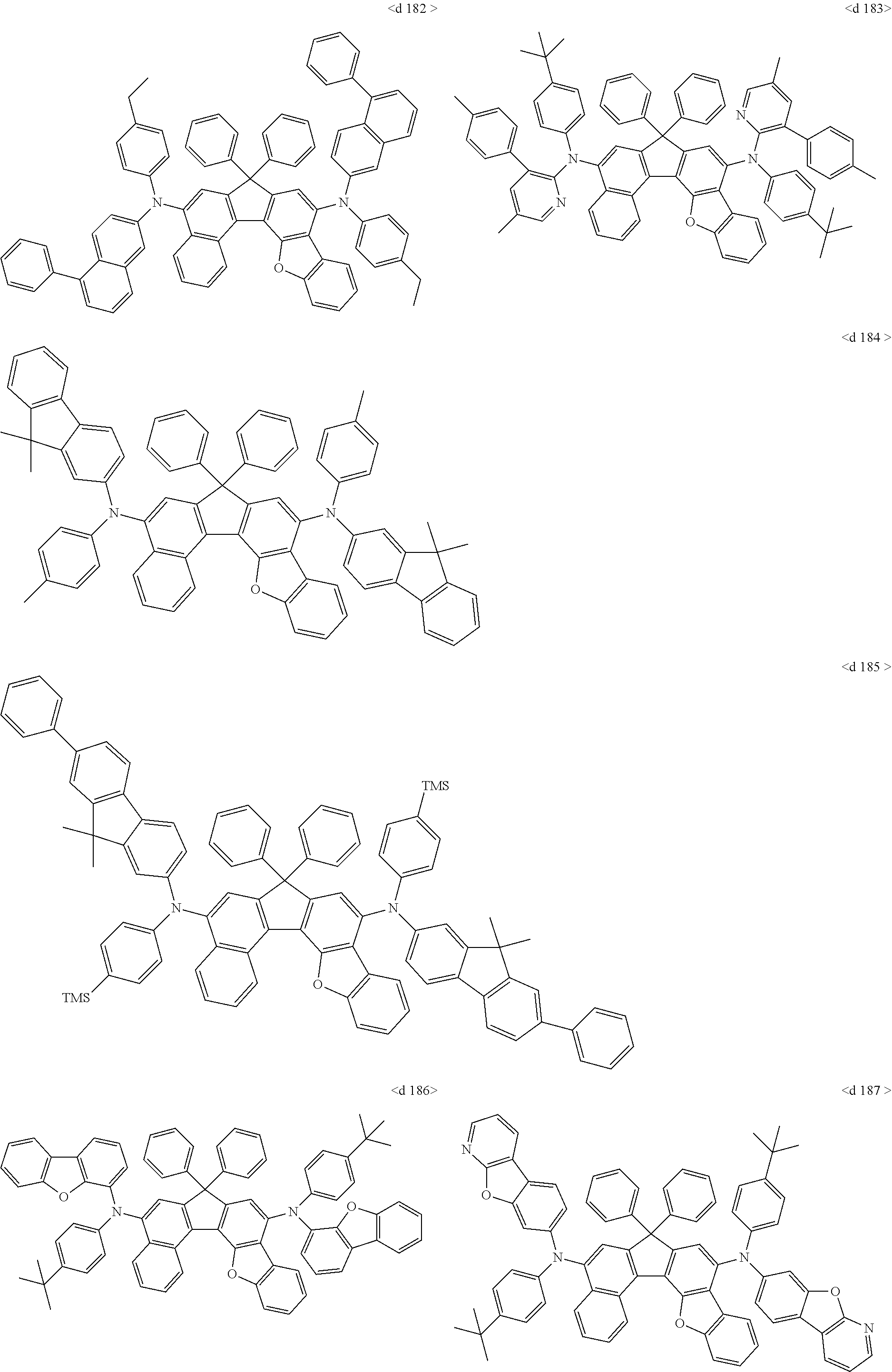

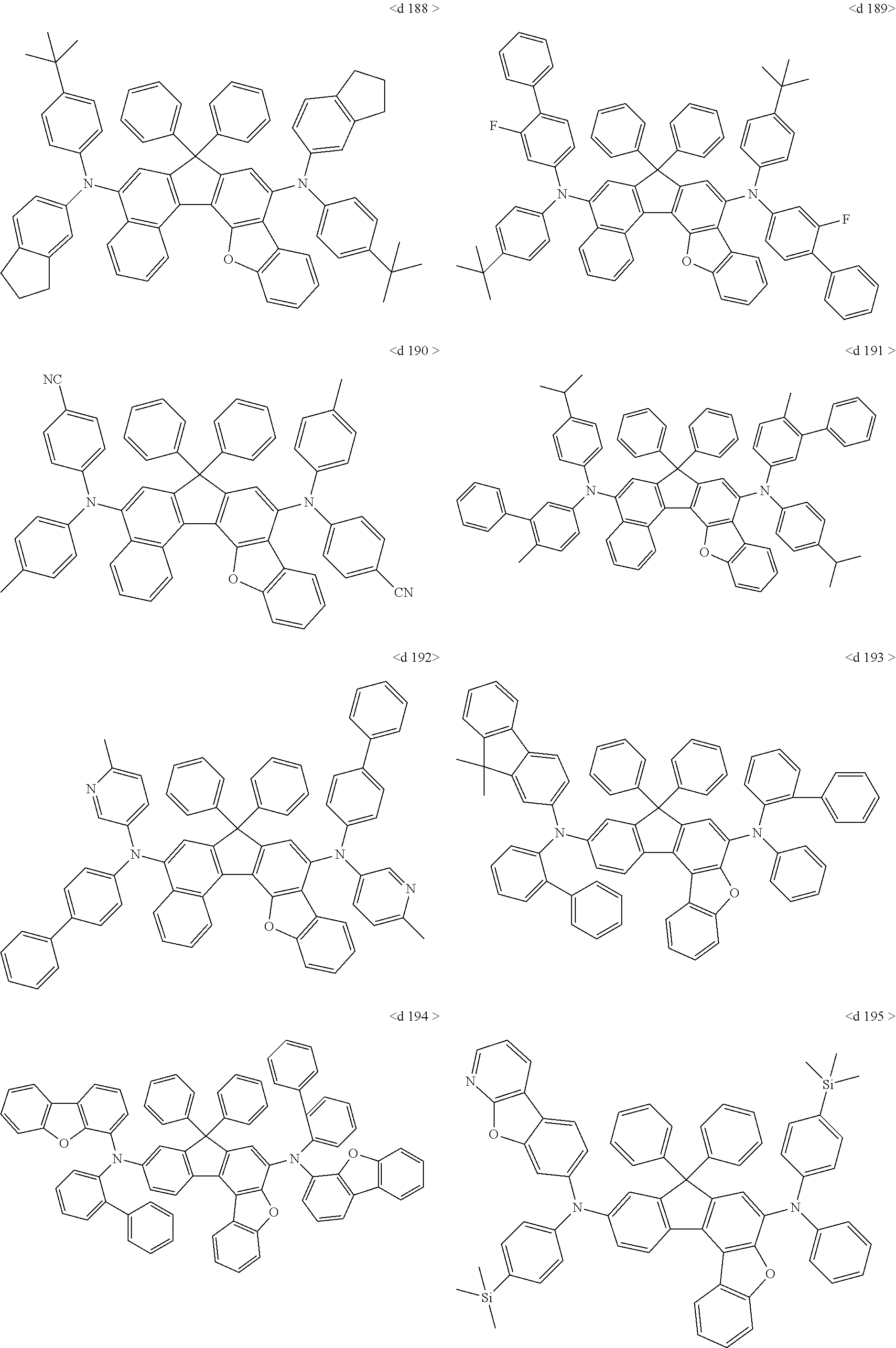

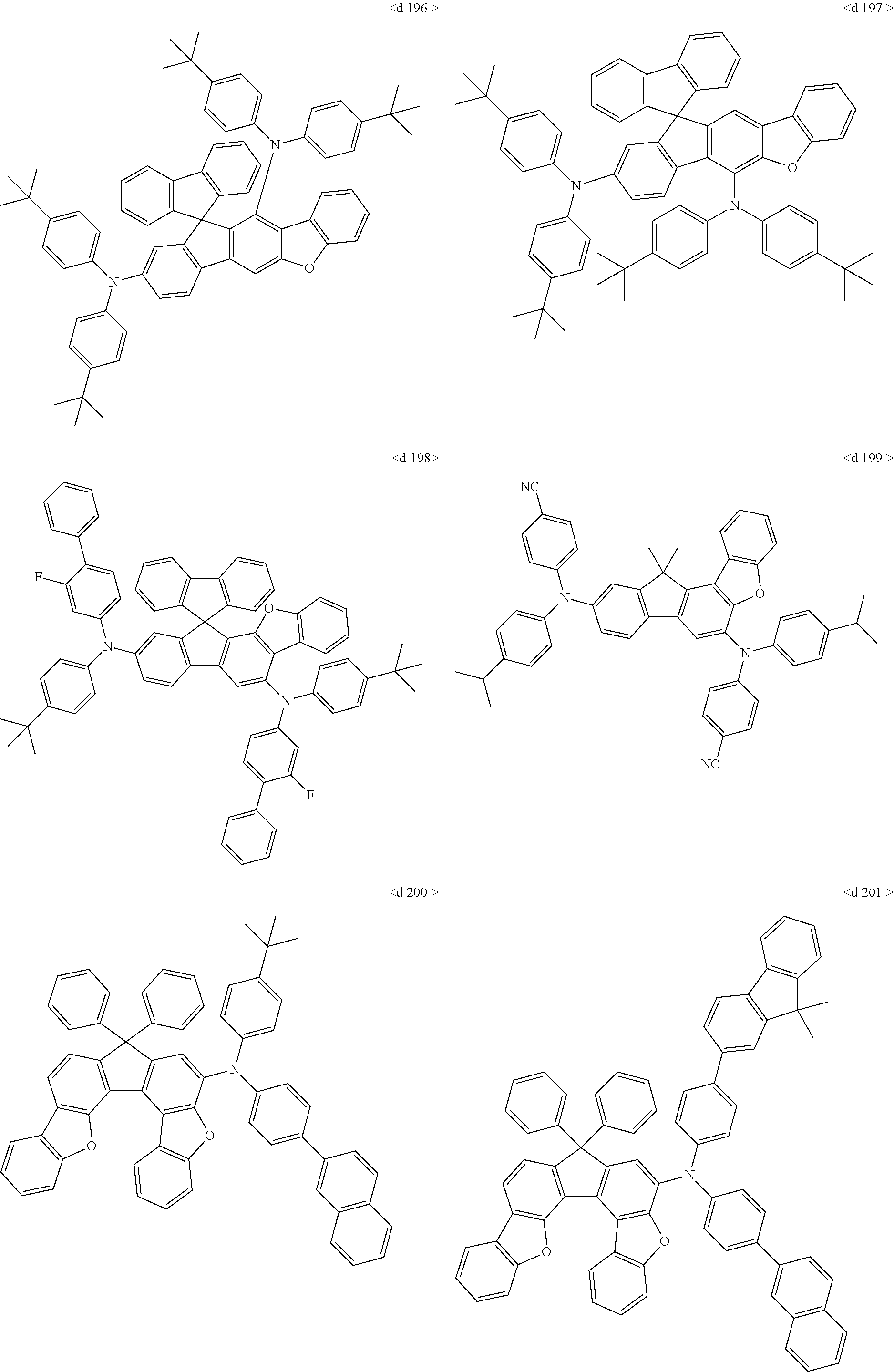

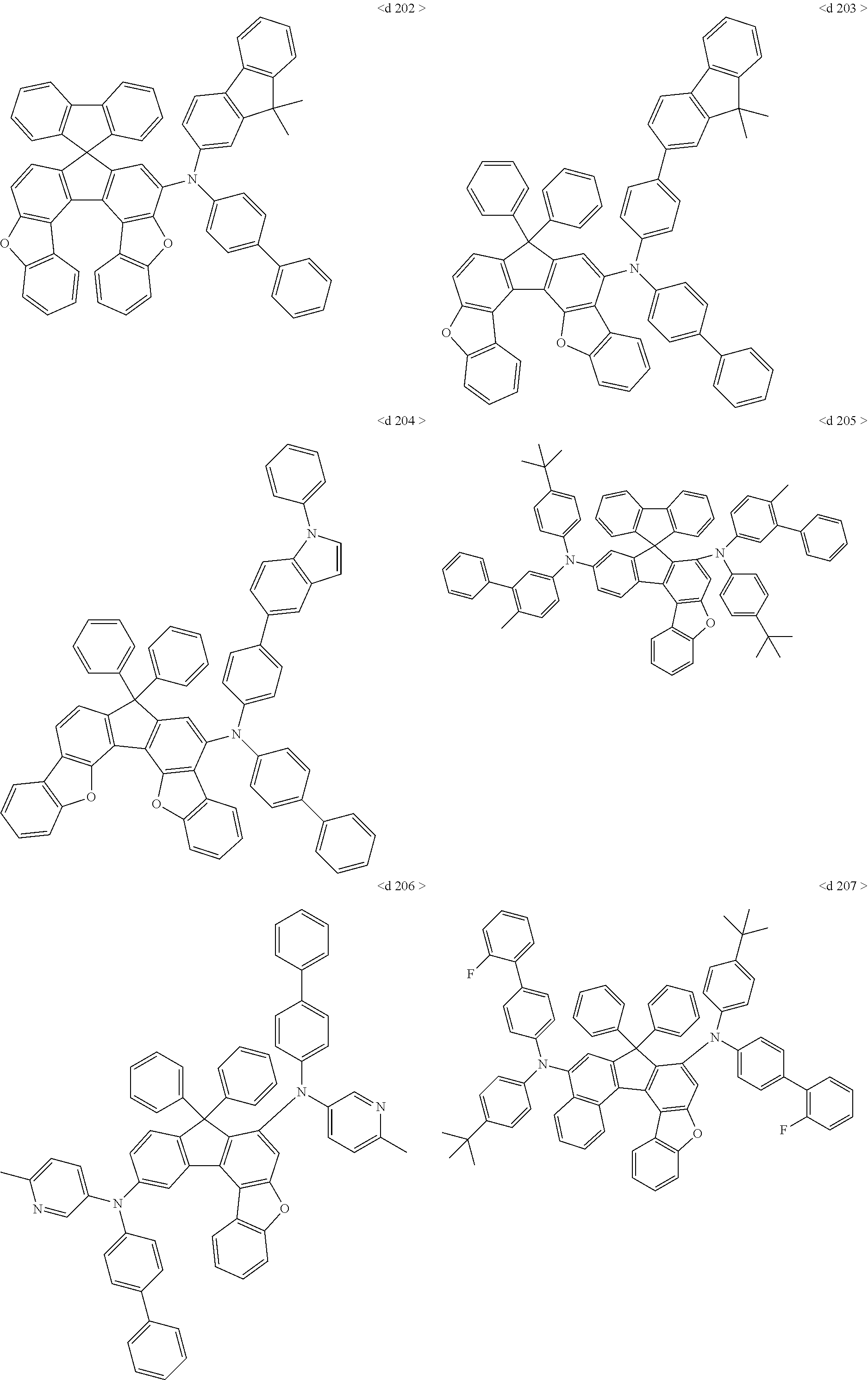

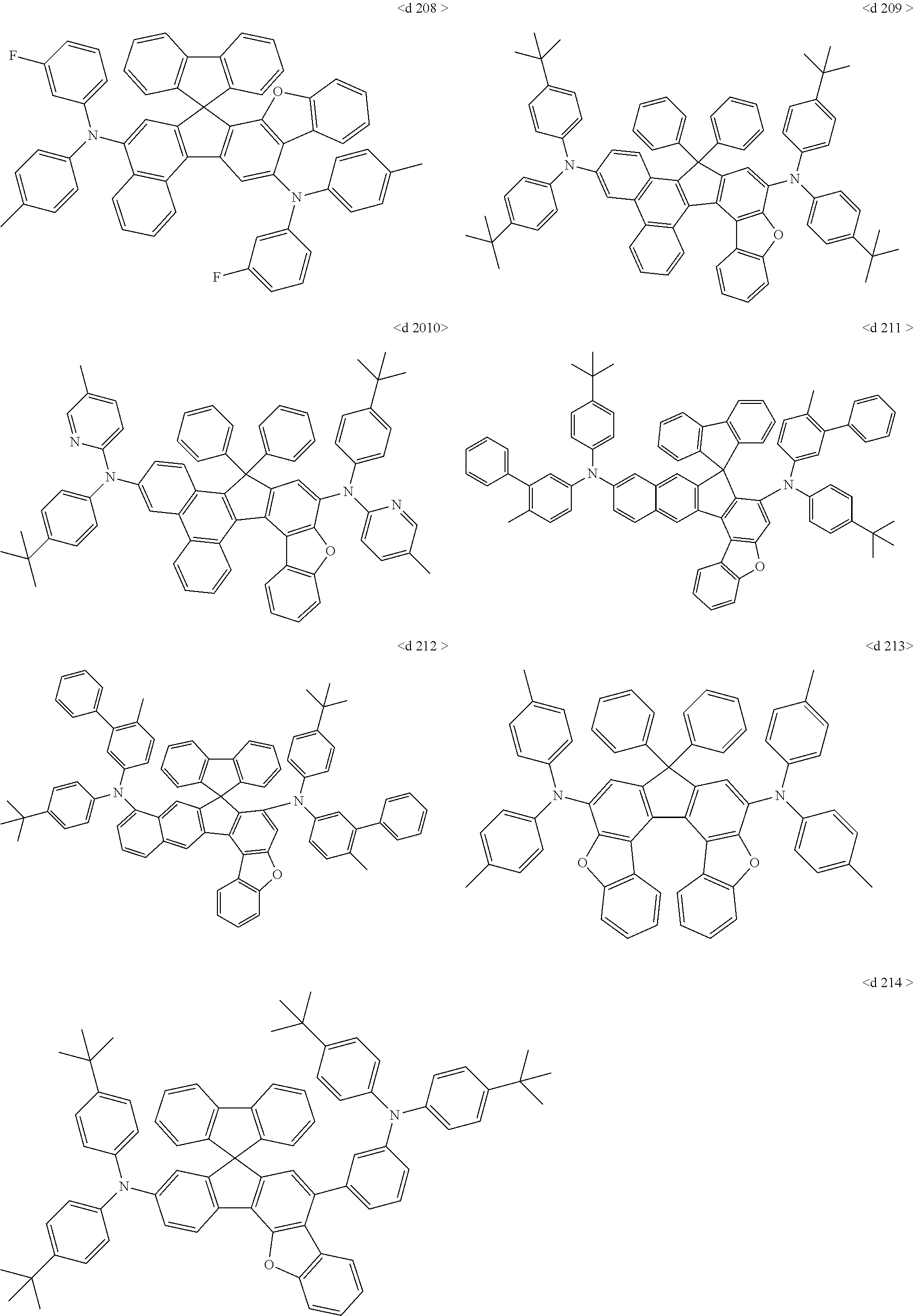

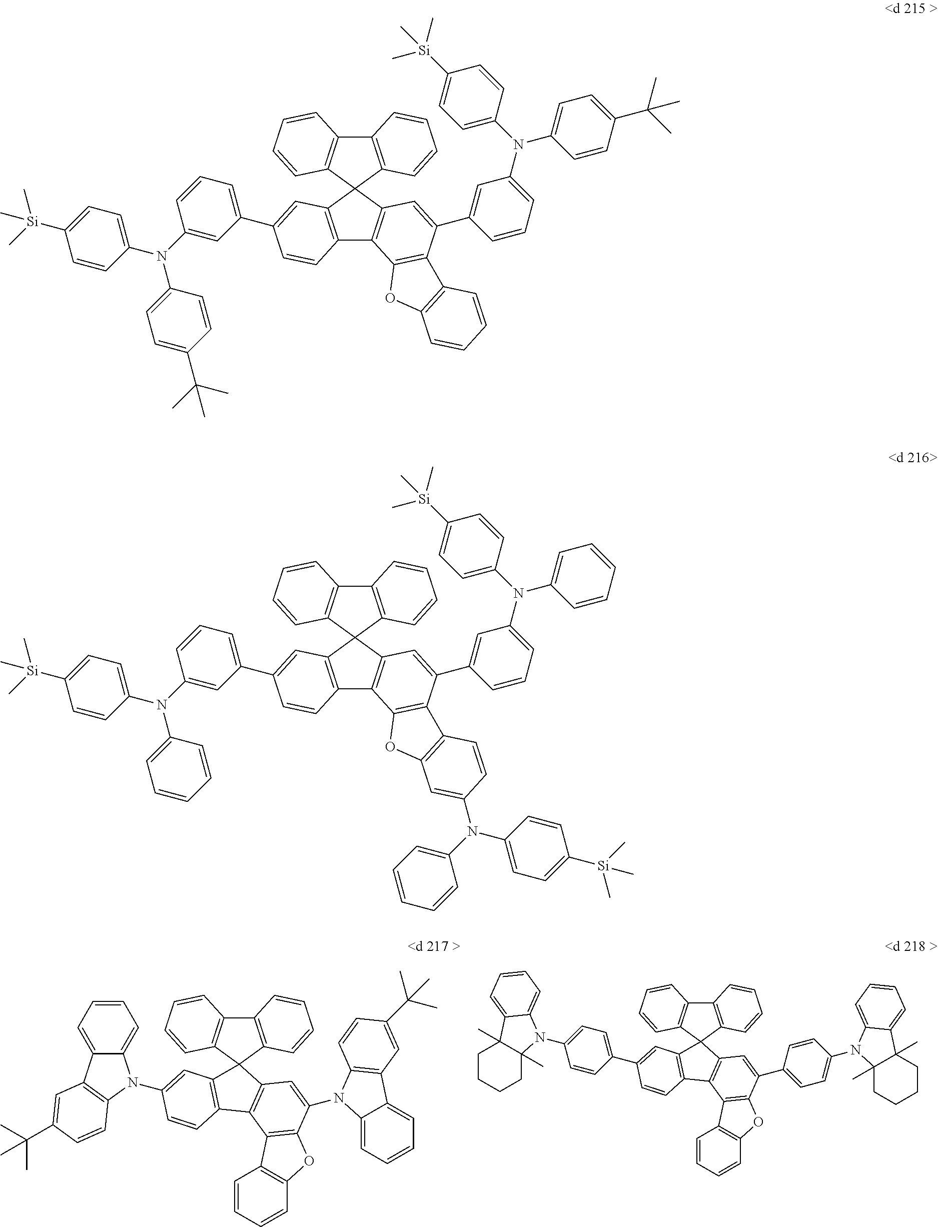

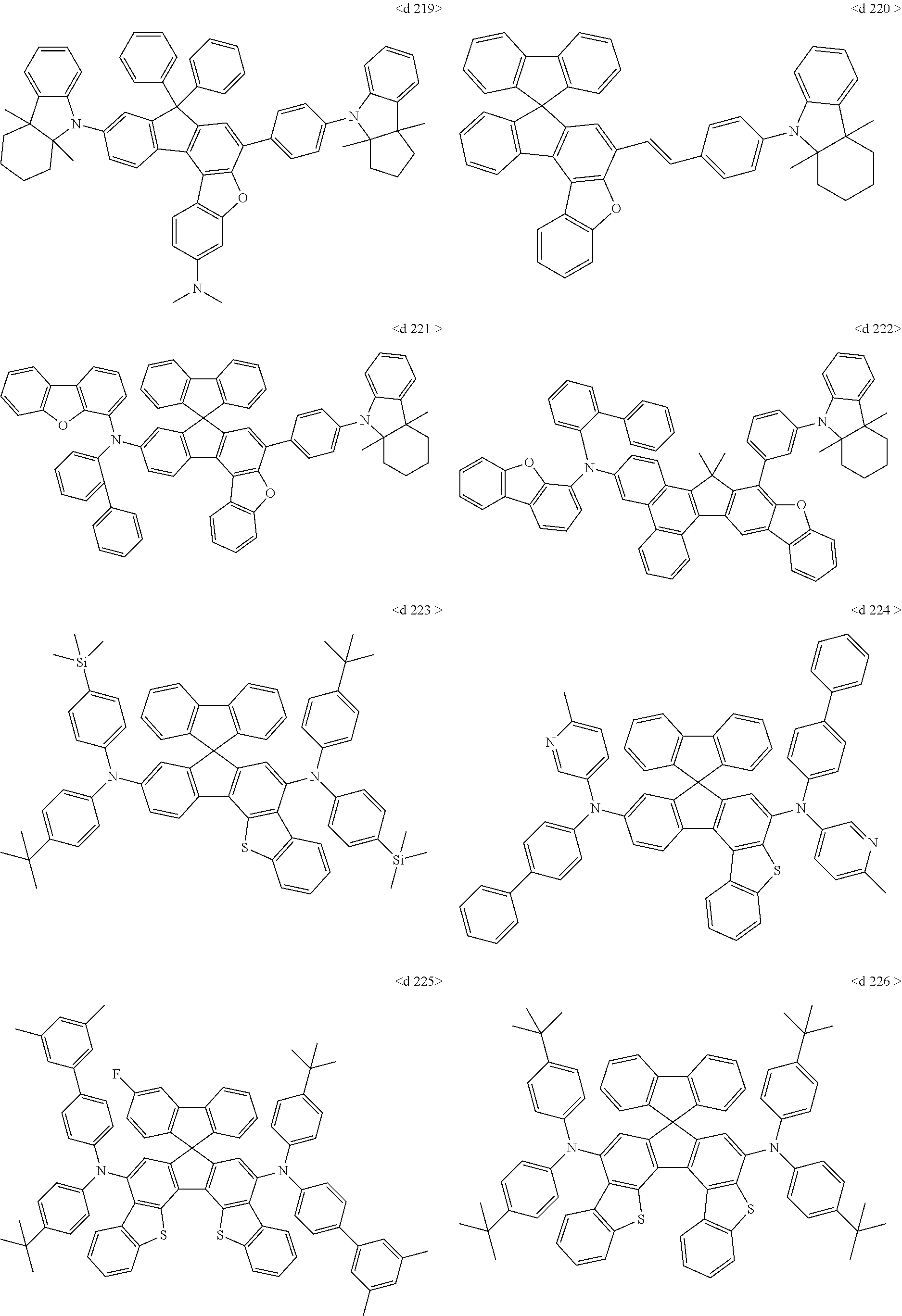

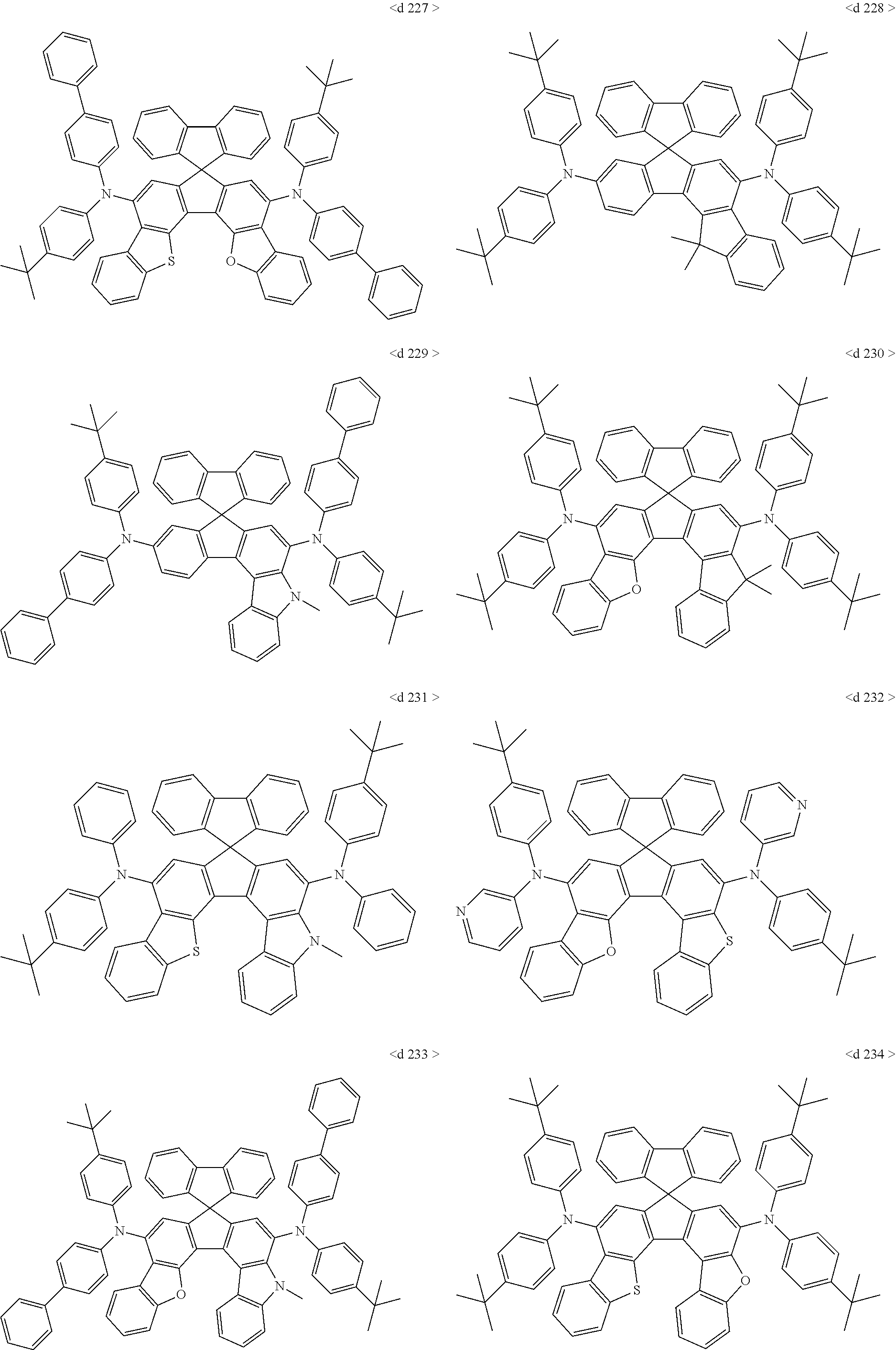

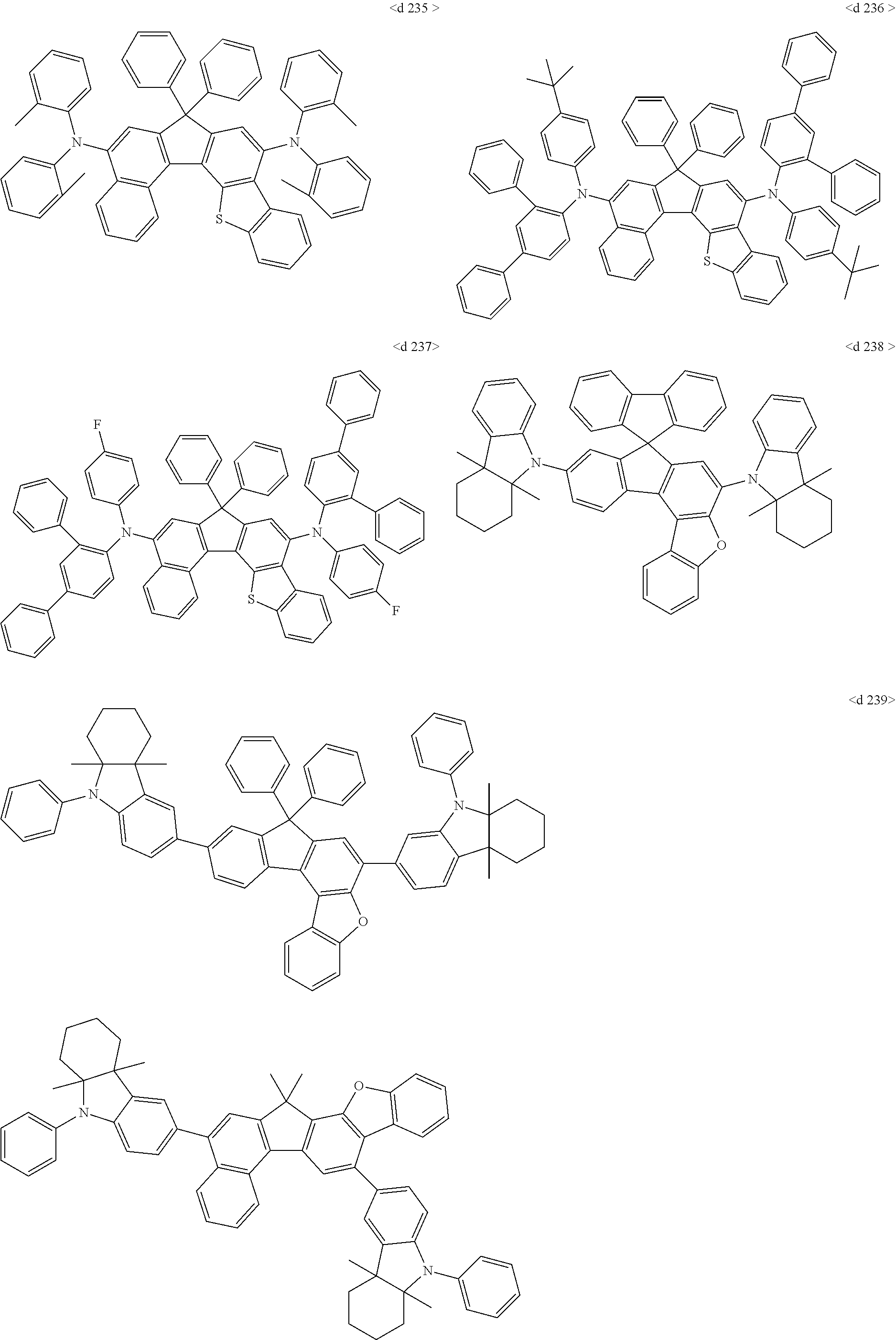

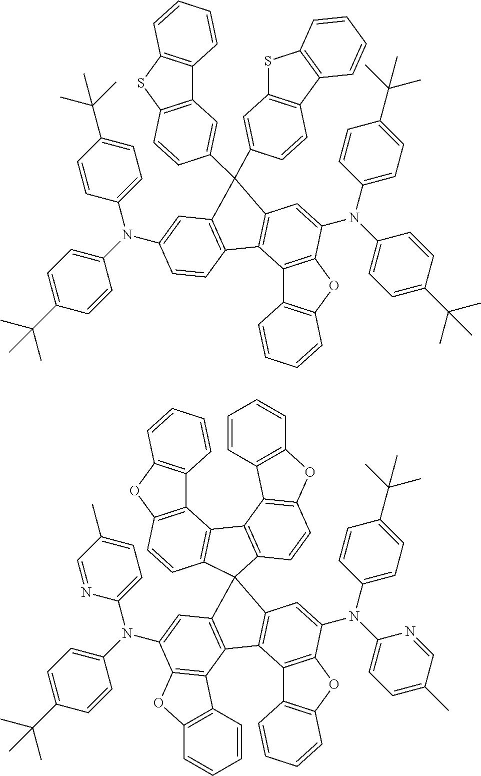

Among the dopant compounds used in the light-emitting layer, the compound represented by Chemical Formula D3 may be any one of the following <D 101> to <D 130>:

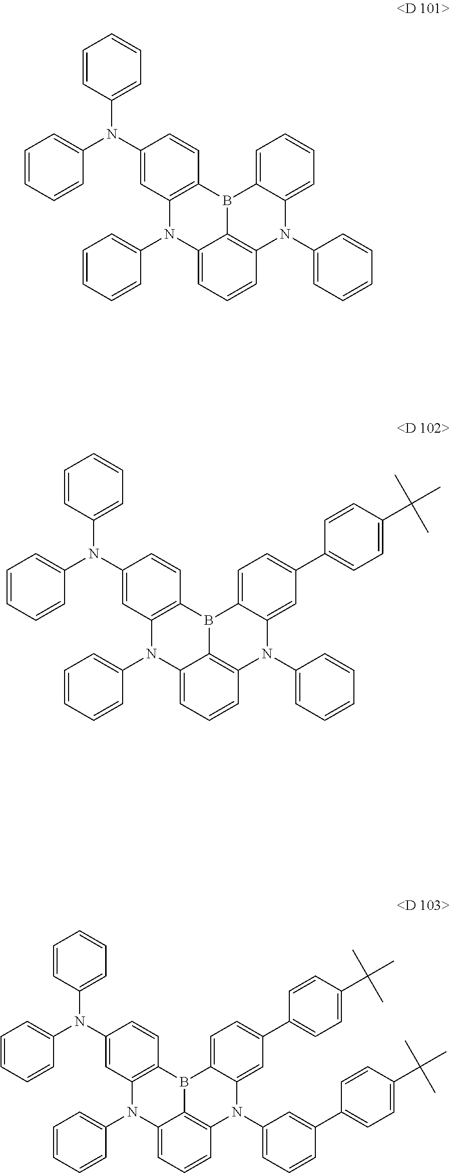 [SMILES: c1ccc(N(c2ccccc2)c2ccc3c(c2)N(c2ccccc2)c2cccc4c2B3c2ccccc2N4c2ccccc2)cc1 ; CC(C)(C)c1ccc(-c2ccc3c(c2)N(c2ccccc2)c2cccc4c2B3c2ccc(N(c3ccccc3)c3ccccc3)cc2N4c2ccccc2)cc1 ; CC(C)(C)c1ccc(-c2cccc(N3c4cc(-c5ccc(C(C)(C)C)cc5)ccc4B4c5ccc(N(c6ccccc6)c6ccccc6)cc5N(c5ccccc5)c5cccc3c54)c2)cc1]

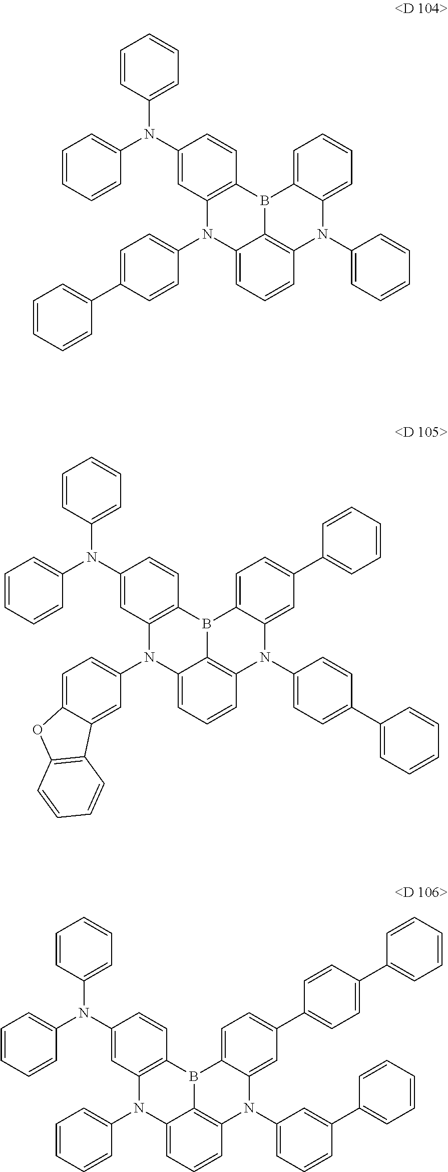 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(-c2ccc(N3c4cc(-c5ccccc5)ccc4B4c5ccc(N(c6ccccc6)c6ccccc6)cc5N(c5ccc6oc7ccccc7c6c5)c5cccc3c54)cc2)cc1 ; c1ccc(-c2ccc(-c3ccc4c(c3)N(c3cccc(-c5ccccc5)c3)c3cccc5c3B4c3ccc(N(c4ccccc4)c4ccccc4)cc3N5c3ccccc3)cc2)cc1]

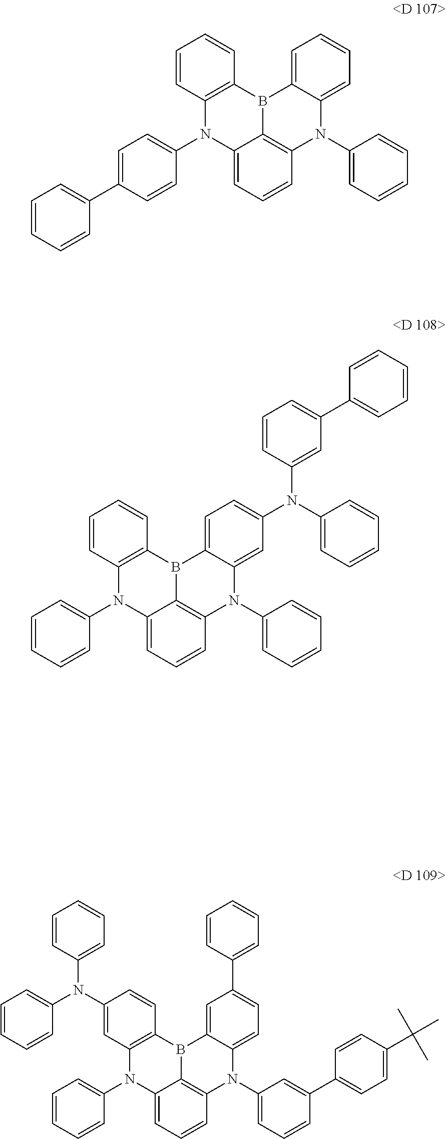 [SMILES: c1ccc(-c2ccc(N3c4ccccc4B4c5ccccc5N(c5ccccc5)c5cccc3c54)cc2)cc1 ; c1ccc(-c2cccc(N(c3ccccc3)c3ccc4c(c3)N(c3ccccc3)c3cccc5c3B4c3ccccc3N5c3ccccc3)c2)cc1 ; CC(C)(C)c1ccc(-c2cccc(N3c4ccc(-c5ccccc5)cc4B4c5ccc(N(c6ccccc6)c6ccccc6)cc5N(c5ccccc5)c5cccc3c54)c2)cc1]

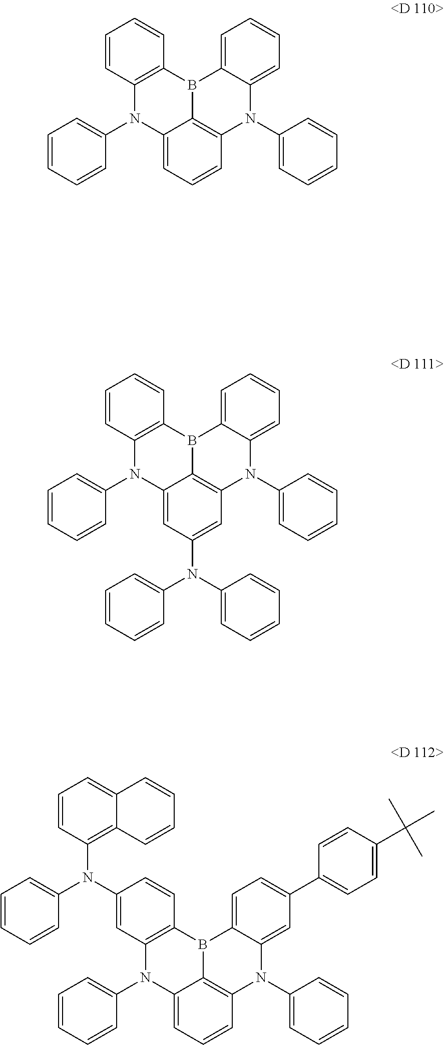 [SMILES: c1ccc(N2c3ccccc3B3c4ccccc4N(c4ccccc4)c4cccc2c43)cc1 ; c1ccc(N(c2ccccc2)c2cc3c4c(c2)N(c2ccccc2)c2ccccc2B4c2ccccc2N3c2ccccc2)cc1 ; CC(C)(C)c1ccc(-c2ccc3c(c2)N(c2ccccc2)c2cccc4c2B3c2ccc(N(c3ccccc3)c3cccc5ccccc35)cc2N4c2ccccc2)cc1]

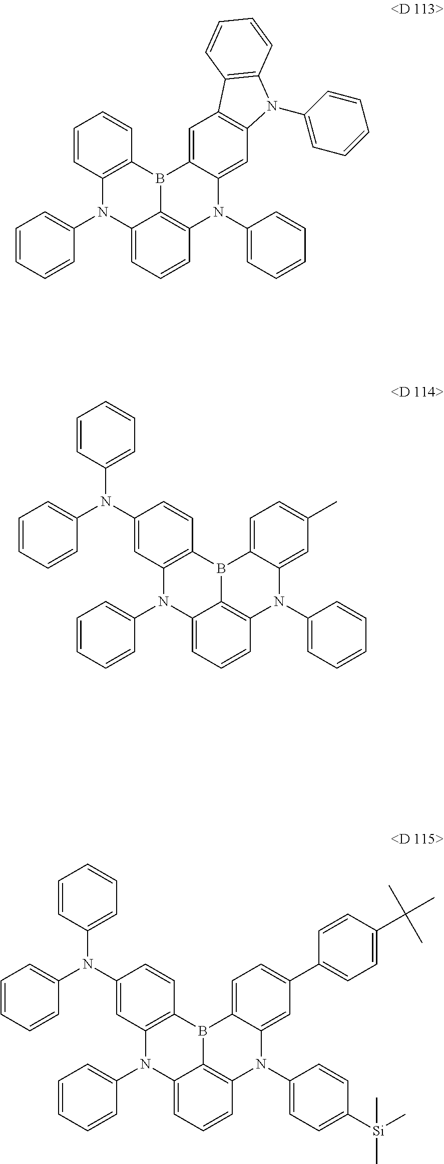 [SMILES: c1ccc(N2c3ccccc3B3c4cc5c6ccccc6n(-c6ccccc6)c5cc4N(c4ccccc4)c4cccc2c43)cc1 ; Cc1ccc2c(c1)N(c1ccccc1)c1cccc3c1B2c1ccc(N(c2ccccc2)c2ccccc2)cc1N3c1ccccc1 ; CC(C)(C)c1ccc(-c2ccc3c(c2)N(c2ccc([Si](C)(C)C)cc2)c2cccc4c2B3c2ccc(N(c3ccccc3)c3ccccc3)cc2N4c2ccccc2)cc1]

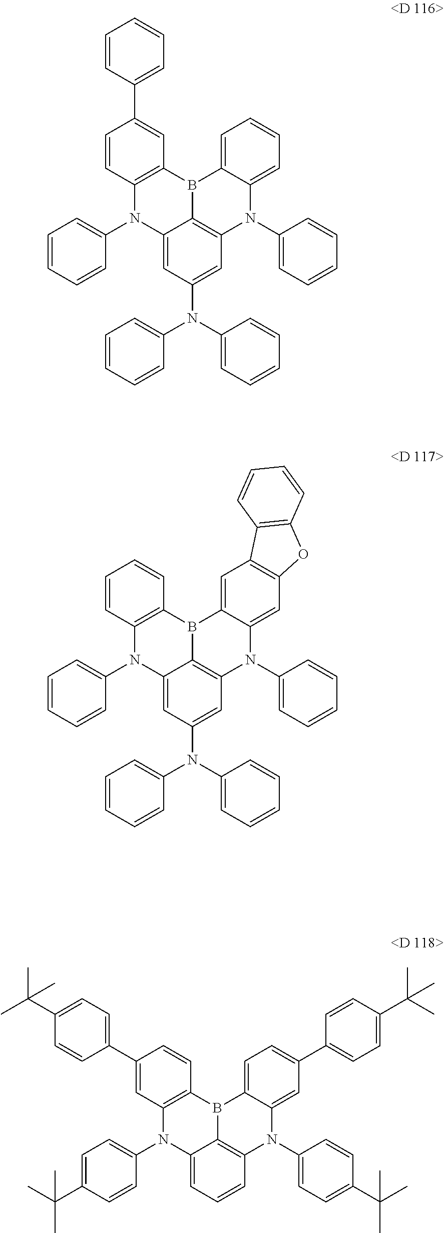 [SMILES: c1ccc(-c2ccc3c(c2)B2c4ccccc4N(c4ccccc4)c4cc(N(c5ccccc5)c5ccccc5)cc(c42)N3c2ccccc2)cc1 ; c1ccc(N(c2ccccc2)c2cc3c4c(c2)N(c2ccccc2)c2cc5oc6ccccc6c5cc2B4c2ccccc2N3c2ccccc2)cc1 ; CC(C)(C)c1ccc(-c2ccc3c(c2)N(c2ccc(C(C)(C)C)cc2)c2cccc4c2B3c2ccc(-c3ccc(C(C)(C)C)cc3)cc2N4c2ccc(C(C)(C)C)cc2)cc1]

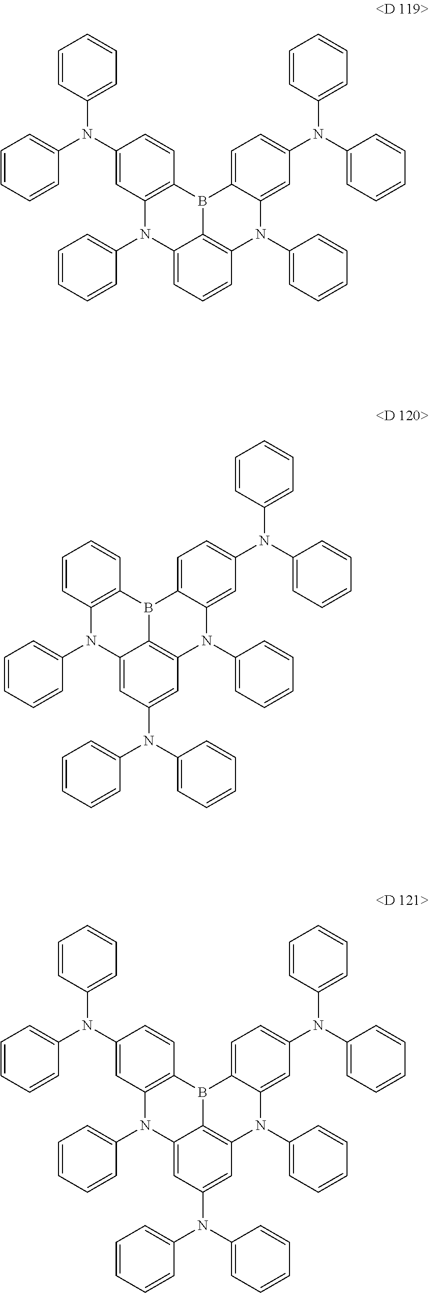 [SMILES: c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cc(N(c4ccccc4)c4ccccc4)cc4c2B3c2ccccc2N4c2ccccc2)cc1 ; c1ccc(N(c2ccccc2)c2ccc3c(c2)N(c2ccccc2)c2cc(N(c4ccccc4)c4ccccc4)cc4c2B3c2ccc(N(c3ccccc3)c3ccccc3)cc2N4c2ccccc2)cc1]

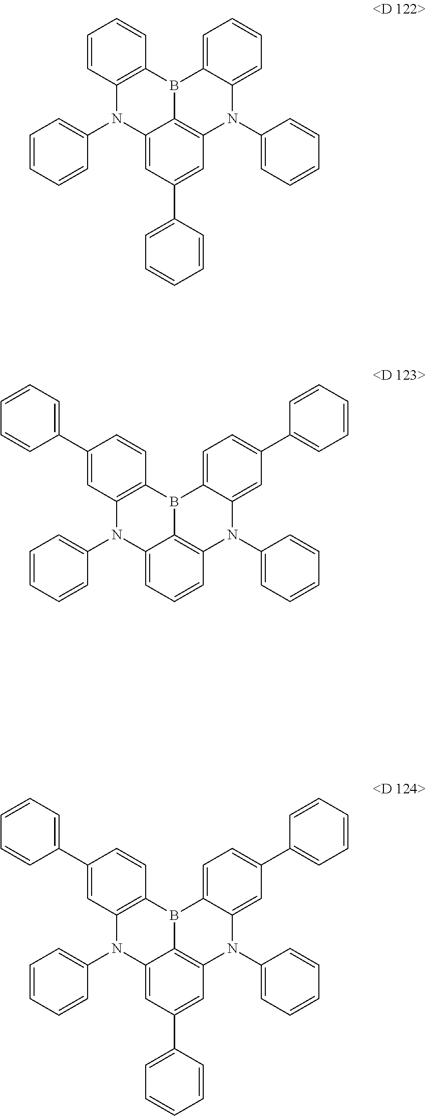 [SMILES: c1ccc(-c2cc3c4c(c2)N(c2ccccc2)c2ccccc2B4c2ccccc2N3c2ccccc2)cc1 ; c1ccc(-c2ccc3c(c2)N(c2ccccc2)c2cccc4c2B3c2ccc(-c3ccccc3)cc2N4c2ccccc2)cc1 ; c1ccc(-c2ccc3c(c2)N(c2ccccc2)c2cc(-c4ccccc4)cc4c2B3c2ccc(-c3ccccc3)cc2N4c2ccccc2)cc1]

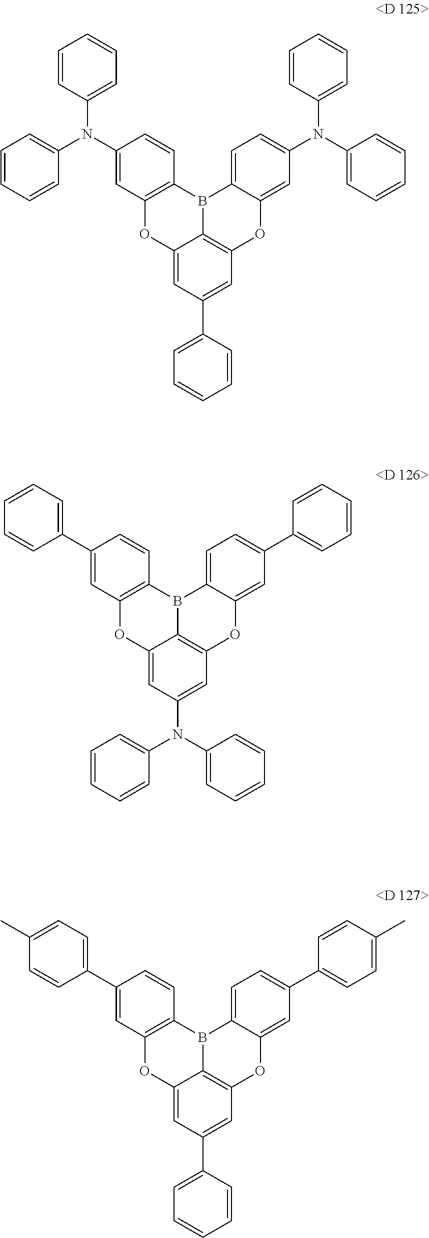

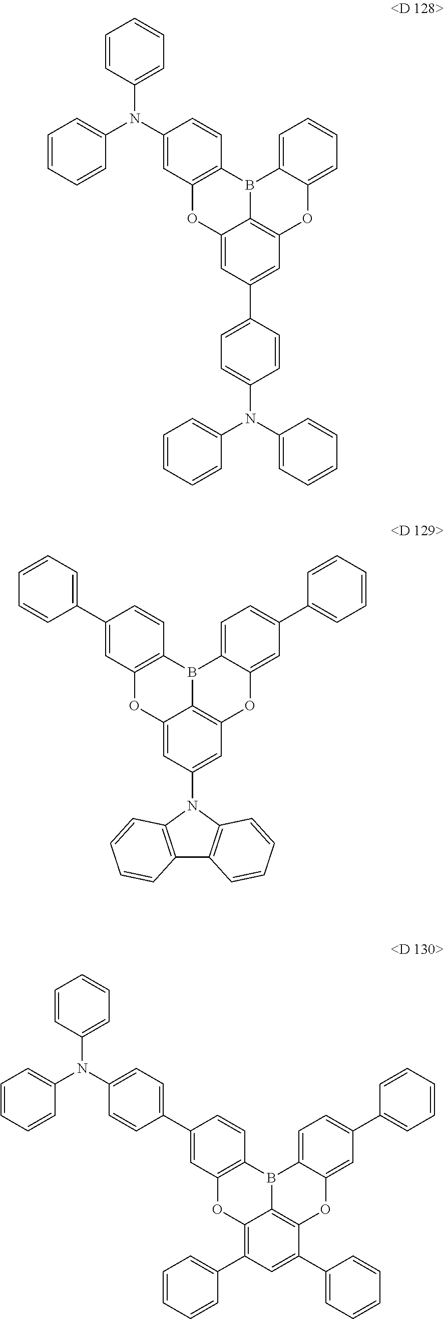 [SMILES: c1ccc(N(c2ccccc2)c2ccc(-c3cc4c5c(c3)Oc3cc(N(c6ccccc6)c6ccccc6)ccc3B5c3ccccc3O4)cc2)cc1 ; c1ccc(-c2ccc3c(c2)Oc2cc(-n4c5ccccc5c5ccccc54)cc4c2B3c2ccc(-c3ccccc3)cc2O4)cc1 ; c1ccc(-c2ccc3c(c2)Oc2c(-c4ccccc4)cc(-c4ccccc4)c4c2B3c2ccc(-c3ccc(N(c5ccccc5)c5ccccc5)cc3)cc2O4)cc1]

Examples of the compound represented by any one of [Chemical Formula D4], [Chemical Formula D5], and [Chemical Formula D8] to [Chemical Formula D10] include the compounds of the following [D 201] to [D 476]:

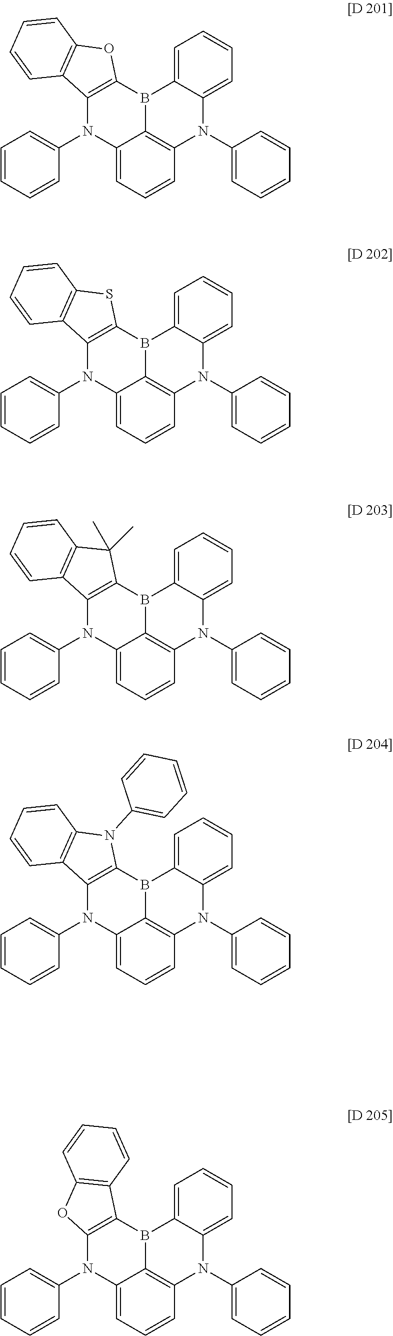 [SMILES: c1ccc(N2c3ccccc3B3c4oc5ccccc5c4N(c4ccccc4)c4cccc2c43)cc1 ; c1ccc(N2c3ccccc3B3c4sc5ccccc5c4N(c4ccccc4)c4cccc2c43)cc1 ; CC1(C)C2=C(c3ccccc31)N(c1ccccc1)c1cccc3c1B2c1ccccc1N3c1ccccc1 ; c1ccc(N2c3ccccc3B3c4c2cccc4N(c2ccccc2)c2c3n(-c3ccccc3)c3ccccc23)cc1 ; c1ccc(N2c3ccccc3B3c4c2cccc4N(c2ccccc2)c2oc4ccccc4c23)cc1]

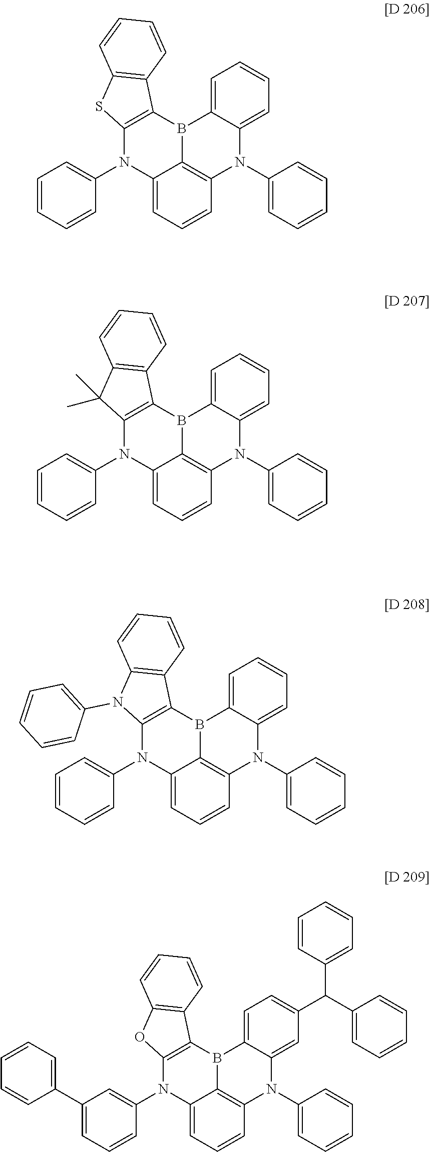 [SMILES: c1ccc(N2c3ccccc3B3c4c2cccc4N(c2ccccc2)c2sc4ccccc4c23)cc1 ; CC1(C)C2=C(B3c4ccccc4N(c4ccccc4)c4cccc(c43)N2c2ccccc2)c2ccccc21 ; c1ccc(N2c3ccccc3B3c4c2cccc4N(c2ccccc2)c2c3c3ccccc3n2-c2ccccc2)cc1 ; c1ccc(-c2cccc(N3c4cccc5c4B(c4ccc(C(c6ccccc6)c6ccccc6)cc4N5c4ccccc4)c4c3oc3ccccc43)c2)cc1]

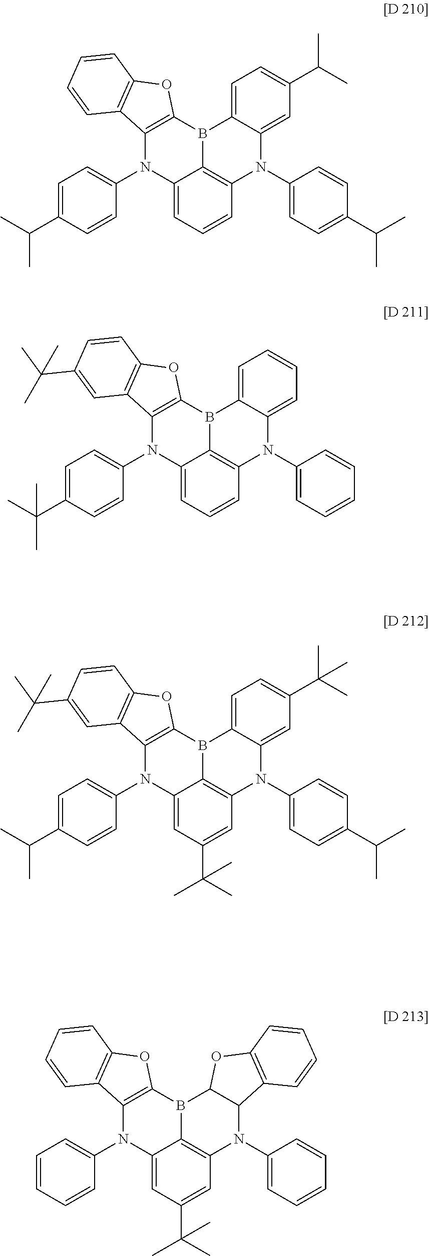

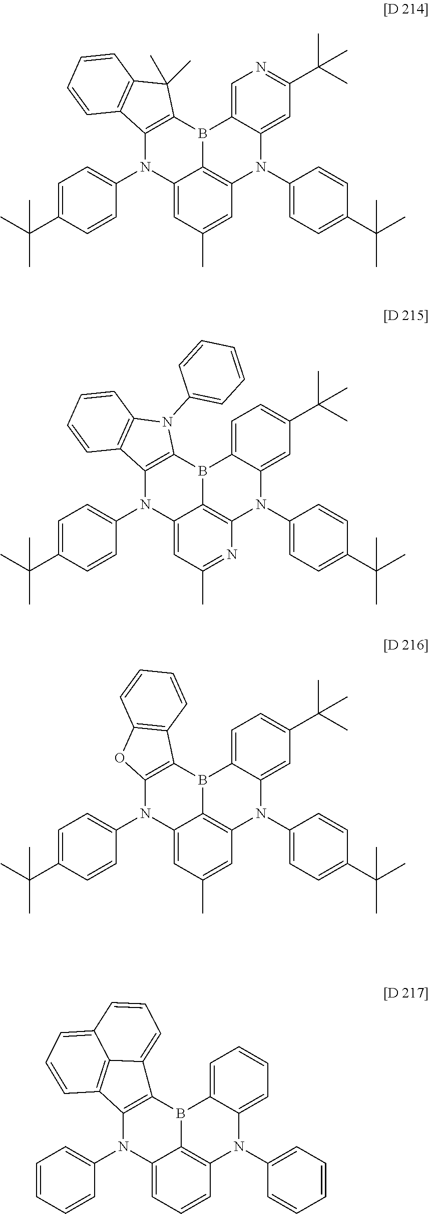

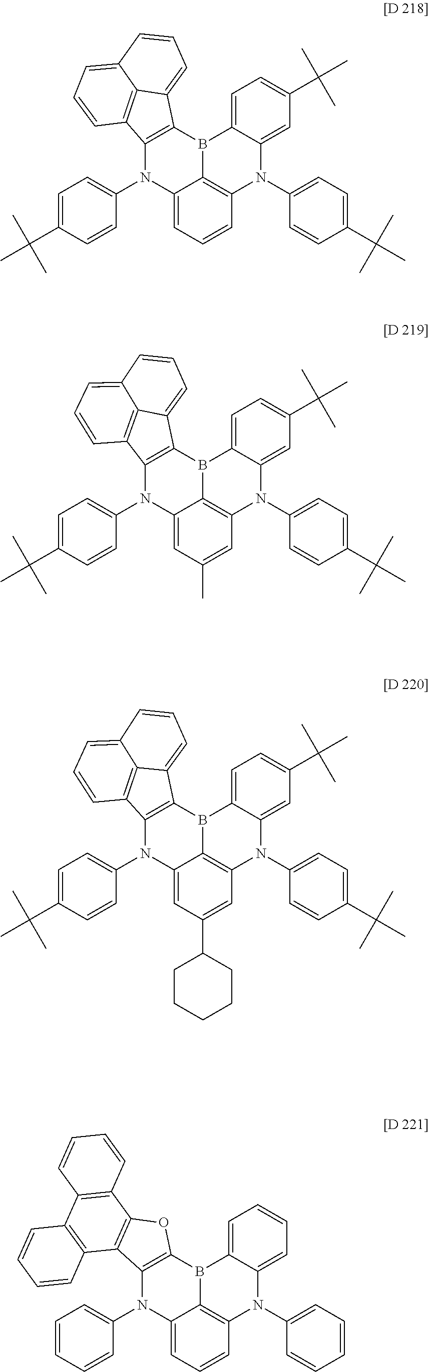

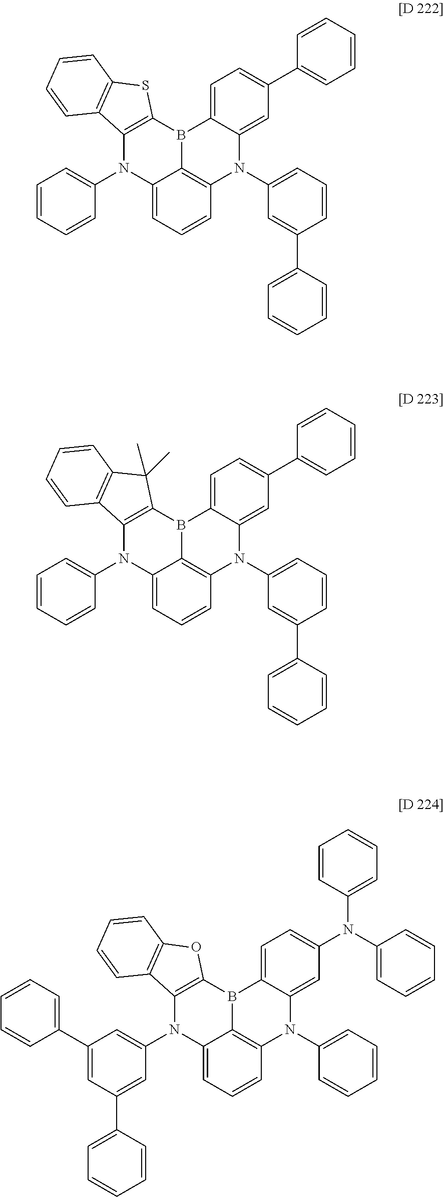 [SMILES: c1ccc(-c2cccc(N3c4cc(-c5ccccc5)ccc4B4c5sc6ccccc6c5N(c5ccccc5)c5cccc3c54)c2)cc1 ; CC1(C)C2=C(c3ccccc31)N(c1ccccc1)c1cccc3c1B2c1ccc(-c2ccccc2)cc1N3c1cccc(-c2ccccc2)c1 ; c1ccc(-c2cc(-c3ccccc3)cc(N3c4cccc5c4B(c4ccc(N(c6ccccc6)c6ccccc6)cc4N5c4ccccc4)c4oc5ccccc5c43)c2)cc1]

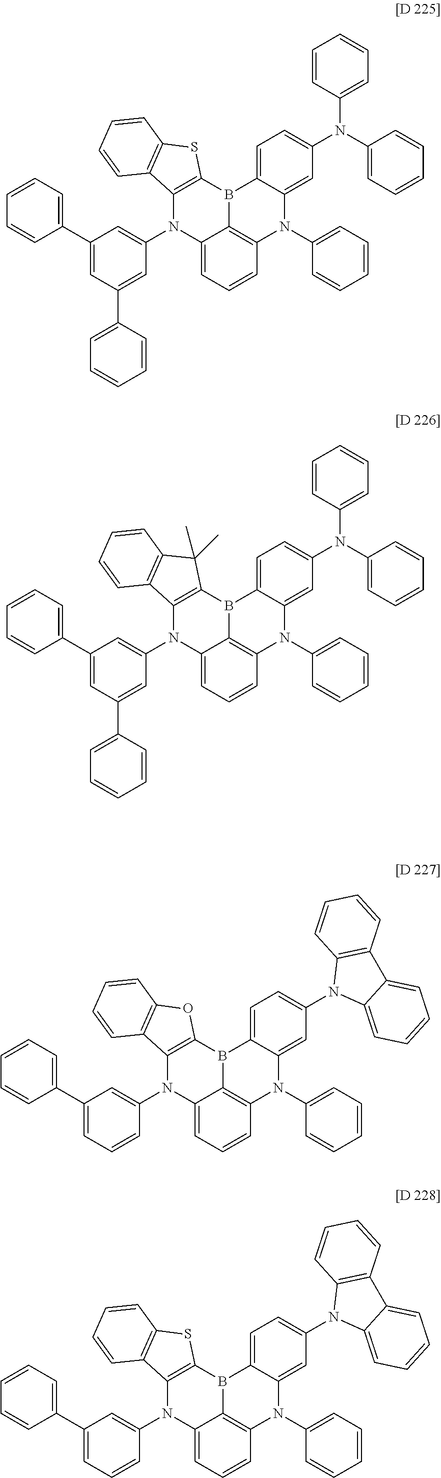 [SMILES: c1ccc(-c2cc(-c3ccccc3)cc(N3c4cccc5c4B(c4ccc(N(c6ccccc6)c6ccccc6)cc4N5c4ccccc4)c4sc5ccccc5c43)c2)cc1 ; CC1(C)C2=C(c3ccccc31)N(c1cc(-c3ccccc3)cc(-c3ccccc3)c1)c1cccc3c1B2c1ccc(N(c2ccccc2)c2ccccc2)cc1N3c1ccccc1 ; c1ccc(-c2cccc(N3c4cccc5c4B(c4ccc(-n6c7ccccc7c7ccccc76)cc4N5c4ccccc4)c4oc5ccccc5c43)c2)cc1 ; c1ccc(-c2cccc(N3c4cccc5c4B(c4ccc(-n6c7ccccc7c7ccccc76)cc4N5c4ccccc4)c4sc5ccccc5c43)c2)cc1]

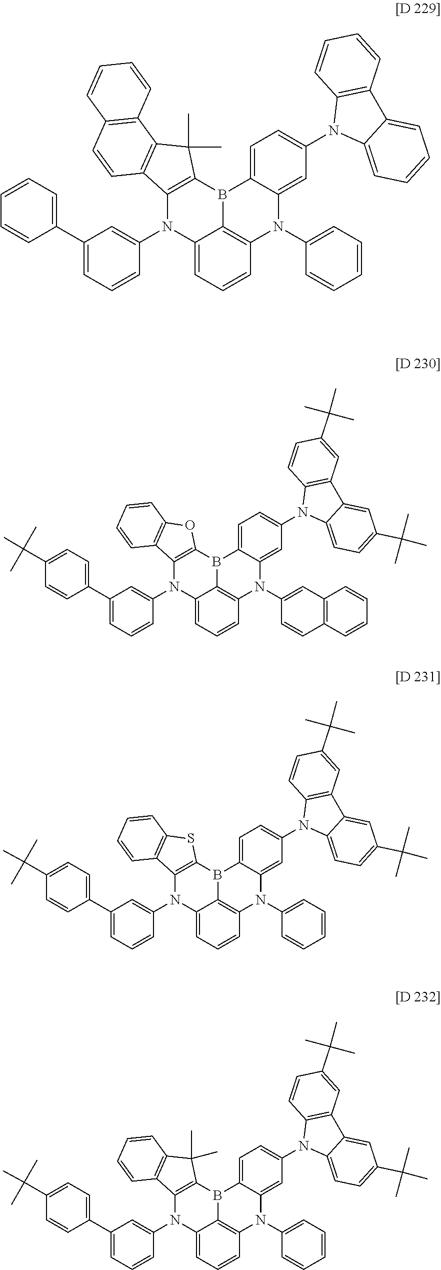

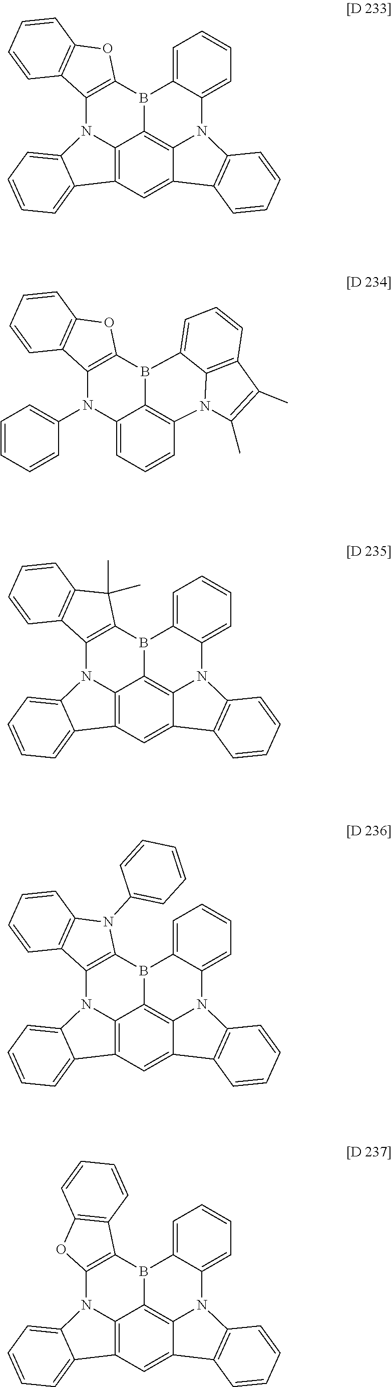 [SMILES: c1ccc2c(c1)B1c3oc4ccccc4c3-n3c4ccccc4c4cc5c6ccccc6n-2c5c1c43 ; Cc1c(C)n2c3c(cccc13)B1c3oc4ccccc4c3N(c3ccccc3)c3cccc-2c31 ; CC1(C)C2=C(c3ccccc31)n1c3ccccc3c3cc4c5ccccc5n5c4c(c31)B2c1ccccc1-5 ; c1ccc(-n2c3c(c4ccccc42)-n2c4ccccc4c4cc5c6ccccc6n6c5c(c42)B3c2ccccc2-6)cc1 ; c1ccc2c(c1)B1c3c(oc4ccccc34)-n3c4ccccc4c4cc5c6ccccc6n-2c5c1c43]

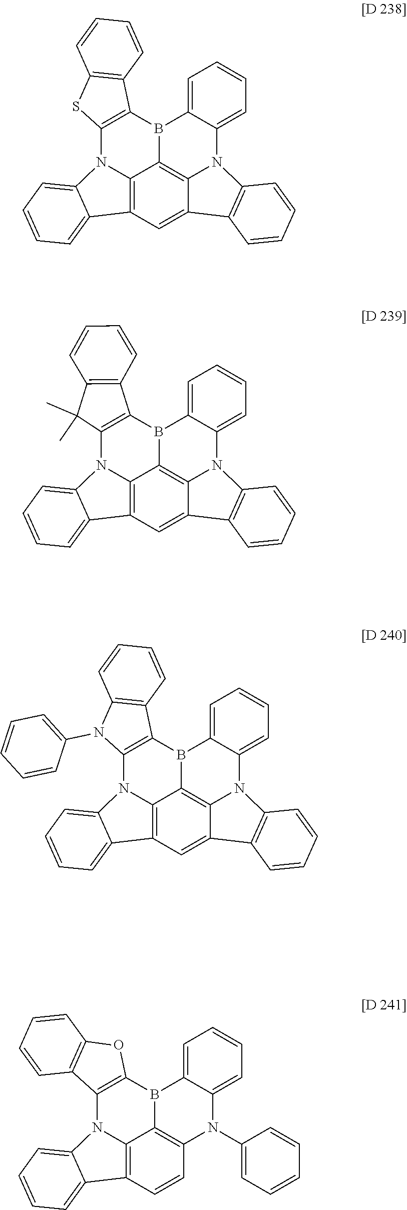 [SMILES: c1ccc2c(c1)B1c3c(sc4ccccc34)-n3c4ccccc4c4cc5c6ccccc6n-2c5c1c43 ; CC1(C)C2=C(B3c4ccccc4-n4c5ccccc5c5cc6c7ccccc7n2c6c3c54)c2ccccc21 ; c1ccc(-n2c3c(c4ccccc42)B2c4ccccc4-n4c5ccccc5c5cc6c7ccccc7n-3c6c2c54)cc1 ; c1ccc(N2c3ccccc3B3c4oc5ccccc5c4-n4c5ccccc5c5ccc2c3c54)cc1]

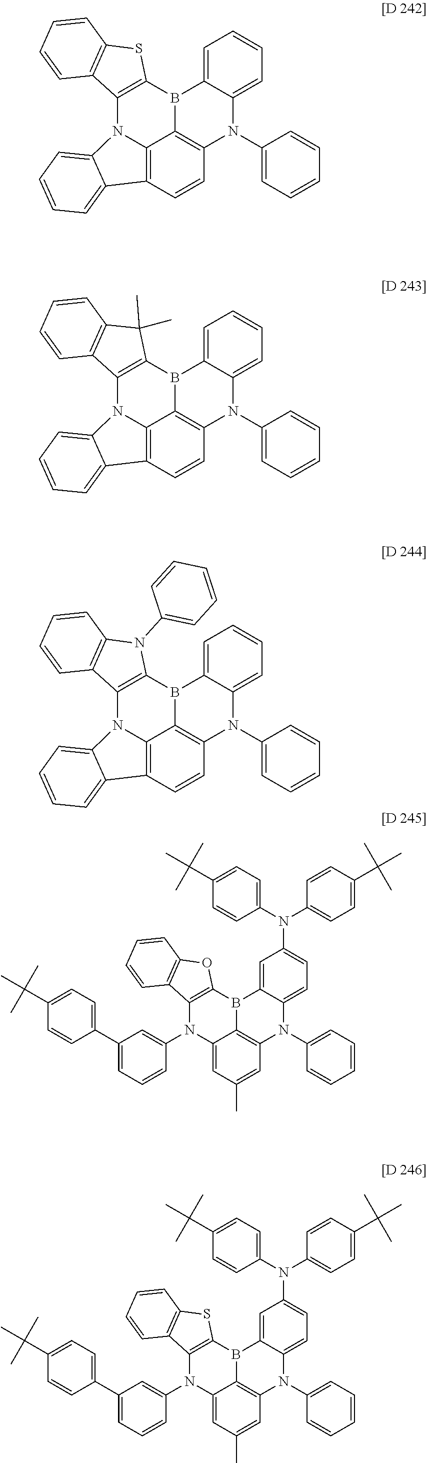

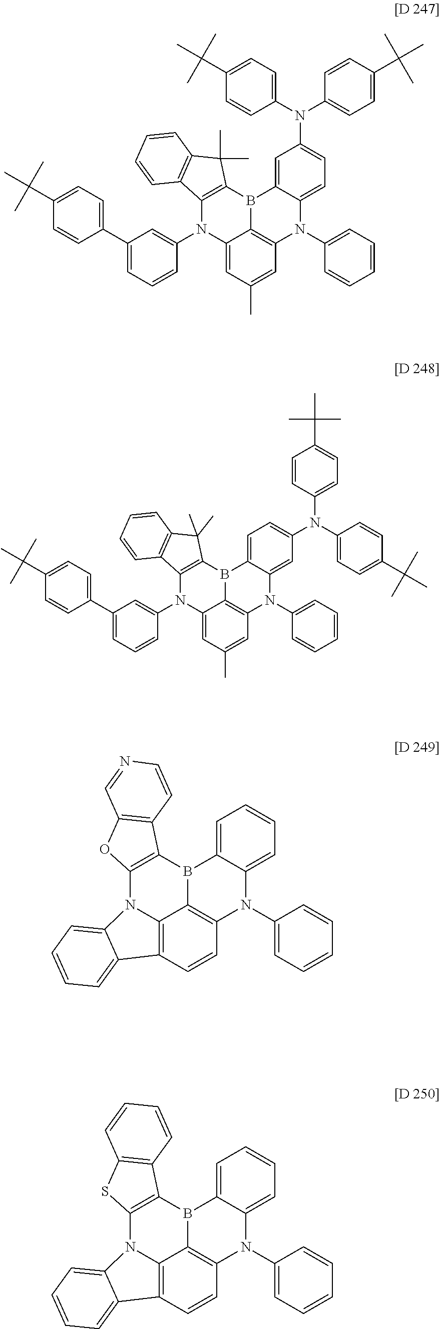

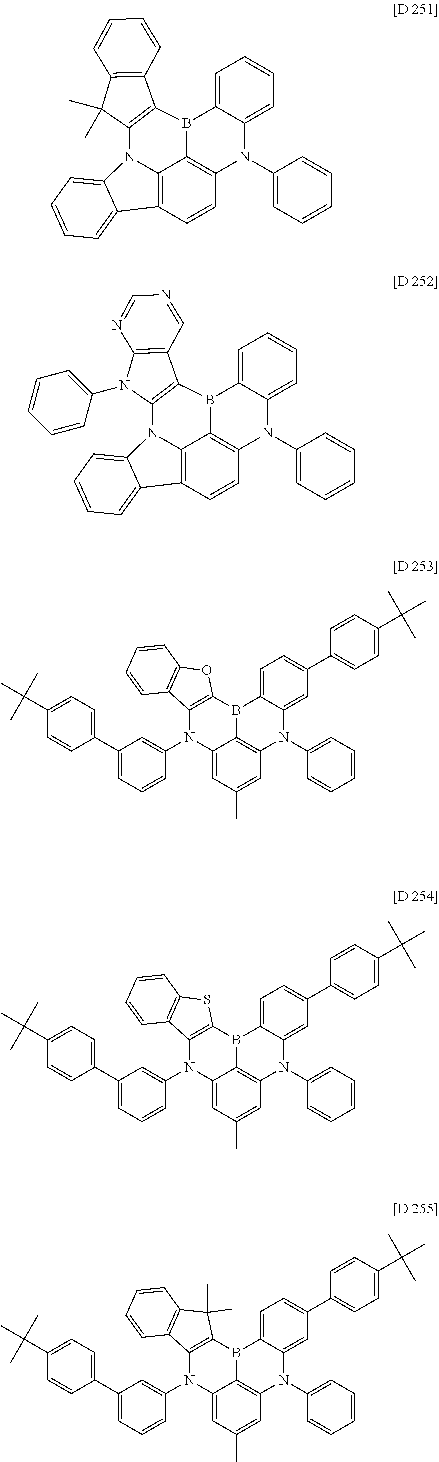

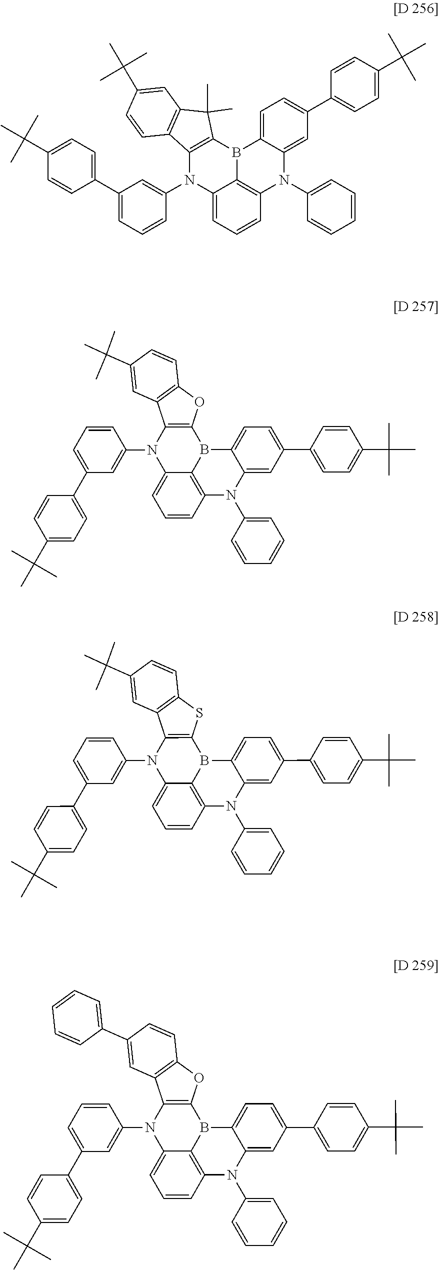

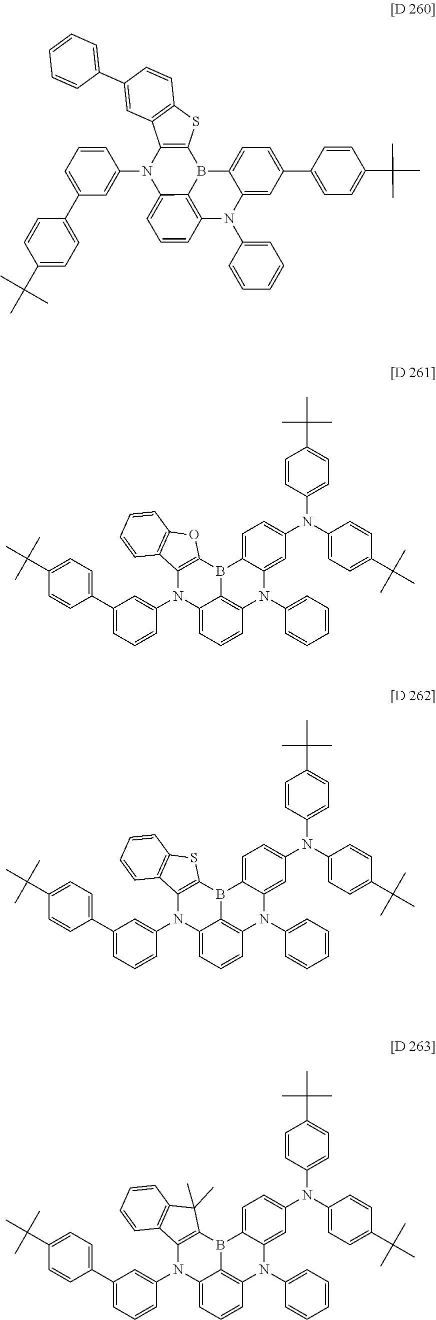

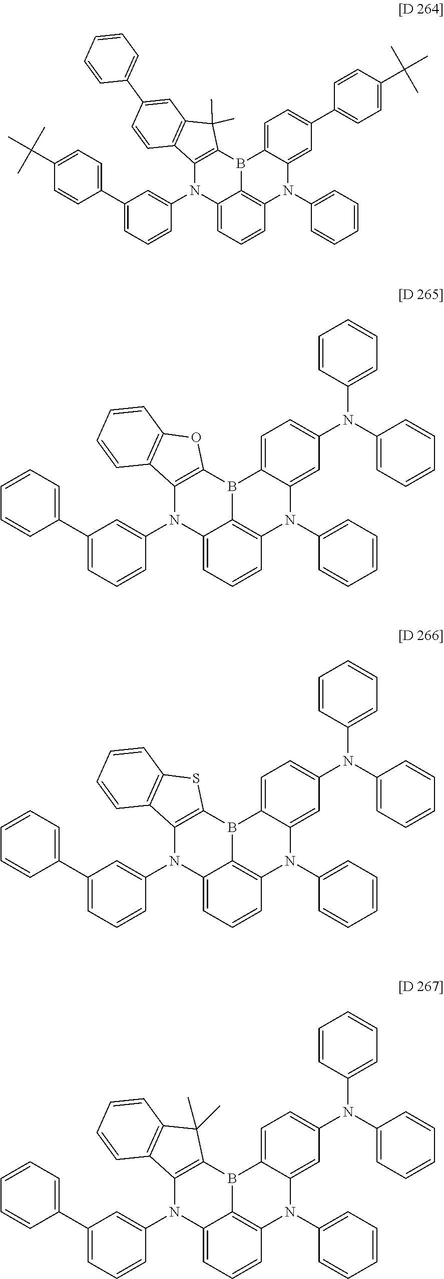 [SMILES: CC(C)(C)c1ccc(-c2cccc(N3C4=C(B5c6ccc(-c7ccc(C(C)(C)C)cc7)cc6N(c6ccccc6)c6cccc3c65)C(C)(C)c3cc(-c5ccccc5)ccc34)c2)cc1 ; c1ccc(-c2cccc(N3c4cccc5c4B(c4ccc(N(c6ccccc6)c6ccccc6)cc4N5c4ccccc4)c4oc5ccccc5c43)c2)cc1 ; c1ccc(-c2cccc(N3c4cccc5c4B(c4ccc(N(c6ccccc6)c6ccccc6)cc4N5c4ccccc4)c4sc5ccccc5c43)c2)cc1 ; CC1(C)C2=C(c3ccccc31)N(c1cccc(-c3ccccc3)c1)c1cccc3c1B2c1ccc(N(c2ccccc2)c2ccccc2)cc1N3c1ccccc1]

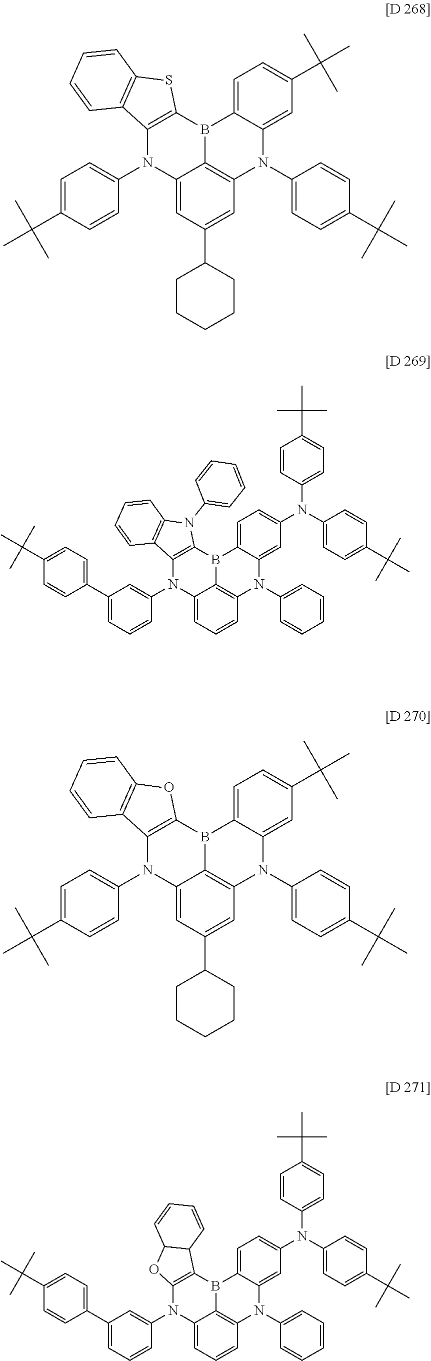

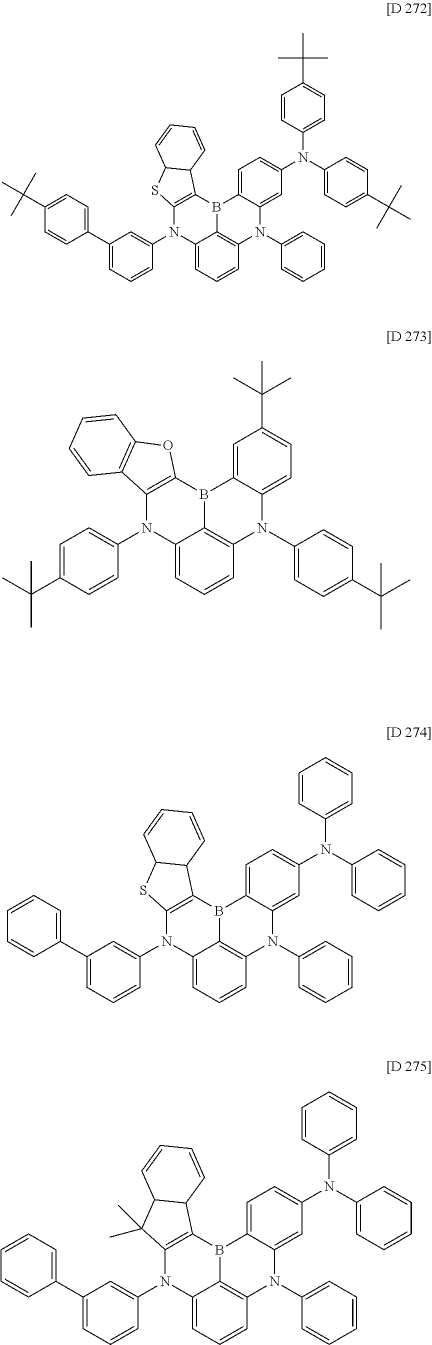

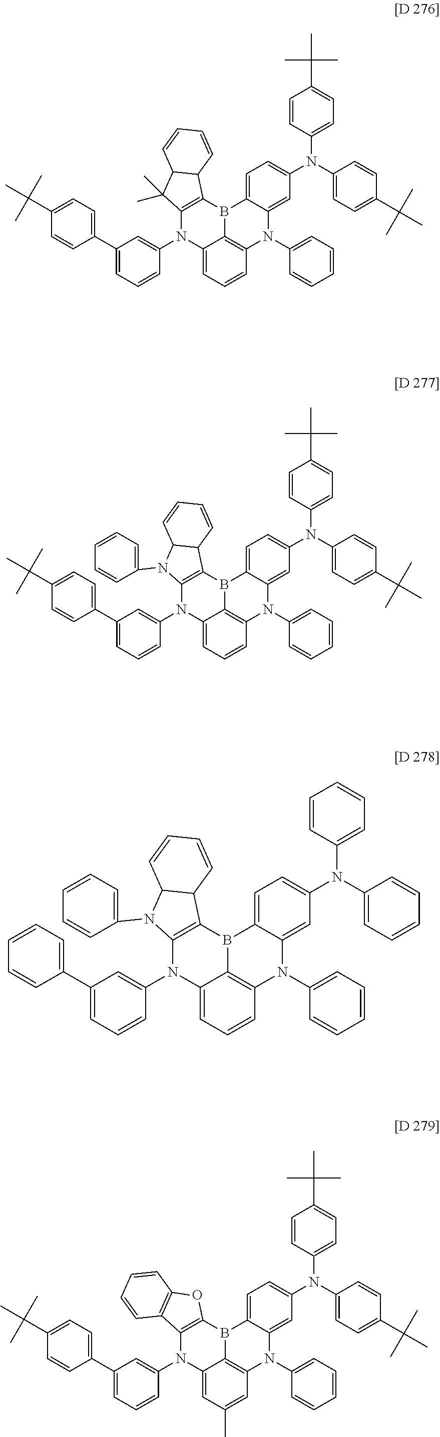

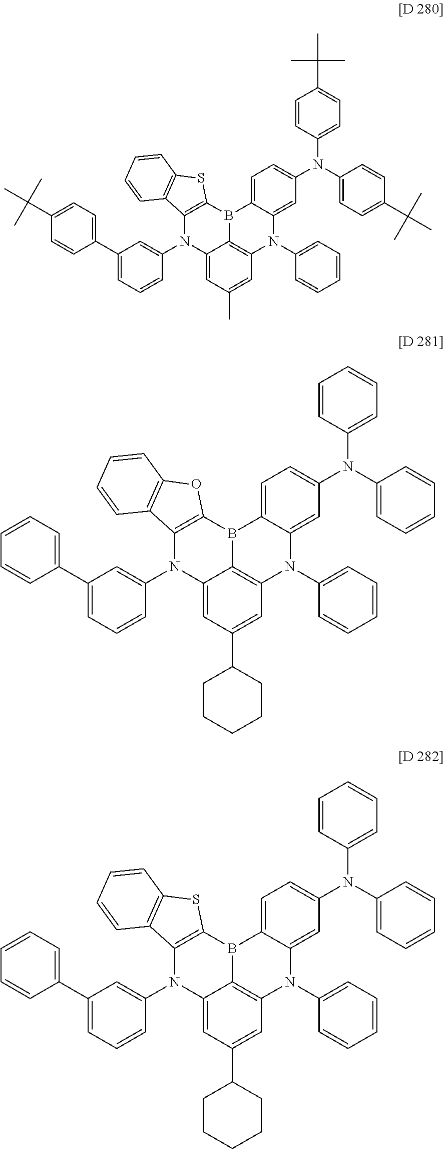 [SMILES: Cc1cc2c3c(c1)N(c1cccc(-c4ccc(C(C)(C)C)cc4)c1)c1c(sc4ccccc14)B3c1ccc(N(c3ccc(C(C)(C)C)cc3)c3ccc(C(C)(C)C)cc3)cc1N2c1ccccc1 ; c1ccc(-c2cccc(N3c4cc(C5CCCCC5)cc5c4B(c4ccc(N(c6ccccc6)c6ccccc6)cc4N5c4ccccc4)c4oc5ccccc5c43)c2)cc1 ; c1ccc(-c2cccc(N3c4cc(C5CCCCC5)cc5c4B(c4ccc(N(c6ccccc6)c6ccccc6)cc4N5c4ccccc4)c4sc5ccccc5c43)c2)cc1]

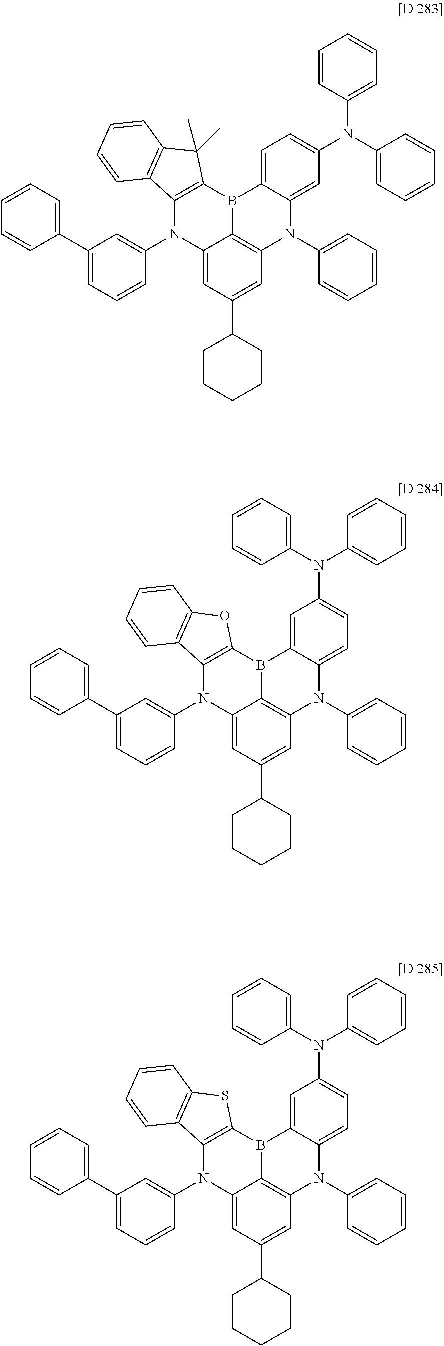 [SMILES: CC1(C)C2=C(c3ccccc31)N(c1cccc(-c3ccccc3)c1)c1cc(C3CCCCC3)cc3c1B2c1ccc(N(c2ccccc2)c2ccccc2)cc1N3c1ccccc1 ; c1ccc(-c2cccc(N3c4cc(C5CCCCC5)cc5c4B(c4cc(N(c6ccccc6)c6ccccc6)ccc4N5c4ccccc4)c4oc5ccccc5c43)c2)cc1 ; c1ccc(-c2cccc(N3c4cc(C5CCCCC5)cc5c4B(c4cc(N(c6ccccc6)c6ccccc6)ccc4N5c4ccccc4)c4sc5ccccc5c43)c2)cc1]

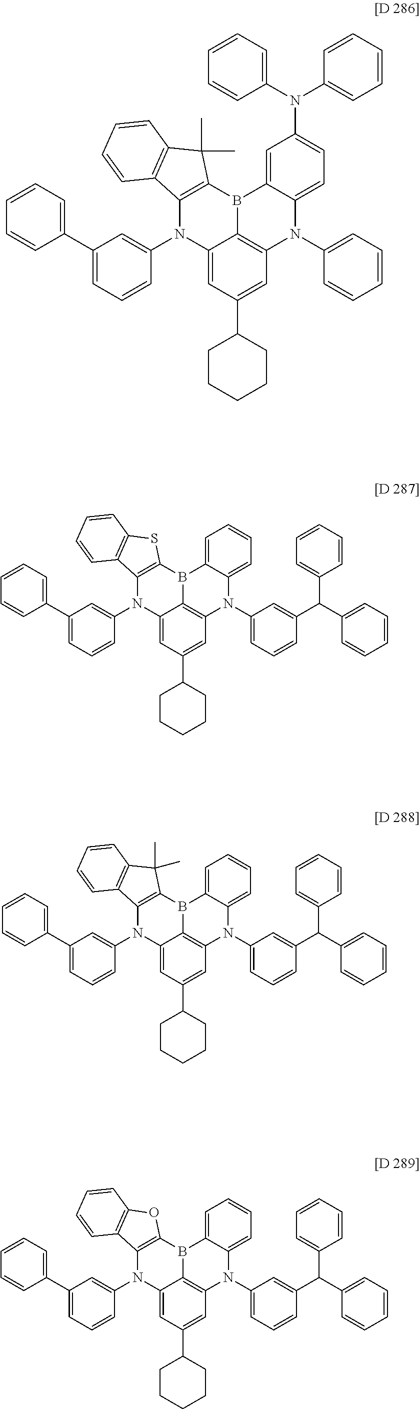 [SMILES: CC1(C)C2=C(c3ccccc31)N(c1cccc(-c3ccccc3)c1)c1cc(C3CCCCC3)cc3c1B2c1cc(N(c2ccccc2)c2ccccc2)ccc1N3c1ccccc1 ; c1ccc(-c2cccc(N3c4cc(C5CCCCC5)cc5c4B(c4ccccc4N5c4cccc(C(c5ccccc5)c5ccccc5)c4)c4sc5ccccc5c43)c2)cc1 ; CC1(C)C2=C(c3ccccc31)N(c1cccc(-c3ccccc3)c1)c1cc(C3CCCCC3)cc3c1B2c1ccccc1N3c1cccc(C(c2ccccc2)c2ccccc2)c1 ; c1ccc(-c2cccc(N3c4cc(C5CCCCC5)cc5c4B(c4ccccc4N5c4cccc(C(c5ccccc5)c5ccccc5)c4)c4oc5ccccc5c43)c2)cc1]

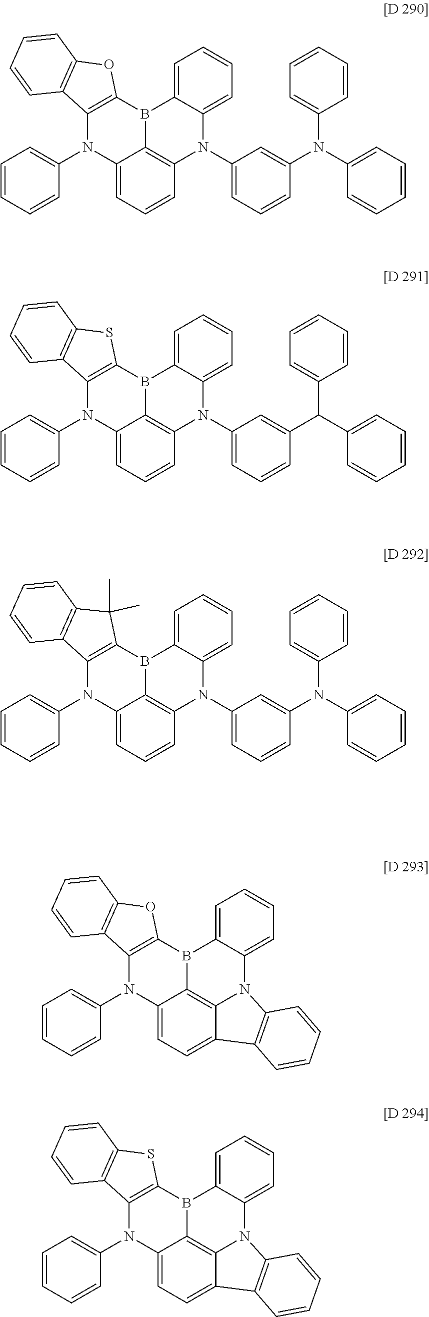 [SMILES: c1ccc(N(c2ccccc2)c2cccc(N3c4ccccc4B4c5oc6ccccc6c5N(c5ccccc5)c5cccc3c54)c2)cc1 ; c1ccc(C(c2ccccc2)c2cccc(N3c4ccccc4B4c5sc6ccccc6c5N(c5ccccc5)c5cccc3c54)c2)cc1 ; CC1(C)C2=C(c3ccccc31)N(c1ccccc1)c1cccc3c1B2c1ccccc1N3c1cccc(N(c2ccccc2)c2ccccc2)c1 ; c1ccc(N2c3ccc4c5ccccc5n5c4c3B(c3ccccc3-5)c3oc4ccccc4c32)cc1 ; c1ccc(N2c3ccc4c5ccccc5n5c4c3B(c3ccccc3-5)c3sc4ccccc4c32)cc1]

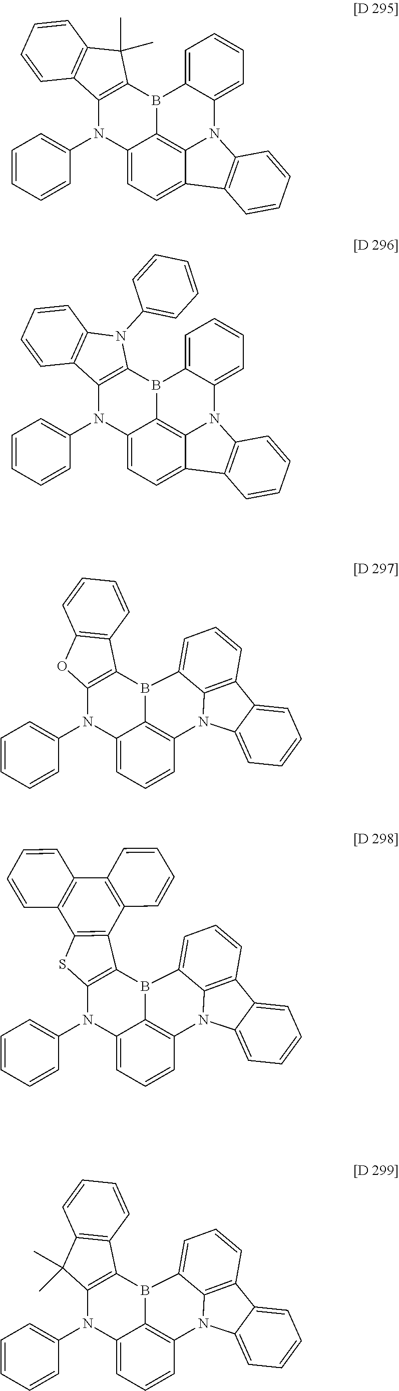 [SMILES: CC1(C)C2=C(c3ccccc31)N(c1ccccc1)c1ccc3c4ccccc4n4c3c1B2c1ccccc1-4 ; c1ccc(N2c3ccc4c5ccccc5n5c4c3B(c3ccccc3-5)c3c2c2ccccc2n3-c2ccccc2)cc1 ; c1ccc(N2c3cccc4c3B(c3c2oc2ccccc32)c2cccc3c5ccccc5n-4c23)cc1 ; c1ccc(N2c3cccc4c3B(c3c2sc2c5ccccc5c5ccccc5c32)c2cccc3c5ccccc5n-4c23)cc1 ; CC1(C)C2=C(B3c4c(cccc4-n4c5ccccc5c5cccc3c54)N2c2ccccc2)c2ccccc21]

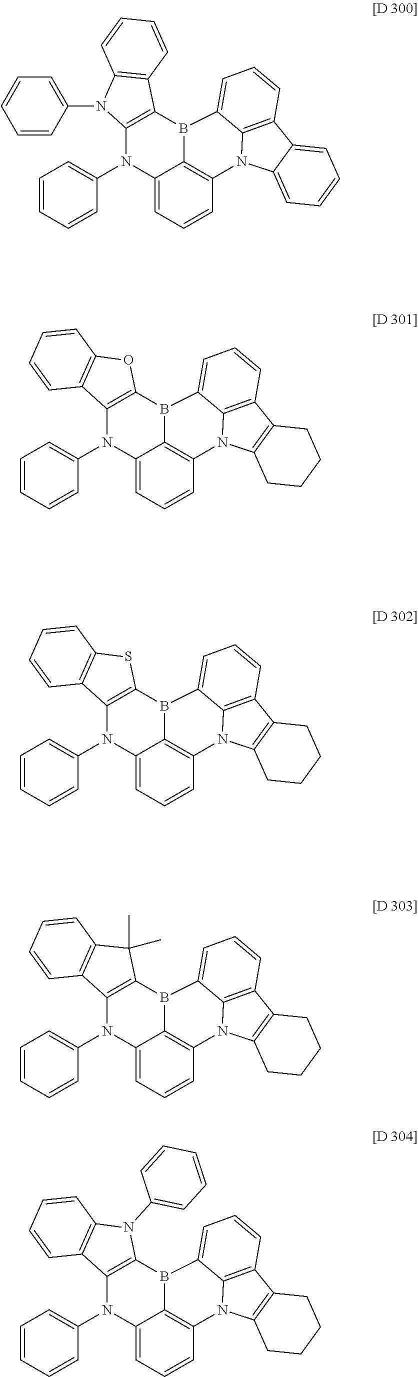 [SMILES: c1ccc(N2c3cccc4c3B(c3c2n(-c2ccccc2)c2ccccc32)c2cccc3c5ccccc5n-4c23)cc1 ; c1ccc(N2c3cccc4c3B(c3oc5ccccc5c32)c2cccc3c5c(n-4c23)CCCC5)cc1 ; c1ccc(N2c3cccc4c3B(c3sc5ccccc5c32)c2cccc3c5c(n-4c23)CCCC5)cc1 ; CC1(C)C2=C(c3ccccc31)N(c1ccccc1)c1cccc3c1B2c1cccc2c4c(n-3c12)CCCC4 ; c1ccc(N2c3cccc4c3B(c3cccc5c6c(n-4c35)CCCC6)c3c2c2ccccc2n3-c2ccccc2)cc1]

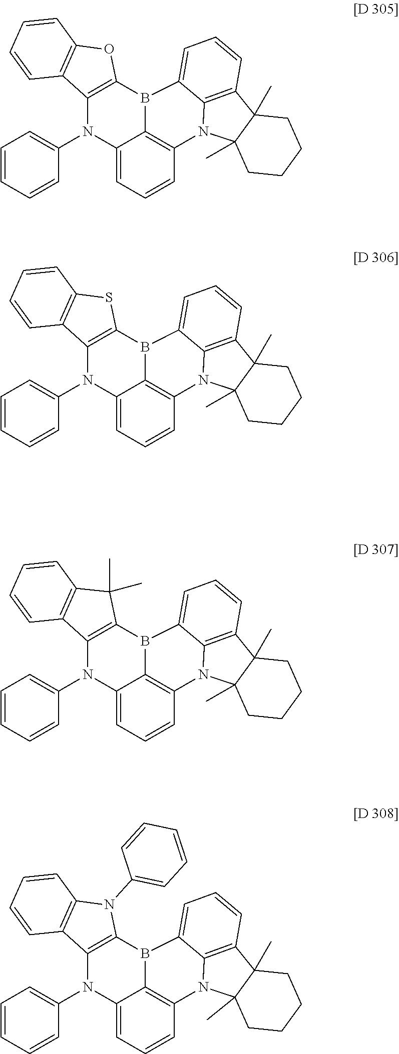 [SMILES: CC12CCCCC1(C)N1c3cccc4c3B(c3cccc2c31)c1oc2ccccc2c1N4c1ccccc1 ; CC12CCCCC1(C)N1c3cccc4c3B(c3cccc2c31)c1sc2ccccc2c1N4c1ccccc1 ; CC1(C)C2=C(c3ccccc31)N(c1ccccc1)c1cccc3c1B2c1cccc2c1N3C1(C)CCCCC21C ; CC12CCCCC1(C)N1c3cccc4c3B(c3cccc2c31)c1c(c2ccccc2n1-c1ccccc1)N4c1ccccc1]

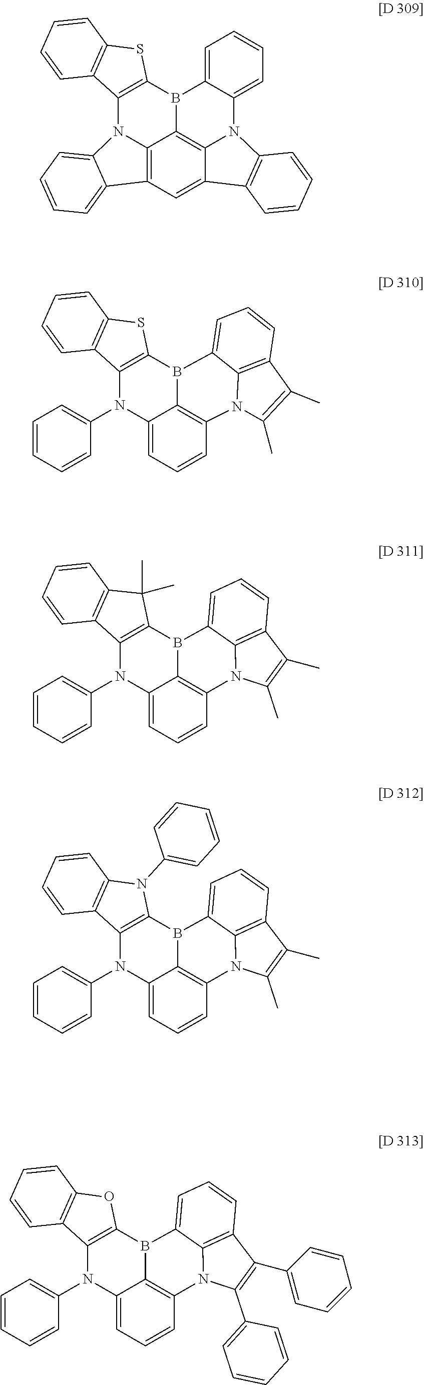 [SMILES: c1ccc2c(c1)B1c3sc4ccccc4c3-n3c4ccccc4c4cc5c6ccccc6n-2c5c1c43 ; Cc1c(C)n2c3c(cccc13)B1c3sc4ccccc4c3N(c3ccccc3)c3cccc-2c31 ; Cc1c(C)n2c3c(cccc13)B1C3=C(c4ccccc4C3(C)C)N(c3ccccc3)c3cccc-2c31 ; Cc1c(C)n2c3c(cccc13)B1c3c(cccc3-2)N(c2ccccc2)c2c1n(-c1ccccc1)c1ccccc21 ; c1ccc(-c2c(-c3ccccc3)n3c4c(cccc24)B2c4oc5ccccc5c4N(c4ccccc4)c4cccc-3c42)cc1]

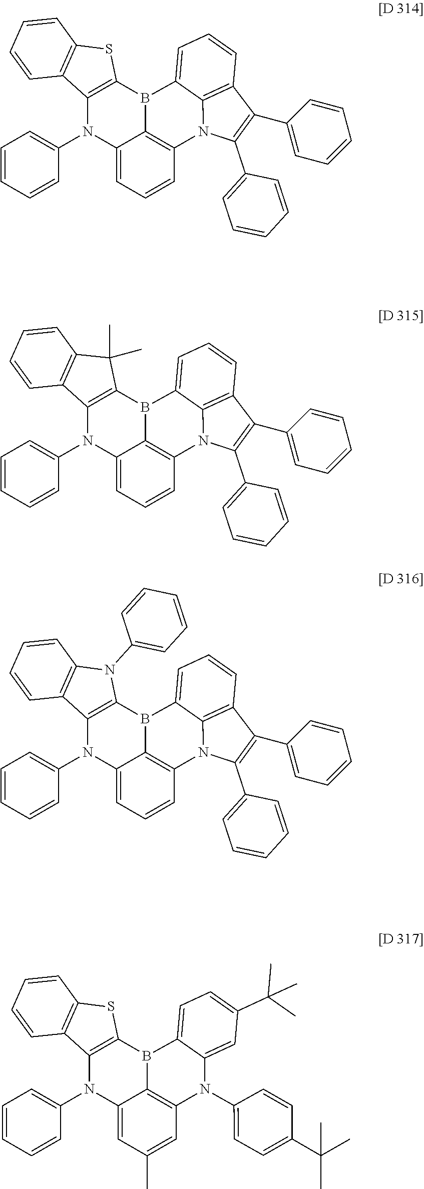 [SMILES: c1ccc(-c2c(-c3ccccc3)n3c4c(cccc24)B2c4sc5ccccc5c4N(c4ccccc4)c4cccc-3c42)cc1 ; CC1(C)C2=C(c3ccccc31)N(c1ccccc1)c1cccc3c1B2c1cccc2c(-c4ccccc4)c(-c4ccccc4)n-3c12 ; c1ccc(-c2c(-c3ccccc3)n3c4c(cccc24)B2c4c(cccc4-3)N(c3ccccc3)c3c2n(-c2ccccc2)c2ccccc32)cc1 ; Cc1cc2c3c(c1)N(c1ccccc1)c1c(sc4ccccc14)B3c1ccc(C(C)(C)C)cc1N2c1ccc(C(C)(C)C)cc1]

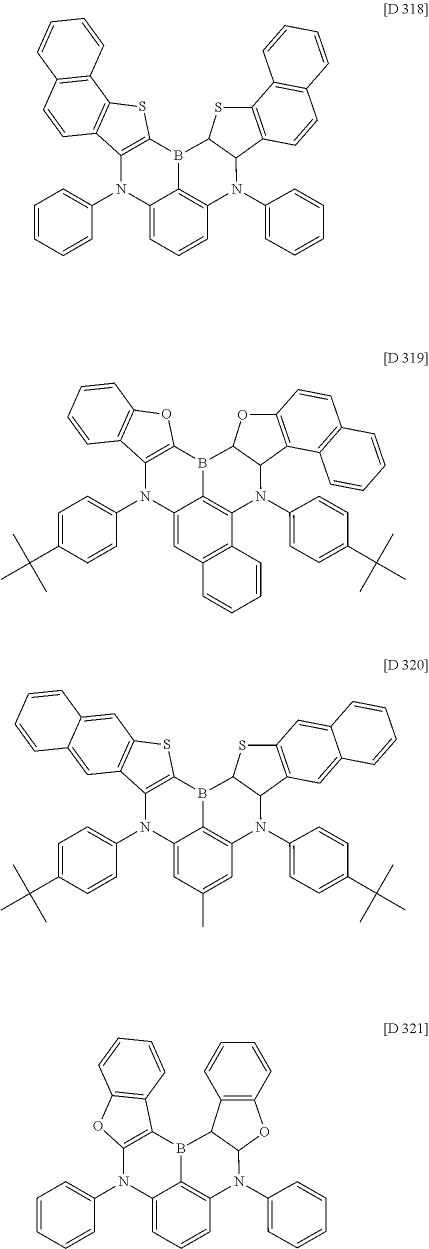

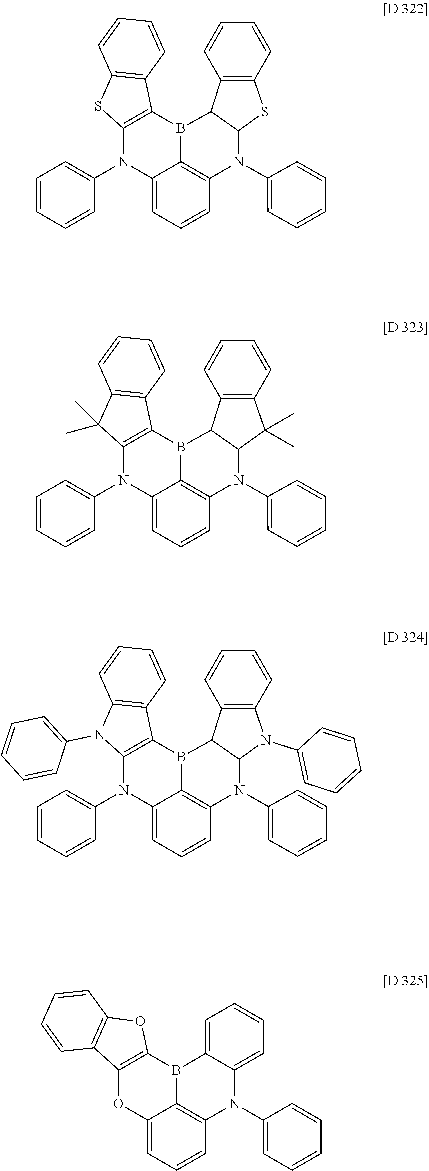 [SMILES: c1ccc(N2c3cccc4c3B(c3c2sc2ccccc32)C2c3ccccc3SC2N4c2ccccc2)cc1 ; CC1(C)C2=C(B3c4c(cccc4N(c4ccccc4)C4C3c3ccccc3C4(C)C)N2c2ccccc2)c2ccccc21 ; c1ccc(N2c3cccc4c3B(c3c2n(-c2ccccc2)c2ccccc32)C2c3ccccc3N(c3ccccc3)C2N4c2ccccc2)cc1 ; c1ccc(N2c3ccccc3B3c4oc5ccccc5c4Oc4cccc2c43)cc1]

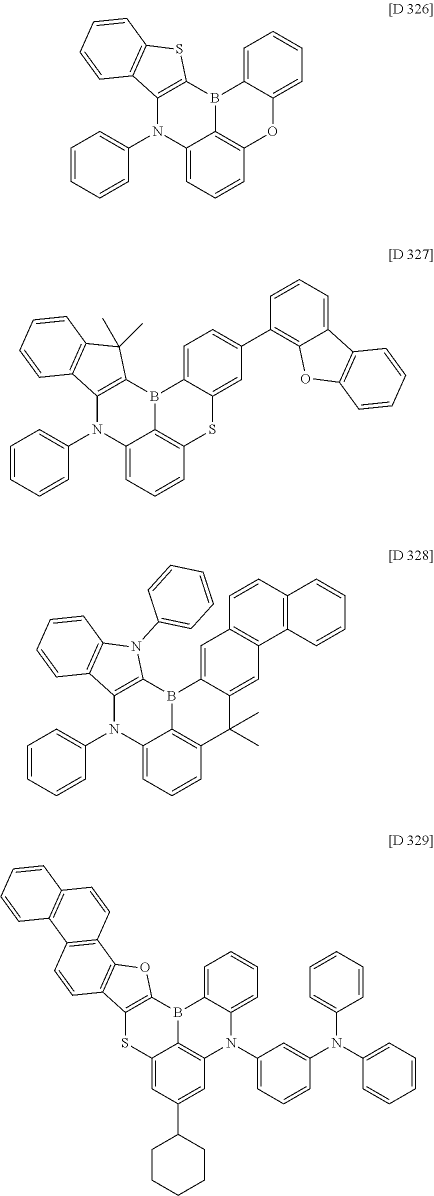 [SMILES: c1ccc(N2c3cccc4c3B(c3ccccc3O4)c3sc4ccccc4c32)cc1 ; CC1(C)C2=C(c3ccccc31)N(c1ccccc1)c1cccc3c1B2c1ccc(-c2cccc4c2oc2ccccc24)cc1S3 ; CC1(C)c2cc3c(ccc4ccccc43)cc2B2c3c(cccc31)N(c1ccccc1)c1c2n(-c2ccccc2)c2ccccc12 ; c1ccc(N(c2ccccc2)c2cccc(N3c4ccccc4B4c5oc6c(ccc7c8ccccc8ccc76)c5Sc5cc(C6CCCCC6)cc3c54)c2)cc1]

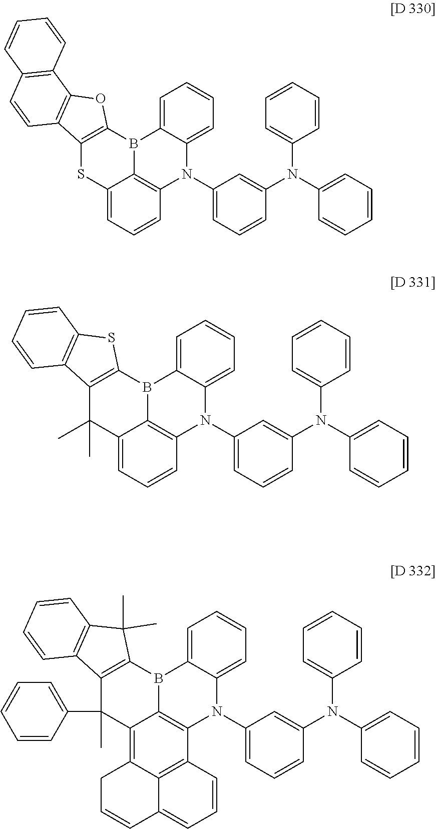 [SMILES: c1ccc(N(c2ccccc2)c2cccc(N3c4ccccc4B4c5oc6c(ccc7ccccc76)c5Sc5cccc3c54)c2)cc1 ; CC1(C)c2cccc3c2B(c2ccccc2N3c2cccc(N(c3ccccc3)c3ccccc3)c2)c2sc3ccccc3c21 ; CC1(C)C2=C(c3ccccc31)C(C)(c1ccccc1)c1c3c(c4cccc5c4c1CC=C5)N(c1cccc(N(c4ccccc4)c4ccccc4)c1)c1ccccc1B23]

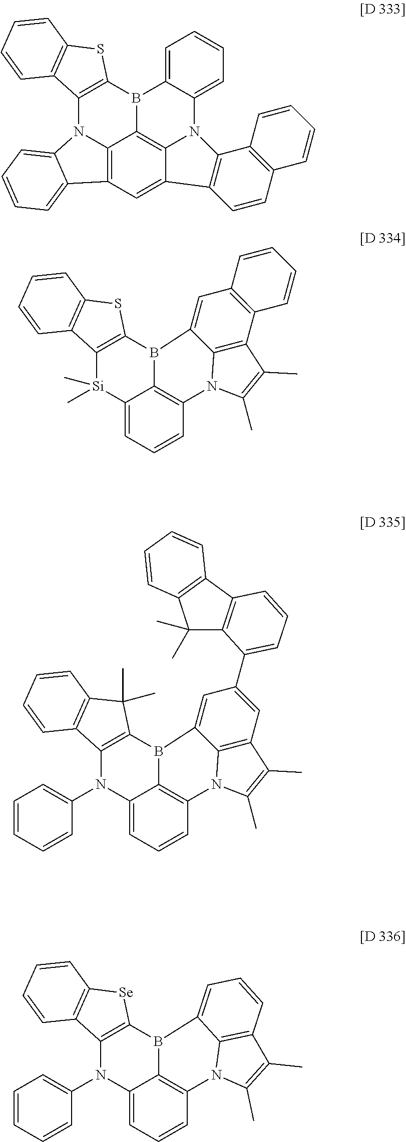

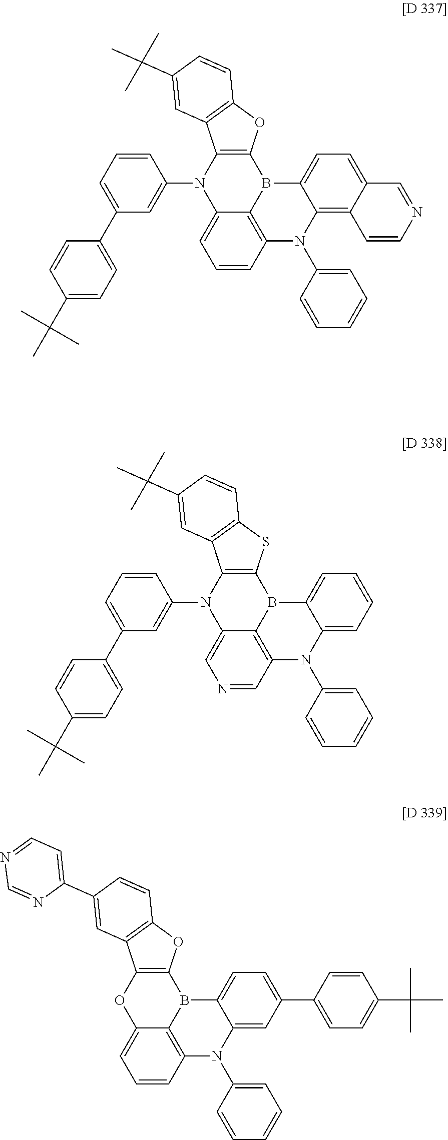 [SMILES: CC(C)(C)c1ccc(-c2cccc(N3c4cccc5c4B(c4ccc6cnccc6c4N5c4ccccc4)c4oc5ccc(C(C)(C)C)cc5c43)c2)cc1 ; CC(C)(C)c1ccc(-c2cccc(N3c4cncc5c4B(c4ccccc4N5c4ccccc4)c4sc5ccc(C(C)(C)C)cc5c43)c2)cc1 ; CC(C)(C)c1ccc(-c2ccc3c(c2)N(c2ccccc2)c2cccc4c2B3c2oc3ccc(-c5ccncn5)cc3c2O4)cc1]

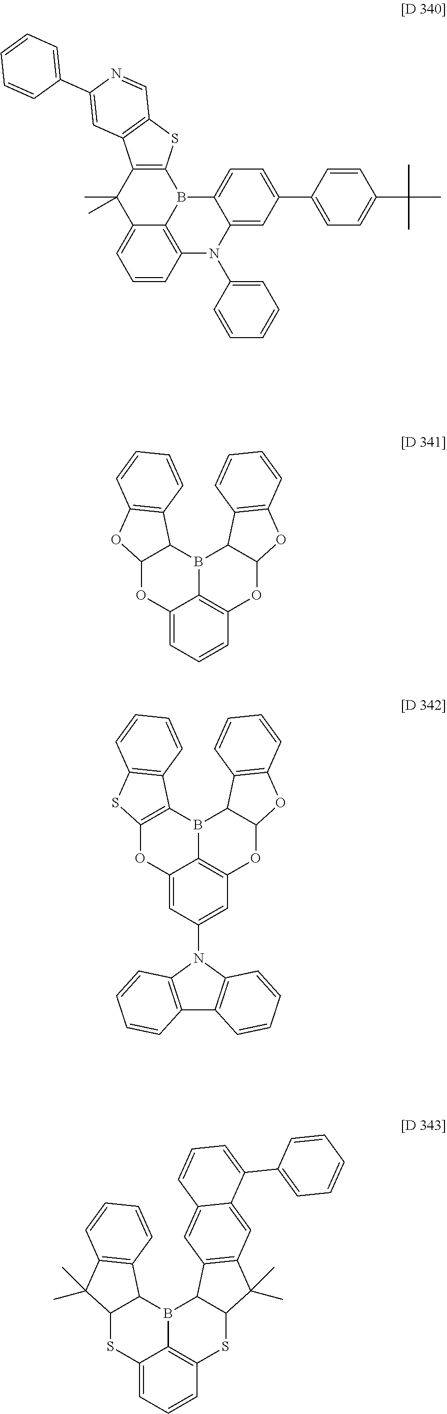

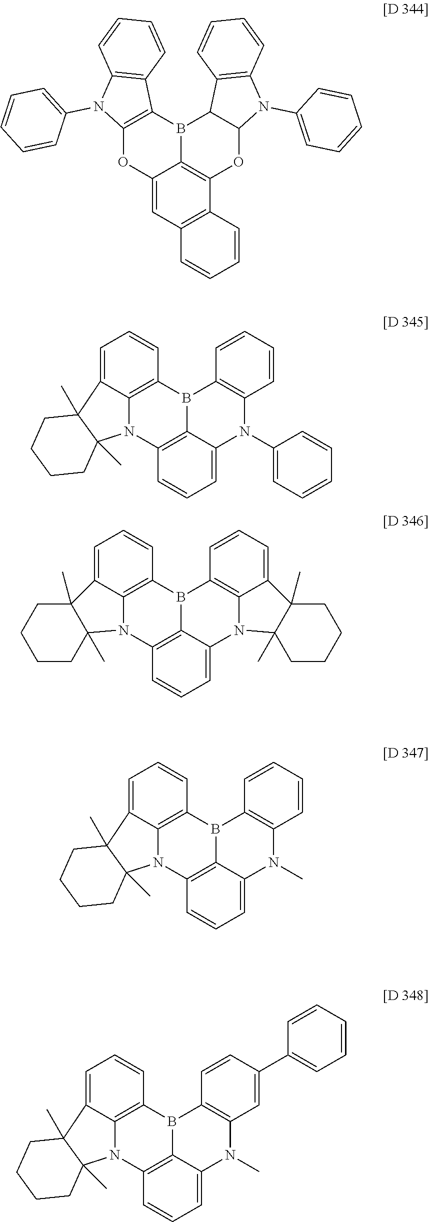 [SMILES: c1ccc(N2c3ccccc3C3B4c5c(cc6ccccc6c5OC32)Oc2c4c3ccccc3n2-c2ccccc2)cc1 ; CC12CCCCC1(C)N1c3cccc4c3B(c3ccccc3N4c3ccccc3)c3cccc2c31 ; CC12CCCCC1(C)N1c3cccc4c3B(c3cccc2c31)c1cccc2c1N4C1(C)CCCCC21C ; CN1c2ccccc2B2c3cccc4c3N(c3cccc1c32)C1(C)CCCCC41C ; CN1c2cc(-c3ccccc3)ccc2B2c3cccc4c3N(c3cccc1c32)C1(C)CCCCC41C]

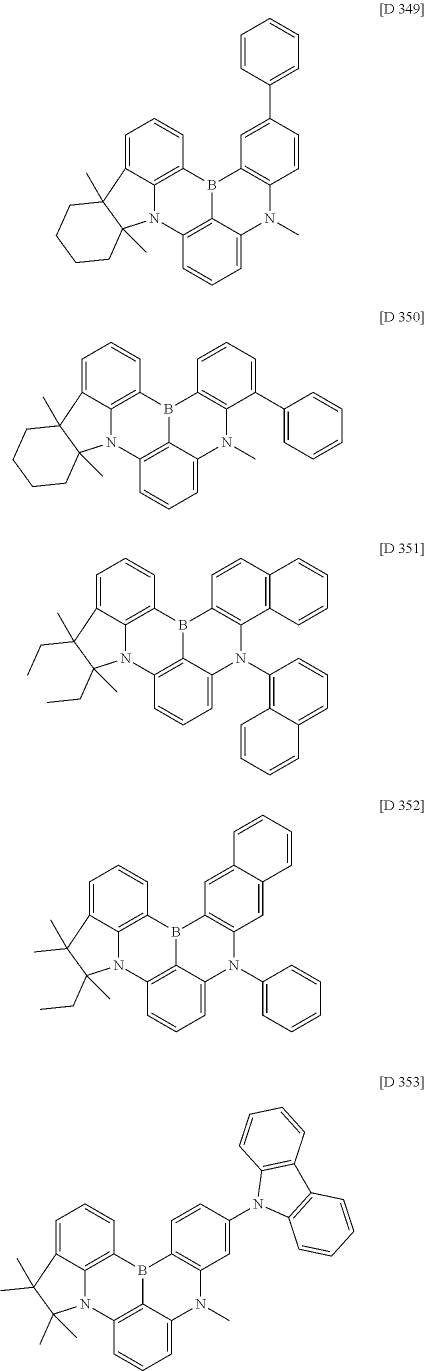

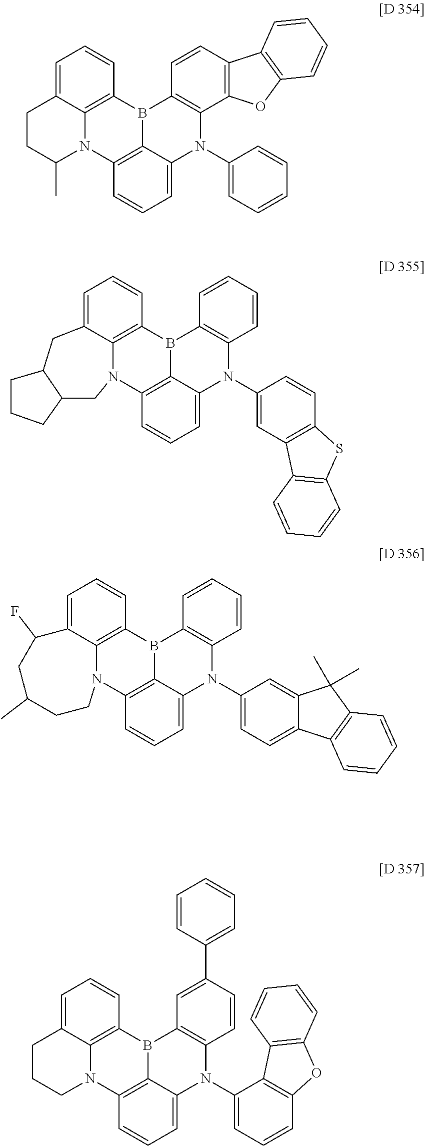 [SMILES: CC1CCc2cccc3c2N1c1cccc2c1B3c1ccc3c(oc4ccccc43)c1N2c1ccccc1 ; c1ccc2c(c1)B1c3cccc4c3N(CC3CCCC3C4)c3cccc(c31)N2c1ccc2sc3ccccc3c2c1 ; CC1CCN2c3cccc4c3B(c3ccccc3N4c3ccc4c(c3)C(C)(C)c3ccccc3-4)c3cccc(c32)C(F)C1 ; c1ccc(-c2ccc3c(c2)B2c4cccc5c4N(CCC5)c4cccc(c42)N3c2cccc3oc4ccccc4c23)cc1]

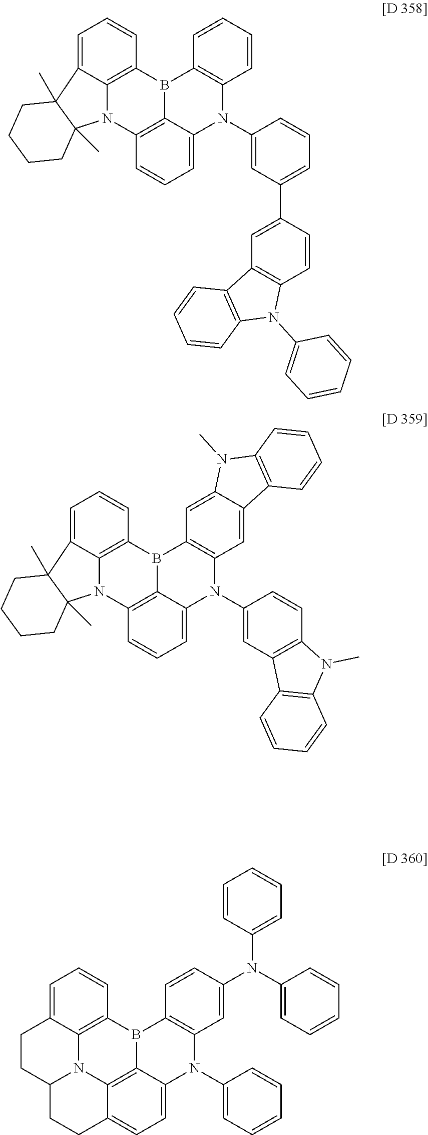

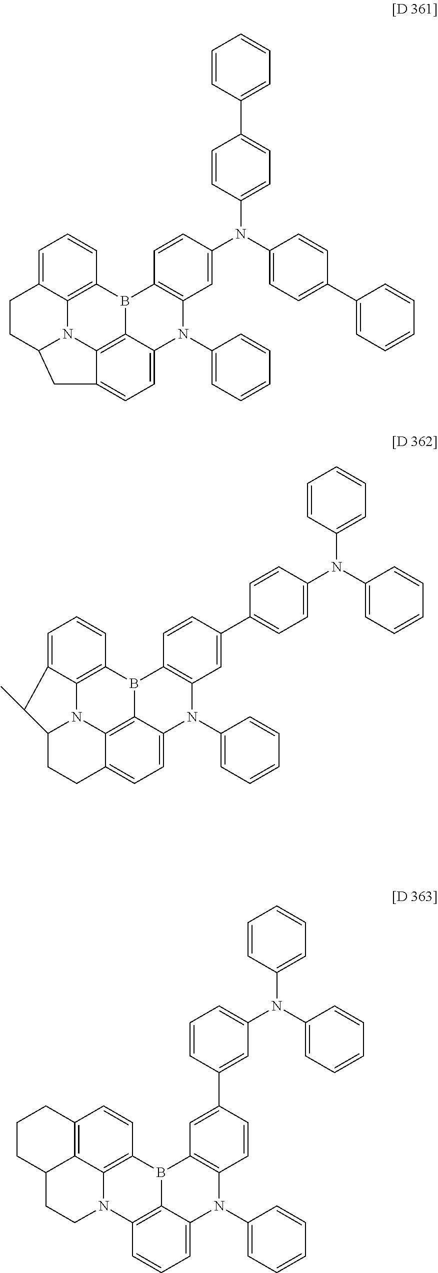 [SMILES: c1ccc(-c2ccc(N(c3ccc(-c4ccccc4)cc3)c3ccc4c(c3)N(c3ccccc3)c3ccc5c6c3B4c3cccc4c3N6C(CC4)C5)cc2)cc1 ; CC1c2cccc3c2N2c4c(ccc5c4B3c3ccc(-c4ccc(N(c6ccccc6)c6ccccc6)cc4)cc3N5c3ccccc3)CCC12 ; c1ccc(N(c2ccccc2)c2cccc(-c3ccc4c(c3)B3c5ccc6c7c5N(CCC7CCC6)c5cccc(c53)N4c3ccccc3)c2)cc1]

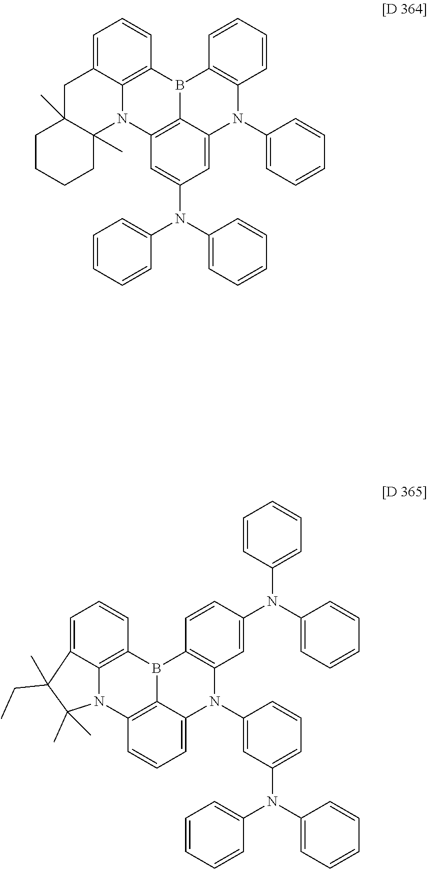 [SMILES: CC12CCCCC1(C)N1c3cc(N(c4ccccc4)c4ccccc4)cc4c3B(c3ccccc3N4c3ccccc3)c3cccc(c31)C2 ; CCC1(C)c2cccc3c2N(c2cccc4c2B3c2ccc(N(c3ccccc3)c3ccccc3)cc2N4c2cccc(N(c3ccccc3)c3ccccc3)c2)C1(C)C]

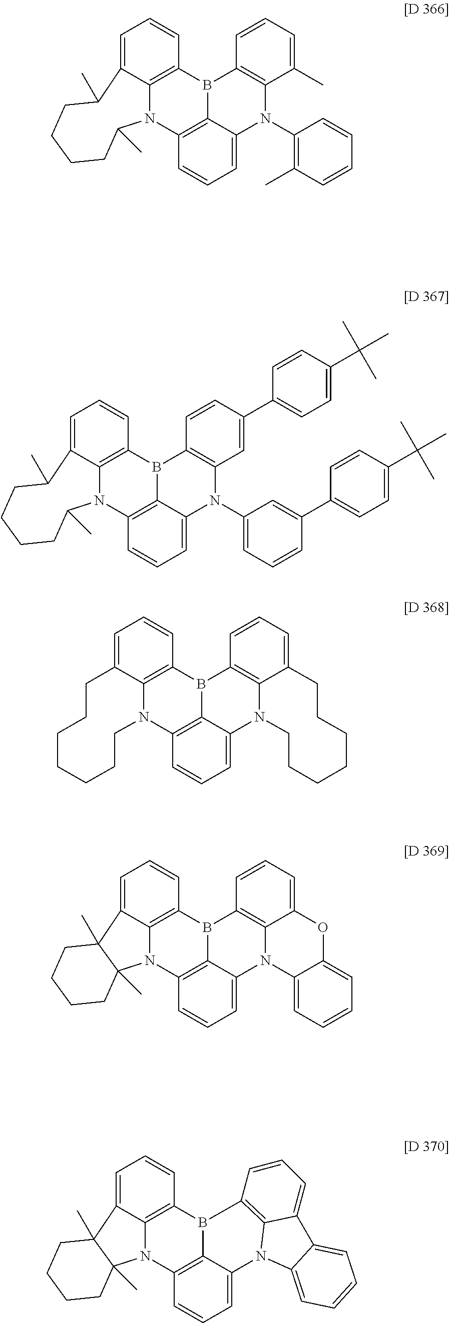 [SMILES: Cc1ccccc1N1c2cccc3c2B(c2cccc(C)c21)c1cccc2c1N3C(C)CCCCC2C ; CC1CCCCC(C)N2c3cccc4c3B(c3ccc(-c5ccc(C(C)(C)C)cc5)cc3N4c3cccc(-c4ccc(C(C)(C)C)cc4)c3)c3cccc1c32 ; c1cc2c3c(c1)B1c4cccc5c4N(CCCCCCC5)c4cccc(c41)N3CCCCCCC2 ; CC12CCCCC1(C)N1c3cccc4c3B(c3cccc5c3N4c3ccccc3O5)c3cccc2c31 ; CC12CCCCC1(C)N1c3cccc4c3B(c3cccc2c31)c1cccc2c3ccccc3n-4c12]

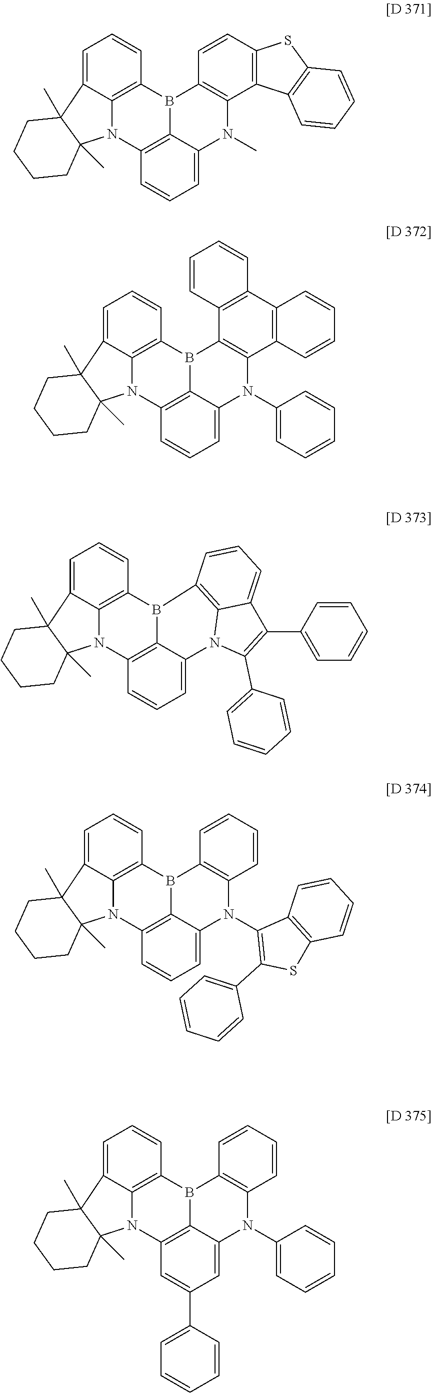 [SMILES: CN1c2cccc3c2B(c2cccc4c2N3C2(C)CCCCC42C)c2ccc3sc4ccccc4c3c21 ; CC12CCCCC1(C)N1c3cccc4c3B(c3cccc2c31)c1c(c2ccccc2c2ccccc12)N4c1ccccc1 ; CC12CCCCC1(C)N1c3cccc4c3B(c3cccc2c31)c1cccc2c(-c3ccccc3)c(-c3ccccc3)n-4c12 ; CC12CCCCC1(C)N1c3cccc4c3B(c3ccccc3N4c3c(-c4ccccc4)sc4ccccc34)c3cccc2c31 ; CC12CCCCC1(C)N1c3cc(-c4ccccc4)cc4c3B(c3ccccc3N4c3ccccc3)c3cccc2c31]

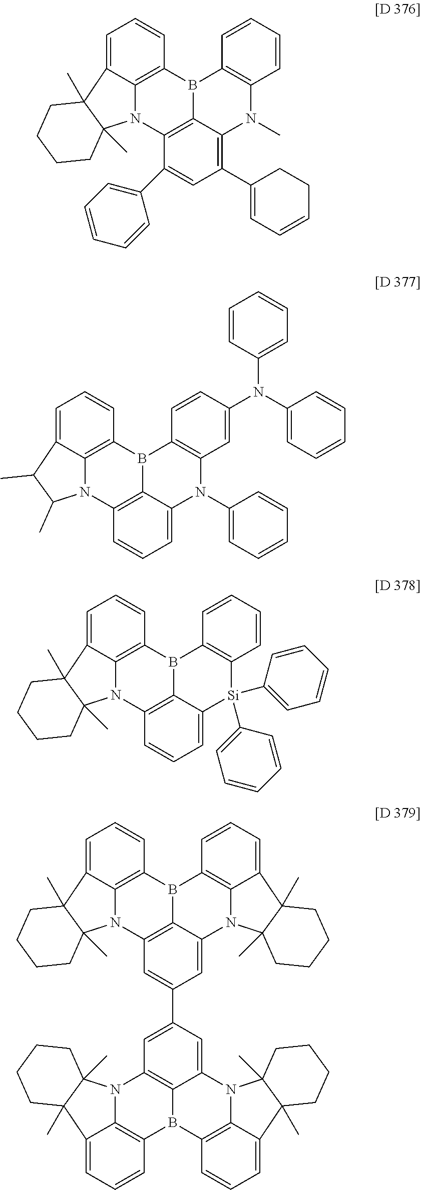

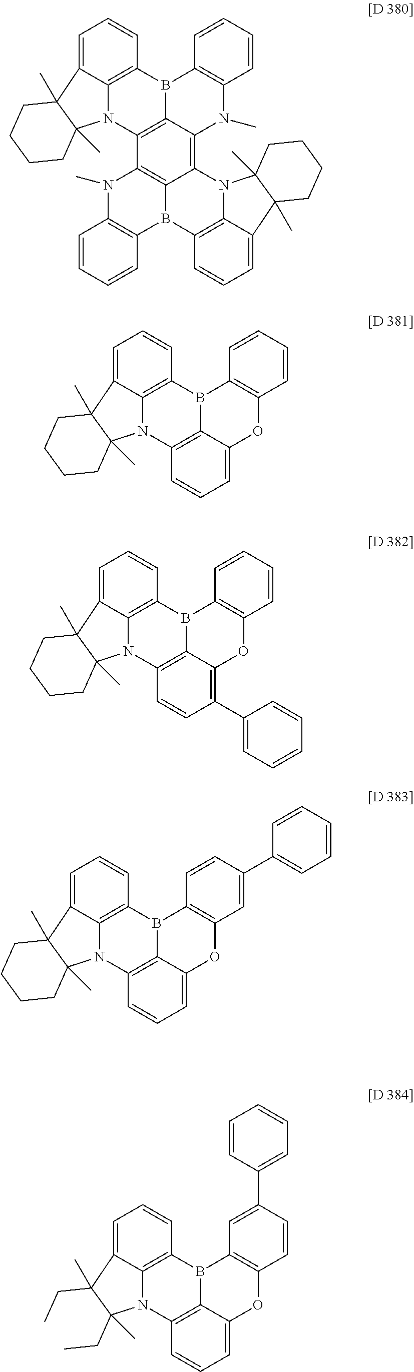 [SMILES: CN1c2ccccc2B2c3cccc4c3N(c3c2c1c1c2c3N(C)c3ccccc3B2c2cccc3c2N1C1(C)CCCCC31C)C1(C)CCCCC41C ; CC12CCCCC1(C)N1c3cccc4c3B(c3ccccc3O4)c3cccc2c31 ; CC12CCCCC1(C)N1c3ccc(-c4ccccc4)c4c3B(c3ccccc3O4)c3cccc2c31 ; CC12CCCCC1(C)N1c3cccc4c3B(c3ccc(-c5ccccc5)cc3O4)c3cccc2c31 ; CCC1(C)c2cccc3c2N(c2cccc4c2B3c2cc(-c3ccccc3)ccc2O4)C1(C)CC]

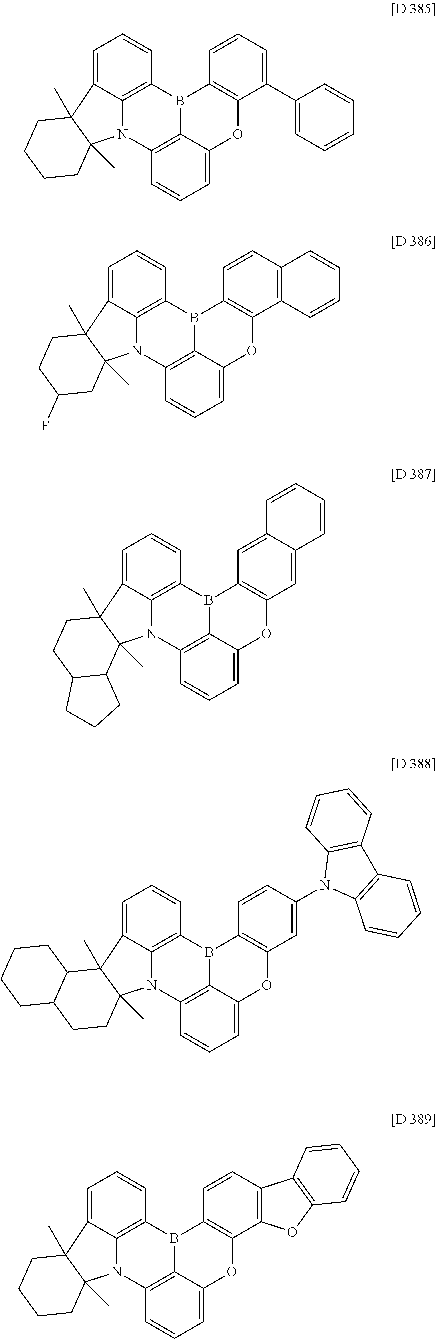 [SMILES: CC12CCCCC1(C)N1c3cccc4c3B(c3cccc(-c5ccccc5)c3O4)c3cccc2c31 ; CC12CCC(F)CC1(C)N1c3cccc4c3B(c3cccc2c31)c1ccc2ccccc2c1O4 ; CC12CCC3CCCC3C1(C)N1c3cccc4c3B(c3cc5ccccc5cc3O4)c3cccc2c31 ; CC12c3cccc4c3N(c3cccc5c3B4c3ccc(-n4c6ccccc6c6ccccc64)cc3O5)C1(C)CCC1CCCCC12 ; CC12CCCCC1(C)N1c3cccc4c3B(c3cccc2c31)c1ccc2c(oc3ccccc32)c1O4]

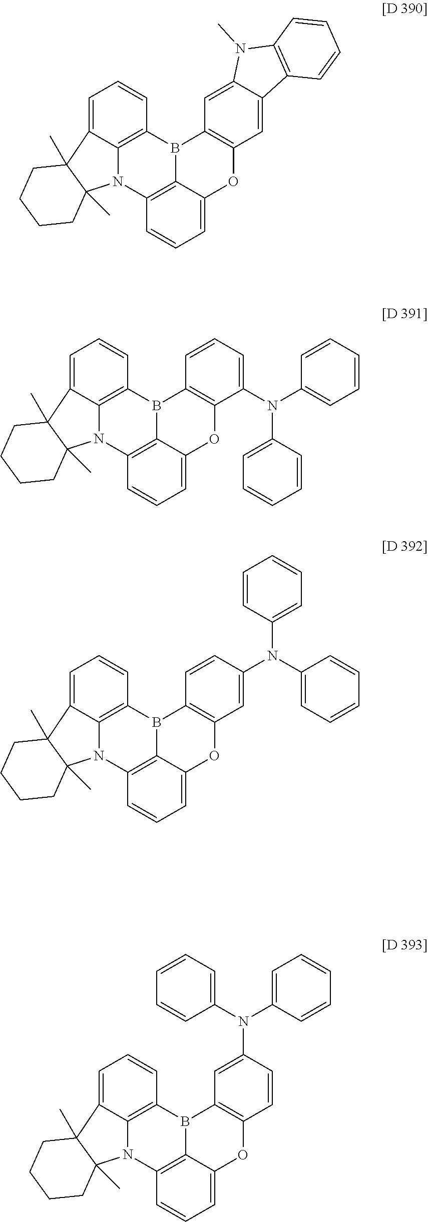 [SMILES: Cn1c2ccccc2c2cc3c(cc21)B1c2cccc4c2N(c2cccc(c21)O3)C1(C)CCCCC41C ; CC12CCCCC1(C)N1c3cccc4c3B(c3cccc(N(c5ccccc5)c5ccccc5)c3O4)c3cccc2c31 ; CC12CCCCC1(C)N1c3cccc4c3B(c3ccc(N(c5ccccc5)c5ccccc5)cc3O4)c3cccc2c31 ; CC12CCCCC1(C)N1c3cccc4c3B(c3cc(N(c5ccccc5)c5ccccc5)ccc3O4)c3cccc2c31]

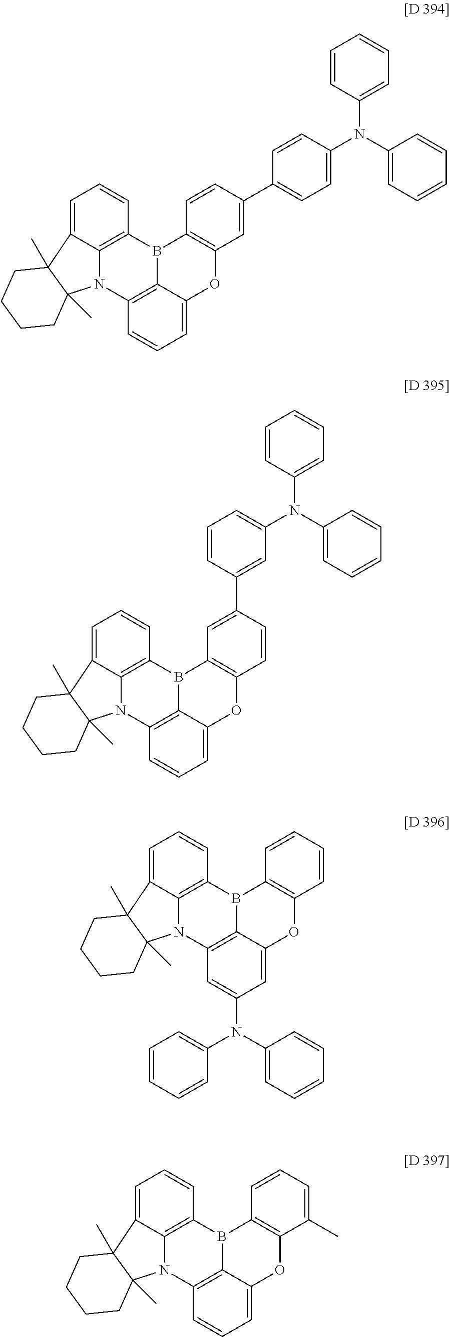 [SMILES: CC12CCCCC1(C)N1c3cccc4c3B(c3ccc(-c5ccc(N(c6ccccc6)c6ccccc6)cc5)cc3O4)c3cccc2c31 ; CC12CCCCC1(C)N1c3cccc4c3B(c3cc(-c5cccc(N(c6ccccc6)c6ccccc6)c5)ccc3O4)c3cccc2c31 ; CC12CCCCC1(C)N1c3cc(N(c4ccccc4)c4ccccc4)cc4c3B(c3ccccc3O4)c3cccc2c31 ; Cc1cccc2c1Oc1cccc3c1B2c1cccc2c1N3C1(C)CCCCC21C]

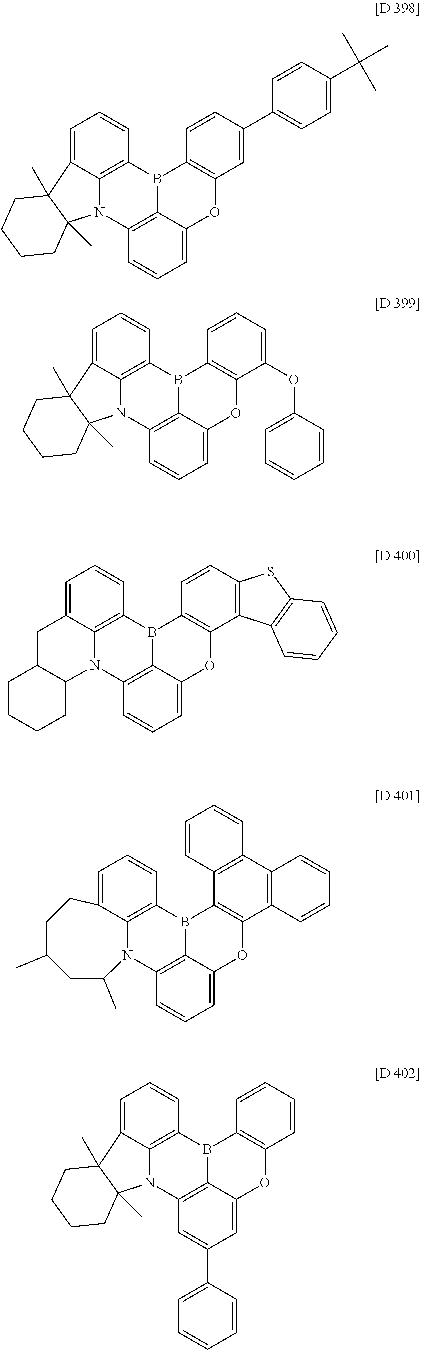

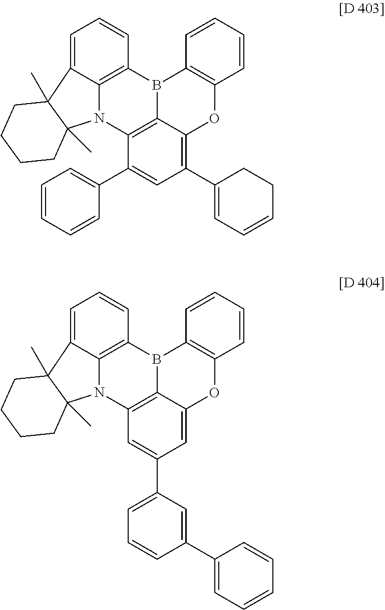 [SMILES: CC12CCCCC1(C)N1c3c(cccc32)B2c3ccccc3Oc3c(C4=CC=CCC4)cc(-c4ccccc4)c1c32 ; CC12CCCCC1(C)N1c3cc(-c4cccc(-c5ccccc5)c4)cc4c3B(c3ccccc3O4)c3cccc2c31]

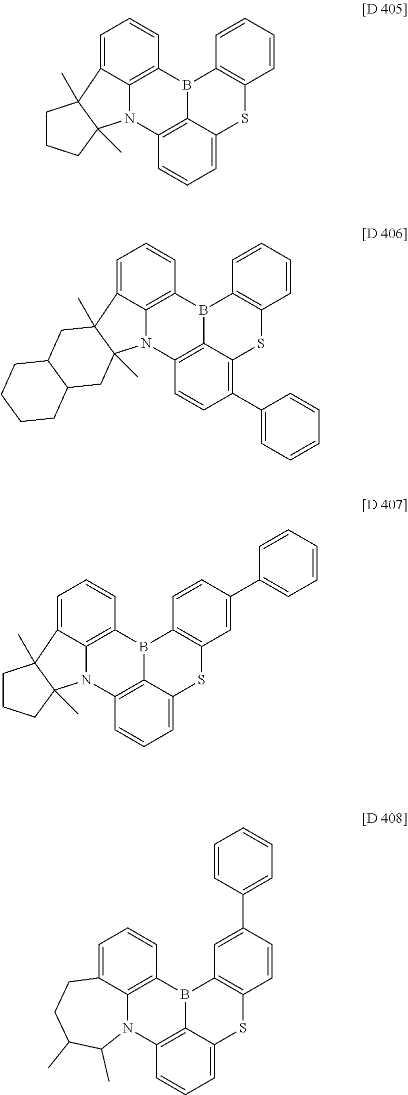 [SMILES: CC12CCCC1(C)N1c3cccc4c3B(c3ccccc3S4)c3cccc2c31 ; CC12CC3CCCCC3CC1(C)N1c3ccc(-c4ccccc4)c4c3B(c3ccccc3S4)c3cccc2c31 ; CC12CCCC1(C)N1c3cccc4c3B(c3ccc(-c5ccccc5)cc3S4)c3cccc2c31 ; CC1CCc2cccc3c2N(c2cccc4c2B3c2cc(-c3ccccc3)ccc2S4)C1C]

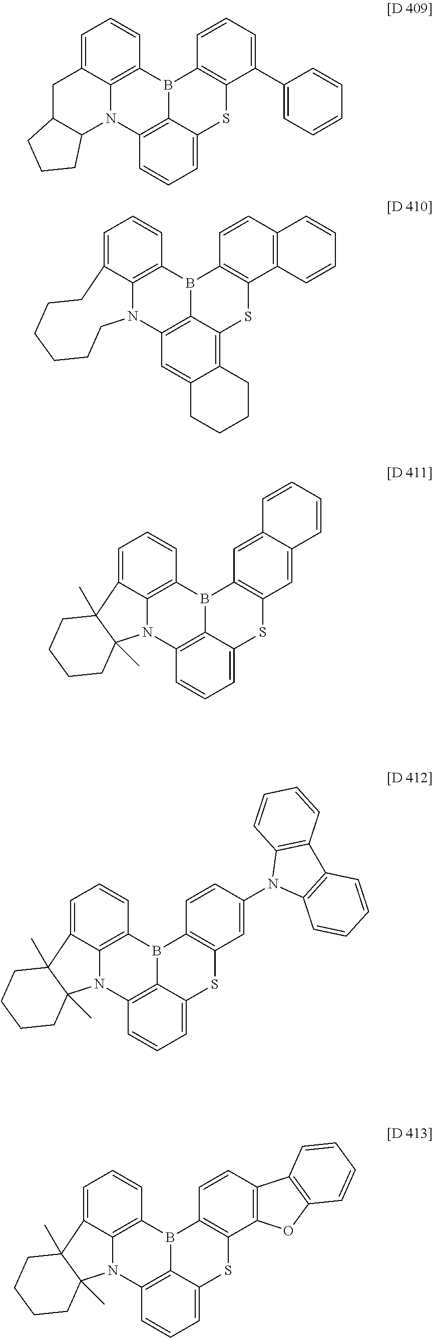 [SMILES: c1ccc(-c2cccc3c2Sc2cccc4c2B3c2cccc3c2N4C2CCCC2C3)cc1 ; c1cc2c3c(c1)B1c4ccc5ccccc5c4Sc4c5c(cc(c41)N3CCCCCC2)CCCC5 ; CC12CCCCC1(C)N1c3cccc4c3B(c3cc5ccccc5cc3S4)c3cccc2c31 ; CC12CCCCC1(C)N1c3cccc4c3B(c3ccc(-n5c6ccccc6c6ccccc65)cc3S4)c3cccc2c31 ; CC12CCCCC1(C)N1c3cccc4c3B(c3cccc2c31)c1ccc2c(oc3ccccc32)c1S4]

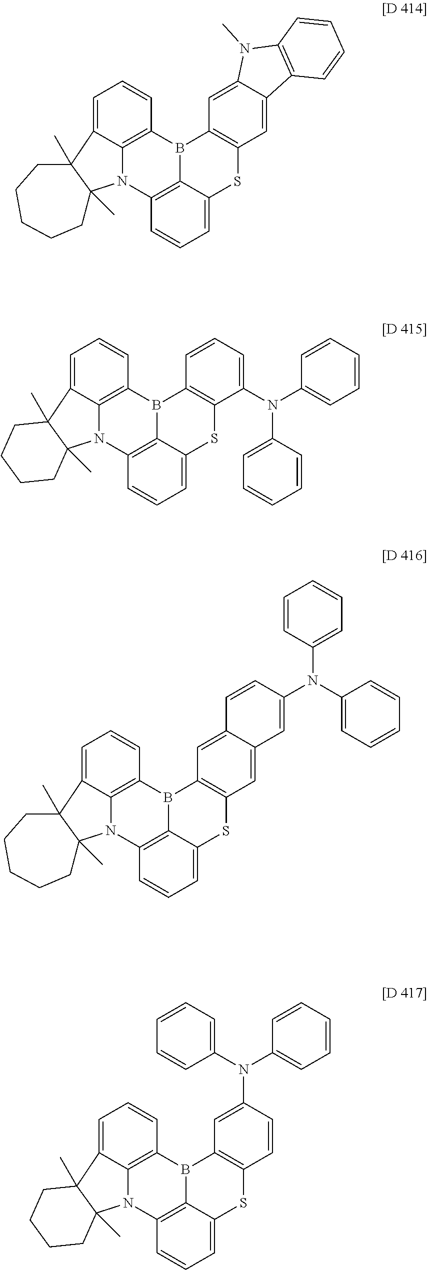 [SMILES: Cn1c2ccccc2c2cc3c(cc21)B1c2cccc4c2N(c2cccc(c21)S3)C1(C)CCCCCC41C ; CC12CCCCC1(C)N1c3cccc4c3B(c3cccc(N(c5ccccc5)c5ccccc5)c3S4)c3cccc2c31 ; CC12CCCCCC1(C)N1c3cccc4c3B(c3cc5ccc(N(c6ccccc6)c6ccccc6)cc5cc3S4)c3cccc2c31 ; CC12CCCCC1(C)N1c3cccc4c3B(c3cc(N(c5ccccc5)c5ccccc5)ccc3S4)c3cccc2c31]

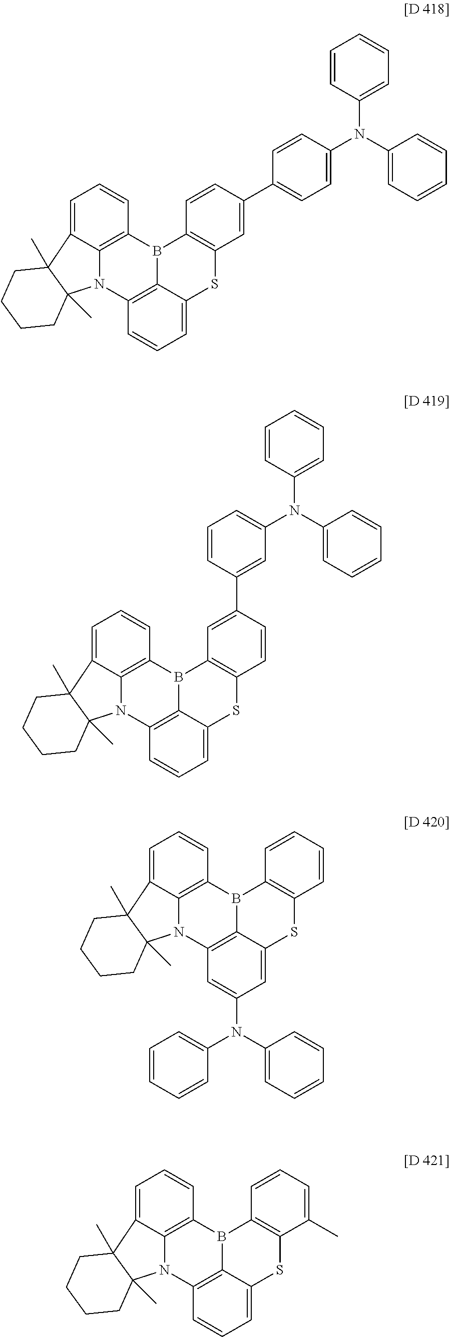 [SMILES: CC12CCCCC1(C)N1c3cccc4c3B(c3ccc(-c5ccc(N(c6ccccc6)c6ccccc6)cc5)cc3S4)c3cccc2c31 ; CC12CCCCC1(C)N1c3cccc4c3B(c3cc(-c5cccc(N(c6ccccc6)c6ccccc6)c5)ccc3S4)c3cccc2c31 ; CC12CCCCC1(C)N1c3cc(N(c4ccccc4)c4ccccc4)cc4c3B(c3ccccc3S4)c3cccc2c31 ; Cc1cccc2c1Sc1cccc3c1B2c1cccc2c1N3C1(C)CCCCC21C]

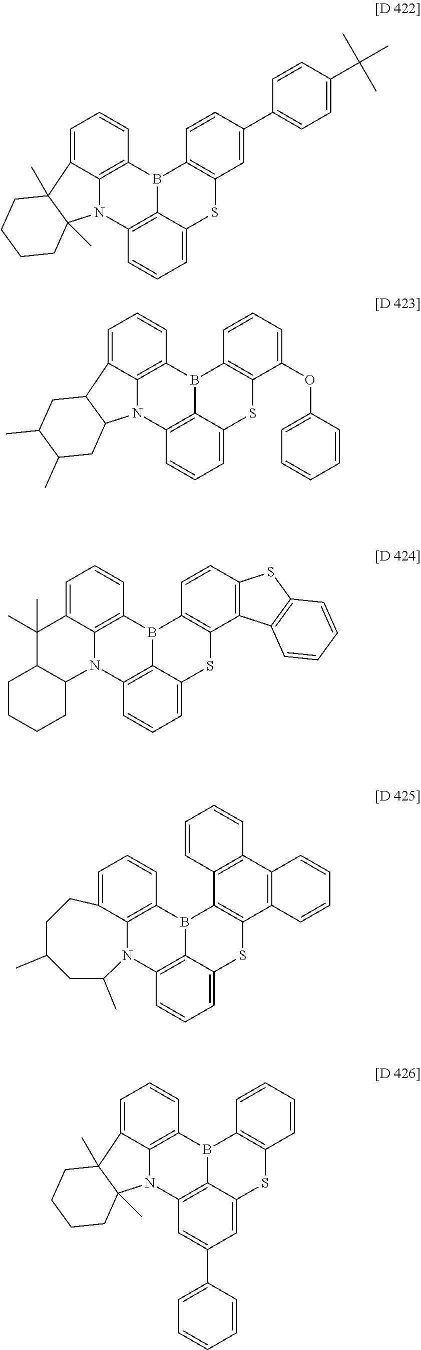

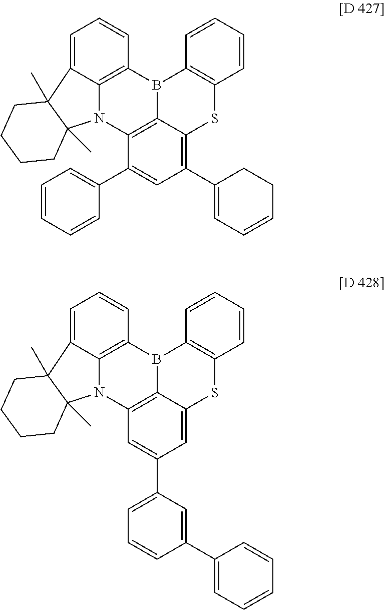 [SMILES: CC12CCCCC1(C)N1c3c(cccc32)B2c3ccccc3Sc3c(C4=CC=CCC4)cc(-c4ccccc4)c1c32 ; CC12CCCCC1(C)N1c3cc(-c4cccc(-c5ccccc5)c4)cc4c3B(c3ccccc3S4)c3cccc2c31]

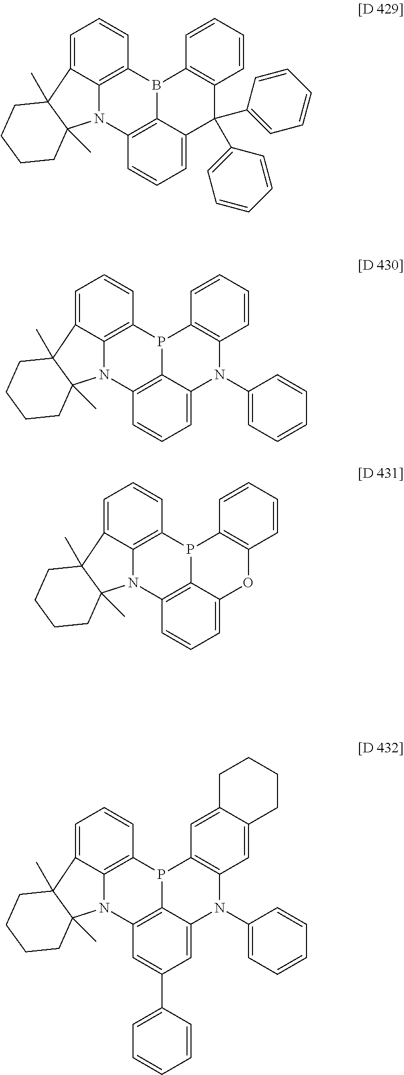 [SMILES: CC12CCCCC1(C)N1c3cccc4c3B(c3ccccc3C4(c3ccccc3)c3ccccc3)c3cccc2c31 ; CC12CCCCC1(C)N1c3cccc4c3P(c3ccccc3N4c3ccccc3)c3cccc2c31 ; CC12CCCCC1(C)N1c3cccc4c3P(c3ccccc3O4)c3cccc2c31 ; CC12CCCCC1(C)N1c3cc(-c4ccccc4)cc4c3P(c3cc5c(cc3N4c3ccccc3)CCCC5)c3cccc2c31]

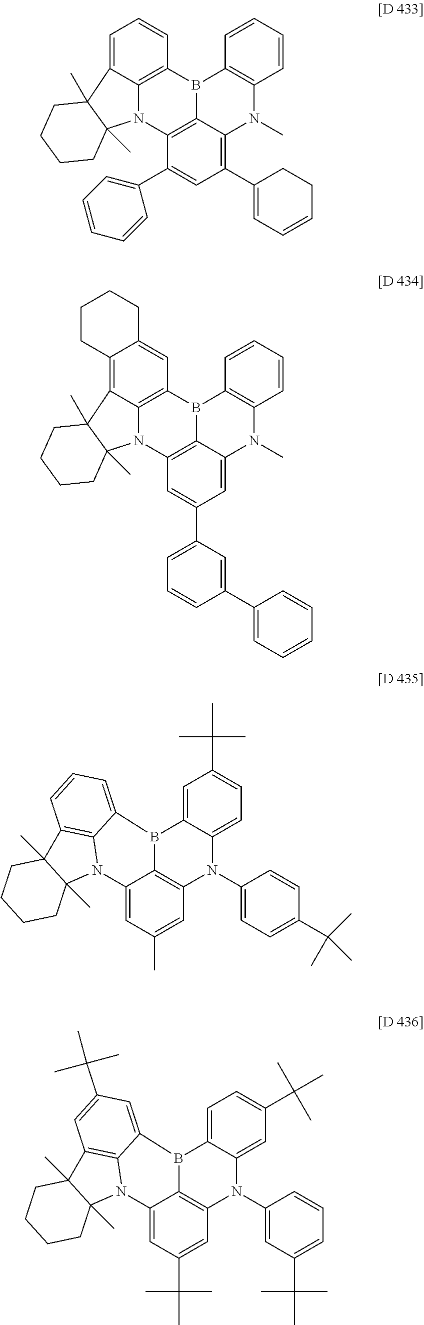 [SMILES: CN1c2ccccc2B2c3cccc4c3N(c3c(-c5ccccc5)cc(C5=CC=CCC5)c1c32)C1(C)CCCCC41C ; CN1c2ccccc2B2c3cc4c(c5c3N(c3cc(-c6cccc(-c7ccccc7)c6)cc1c32)C1(C)CCCCC51C)CCCC4 ; Cc1cc2c3c(c1)N1c4c(cccc4C4(C)CCCCC14C)B3c1cc(C(C)(C)C)ccc1N2c1ccc(C(C)(C)C)cc1 ; CC(C)(C)c1cccc(N2c3cc(C(C)(C)C)ccc3B3c4cc(C(C)(C)C)cc5c4N(c4cc(C(C)(C)C)cc2c43)C2(C)CCCCC52C)c1]

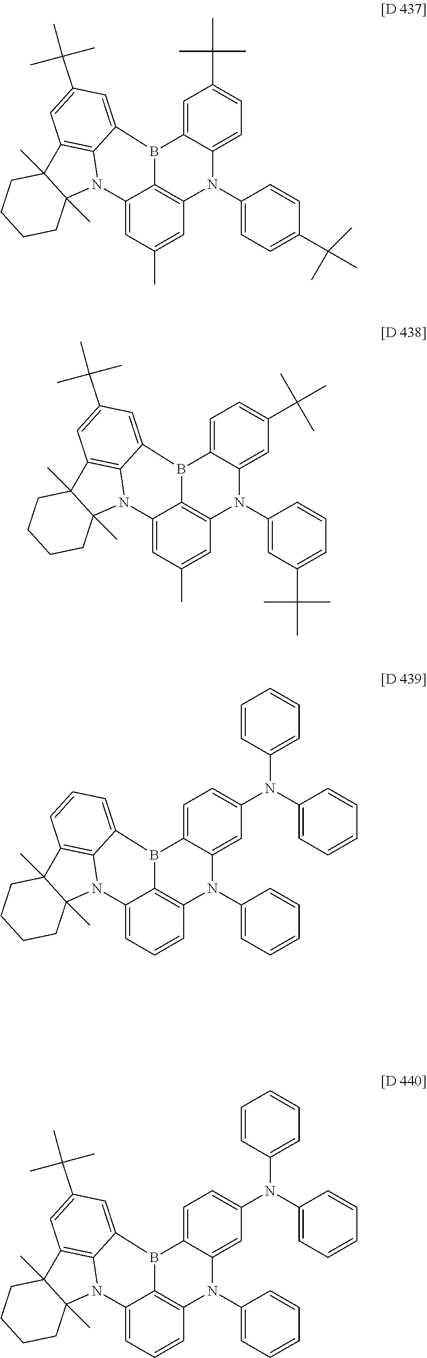 [SMILES: Cc1cc2c3c(c1)N1c4c(cc(C(C)(C)C)cc4C4(C)CCCCC14C)B3c1cc(C(C)(C)C)ccc1N2c1ccc(C(C)(C)C)cc1 ; Cc1cc2c3c(c1)N1c4c(cc(C(C)(C)C)cc4C4(C)CCCCC14C)B3c1ccc(C(C)(C)C)cc1N2c1cccc(C(C)(C)C)c1 ; CC12CCCCC1(C)N1c3cccc4c3B(c3ccc(N(c5ccccc5)c5ccccc5)cc3N4c3ccccc3)c3cccc2c31 ; CC(C)(C)c1cc2c3c(c1)C1(C)CCCCC1(C)N3c1cccc3c1B2c1ccc(N(c2ccccc2)c2ccccc2)cc1N3c1ccccc1]

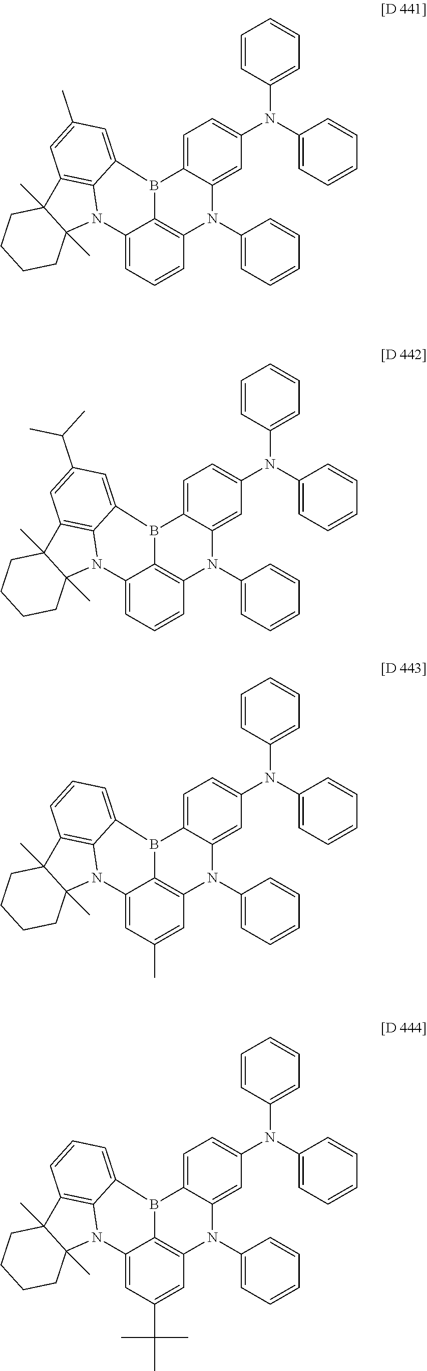 [SMILES: Cc1cc2c3c(c1)C1(C)CCCCC1(C)N3c1cccc3c1B2c1ccc(N(c2ccccc2)c2ccccc2)cc1N3c1ccccc1 ; CC(C)c1cc2c3c(c1)C1(C)CCCCC1(C)N3c1cccc3c1B2c1ccc(N(c2ccccc2)c2ccccc2)cc1N3c1ccccc1 ; Cc1cc2c3c(c1)N1c4c(cccc4C4(C)CCCCC14C)B3c1ccc(N(c3ccccc3)c3ccccc3)cc1N2c1ccccc1 ; CC(C)(C)c1cc2c3c(c1)N1c4c(cccc4C4(C)CCCCC14C)B3c1ccc(N(c3ccccc3)c3ccccc3)cc1N2c1ccccc1]

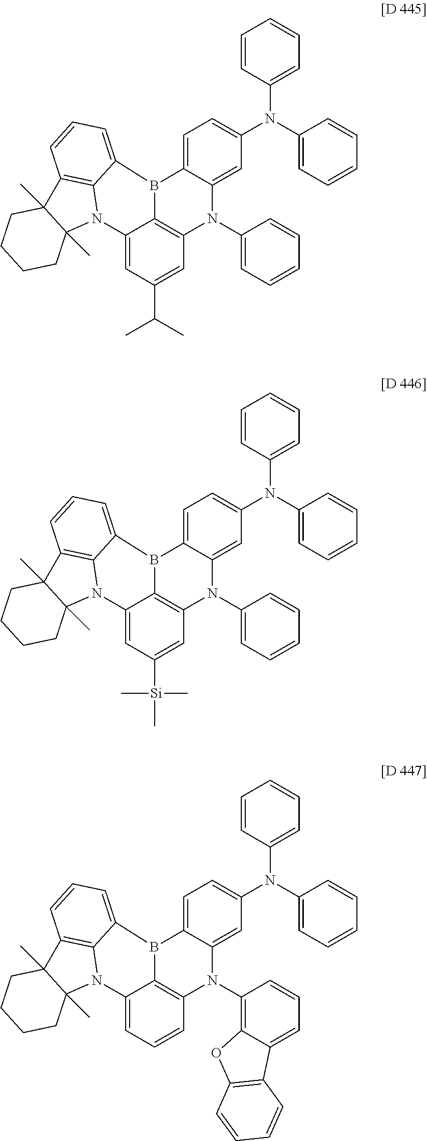 [SMILES: CC(C)c1cc2c3c(c1)N1c4c(cccc4C4(C)CCCCC14C)B3c1ccc(N(c3ccccc3)c3ccccc3)cc1N2c1ccccc1 ; CC12CCCCC1(C)N1c3cc([Si](C)(C)C)cc4c3B(c3ccc(N(c5ccccc5)c5ccccc5)cc3N4c3ccccc3)c3cccc2c31 ; CC12CCCCC1(C)N1c3cccc4c3B(c3ccc(N(c5ccccc5)c5ccccc5)cc3N4c3cccc4c3oc3ccccc34)c3cccc2c31]

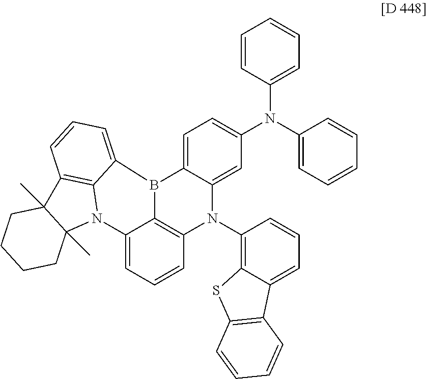 [SMILES: CC12CCCCC1(C)N1c3cccc4c3B(c3ccc(N(c5ccccc5)c5ccccc5)cc3N4c3cccc4c3sc3ccccc34)c3cccc2c31]

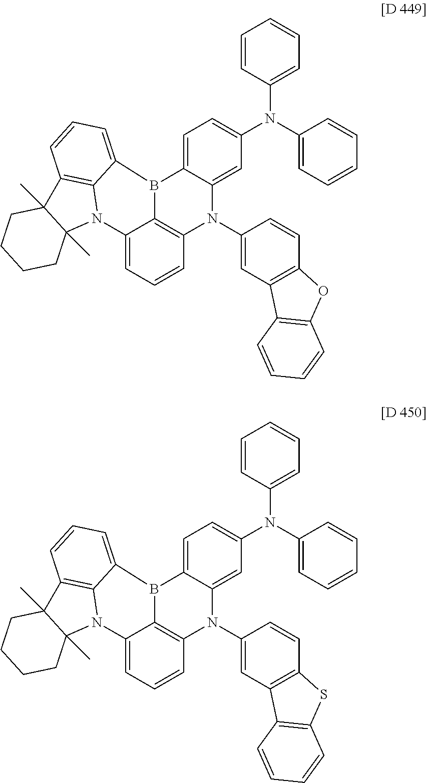 [SMILES: CC12CCCCC1(C)N1c3cccc4c3B(c3ccc(N(c5ccccc5)c5ccccc5)cc3N4c3ccc4oc5ccccc5c4c3)c3cccc2c31 ; CC12CCCCC1(C)N1c3cccc4c3B(c3ccc(N(c5ccccc5)c5ccccc5)cc3N4c3ccc4sc5ccccc5c4c3)c3cccc2c31]

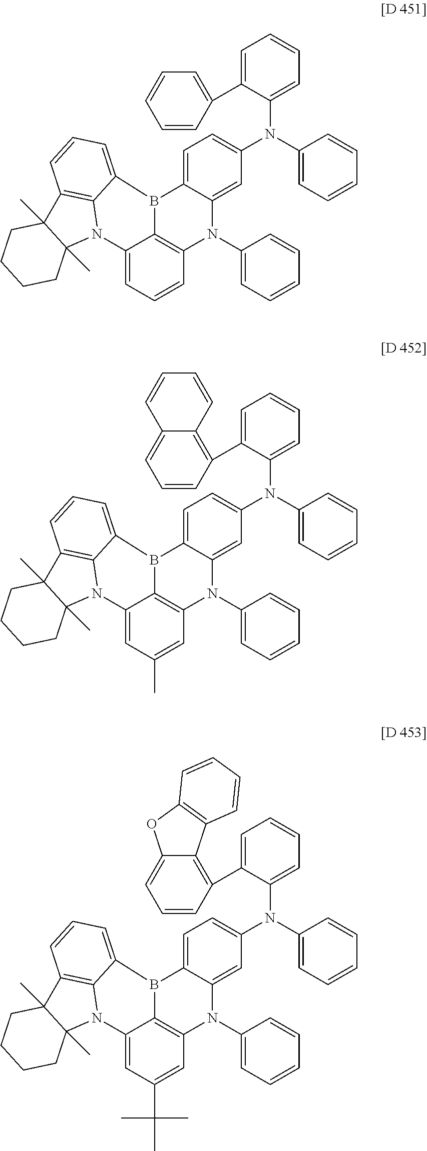 [SMILES: CC12CCCCC1(C)N1c3cccc4c3B(c3ccc(N(c5ccccc5)c5ccccc5-c5ccccc5)cc3N4c3ccccc3)c3cccc2c31 ; Cc1cc2c3c(c1)N1c4c(cccc4C4(C)CCCCC14C)B3c1ccc(N(c3ccccc3)c3ccccc3-c3cccc4ccccc34)cc1N2c1ccccc1 ; CC(C)(C)c1cc2c3c(c1)N1c4c(cccc4C4(C)CCCCC14C)B3c1ccc(N(c3ccccc3)c3ccccc3-c3cccc4oc5ccccc5c34)cc1N2c1ccccc1]

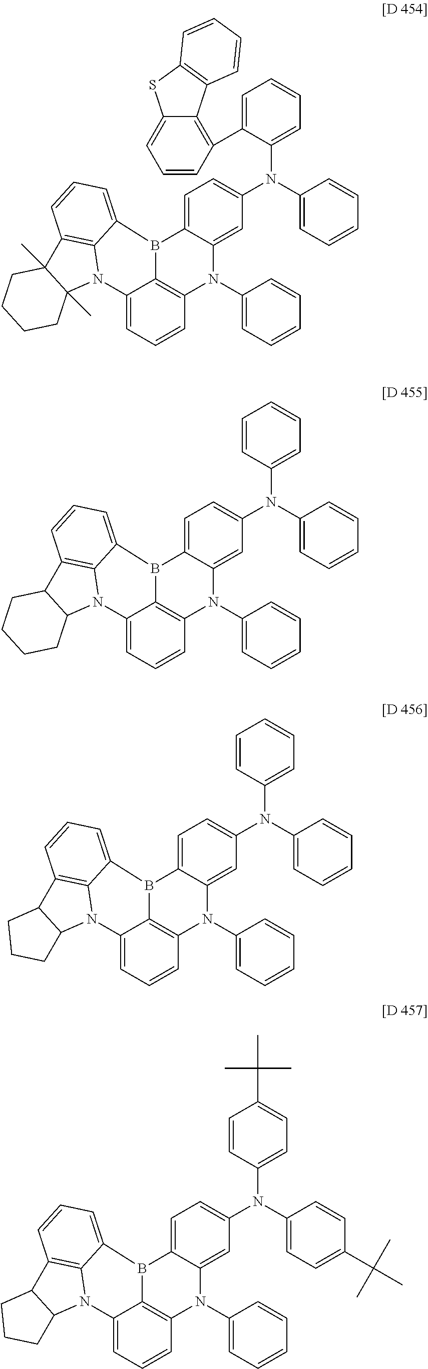 [SMILES: CC12CCCCC1(C)N1c3cccc4c3B(c3ccc(N(c5ccccc5)c5ccccc5-c5cccc6sc7ccccc7c56)cc3N4c3ccccc3)c3cccc2c31 ; c1ccc(N(c2ccccc2)c2ccc3c(c2)N(c2ccccc2)c2cccc4c2B3c2cccc3c2N4C2CCCCC32)cc1 ; c1ccc(N(c2ccccc2)c2ccc3c(c2)N(c2ccccc2)c2cccc4c2B3c2cccc3c2N4C2CCCC32)cc1 ; CC(C)(C)c1ccc(N(c2ccc(C(C)(C)C)cc2)c2ccc3c(c2)N(c2ccccc2)c2cccc4c2B3c2cccc3c2N4C2CCCC32)cc1]

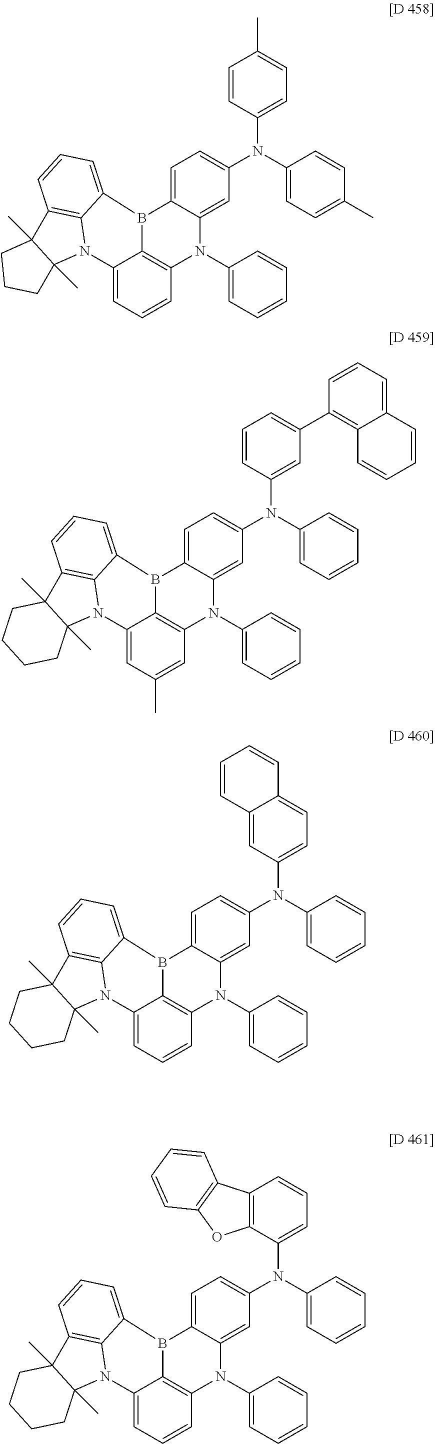 [SMILES: Cc1ccc(N(c2ccc(C)cc2)c2ccc3c(c2)N(c2ccccc2)c2cccc4c2B3c2cccc3c2N4C2(C)CCCC32C)cc1 ; Cc1cc2c3c(c1)N1c4c(cccc4C4(C)CCCCC14C)B3c1ccc(N(c3ccccc3)c3cccc(-c4cccc5ccccc45)c3)cc1N2c1ccccc1 ; CC12CCCCC1(C)N1c3cccc4c3B(c3ccc(N(c5ccccc5)c5ccc6ccccc6c5)cc3N4c3ccccc3)c3cccc2c31 ; CC12CCCCC1(C)N1c3cccc4c3B(c3ccc(N(c5ccccc5)c5cccc6c5oc5ccccc56)cc3N4c3ccccc3)c3cccc2c31]

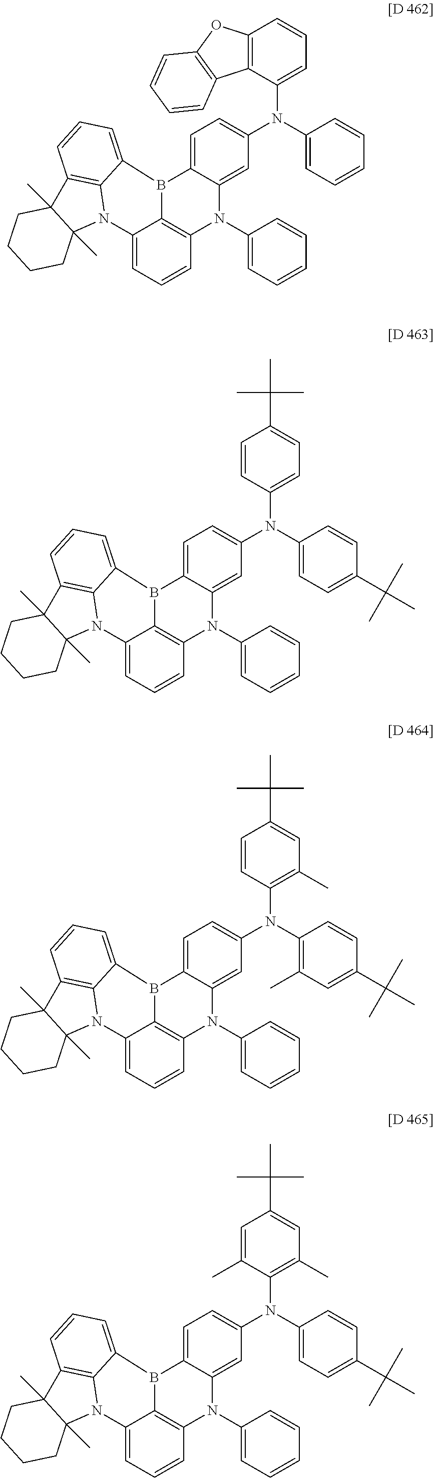 [SMILES: CC12CCCCC1(C)N1c3cccc4c3B(c3ccc(N(c5ccccc5)c5cccc6oc7ccccc7c56)cc3N4c3ccccc3)c3cccc2c31 ; CC(C)(C)c1ccc(N(c2ccc(C(C)(C)C)cc2)c2ccc3c(c2)N(c2ccccc2)c2cccc4c2B3c2cccc3c2N4C2(C)CCCCC32C)cc1 ; Cc1cc(C(C)(C)C)ccc1N(c1ccc2c(c1)N(c1ccccc1)c1cccc3c1B2c1cccc2c1N3C1(C)CCCCC21C)c1ccc(C(C)(C)C)cc1C ; Cc1cc(C(C)(C)C)cc(C)c1N(c1ccc(C(C)(C)C)cc1)c1ccc2c(c1)N(c1ccccc1)c1cccc3c1B2c1cccc2c1N3C1(C)CCCCC21C]

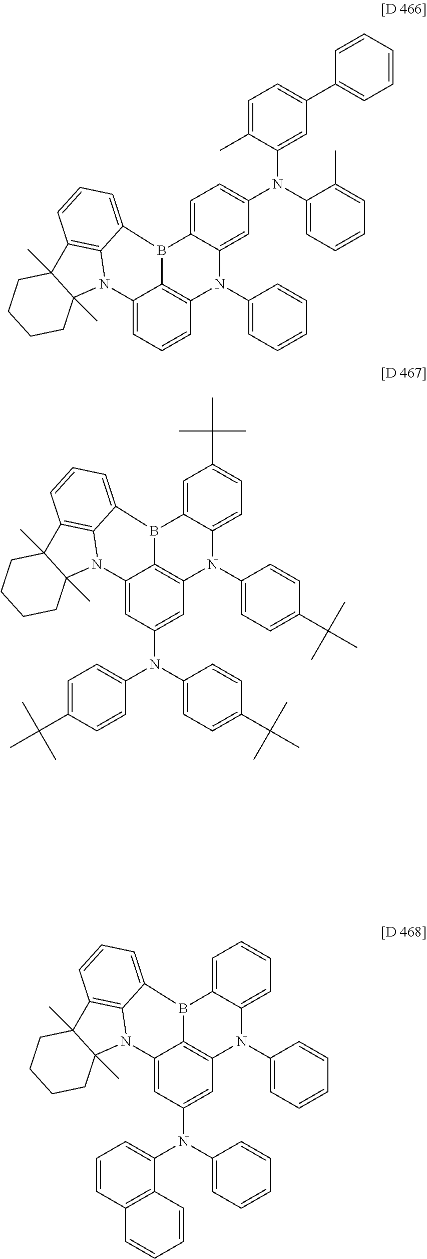 [SMILES: Cc1ccccc1N(c1ccc2c(c1)N(c1ccccc1)c1cccc3c1B2c1cccc2c1N3C1(C)CCCCC21C)c1cc(-c2ccccc2)ccc1C ; CC(C)(C)c1ccc(N(c2ccc(C(C)(C)C)cc2)c2cc3c4c(c2)N2c5c(cccc5C5(C)CCCCC25C)B4c2cc(C(C)(C)C)ccc2N3c2ccc(C(C)(C)C)cc2)cc1 ; CC12CCCCC1(C)N1c3cc(N(c4ccccc4)c4cccc5ccccc45)cc4c3B(c3ccccc3N4c3ccccc3)c3cccc2c31]

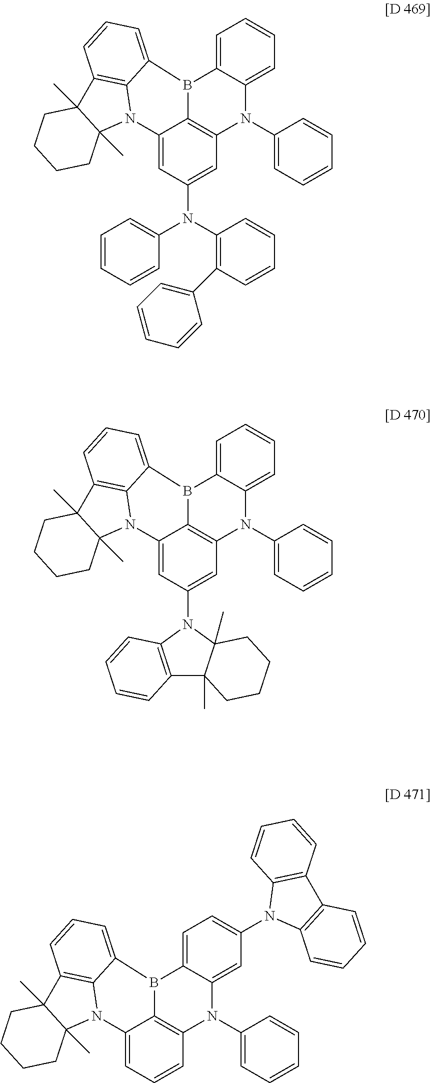 [SMILES: CC12CCCCC1(C)N1c3cc(N(c4ccccc4)c4ccccc4-c4ccccc4)cc4c3B(c3ccccc3N4c3ccccc3)c3cccc2c31 ; CC12CCCCC1(C)N(c1cc3c4c(c1)N1c5c(cccc5C5(C)CCCCC15C)B4c1ccccc1N3c1ccccc1)c1ccccc12 ; CC12CCCCC1(C)N1c3cccc4c3B(c3ccc(-n5c6ccccc6c6ccccc65)cc3N4c3ccccc3)c3cccc2c31]

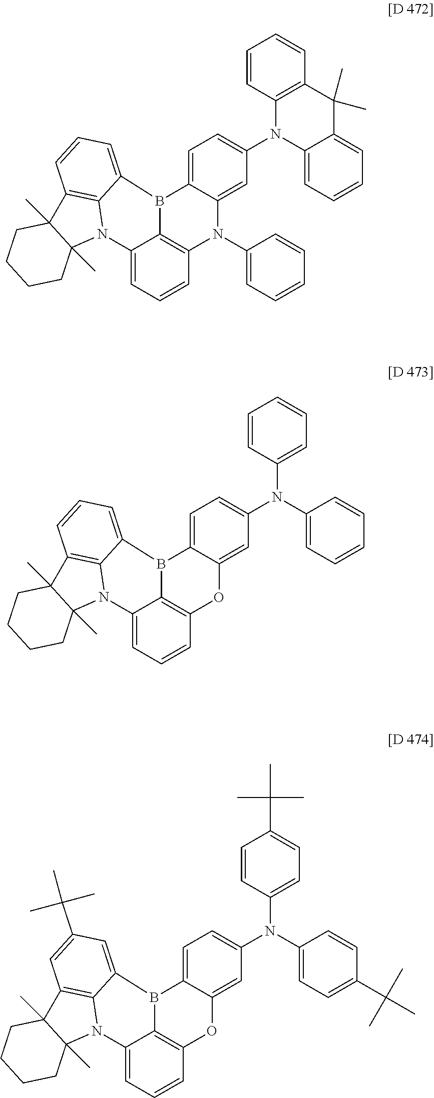 [SMILES: CC1(C)c2ccccc2N(c2ccc3c(c2)N(c2ccccc2)c2cccc4c2B3c2cccc3c2N4C2(C)CCCCC32C)c2ccccc21 ; CC12CCCCC1(C)N1c3cccc4c3B(c3ccc(N(c5ccccc5)c5ccccc5)cc3O4)c3cccc2c31 ; CC(C)(C)c1ccc(N(c2ccc(C(C)(C)C)cc2)c2ccc3c(c2)Oc2cccc4c2B3c2cc(C(C)(C)C)cc3c2N4C2(C)CCCCC32C)cc1]

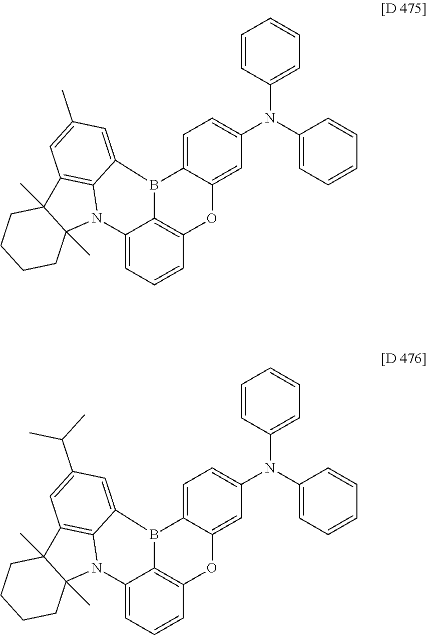 [SMILES: Cc1cc2c3c(c1)C1(C)CCCCC1(C)N3c1cccc3c1B2c1ccc(N(c2ccccc2)c2ccccc2)cc1O3 ; CC(C)c1cc2c3c(c1)C1(C)CCCCC1(C)N3c1cccc3c1B2c1ccc(N(c2ccccc2)c2ccccc2)cc1O3]

Among the dopant compounds useful in the light-emitting layer according to the present disclosure, examples of the compound represented by Chemical Formula D6 or Chemical Formula D7 include the following <D 501> to <D 587>:

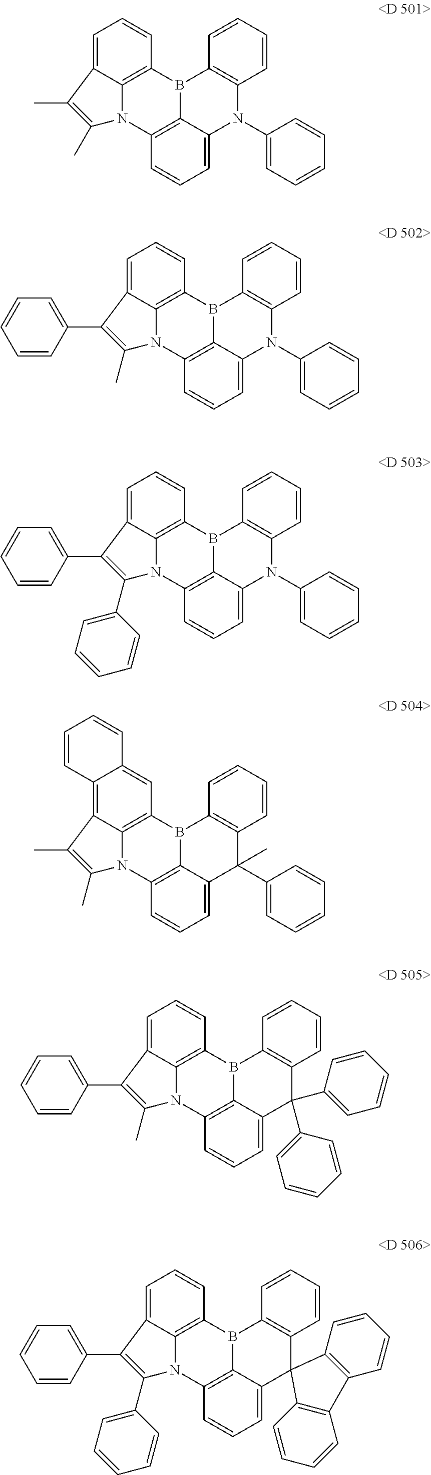

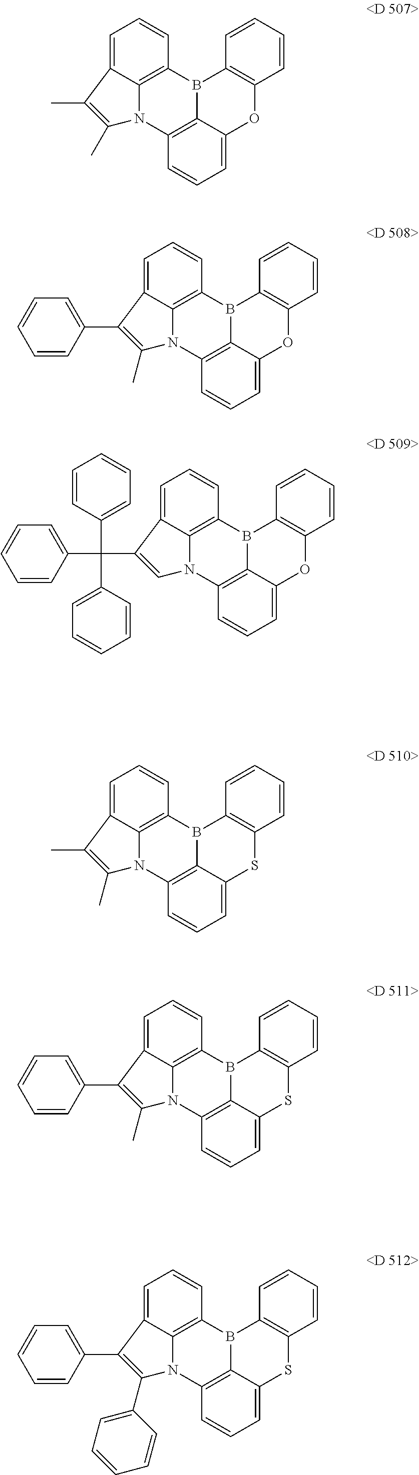 [SMILES: Cc1c(C)n2c3c(cccc13)B1c3ccccc3Oc3cccc-2c31 ; Cc1c(-c2ccccc2)c2cccc3c2n1-c1cccc2c1B3c1ccccc1O2 ; c1ccc(C(c2ccccc2)(c2ccccc2)c2cn3c4c(cccc24)B2c4ccccc4Oc4cccc-3c42)cc1 ; Cc1c(C)n2c3c(cccc13)B1c3ccccc3Sc3cccc-2c31 ; Cc1c(-c2ccccc2)c2cccc3c2n1-c1cccc2c1B3c1ccccc1S2 ; c1ccc(-c2c(-c3ccccc3)n3c4c(cccc24)B2c4ccccc4Sc4cccc-3c42)cc1]

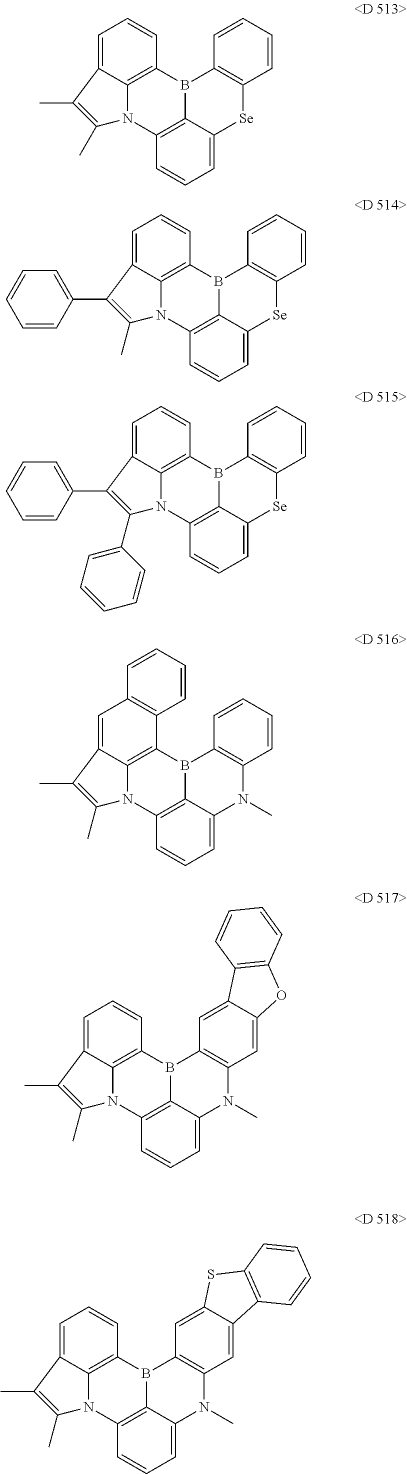

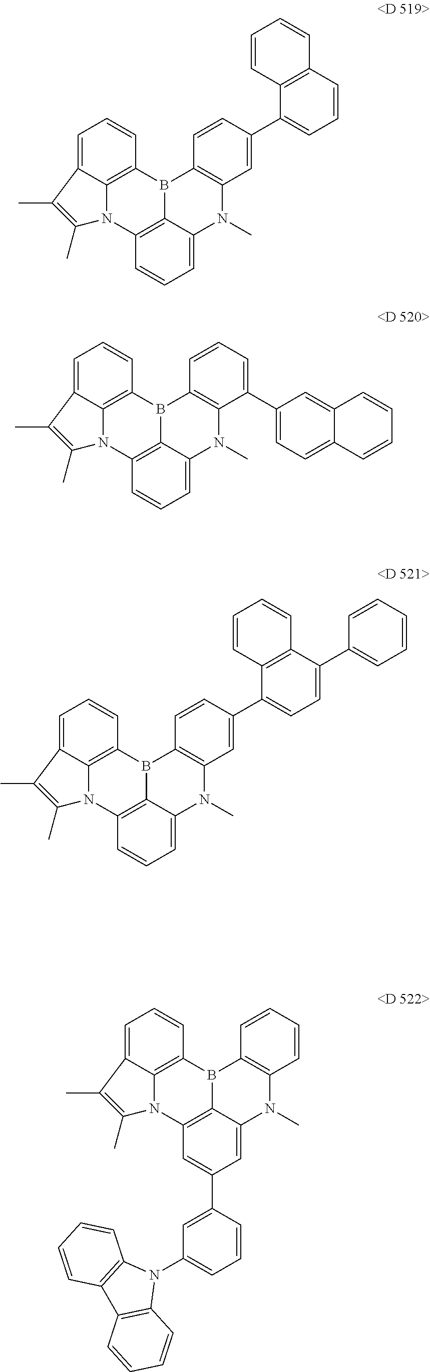 [SMILES: Cc1c(C)n2c3c(cccc13)B1c3ccc(-c4cccc5ccccc45)cc3N(C)c3cccc-2c31 ; Cc1c(C)n2c3c(cccc13)B1c3cccc(-c4ccc5ccccc5c4)c3N(C)c3cccc-2c31 ; Cc1c(C)n2c3c(cccc13)B1c3ccc(-c4ccc(-c5ccccc5)c5ccccc45)cc3N(C)c3cccc-2c31 ; Cc1c(C)n2c3c(cccc13)B1c3ccccc3N(C)c3cc(-c4cccc(-n5c6ccccc6c6ccccc65)c4)cc-2c31]

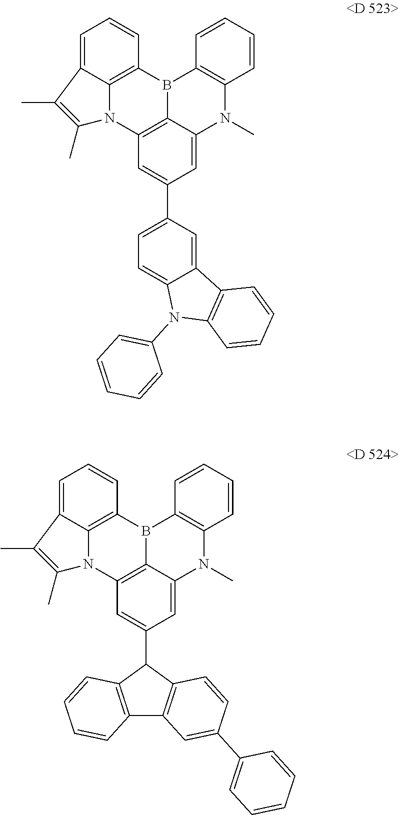 [SMILES: Cc1c(C)n2c3c(cccc13)B1c3ccccc3N(C)c3cc(-c4ccc5c(c4)c4ccccc4n5-c4ccccc4)cc-2c31 ; Cc1c(C)n2c3c(cccc13)B1c3ccccc3N(C)c3cc(C4c5ccccc5-c5cc(-c6ccccc6)ccc54)cc-2c31]

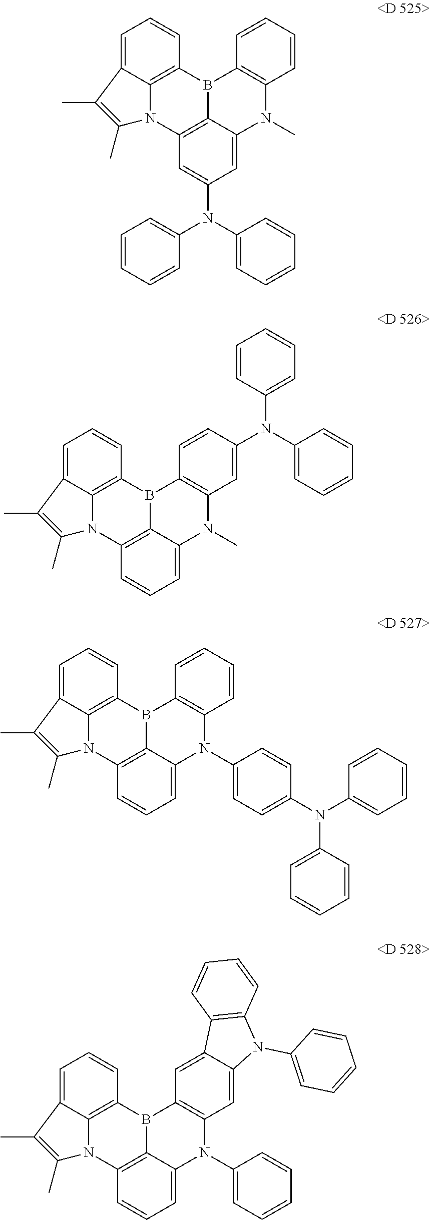

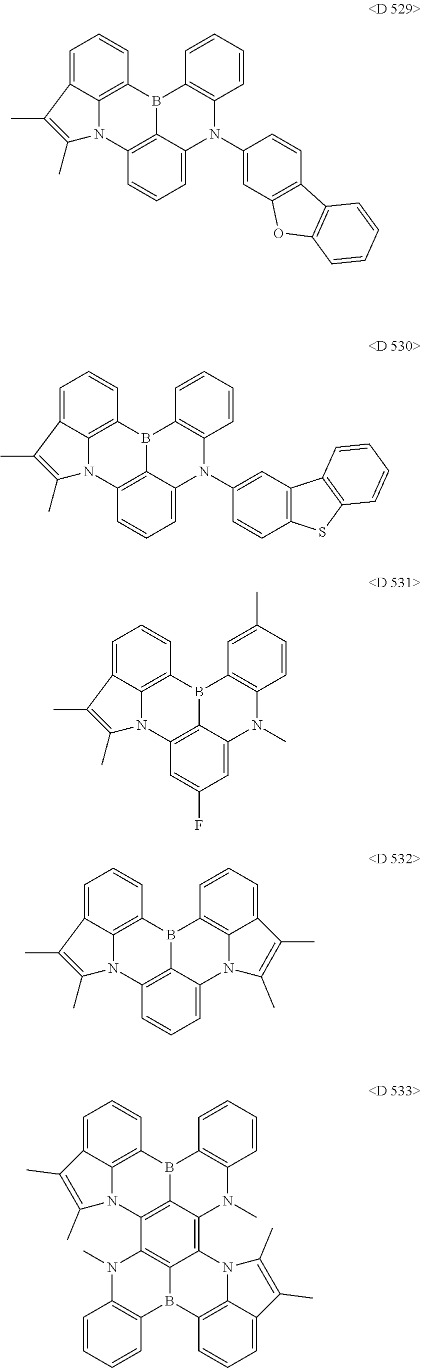

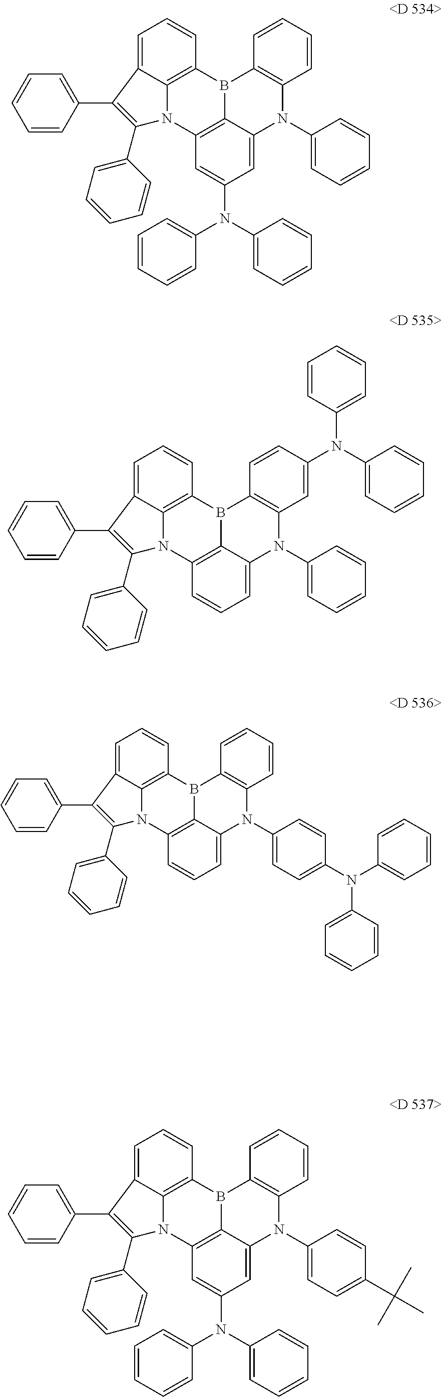 [SMILES: c1ccc(-c2c(-c3ccccc3)n3c4c(cccc24)B2c4ccccc4N(c4ccccc4)c4cc(N(c5ccccc5)c5ccccc5)cc-3c42)cc1 ; c1ccc(-c2c(-c3ccccc3)n3c4c(cccc24)B2c4ccc(N(c5ccccc5)c5ccccc5)cc4N(c4ccccc4)c4cccc-3c42)cc1 ; c1ccc(-c2c(-c3ccccc3)n3c4c(cccc24)B2c4ccccc4N(c4ccc(N(c5ccccc5)c5ccccc5)cc4)c4cccc-3c42)cc1 ; CC(C)(C)c1ccc(N2c3ccccc3B3c4c2cc(N(c2ccccc2)c2ccccc2)cc4-n2c(-c4ccccc4)c(-c4ccccc4)c4cccc3c42)cc1]

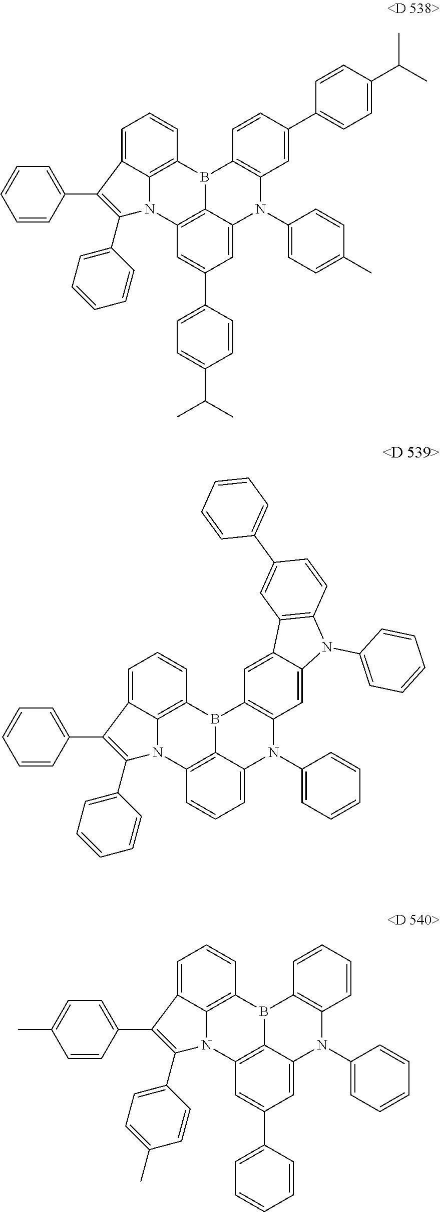

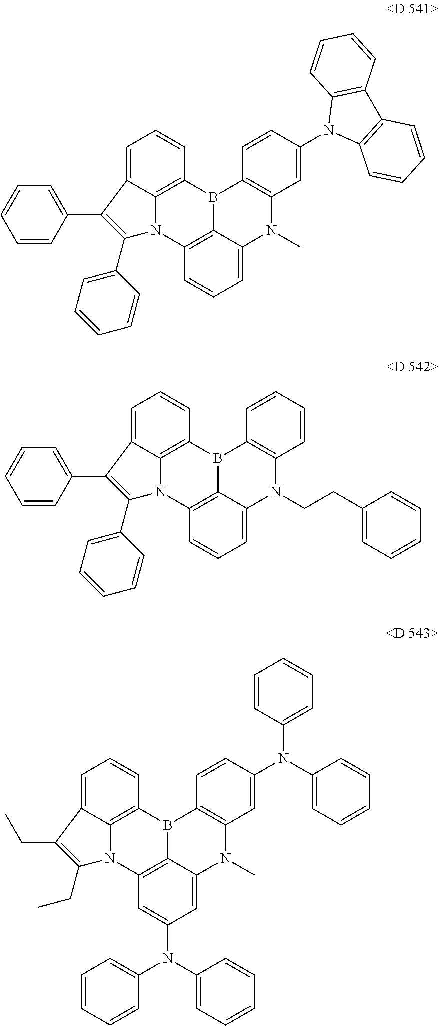 [SMILES: CN1c2cc(-n3c4ccccc4c4ccccc43)ccc2B2c3c1cccc3-n1c(-c3ccccc3)c(-c3ccccc3)c3cccc2c31 ; c1ccc(CCN2c3ccccc3B3c4c2cccc4-n2c(-c4ccccc4)c(-c4ccccc4)c4cccc3c42)cc1 ; CCc1c(CC)n2c3c(cccc13)B1c3ccc(N(c4ccccc4)c4ccccc4)cc3N(C)c3cc(N(c4ccccc4)c4ccccc4)cc-2c31]

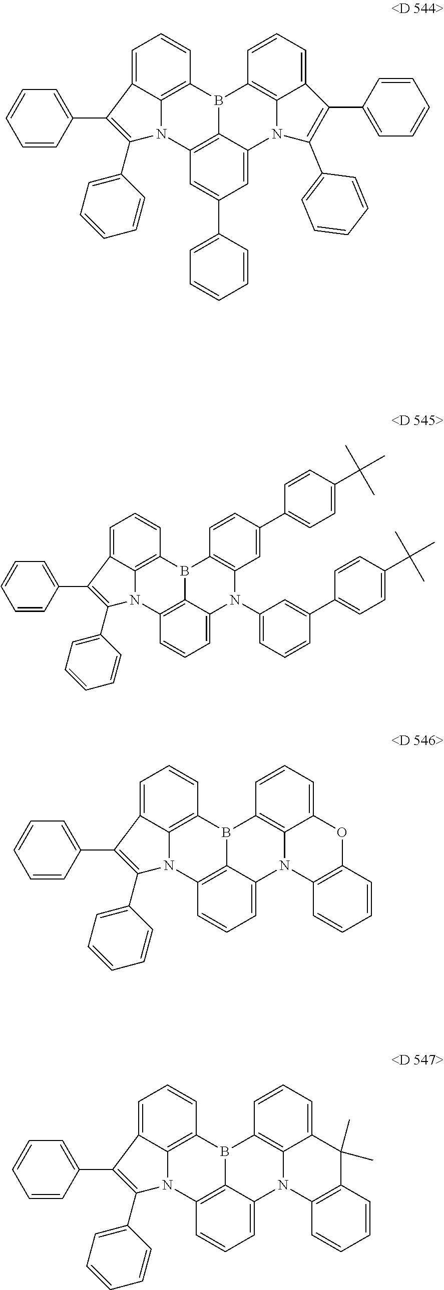 [SMILES: c1ccc(-c2cc3c4c(c2)-n2c(-c5ccccc5)c(-c5ccccc5)c5cccc(c52)B4c2cccc4c(-c5ccccc5)c(-c5ccccc5)n-3c24)cc1 ; CC(C)(C)c1ccc(-c2cccc(N3c4cc(-c5ccc(C(C)(C)C)cc5)ccc4B4c5c3cccc5-n3c(-c5ccccc5)c(-c5ccccc5)c5cccc4c53)c2)cc1 ; c1ccc(-c2c(-c3ccccc3)n3c4c(cccc24)B2c4cccc5c4N(c4ccccc4O5)c4cccc-3c42)cc1 ; CC1(C)c2ccccc2N2c3cccc4c3B(c3cccc1c32)c1cccc2c(-c3ccccc3)c(-c3ccccc3)n-4c12]

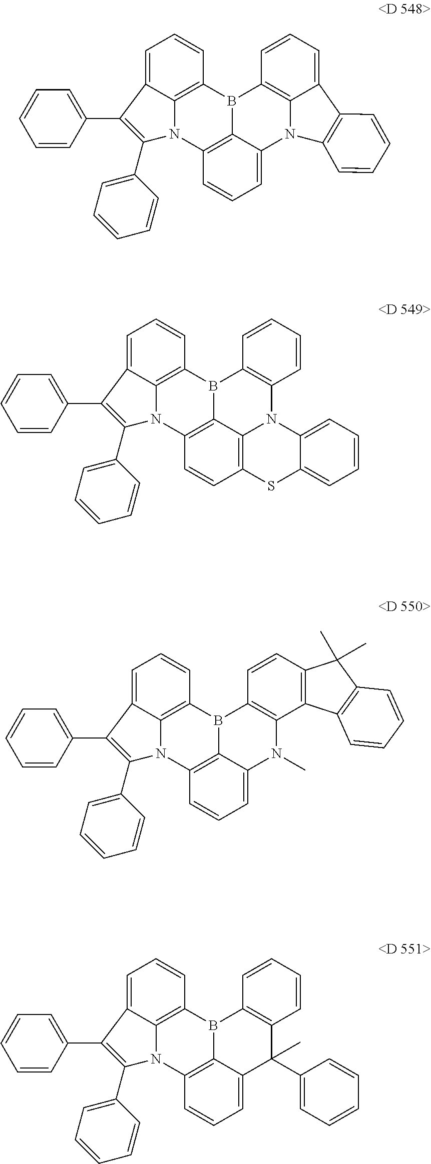 [SMILES: c1ccc(-c2c(-c3ccccc3)n3c4c(cccc24)B2c4c-3cccc4-n3c4ccccc4c4cccc2c43)cc1 ; c1ccc(-c2c(-c3ccccc3)n3c4c(cccc24)B2c4ccccc4N4c5ccccc5Sc5ccc-3c2c54)cc1 ; CN1c2cccc3c2B(c2ccc4c(c21)-c1ccccc1C4(C)C)c1cccc2c(-c4ccccc4)c(-c4ccccc4)n-3c12 ; CC1(c2ccccc2)c2ccccc2B2c3c(cccc31)-n1c(-c3ccccc3)c(-c3ccccc3)c3cccc2c31]

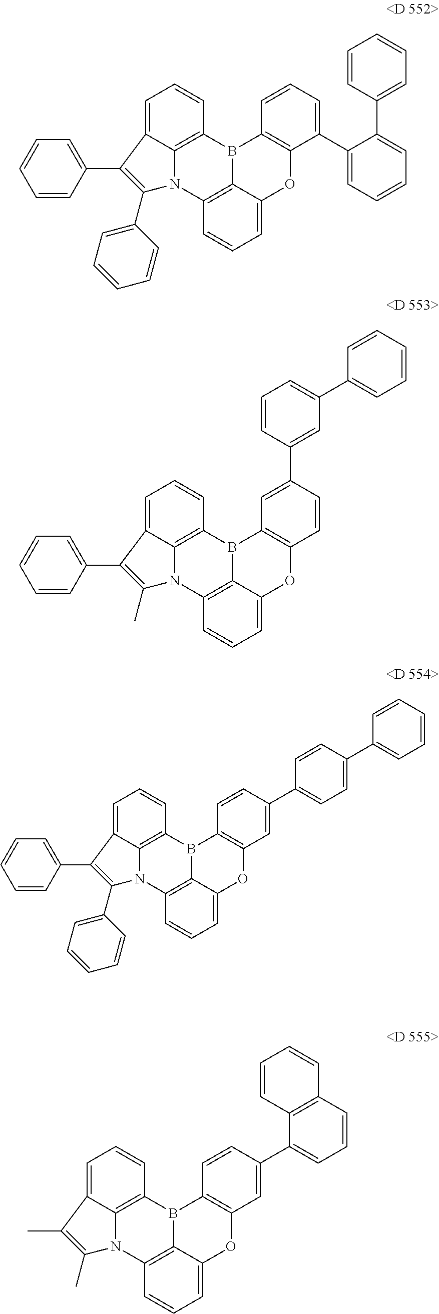 [SMILES: c1ccc(-c2ccccc2-c2cccc3c2Oc2cccc4c2B3c2cccc3c(-c5ccccc5)c(-c5ccccc5)n-4c23)cc1 ; Cc1c(-c2ccccc2)c2cccc3c2n1-c1cccc2c1B3c1cc(-c3cccc(-c4ccccc4)c3)ccc1O2 ; c1ccc(-c2ccc(-c3ccc4c(c3)Oc3cccc5c3B4c3cccc4c(-c6ccccc6)c(-c6ccccc6)n-5c34)cc2)cc1 ; Cc1c(C)n2c3c(cccc13)B1c3ccc(-c4cccc5ccccc45)cc3Oc3cccc-2c31]

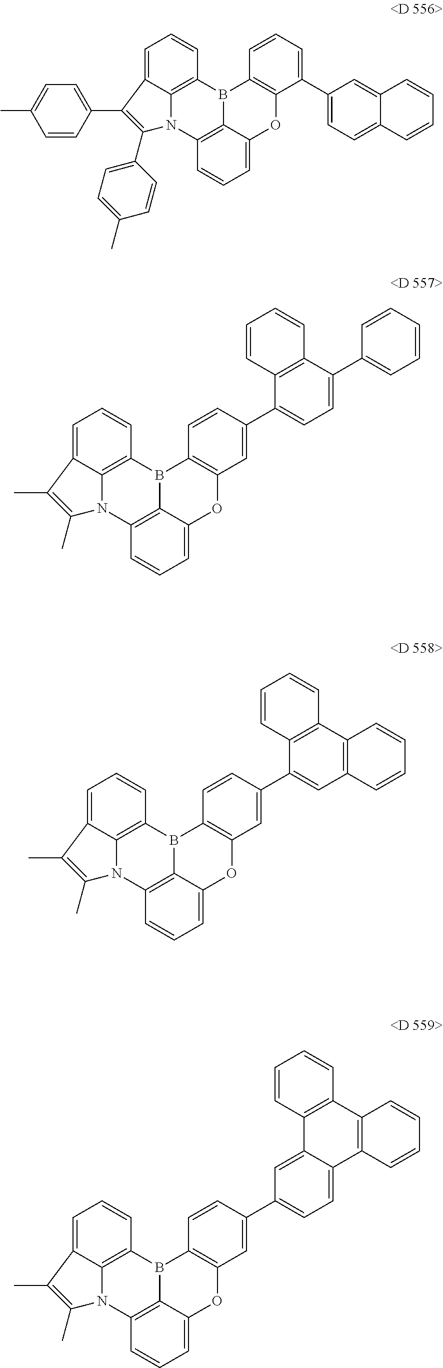 [SMILES: Cc1ccc(-c2c(-c3ccc(C)cc3)n3c4c(cccc24)B2c4cccc(-c5ccc6ccccc6c5)c4Oc4cccc-3c42)cc1 ; Cc1c(C)n2c3c(cccc13)B1c3ccc(-c4ccc(-c5ccccc5)c5ccccc45)cc3Oc3cccc-2c31 ; Cc1c(C)n2c3c(cccc13)B1c3ccc(-c4cc5ccccc5c5ccccc45)cc3Oc3cccc-2c31 ; Cc1c(C)n2c3c(cccc13)B1c3ccc(-c4ccc5c6ccccc6c6ccccc6c5c4)cc3Oc3cccc-2c31]

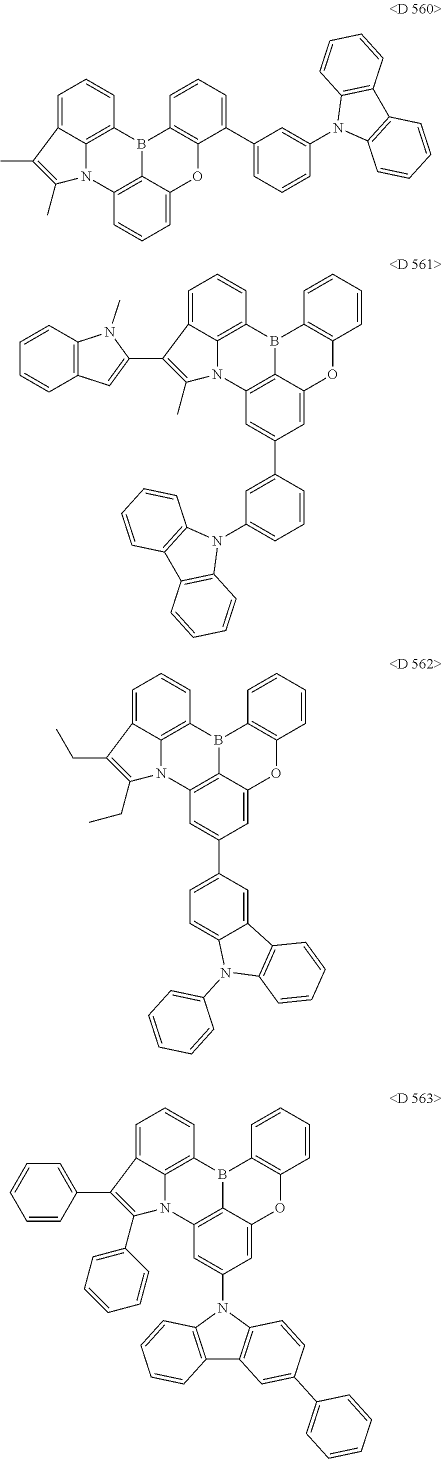 [SMILES: Cc1c(C)n2c3c(cccc13)B1c3cccc(-c4cccc(-n5c6ccccc6c6ccccc65)c4)c3Oc3cccc-2c31 ; Cc1c(-c2cc3ccccc3n2C)c2cccc3c2n1-c1cc(-c2cccc(-n4c5ccccc5c5ccccc54)c2)cc2c1B3c1ccccc1O2 ; CCc1c(CC)n2c3c(cccc13)B1c3ccccc3Oc3cc(-c4ccc5c(c4)c4ccccc4n5-c4ccccc4)cc-2c31 ; c1ccc(-c2ccc3c(c2)c2ccccc2n3-c2cc3c4c(c2)-n2c(-c5ccccc5)c(-c5ccccc5)c5cccc(c52)B4c2ccccc2O3)cc1]

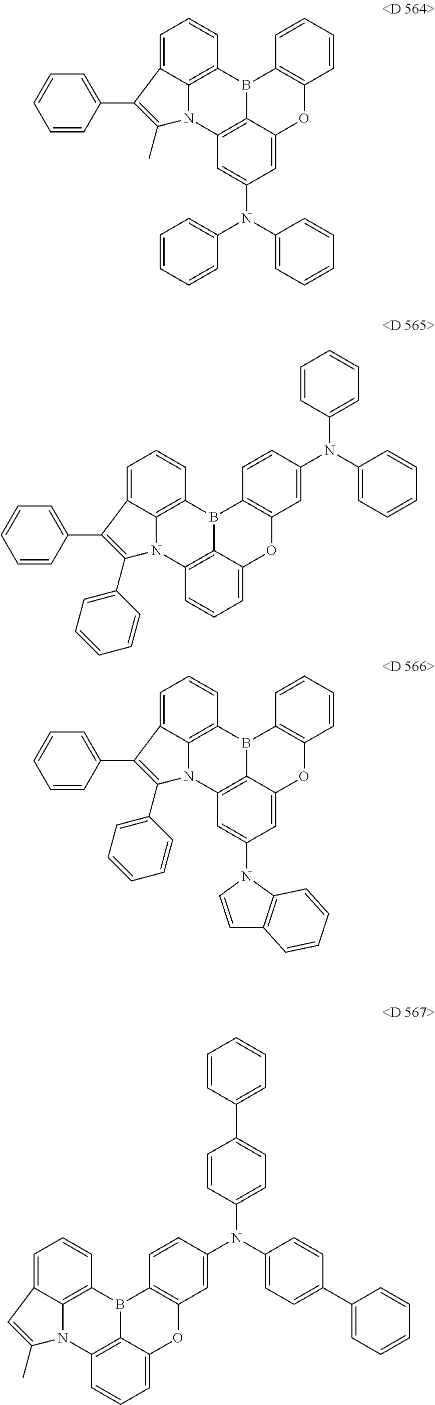 [SMILES: Cc1c(-c2ccccc2)c2cccc3c2n1-c1cc(N(c2ccccc2)c2ccccc2)cc2c1B3c1ccccc1O2 ; c1ccc(-c2c(-c3ccccc3)n3c4c(cccc24)B2c4ccc(N(c5ccccc5)c5ccccc5)cc4Oc4cccc-3c42)cc1 ; c1ccc(-c2c(-c3ccccc3)n3c4c(cccc24)B2c4ccccc4Oc4cc(-n5ccc6ccccc65)cc-3c42)cc1 ; Cc1cc2cccc3c2n1-c1cccc2c1B3c1ccc(N(c3ccc(-c4ccccc4)cc3)c3ccc(-c4ccccc4)cc3)cc1O2]

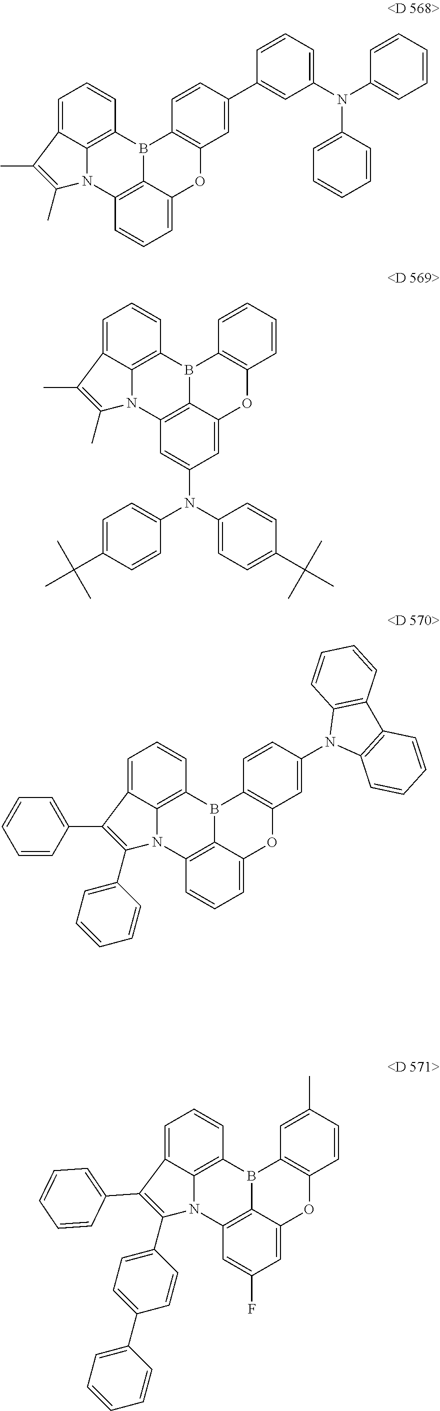 [SMILES: Cc1c(C)n2c3c(cccc13)B1c3ccc(-c4cccc(N(c5ccccc5)c5ccccc5)c4)cc3Oc3cccc-2c31 ; Cc1c(C)n2c3c(cccc13)B1c3ccccc3Oc3cc(N(c4ccc(C(C)(C)C)cc4)c4ccc(C(C)(C)C)cc4)cc-2c31 ; c1ccc(-c2c(-c3ccccc3)n3c4c(cccc24)B2c4ccc(-n5c6ccccc6c6ccccc65)cc4Oc4cccc-3c42)cc1 ; Cc1ccc2c(c1)B1c3c(cc(F)cc3-n3c(-c4ccc(-c5ccccc5)cc4)c(-c4ccccc4)c4cccc1c43)O2]

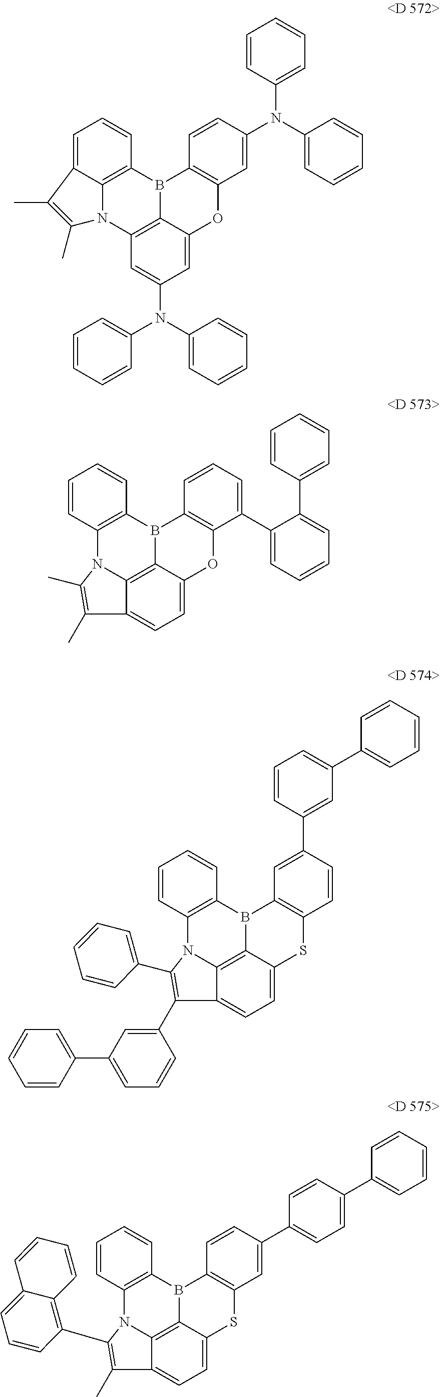 [SMILES: Cc1c(C)n2c3c(cccc13)B1c3ccc(N(c4ccccc4)c4ccccc4)cc3Oc3cc(N(c4ccccc4)c4ccccc4)cc-2c31 ; Cc1c(C)n2c3c4c(ccc13)Oc1c(cccc1-c1ccccc1-c1ccccc1)B4c1ccccc1-2 ; c1ccc(-c2cccc(-c3ccc4c(c3)B3c5ccccc5-n5c(-c6ccccc6)c(-c6cccc(-c7ccccc7)c6)c6ccc(c3c65)S4)c2)cc1 ; Cc1c(-c2cccc3ccccc23)n2c3c4c(ccc13)Sc1cc(-c3ccc(-c5ccccc5)cc3)ccc1B4c1ccccc1-2]

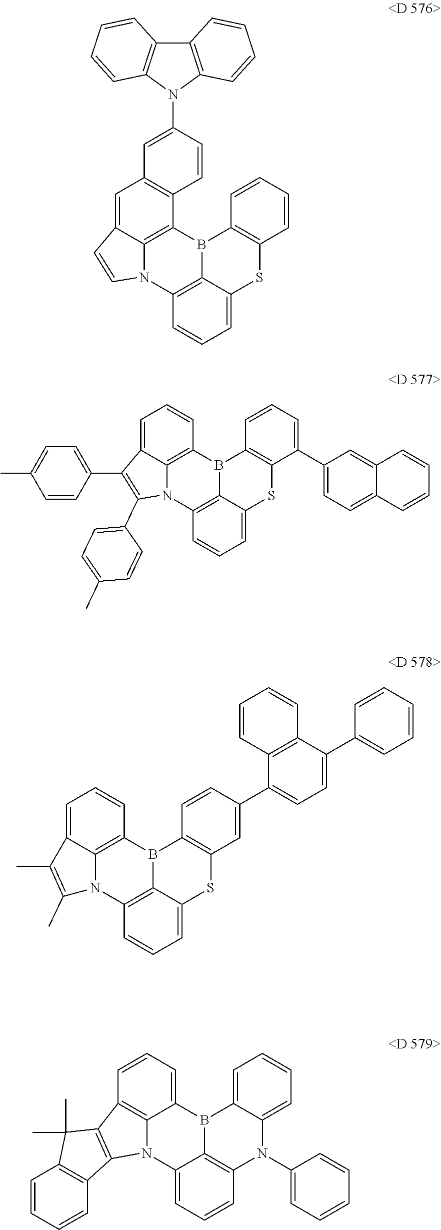 [SMILES: c1ccc2c(c1)Sc1cccc3c1B2c1c2ccc(-n4c5ccccc5c5ccccc54)cc2cc2ccn-3c12 ; Cc1ccc(-c2c(-c3ccc(C)cc3)n3c4c(cccc24)B2c4cccc(-c5ccc6ccccc6c5)c4Sc4cccc-3c42)cc1 ; Cc1c(C)n2c3c(cccc13)B1c3ccc(-c4ccc(-c5ccccc5)c5ccccc45)cc3Sc3cccc-2c31 ; CC1(C)c2ccccc2-c2c1c1cccc3c1n2-c1cccc2c1B3c1ccccc1N2c1ccccc1]

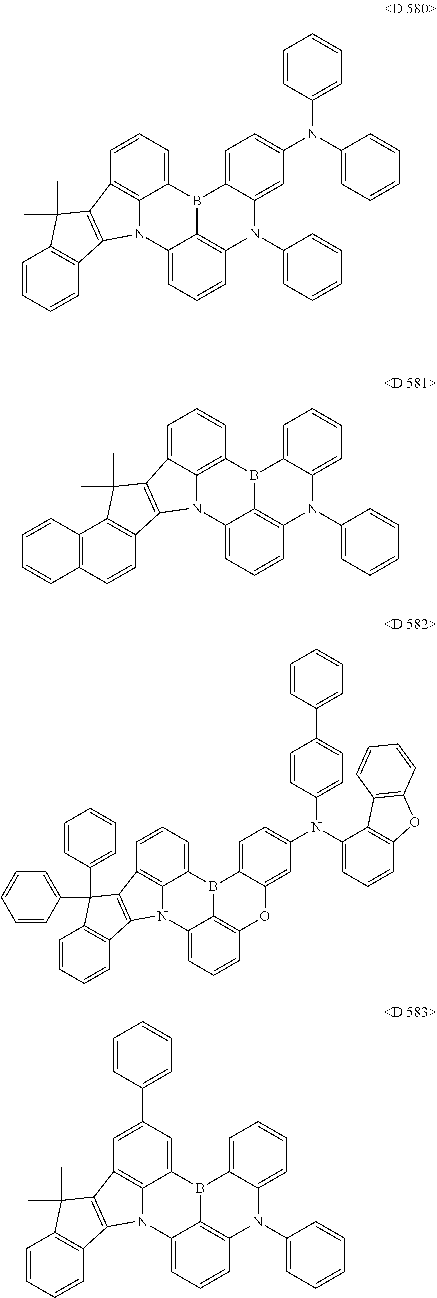 [SMILES: CC1(C)c2ccccc2-c2c1c1cccc3c1n2-c1cccc2c1B3c1ccc(N(c3ccccc3)c3ccccc3)cc1N2c1ccccc1 ; CC1(C)c2c(ccc3ccccc23)-c2c1c1cccc3c1n2-c1cccc2c1B3c1ccccc1N2c1ccccc1 ; c1ccc(-c2ccc(N(c3ccc4c(c3)Oc3cccc5c3B4c3cccc4c6c(n-5c34)-c3ccccc3C6(c3ccccc3)c3ccccc3)c3cccc4oc5ccccc5c34)cc2)cc1 ; CC1(C)c2ccccc2-c2c1c1cc(-c3ccccc3)cc3c1n2-c1cccc2c1B3c1ccccc1N2c1ccccc1]

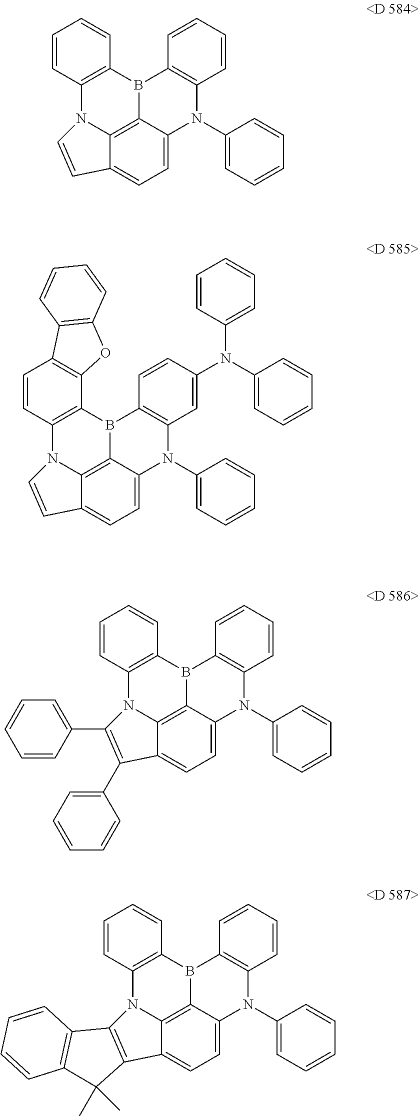 [SMILES: c1ccc(N2c3ccccc3B3c4ccccc4-n4ccc5ccc2c3c54)cc1 ; c1ccc(N(c2ccccc2)c2ccc3c(c2)N(c2ccccc2)c2ccc4ccn5c4c2B3c2c-5ccc3c2oc2ccccc23)cc1 ; c1ccc(-c2c(-c3ccccc3)n3c4c5c(ccc24)N(c2ccccc2)c2ccccc2B5c2ccccc2-3)cc1 ; CC1(C)c2ccccc2-c2c1c1ccc3c4c1n2-c1ccccc1B4c1ccccc1N3c1ccccc1]

The content of the dopant in the light-emitting layer may range from about 0.01 to 20 parts by weight, based on 100 parts by weight of the host, but is not limited thereto.

In addition to the above-mentioned dopants and hosts, the light-emitting layer may further include various hosts and dopant materials.

Below, the organic light-emitting diode of the present disclosure will be explained with reference to the drawing.

FIGURE is a schematic cross-sectional view of the structure of an organic light-emitting diode according to an embodiment of the present disclosure.

As shown in FIGURE, the organic light-emitting diode according to an embodiment of the present disclosure sequentially includes: an anode (20); a hole transport layer (40); a light-emitting layer (50) containing a host and a dopant; an electron transport layer (60); and a cathode (80), wherein the anode and the cathode serve as a first electrode and a second electrode, respectively, with the interposition of the hole transport layer between the anode and the light-emitting layer, and the electron transport layer between the light-emitting layer and the cathode.

Furthermore, the organic light-emitting diode according to an embodiment of the present disclosure may include a hole injection layer (30) between the anode (20) and the hole transport layer (40), and an electron injection layer (70) between the electron transport layer (60) and the cathode (80).

Reference is made to FIGURE with regard to the organic light emitting diode of the present disclosure and the fabrication method therefor.

First, a substrate (10) is coated with an anode electrode material to form an anode (20). So long as it is used in a typical organic electroluminescence (EL) device, any substrate may be used as the substrate (10). Preferable is an organic substrate or transparent plastic substrate that exhibits excellent transparency, surface smoothness, ease of handling, and waterproofness. As the anode electrode material, indium tin oxide (ITO), indium zinc oxide (IZO), tin oxide (SnO₂), or zinc oxide (ZnO), which are transparent and superior in terms of conductivity, may be used.

A hole injection layer material is applied on the anode (20) by thermal deposition in a vacuum or by spin coating to form a hole injection layer (30). Subsequently, thermal deposition in a vacuum or by spin coating may also be conducted to form a hole transport layer (40) with a hole transport layer material on the hole injection layer (30).

So long as it is typically used in the art, any material may be selected for the hole injection layer without particular limitations thereto. Examples include, but are not limited to, 2-TNATA [4,4′,4″-tris(2-naphthylphenyl-phenylamino)-triphenylamine], NPD [N,N′-di(1-naphthyl)-N,N′-diphenylbenzidine)], TPD [N, N′-diphenyl-N,N′-bis(3-methylphenyl)-1,1′-biphenyl-4,4′-diamine], and DNTPD [N,N′-diphenyl-N, N′-bis-[4-(phenyl-m-tolyl-amino)-phenyl]-biphenyl-4,4′-diamine], etc.

Any material that is typically used in the art may be selected for the hole transport layer without particular limitations thereto. Examples include, but are not limited to, N,N′-bis(3-methylphenyl)-N,N′-diphenyl-[1,1-bipheny]-4,4′-diamine (TPD) or N,N′-di(naphthalen-1-yl)-N,N′-diphenylbenzidine (α-NPD).

In an embodiment of the present disclosure, an electron blocking layer may be additionally disposed on the hole transport layer. Functioning to prevent the electrons injected from the electron injection layer from entering the hole transport layer from the light-emitting layer, the electron blocking layer is adapted to increase the life span and luminous efficiency of the diode. The electron blocking layer may be formed at a suitable position between the light emitting layer and the hole injection layer. Particularly, the electron blocking layer may be formed between the light emitting layer and the hole transport layer.

Next, the light-emitting layer (50) may be deposited on the hole transport layer (40) or the electron blocking layer by deposition in a vacuum or by spin coating.

Herein, the light-emitting layer may contain a host and a dopant and the materials are as described above.

In some embodiments of the present disclosure, the light-emitting layer particularly ranges in thickness from 50 to 2,000 Å.

Meanwhile, the electron transport layer (60) is applied on the light-emitting layer by deposition in a vacuum and spin coating.

A material for use in the electron transport layer functions to stably carry the electrons injected from the electron injection electrode (cathode), and may be an electron transport material known in the art. Examples of the electron transport material known in the art include quinoline derivatives, particularly, tris(8-quinolinolate)aluminum (Alq₃), Liq, TAZ, BAlq, beryllium bis(benzoquinolin-10-olate) (Bebq2), Compound 201, Compound 202, BCP, and oxadiazole derivatives such as PBD, BMD, and BND, etc., but are not limited thereto:

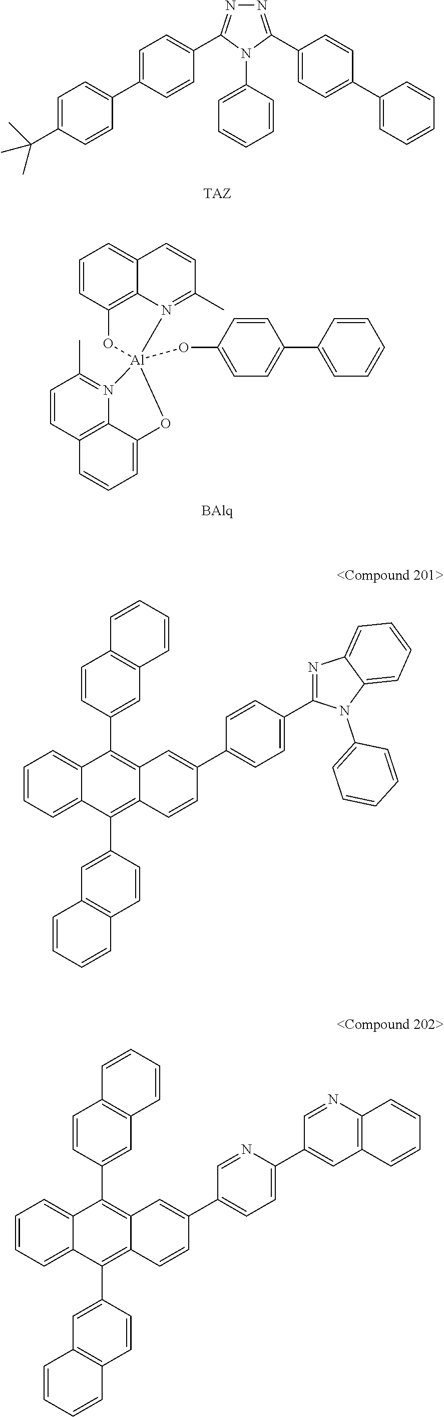

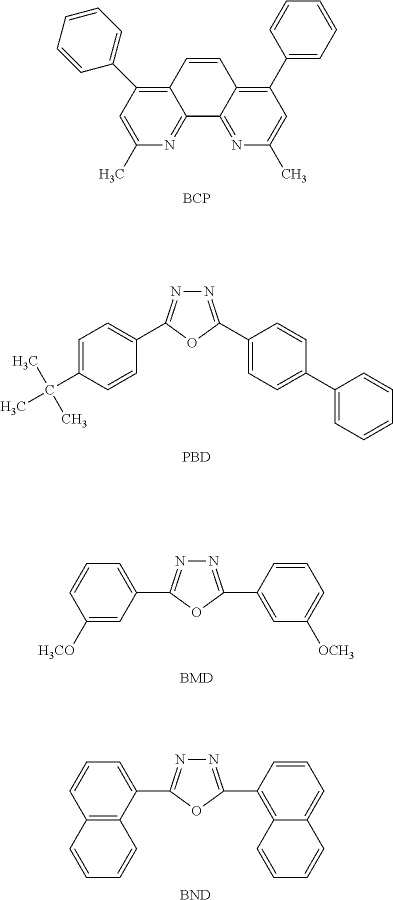

In the organic light emitting diode of the present disclosure, an electron injection layer (EIL) that functions to facilitate electron injection from the cathode may be deposited on the electron transport layer. The material for the EIL is not particularly limited.

Any material that is conventionally used in the art can be available for the electron injection layer without particular limitations. Examples include CsF, NaF, LiF, Li₂O, and Bao, etc. Deposition conditions for the electron injection layer may vary, depending on compounds used, but may be generally selected from condition scopes that are almost the same as for the formation of hole injection layers.

The electron injection layer may range in thickness from about 1 Å to about 100 Å, and particularly from about 3 Å to about 90 Å. Given the thickness range for the electron injection layer, the diode can exhibit satisfactory electron injection properties without actually elevating a driving voltage.

In order to facilitate electron injection, the cathode may be made of a material having a small work function, such as metal or metal alloy such as lithium (Li), magnesium (Mg), calcium (Ca), an alloy aluminum (Al) thereof, aluminum-lithium (Al—Li), magnesium-indium (Mg—In), and magnesium-silver (Mg—Ag). Alternatively, ITO or IZO may be employed to form a transparent cathode for an organic light-emitting diode.

Moreover, the organic light-emitting diode of the present disclosure may further comprise a light-emitting layer containing a blue, green, or red luminescent material that emits radiations in the wavelength range of 380 nm to 800 nm. That is, the light-emitting layer in the present disclosure has a multi-layer structure wherein the blue, green, or red luminescent material may be a fluorescent material or a phosphorescent material.

Furthermore, at least one selected from among the layers may be deposited using a single-molecule deposition process or a solution process.

Here, the deposition process is a process by which a material is vaporized in a vacuum or at a low pressure and deposited to form a layer, and the solution process is a method in which a material is dissolved in a solvent and applied for the formation of a thin film by means of inkjet printing, roll-to-roll coating, screen printing, spray coating, dip coating, spin coating, etc.

Additionally, the organic light-emitting diode of the present disclosure may be applied to a device selected from among a flat panel display device, a flexible display device, a monochrome or grayscale flat illumination device, a monochrome or grayscale flexible illumination device, a vehicle display device, and a display device for virtual or augmented reality.

A better understanding of the present disclosure may be obtained through the following examples which are set forth to illustrate, but are not to be construed as limiting the present disclosure.

### EXAMPLES

### Synthetic Example 1. Synthesis of Compound 35

### Synthesis Example 1-1. Synthesis of <1-a>

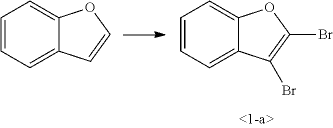 [SMILES: c1ccc2occc2c1 ; Brc1oc2ccccc2c1Br]

In a 250-ml round-bottom flask, 2,3-benzofuran (10 g, 0.085 mol), bromine (27.05 g, 0.169 mol), calcium acetate (16.62 g, 0.169 mol), and dichloromethane (240 ml) were refluxed together for 5 hours. After completion of the reaction, the reaction mixture was cooled to room temperature. An aqueous sodium thiosulfate solution was poured to the reaction mixture which was then stirred for 1 hour before extraction with dichloromethane and water. The organic layer thus formed was concentrated in a vacuum. Purification through column chromatography afforded <1-a> (14.0 g, yield 59.8%).

### Synthesis Example 1-2. Synthesis of <1-b>

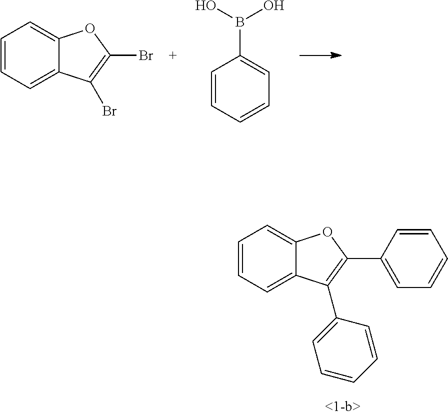 [SMILES: Brc1oc2ccccc2c1Br ; OB(O)c1ccccc1 ; c1ccc(-c2oc3ccccc3c2-c2ccccc2)cc1]

In a 250-ml round-bottom flask, <1-a> (14.0 g, 0.051 mol), 0.101 mol), phenylboronic acid (12.37 g, tetrakis(triphenylphosphine) palladium (1.76 g, 0.002 mol), potassium carbonate (17.53 g, 0.127 mol), and dioxane (140 ml) were refluxed together for 12 hours. After completion of the reaction, the reaction mixture was cooled to room temperature and concentrated in a vacuum. Purification through column chromatography afforded <1-b> (8.2 g, yield 59.8%).

### Synthesis Example 1-3. Synthesis of <1-c>

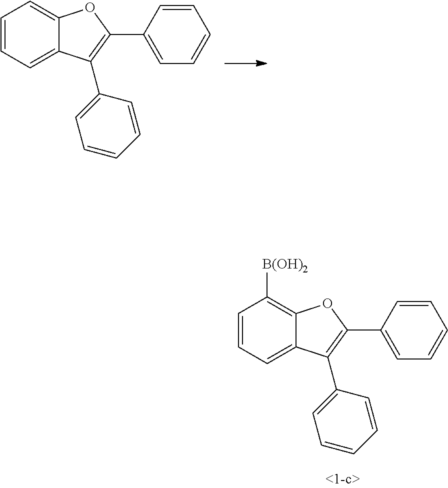 [SMILES: c1ccc(-c2oc3ccccc3c2-c2ccccc2)cc1 ; OB(O)c1cccc2c(-c3ccccc3)c(-c3ccccc3)oc12]

In a 500-ml round-bottom flask, <1-b> (8.2 g, 0.030 mol) and tetrahydrofuran (80 ml) were stirred together under a nitrogen atmosphere. After chilling to −78° C., n-butyl lithium (2.14 g, 0.033 mol) was dropwise added. The temperature was elevated to room temperature before stirring for 15 hours. The temperature was decreased again to −78° C. and trimethylborate (6.3 g, 0.061 mol) was dropwise added. The mixture was warmed to room temperature and stirred for 2 hours. Stirring was continued for an additional 1 hour after addition of 2 M hydrochloric acid (50 ml). After extraction with ethyl acetate, the organic layer thus formed was concentrated, added with heptane, and filtered to afford <1-c> (5.2 g, yield 54.7%).

### Synthesis Example 1-4. Synthesis of [Compound 35]

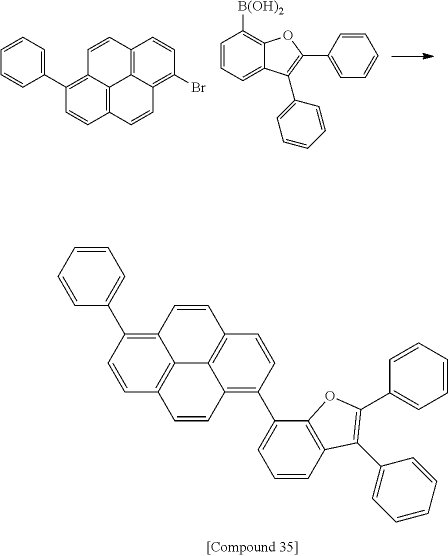

In a 250-ml round-bottom flask, <1-c> (5.2 g, 0.017 mol), 1-bromo-6-phenylpyrene (4.93 g, 0.014 mol), tetrakis(triphenylphosphine) palladium (0.48 g, 0.0004 mol), potassium carbonate (4.77 g, 0.034 mol), toluene (35 ml), ethanol (15 ml), and water (10 ml) were refluxed together for 3 hours. After completion of the reaction, the reaction mixture was added with methanol (1400 ml) and filtered. The solid thus obtained was dissolved in dichloromethane and concentrated in a vacuum, followed by purification through column chromatography to afford [Compound 35] (4.3 g, yield 57.3%).

MS (MALDI-TOF): m/z 546.20 [M⁺]

### Synthetic Example 2. Synthesis of [Compound 24]

### Synthetic Example 2-1. Synthesis of <2-a>

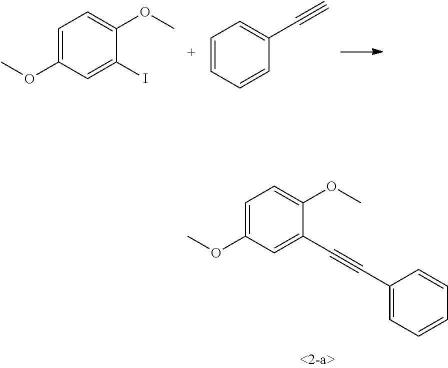

In a 1000-ml round-bottom flask, phenyl acetylene (29 g, 0.284 mol), bis(triphenylphosphine) palladium dichloride (2.7 g), copper iodide (0.7 g, 0.004 mol), triphenyl phosphine (5 g, 0.019 mol), and triethyl amine (500 ml) were stirred together. At room temperature, 1-iodo-2,5-dimethoxybenzene (50 g, 0.189 mol) was dropwise added, followed by stirring until completion of the reaction. After completion of the reaction, the reaction mixture was filtered and the filtrate was concentrated and purified through column chromatography to afford <2-a> (42 g, yield 93.1%).

### Synthetic Example 2-2. Synthesis of <2-b>

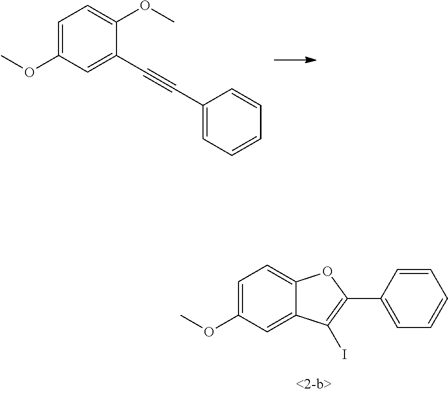 [SMILES: COc1ccc(OC)c(C#Cc2ccccc2)c1 ; COc1ccc2oc(-c3ccccc3)c(I)c2c1]

In a 1000-ml round-bottom flask, <2-a> (40 g, 0.168 mol) and dichloromethane (400 ml) were stirred together at room temperature. The mixture was slowly added with drops of iodomonochloride (32.7 g, 0.201 mol) and stirred until completion of the reaction. After completion of the reaction, the reaction mixture was added with water (500 ml) and subjected to extraction with dichloromethane and water. The organic layer thus formed was dehydrated, concentrated in a vacuum, and purified by column chromatography to afford <2-b> (52 g, yield 88.4%).

### Synthetic Example 2-3. Synthesis of <2-c>

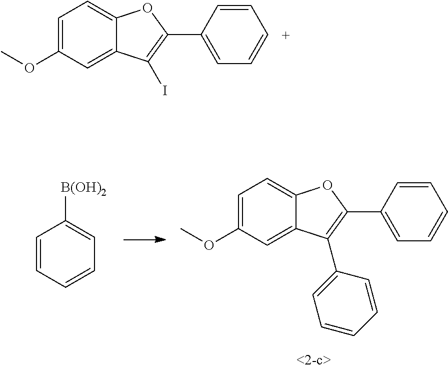 [SMILES: COc1ccc2oc(-c3ccccc3)c(I)c2c1 ; OB(O)c1ccccc1 ; COc1ccc2oc(-c3ccccc3)c(-c3ccccc3)c2c1]

In a 1000-ml round-bottom flask, <2-b> (50 g, 0.143 mol), phenyl boronic acid (20.9 g, 0.171 mol), tetrakis(triphenylphosphine) palladium 8.26 g, 0.007 mol), potassium phosphate (60.6 g, 0.286 mol), and 1,4-dioxane (400 ml) were refluxed together for 12 hours. After completion of the reaction, the reaction mixture was cooled to room temperature and subjected to extraction with ethyl acetate and water. Subsequently, the organic layer thus formed was concentrated in a vacuum, and purified by column chromatography to afford <2-c> (42 g, yield 67.1%).

### Synthetic Example 2-4. Synthesis of <2-d>

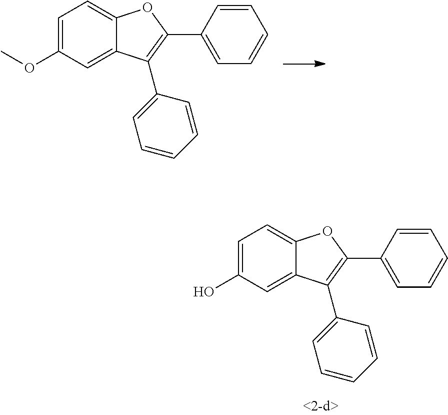 [SMILES: COc1ccc2oc(-c3ccccc3)c(-c3ccccc3)c2c1 ; Oc1ccc2oc(-c3ccccc3)c(-c3ccccc3)c2c1]

In a 500-ml round-bottom flask, <2-c> (42 g, 0.140 mol) and dichloromethane (210 ml) were cooled to 0° C. or lower under a nitrogen atmosphere. When the temperature was decreased to 0° C. or less, drops of boron tribromide (70 g, 0.280 mol) was slowly added. Thereafter, the mixture was warmed to room temperature and stirred until completion of the reaction. After completion of the reaction, water (500 ml) was slowly poured to the reaction mixture which was then subjected to extraction with dichloromethane and water. Subsequently, the organic layer thus formed was dehydrated, concentrated in. a vacuum, and purified by column chromatography to afford <2-d> (34 g, yield 89%).

### Synthetic Example 2-5. Synthesis of <2-e>

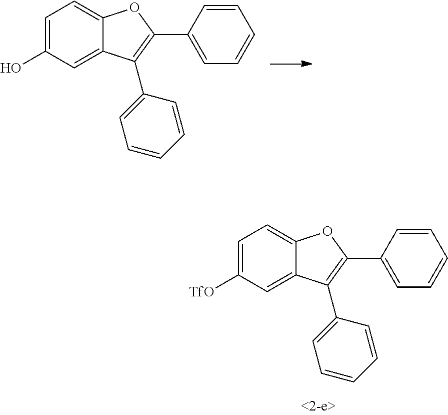 [SMILES: Oc1ccc2oc(-c3ccccc3)c(-c3ccccc3)c2c1 ; O=S(=O)(Oc1ccc2oc(-c3ccccc3)c(-c3ccccc3)c2c1)C(F)(F)F]

In a 1000-ml round-bottom flask, <2-d> (34 g, 0.119 mol), pyridine (12.2 g, 0.154 mol), and dichloromethane (340 ml) were cooled together to 0° C. or lower under a nitrogen atmosphere. At 0° C. or less, trifluoromethane sulfonic acid (36.9 g, 0.131 mol) was dropwise added. Thereafter, the mixture was warmed to room temperature and stirred until completion of the reaction. After completion of the reaction, the reaction mixture was added with water (500 ml) and stirred for 30 minutes, and subjected to extraction with dichloromethane and water. Concentrated in a vacuum and purification by column chromatography afforded <2-e> (48 g, yield 96.6%).

### Synthetic Example 2-6. Synthesis of <2-f>

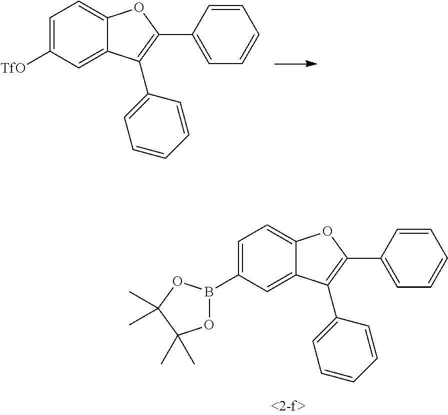 [SMILES: O=S(=O)(Oc1ccc2oc(-c3ccccc3)c(-c3ccccc3)c2c1)C(F)(F)F ; CC1(C)OB(c2ccc3oc(-c4ccccc4)c(-c4ccccc4)c3c2)OC1(C)C]

In a 1000-ml round-bottom flask, <2-e> (48 g, 0.115 mol), bis(pinacolato)diboron (37.9 g, 0.149 mol), bis(diphenylphosphino) ferrocene dichloropalladium (1.9 g, 0.002 mol), calcium acetate (22.5 g, 0.229 mol), and 1,4-dioxane (480 ml) were refluxed together for 12 hours. After completion of the reaction, the reaction mixture was cooled to room temperature and filtered through celite. The filtrate was concentrated and purified by column chromatography to afford <2-f> (35 g, yield 77%).

### Synthetic Example 2-7. Synthesis of <2-g>

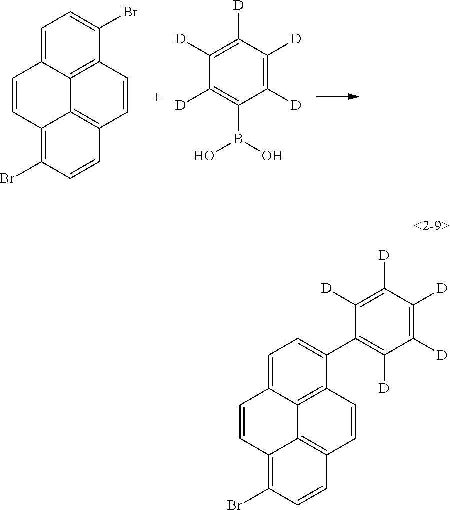 [SMILES: Brc1ccc2ccc3c(Br)ccc4ccc1c2c43 ; [2H]c1c([2H])c([2H])c(B(O)O)c([2H])c1[2H] ; [2H]c1c([2H])c([2H])c(-c2ccc3ccc4c(Br)ccc5ccc2c3c54)c([2H])c1[2H]]

In a 3000-ml round-bottom flask purged with nitrogen, 1,6-dibromopyrene (100 g, 0.278 mol), phenylboronic acid (D5, 35.3 g, 0.278 mol), tetrakis(triphenylphosphine) palladium (Pd [PPh₃]₄) (6.4 g, 0.006 mol), sodium carbonate (88.3 g, 0.833 mol), toluene (1400 ml), and water (420 ml) were refluxed together for 9 hours. After completion of the reaction, the reaction mixture was cooled to room temperature and the solid thus formed was filtered out. The remaining solution was subjected to extraction with ethyl acetate and water. The organic layer thus formed was dehydrated. After concentration in a vacuum, column chromatography isolated <2-g> (46 g, yield 45.7%).

### Synthetic Example 2-8. Synthesis of [Compound 24]

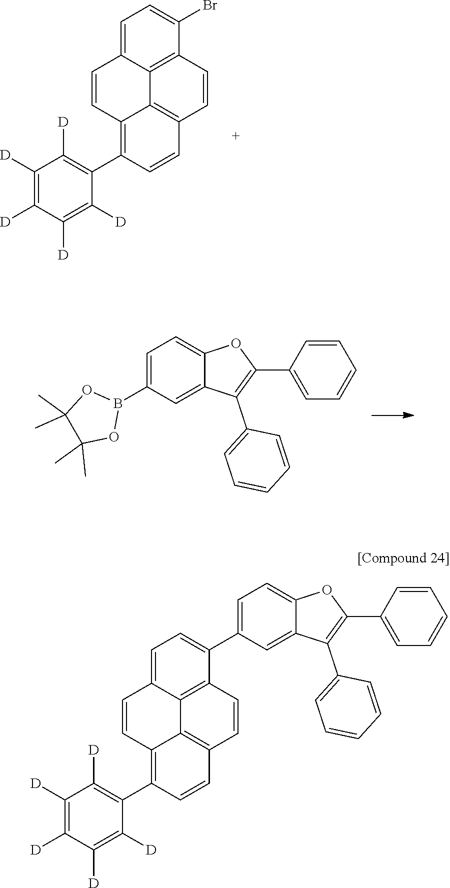 [SMILES: [2H]c1c([2H])c([2H])c(-c2ccc3ccc4c(Br)ccc5ccc2c3c54)c([2H])c1[2H] ; CC1(C)OB(c2ccc3oc(-c4ccccc4)c(-c4ccccc4)c3c2)OC1(C)C ; [2H]c1c([2H])c([2H])c(-c2ccc3ccc4c(-c5ccc6oc(-c7ccccc7)c(-c7ccccc7)c6c5)ccc5ccc2c3c54)c([2H])c1[2H]]

In a 500-ml round-bottom flask, <2-g> (13 g, 0.036 mol), <2-f> (14.9 g, 0.038 mol), tetrakis(triphenylphosphine) palladium (0.8 g, 0.001 mol), potassium carbonate (8.4 g, 0.061 mol), toluene (90 ml), ethanol (23 ml), and water (30 ml) were refluxed together for 6 hours. After completion of the reaction, the reaction mixture was cooled to room temperature and subjected to extraction with ethyl acetate and water. The organic layer was concentrated in a vacuum, followed by recrystallization in dichloromethane and methanol to afford [Chemical Formula 24] (12 g, yield 60.6%).

MS (MALDI-TOF): m/z 551.23 [M⁺]

### Synthetic Example 3. Synthesis of [Compound 29]

### Synthetic Example 3-1. Synthesis of <3-a>

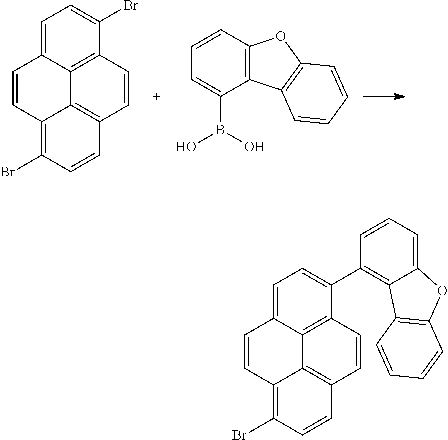 [SMILES: Brc1ccc2ccc3c(Br)ccc4ccc1c2c43 ; OB(O)c1cccc2oc3ccccc3c12 ; Brc1ccc2ccc3c(-c4cccc5oc6ccccc6c45)ccc4ccc1c2c43]

### 3-a

The same procedure as in Synthesis Example 2-7 was carried out, with the exception of using dibenzofuran-1-boronic acid instead of phenyl boronic acid (D5), to afford <3-a> (yield 43.7%).

### Synthetic Example 3-2. Synthesis of [Compound 29]

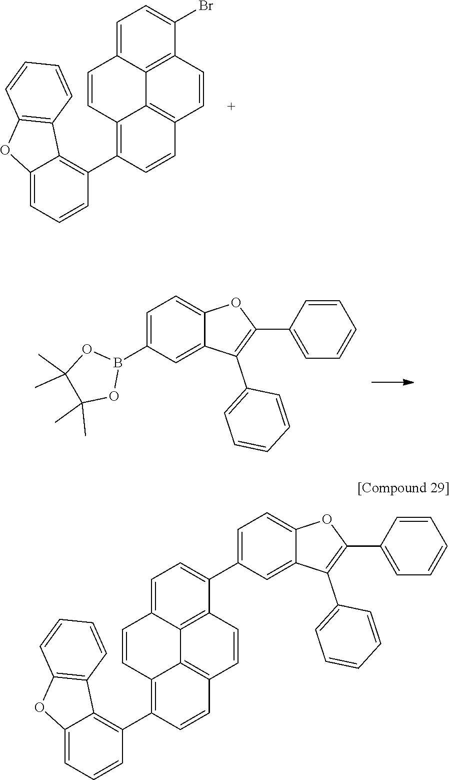 [SMILES: Brc1ccc2ccc3c(-c4cccc5oc6ccccc6c45)ccc4ccc1c2c43 ; CC1(C)OB(c2ccc3oc(-c4ccccc4)c(-c4ccccc4)c3c2)OC1(C)C ; c1ccc(-c2oc3ccc(-c4ccc5ccc6c(-c7cccc8oc9ccccc9c78)ccc7ccc4c5c76)cc3c2-c2ccccc2)cc1]

The same procedure as in Synthesis Example 2-8 was carried out, with the exception of using <3-a> instead of <2-g>, to afford [Compound 29] (yield 62.4%).

MS (MALDI-TOF): m/z 636.21 [M⁺]

### Synthetic Example 4. Synthesis of [Compound 48]

### Synthetic Example 4-1. Synthesis of <4-a>

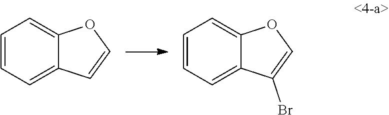 [SMILES: c1ccc2occc2c1 ; Brc1coc2ccccc12]

In a 250-ml round-bottom flask, 2,3-benzofuran (10.0 g, 0.085 mol) and dichloromethane (240 ml) were stirred together under a nitrogen atmosphere. The mixture was cooled to −10° C. before addition of drops of bromine (14.8 g, 0.093 mol). The mixture was stirred for 1 hour. After completion of the reaction, the temperature was elevated to room temperature and an aqueous sodium thiosulfate solution was poured and stirred for 1 hour. After extraction with dichloromethane and water, the organic layer was concentrated in a vacuum. In a 500-ml round-bottom flask, the concentrate was added with ethanol (100 ml) and stirred. After chilling to −10° C., an aqueous potassium hydroxide (200 ml) was dropwise added. Then, the reaction mixture was refluxed for 4 hours, cooled to room temperature, and subjected to extraction with dichloromethane and water. The organic layer thus formed was concentrated in a vacuum and purified by column chromatography to afford <4-a> (15.0 g, yield 89.8%).

### Synthetic Example 4-2. Synthesis of <4-b>

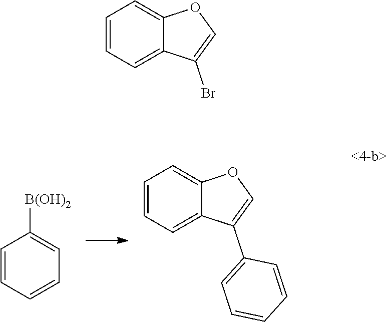 [SMILES: Brc1coc2ccccc12 ; OB(O)c1ccccc1 ; c1ccc(-c2coc3ccccc23)cc1]

In a 250-ml round-bottom flask, <4-a> (15.0 g, 0.076 mol), phenyl boronic acid (11.14 g, 0.091 mol), palladium (II) acetate (0.34 g, 0.002 mol), tri-tert-butylphosphonium tetrafluoroborate (0.44 g, 0.002 mol), and butanol (150 ml) were stirred together for 1 hour. An aqueous sodium hydroxide solution was dropwise added, followed by stirring for 15 minutes. After completion of the reaction, extraction with ethyl acetate and water was conducted. The organic layer thus formed was concentrated in a vacuum and purified by column chromatography to afford <4-b> (14.3 g, yield 97.2%).

### Synthetic Example 4-3. Synthesis of <4-c>

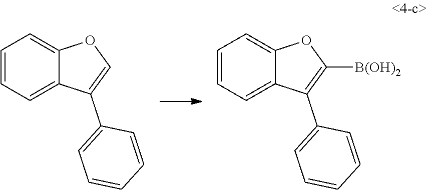 [SMILES: c1ccc(-c2coc3ccccc23)cc1 ; OB(O)c1oc2ccccc2c1-c1ccccc1]

In a 500-ml round-bottom flask, <4-b> (14.3 g, 0.074 mol) and THE (120 ml) were stirred together under a nitrogen atmosphere. After the temperature was decreased to −78° C., n-butyl lithium (1.6M) (55.2 ml, (0.088 mol) was slowly added in a dropwise manner while the temperature was maintained. Thereafter, the mixture was stirred for 3 hours and then, trimethyl borate (11.5 ml, 0.103 mol) was slowly added in a dropwise manner at the same temperature. Subsequently, the temperature was elevated to room temperature and stirring was continued until completion of the reaction. After completion of the reaction, the reaction mixture was slowly added with drops of 2N HCl and stirred for 1 hour. Afterward, extraction was conducted with ethyl acetate and water, and the organic layer thus formed was washed twice with water. The organic layer was dehydrated, concentrated in a vacuum, and recrystallized in heptane to afford <4-c> (11 g, yield 62.8%).

### Synthetic Example 4-4. Synthesis of <4-d>

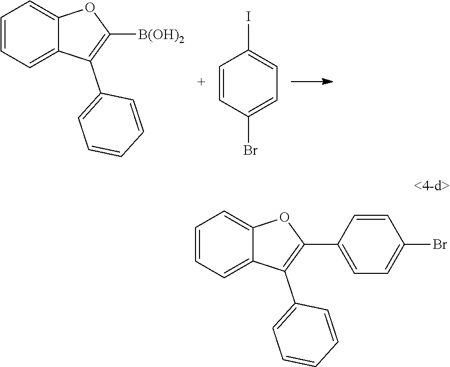 [SMILES: OB(O)c1oc2ccccc2c1-c1ccccc1 ; Brc1ccc(I)cc1 ; Brc1ccc(-c2oc3ccccc3c2-c2ccccc2)cc1]

In a 300-ml round-bottom flask, <4-c> (11 g, 0.046 mol), 1-bromo-4-iodobenzene (15.7 g, 0.055 mol), tetrakis(triphenylphosphine) palladium 1.1 g, 0.001 mol), potassium carbonate 10.9 g, 0.079 mol), toluene (77 ml), ethanol (20 ml), and water (40 ml) were refluxed for 6 hours. After completion of the reaction, the reaction mixture was cooled to room temperature and subjected to extraction with ethyl acetate and water. The organic layer thus formed was dehydrated, concentrated in a vacuum, and purified by column chromatography to afford <4-d> (13 g, yield 80.6%).

### Synthetic Example 4-5. Synthesis of <4-e>

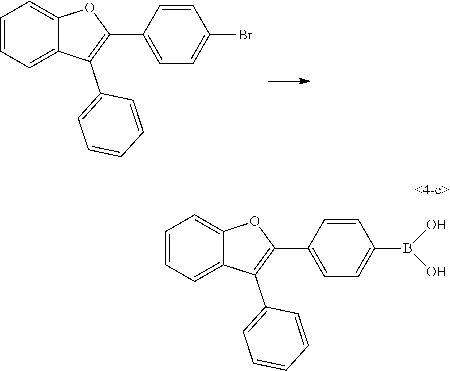 [SMILES: Brc1ccc(-c2oc3ccccc3c2-c2ccccc2)cc1 ; OB(O)c1ccc(-c2oc3ccccc3c2-c2ccccc2)cc1]

The same procedure as in Synthesis Example 4-3 was carried out, with the exception of using <4-d> instead of <4-b>, to afford <4-e> (yield 77%).

### Synthetic Example 4-6. Synthesis of [Compound 48]

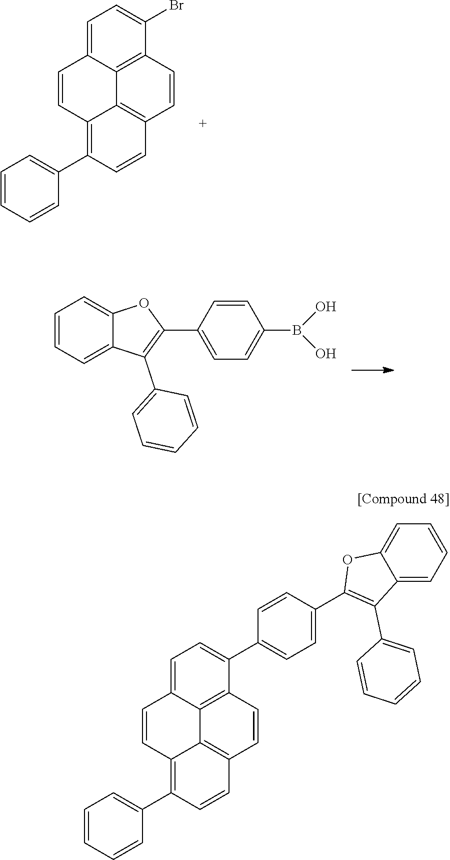 [SMILES: Brc1ccc2ccc3c(-c4ccccc4)ccc4ccc1c2c43 ; OB(O)c1ccc(-c2oc3ccccc3c2-c2ccccc2)cc1 ; c1ccc(-c2c(-c3ccc(-c4ccc5ccc6c(-c7ccccc7)ccc7ccc4c5c76)cc3)oc3ccccc23)cc1]

The same procedure as in Synthesis Example 1-4 was carried out, with the exception of using <4-e> instead of <1-c>, to afford [Compound 48] (yield 43%).

MS (MALDI-TOF): m/z 546.20 [M⁺]

### Synthetic Example 5. Synthesis of [Compound 62]

### Synthetic Example 5-1. Synthesis of <5-a>

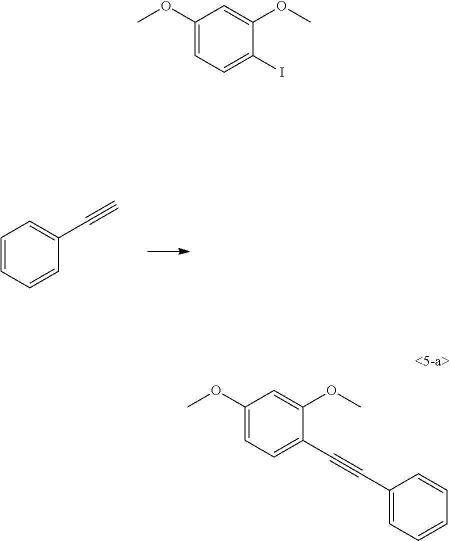

The same procedure as in Synthesis Example 2-1 was carried out, with the exception of using 1-iodo-2,4-dimethoxybenzene instead of 1-iodo-2,5-dimethoxybenzene, to afford <5-a> (yield 88.6%).

### Synthetic Example 5-2. Synthesis of <5-b>

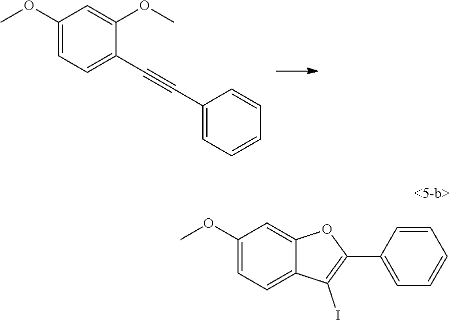 [SMILES: COc1ccc(C#Cc2ccccc2)c(OC)c1 ; COc1ccc2c(I)c(-c3ccccc3)oc2c1]

The same procedure as in Synthesis Example 2-2 was carried out, with the exception of using <5-a> instead of <2-a>, to afford <5-b> (yield 83.3%).

### Synthetic Example 5-3. Synthesis of <5-c>

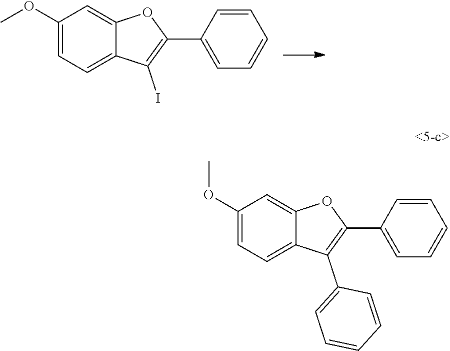 [SMILES: COc1ccc2c(I)c(-c3ccccc3)oc2c1 ; COc1ccc2c(-c3ccccc3)c(-c3ccccc3)oc2c1]

The same procedure as in Synthesis Example 2-3 was carried out, with the exception of using <5-b> instead of <2-b>, to afford <5-c> (yield 85.7%).

### Synthetic Example 5-4. Synthesis of <5-d>

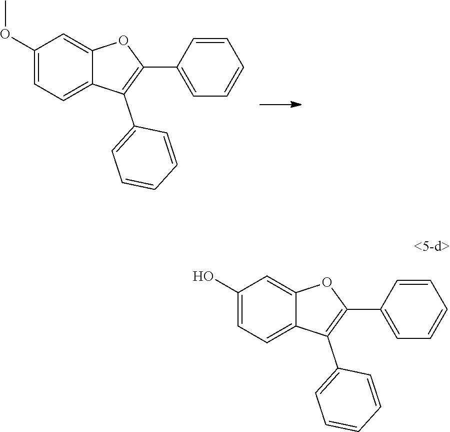 [SMILES: COc1ccc2c(-c3ccccc3)c(-c3ccccc3)oc2c1 ; Oc1ccc2c(-c3ccccc3)c(-c3ccccc3)oc2c1]

The same procedure as in Synthesis Example 2-4 was carried out, with the exception of using <5-c> instead of <2-c>, to afford <5-d> (yield 78.7%).

### Synthetic Example 5-5. Synthesis of <5-e>

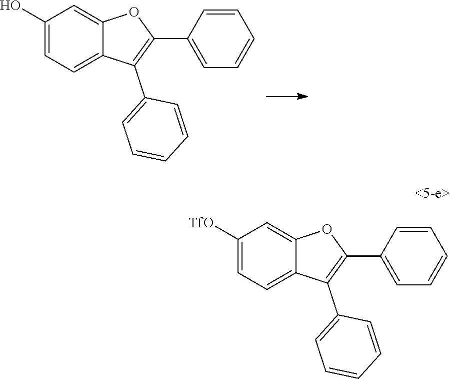 [SMILES: Oc1ccc2c(-c3ccccc3)c(-c3ccccc3)oc2c1 ; O=S(=O)(Oc1ccc2c(-c3ccccc3)c(-c3ccccc3)oc2c1)C(F)(F)F]

The same procedure as in Synthesis Example 2-5 was carried out, with the exception of using <5-d> instead of <2-d>, to afford <5-e> (yield 76.1%).

### Synthetic Example 5-6. Synthesis of <5-f>

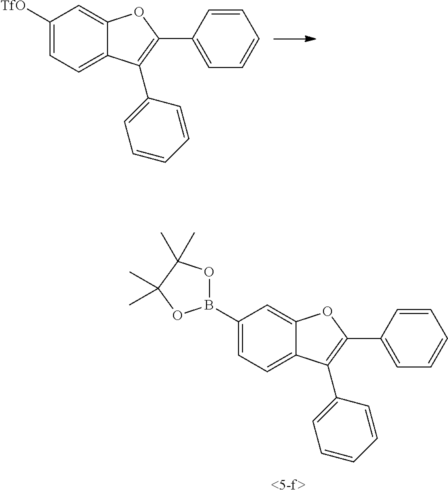 [SMILES: O=S(=O)(Oc1ccc2c(-c3ccccc3)c(-c3ccccc3)oc2c1)C(F)(F)F ; CC1(C)OB(c2ccc3c(-c4ccccc4)c(-c4ccccc4)oc3c2)OC1(C)C]

The same procedure as in Synthesis Example 2-6 was carried out, with the exception of using <5-e> instead of <2-e>, to afford <5-f> (yield 77.4%).

### Synthetic Example 5-6. Synthesis of [Compound 62]

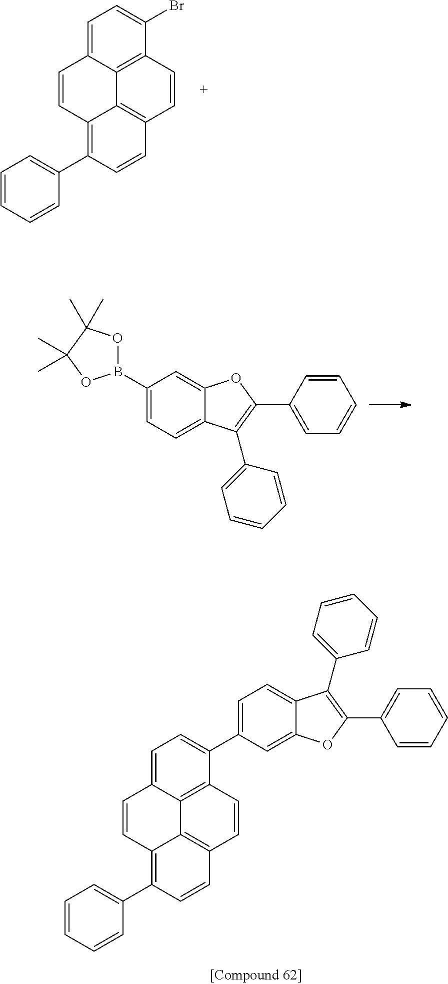 [SMILES: Brc1ccc2ccc3c(-c4ccccc4)ccc4ccc1c2c43 ; CC1(C)OB(c2ccc3c(-c4ccccc4)c(-c4ccccc4)oc3c2)OC1(C)C ; c1ccc(-c2oc3cc(-c4ccc5ccc6c(-c7ccccc7)ccc7ccc4c5c76)ccc3c2-c2ccccc2)cc1]

The same procedure as in Synthesis Example 1-4 was carried out, with the exception of using <5-f> instead of <1-c>, to afford [Compound 62] (yield 43%).

MS (MALDI-TOF): m/z 546.20 [M⁺]

### Synthetic Example 6. Synthesis of [Compound 79]

### Synthetic Example 6-1. Synthesis of <6-a>

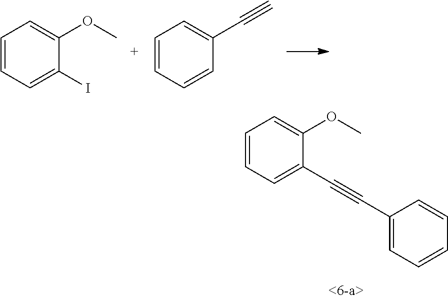

The same procedure as in Synthesis Example 2-1 was carried out, with the exception of using 1-iodoanisole instead of 1-iodo-2,5-dimethoxybenzene, to afford <6-a> (yield 96.6%).

### Synthetic Example 6-2. Synthesis of <6-b>

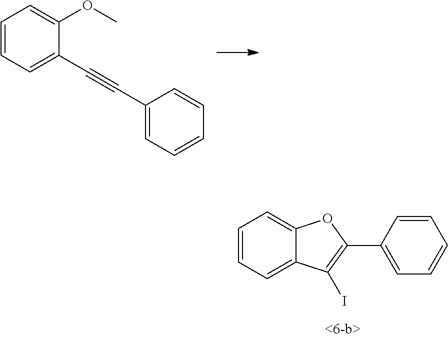 [SMILES: COc1ccccc1C#Cc1ccccc1 ; Ic1c(-c2ccccc2)oc2ccccc12]

The same procedure as in Synthesis Example 2-2 was carried out, with the exception of using <6-a> instead of <2-a>, to afford <6-b> (yield 72.6%).

### Synthetic Example 6-3. Synthesis of <6-c>

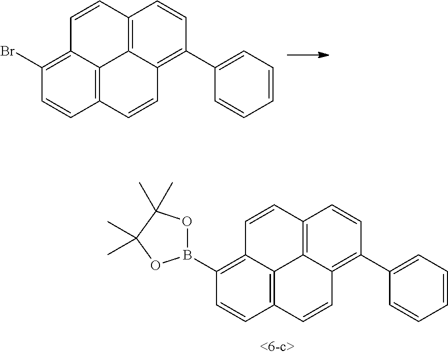 [SMILES: Brc1ccc2ccc3c(-c4ccccc4)ccc4ccc1c2c43 ; CC1(C)OB(c2ccc3ccc4c(-c5ccccc5)ccc5ccc2c3c54)OC1(C)C]

In a 500-ml round-bottom flask, 1-bromo-6-phenylpyrene (14.5 g, 0.041 mol), bis(pinacolato)diboron (12.37 g, 0.049 mol), bis(diphenylphosphino) ferrocene dichloropalladium (0.99 g, 0.001 mol), potassium acetate (11.95 g, 0.122 mol), and dioxane (140 ml) were refluxed together for 5 hours. After completion of the reaction, the reaction mixture was cooled to room temperature and filtered through celite. The filtrate was concentrated in a vacuum and purified by column chromatography to afford <6-c> (9.0 g, yield 54.8%).

### Synthetic Example 6-4. Synthesis of [Compound 79]

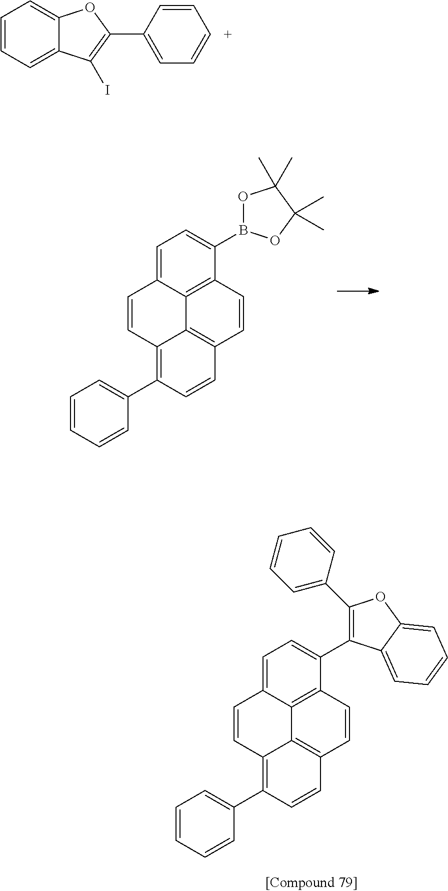 [SMILES: Ic1c(-c2ccccc2)oc2ccccc12 ; CC1(C)OB(c2ccc3ccc4c(-c5ccccc5)ccc5ccc2c3c54)OC1(C)C ; c1ccc(-c2oc3ccccc3c2-c2ccc3ccc4c(-c5ccccc5)ccc5ccc2c3c54)cc1]

In a 300-ml round-bottom flask, <6-b> (15 g, 0.054 mol), <6-c> (20.7 g, 0.082 mol), tetrakis(triphenylphosphine) palladium (6.3 g, 0.005 mol), potassium carbonate (15 g, 0.109 mol), and 1,4-dioxane (150 ml) were refluxed together for 12 hours. After completion of the reaction, the reaction mixture was cooled to room temperature and subjected to extraction with ethyl acetate and water. The organic layer thus formed was concentrated in a vacuum and purified by column chromatography to afford [Compound 79] (7.6 g, yield 35.3%).

MS (MALDI-TOF): m/z 470.17 [M⁺]

### Synthetic Example 7. Synthesis of [Compound 82]

### Synthetic Example 7-1. Synthesis of <7-a>

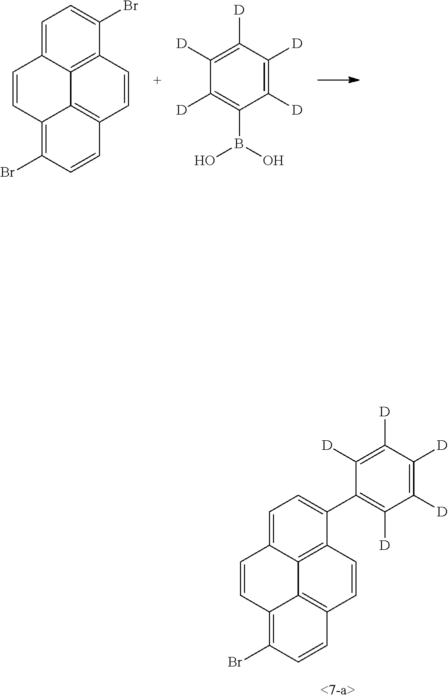 [SMILES: Brc1ccc2ccc3c(Br)ccc4ccc1c2c43 ; [2H]c1c([2H])c([2H])c(B(O)O)c([2H])c1[2H] ; [2H]c1c([2H])c([2H])c(-c2ccc3ccc4c(Br)ccc5ccc2c3c54)c([2H])c1[2H]]

in a 3000-ml round-bottom flask purged with nitrogen, 1,6-dibromopyrene (100 g, 0.278 mol), phenyl boronic acid (D5) (35.3 g, 0.278 mol), tetrakis(triphenylphosphine) palladium (Pd [PPh₃]₄) (6.4 g, 0.006 mol), sodium carbonate (88.3 g, 0.833 mol), toluene (1400 ml), and water (420 ml) were refluxed together for 9 hours. After completion of the reaction, the reaction mixture was cooled to room temperature and the solid thus formed was filtered out. The remaining solution was subjected to extraction with ethyl acetate and water. The organic layer thus formed was dehydrated. After concentration in a vacuum, column chromatography afforded <7-a> (46 g, yield 45.7%).

### Synthetic Example 7-2. Synthesis of <7-b>

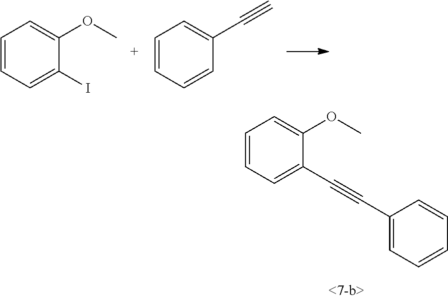

In a 2000-ml round-bottom flask, 1-iodoanisole (100 g, 0.427 mol), phenyl acetylene (87.3 g, 0.855 mol), tetrakis(triphenylphosphine) palladium (14.8 g, 0.013 mol), copper iodide (4.1 g, 0.021 mol), triphenyl phosphine (2.2 g, 0.009 mol), and triethyl amine (1000 ml) were refluxed together for 6 hours. After completion of the reaction, the reaction mixture was cooled to room temperature and filtered through celite, and the filtrate was concentrated and purified through column chromatography to afford <7-b> (79 g, yield 88.8%).

### Synthetic Example 7-3. Synthesis of <7-c>

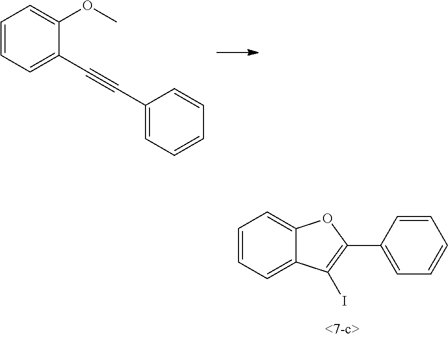 [SMILES: COc1ccccc1C#Cc1ccccc1 ; Ic1c(-c2ccccc2)oc2ccccc12]

In a 2000-ml round-bottom flask, <7-b> (79 g, 0.379 mol) and dichloromethane (790 ml) were stirred together at room temperature. The mixture was added drops of iodomonochloride (73.9 g) at room temperature while being stirred. Stirring was continued at room temperature until completion of the reaction. After completion of the reaction, the reaction mixture was added with an aqueous saturated sodium thiosulfate solution. Stirring for 20 minutes was followed by extraction with dichloromethane and water. The organic layer thus formed was concentrated by column and purified chromatography to afford <7-c> (83 g, yield 68.3%).

### Synthetic Example 7-4. Synthesis of <7-d>

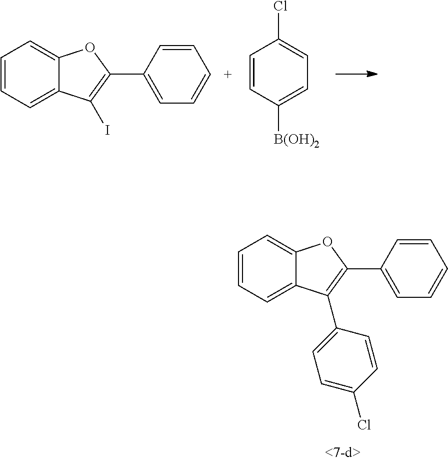

In a 500-ml round-bottom flask, <7-c> (34.2 g, 0.107 mol), 4-chlorophenyl boronic acid (18.4 g, 0.118 mol), tetrakis(triphenylphosphine) palladium (2.5 g, 0.002 mol), potassium carbonate (29.5 g, 0.214 mol), and N, N-dimethylformamide (270 ml) were refluxed for 24 hours. After completion of the reaction, the reaction mixture was cooled to room temperature and subjected to extraction with ethyl acetate and water. The organic layer thus formed was concentrated in a vacuum and purified by column chromatography to afford <7-d> (23.1 g, yield 70.9%).

### Synthetic Example 7-5. Synthesis of <7-e>

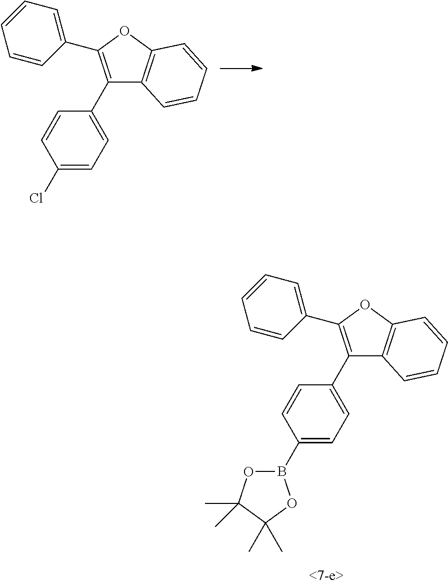 [SMILES: Clc1ccc(-c2c(-c3ccccc3)oc3ccccc23)cc1 ; CC1(C)OB(c2ccc(-c3c(-c4ccccc4)oc4ccccc34)cc2)OC1(C)C]

In a 500-ml round-bottom flask, <7-d> (23.1 g, 0.076 mol), bis(pinacolato)diboron (38.5 g, 0.152 mol), palladium acetate (0.9 g, 0.004 mol), potassium phosphate (40.2 g, 0.189 mol), SPhos (4.05 g, 0.010 mol), and 1,4-dioxane (230 ml) were refluxed together for 6 hours. After completion of the reaction, the reaction mixture was cooled to room temperature and filtered through celite. The filtrate was concentrated in a vacuum and purified by column chromatography to afford <7-e> (15.5 g, yield 51.6%).

### Synthetic Example 7-6. Synthesis of [Compound 82]

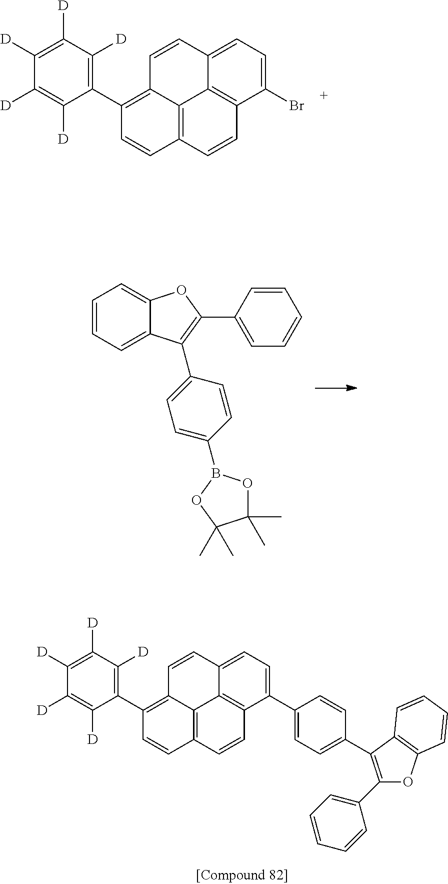 [SMILES: [2H]c1c([2H])c([2H])c(-c2ccc3ccc4c(Br)ccc5ccc2c3c54)c([2H])c1[2H] ; CC1(C)OB(c2ccc(-c3c(-c4ccccc4)oc4ccccc34)cc2)OC1(C)C ; [2H]c1c([2H])c([2H])c(-c2ccc3ccc4c(-c5ccc(-c6c(-c7ccccc7)oc7ccccc67)cc5)ccc5ccc2c3c54)c([2H])c1[2H]]

In a 500-ml round-bottom flask, <7-a> (11.5 g, 0.032 mol), <7-e> (13.2 g, 0.038 mol), tetrakis(triphenylphosphine) palladium (0.7 g, 0.001 mol). potassium carbonate (7.5 g, 0.054 mol), toluene (80 ml), ethanol (20 ml), and water (27 ml) were refluxed for 6 hours. After completion of the reaction, the reaction mixture was cooled to room temperature and subjected to extraction with ethyl acetate and water. The organic layer thus formed was concentrated in a vacuum and recrystallized in dichloromethane and methanol to afford [Compound 82] (10.4 g, yield 59.4%).

MS (MALDI-TOF): m/z 551.23 [M⁺]

### Synthetic Example 8. Synthesis of [Compound 97]

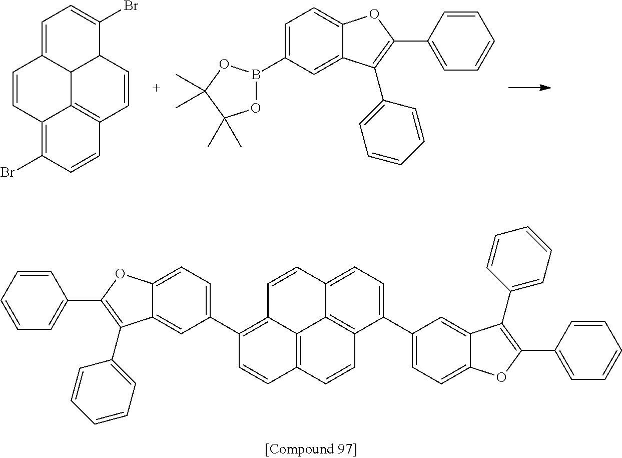 [SMILES: BrC1=CC=C2C=Cc3c(Br)ccc4c3C2C1C=C4 ; CC1(C)OB(c2ccc3oc(-c4ccccc4)c(-c4ccccc4)c3c2)OC1(C)C ; c1ccc(-c2oc3ccc(-c4ccc5ccc6c(-c7ccc8oc(-c9ccccc9)c(-c9ccccc9)c8c7)ccc7ccc4c5c76)cc3c2-c2ccccc2)cc1]

In a 300-ml round-bottom flask, 1,6-dibromopyrene (10 g, 0.028 mol), <2-f> (19.2 g, 0.061), tetrakis(triphenylphosphine) palladium (3.2 g, 0.003 mol), potassium carbonate (15.4 g, 0.111 mol), toluene (70 ml), ethanol (18 ml), and water (55 ml) were refluxed together for 12 hours. After completion of the reaction, the reaction mixture was cooled to room temperature subjected to extraction with ethyl acetate and water. The organic layer thus formed was concentrated in a vacuum and then purified by column chromatography to afford [Compound 97] (7.6 g, yield 58.5%).

MS (MALDI-TOF): m/z 738.26 [M⁺]

### Synthetic Example 9. Synthesis of [Compound 100]

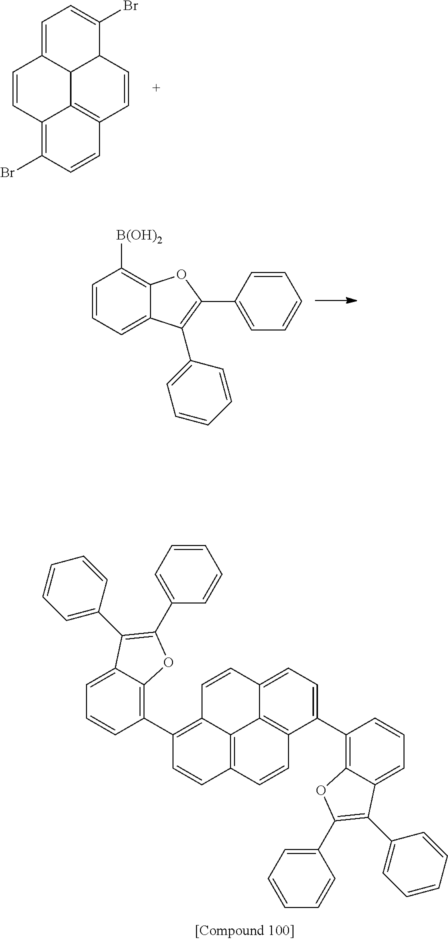 [SMILES: BrC1=CC=C2C=Cc3c(Br)ccc4c3C2C1C=C4 ; OB(O)c1cccc2c(-c3ccccc3)c(-c3ccccc3)oc12 ; c1ccc(-c2oc3c(-c4ccc5ccc6c(-c7cccc8c(-c9ccccc9)c(-c9ccccc9)oc78)ccc7ccc4c5c76)cccc3c2-c2ccccc2)cc1]

In a 300-ml round-bottom flask, 1,6-dibromopyrene (10 g, 0.028 mol), <1-c> (19.2 g, 0.061 mol), tetrakis(triphenylphosphine) palladium (3.2 g, 0.003 mol), potassium carbonate (15.4 g, 0.111 mol), toluene (70 ml), ethanol (18 ml), and water (55 ml) were refluxed together for 12 hours. After completion of the reaction, the reaction mixture was cooled to room temperature and subjected to extraction with ethyl acetate and water. The organic layer was concentrated in a vacuum and purified by column chromatography to afford [Compound 100] (16 g, yield 52%).

MS (MALDI-TOF): m/z 738.26 [M⁺]

### Examples 1 TO 10: Fabrication of Organic Light-Emitting Diodes

An ITO glass substrate was patterned to have a translucent area of 2 mm×2 mm and cleansed. The ITO glass was mounted in a vacuum chamber that was then set to have a base pressure of 1×10−7 torr. On the ITO glass substrate, films were sequentially formed of DNTPD (700 Å) and x-NPD (300 Å). Subsequently, a light-emitting layer (300 Å) was formed of a combination of the host according to the present disclosure and the dopant (BD) (3 wt %) described below. Then, [Chemical Formula E-1] and [Chemical Formula E-2] were deposited at a weight ratio of 1:1 to form an electron transport layer (300 Å) on which an electron injection layer of [Chemical Formula E-2] (10 Å) was formed and then covered with an Al (1,000 Å) to fabricate an organic light-emitting diode. The organic light-emitting diodes thus obtained were measured at 0.4 mA for luminescence properties.

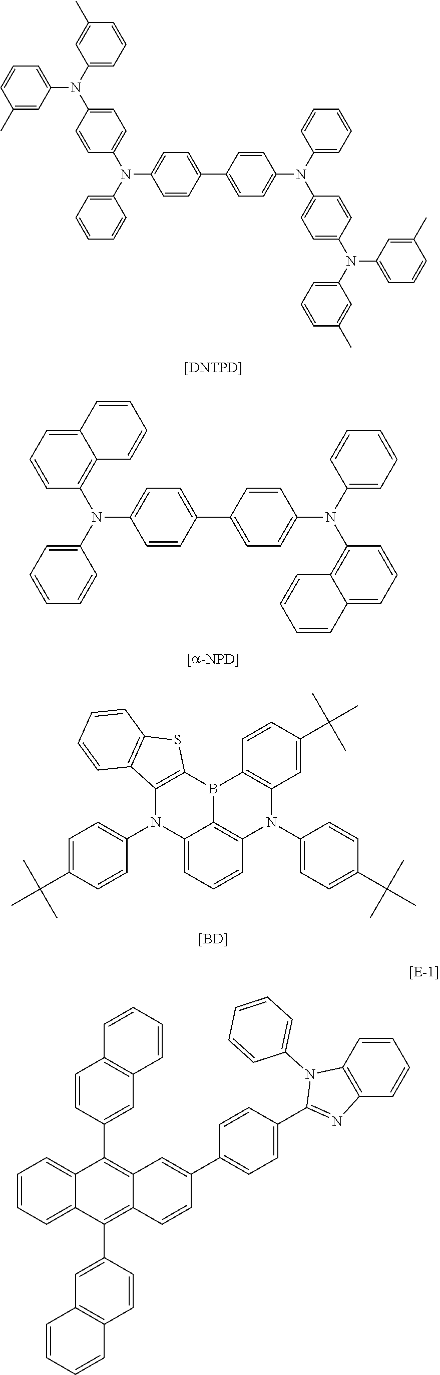

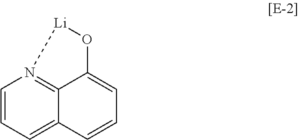

### Comparative Examples 1 AND 2

Organic light emitting diodes were fabricated in the same manner as in the Examples, with the exception of using [BH1] and [BH2] as hosts instead of the compounds according to the present disclosure. The luminescence of the organic light-emitting diodes thus obtained was measured at 0.4 mA. Structures of compounds [BH1] and [BH2] are as follows:

TABLE 1

| | | | | [BH1] |
|---|---|---|---|---|
| 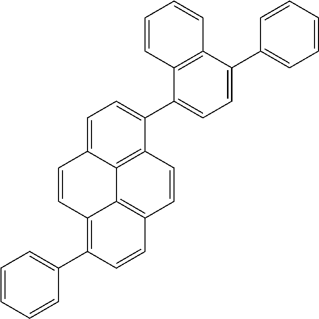 [SMILES: c1ccc(-c2ccc(-c3ccc4ccc5c(-c6ccccc6)ccc6ccc3c4c65)c3ccccc23)cc1] | | | | |
| | | | | [BH2] |
| 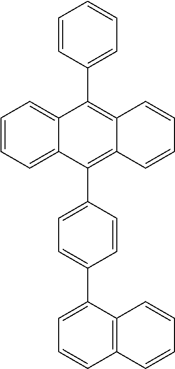 [SMILES: c1ccc(-c2c3ccccc3c(-c3ccc(-c4cccc5ccccc45)cc3)c3ccccc23)cc1] | | | | |
| | Host | V | EQE | T97 |
| C. Ex. 1 | BH1 | 3.9 | 10.3 | 148 |
| C. Ex. 2 | BH2 | 3.84 | 10.42 | 144 |
| Ex. 1 | Compound 48 | 3.73 | 11.42 | 210 |
| Ex. 2 | Compound 35 | 3.7 | 11.93 | 232 |
| Ex. 3 | Compound 24 | 3.6 | 12.19 | 262 |
| Ex. 4 | Compound 29 | 3.68 | 12.22 | 255 |
| Ex. 5 | Compound 62 | 3.73 | 11.88 | 230 |
| Ex. 6 | Compound 79 | 3.64 | 12.01 | 243 |
| Ex. 7 | Compound 82 | 3.75 | 13.33 | 300 |
| Ex. 8 | Compound 97 | 3.75 | 12.11 | 238 |
| Ex. 9 | Compound 100 | 3.76 | 11.97 | 236 |
| Ex. 10 | Compound 157 | 3.79 | 10.98 | 152 |

As is understood from data of Table 1, the organic light-emitting compounds of the present disclosure exhibited better properties such as low driving voltage, high efficiency, and long lifespan, compared to conventional compounds of Comparative Examples 1 and 2, thus finding high applications in the organic light-emitting diode field.

### INDUSTRIAL APPLICABILITY

Compared to conventional compounds, the compounds of the present disclosure allow organic light-emitting diodes to exhibit superiority in terms of luminous efficiency, driving voltage, and longevity and thus can be highly applicable in organic light-emitting diodes and related industries.

1. An organic light-emitting compound, represented by the following Chemical Formula A: 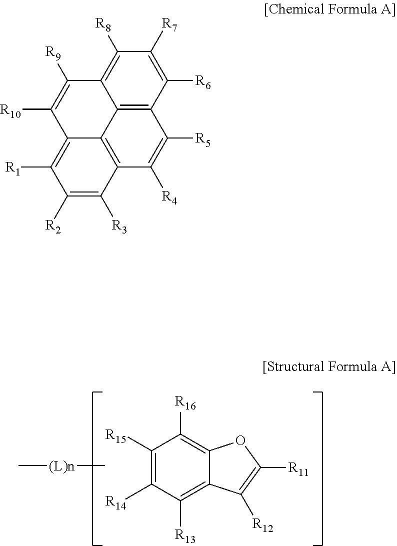
wherein,
R₁ to R₁₀, which are same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, at least one of R₁ to R₁₀ being a single bond that is linked to linker L in Structural Formula A to connect the pyrene ring moiety to the moiety represented by Structural Formula A;
if existing to connect with the pyrene ring moiety, two or more moieties of Structural Formula A are same or different;
the linker L in Structural Formula A is selected from a single bond, a substituted or unsubstituted arylene of 6 to 20 carbon atoms, and a substituted or unsubstituted heteroarylene of 2 to 20 carbon atoms;
n is an integer of 0 to 2, wherein when n is 2, the corresponding linkers L's are same or different;
R₁₁ to R₁₆, which is same or different, are each independently selected from a hydrogen atom, a deuterium atom, a halogen, a cyano, a nitro, a substituted or unsubstituted alkyl of 1 to 30 carbon atoms, a substituted or unsubstituted cycloalkyl of 3 to 30 carbon atoms, a substituted or unsubstituted heterocycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, and a substituted or unsubstituted arylsilyl of 6 to 30 carbon atoms, one of R₁₁ to R₁₆ being a single bond linked to the linker L; and
R₁₃ to R₁₆ can each further form mono- or polycyclic aliphatic or aromatic ring with an adjacent radical thereto,
wherein the term ‘substituted’ in the expression “a substituted or unsubstituted” means having at least one substituent selected from the group consisting of a deuterium atom, a cyano, a halogen, a hydroxy, a nitro, an alkyl of 1 to 24 carbon atoms, a hydrogenated alkyl of 1 to 24 carbon atoms, an alkenyl of 1 to 24 carbon atoms, an alkynyl of 1 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

2. The organic light-emitting compound of claim 1, wherein at least one of the substituents R₁, R₃, R₆, and R₈ on the pyrene moiety in Chemical Formula A is a single bond linked to the linker L.

3. The organic light-emitting compound of claim 1, wherein the substituent R₁ is be a single bond linked to the linker L within the moiety of Structural Formula A, and the substituent R₆ is any one selected from a hydrogen atom, a deuterium atom, a substituted or unsubstituted aryl of 6 to 18 carbon atoms, and a substituted or unsubstituted heteroaryl of 2 to 30 carbon atoms.

4. The organic light-emitting compound of claim 1, wherein the substituents R₁ and R₆ are same or different and are each a single bond linked to the linker L in Structural Formula A.

5. The organic light-emitting compound of claim 1, wherein the compound represented by Chemical Formula A bears at least one deuterium atom.

6. The organic light-emitting compound of claim 5, wherein at least one of R₁, R₃, R₆, and R₈ is a substituent bearing a deuterium atom, or at least one of R₁₁ to R₁₆ is a substituent bearing a deuterium atom.

7. The organic light-emitting compound of claim 1, wherein one of R₁₃ to R₁₆ within Structural Formula A is a single bond linked to the linker L.

8. The organic light-emitting compound of claim 1, wherein R₁₂ within Structural Formula A is a single bond linked to the linker L.

9. The organic light-emitting compound of claim 1, wherein the linker L is a single bond or any one selected from the following Structural Formulas 1 to 5: 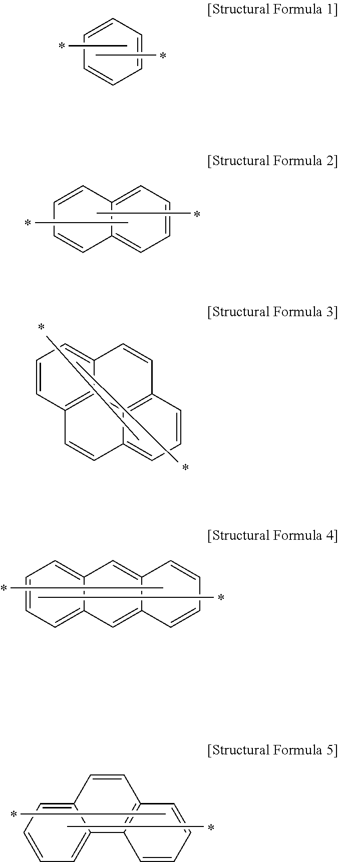
wherein each of the unsubstituted carbon atoms of the aromatic ring moieties can be bound with a hydrogen atom or a deuterium atom.

10. The organic light-emitting compound of claim 8, wherein the linker L in Chemical Formula A is a single bond.

11. The organic light-emitting compound of claim 8, wherein the linker L in Chemical Formula A is a substituted or unsubstituted aryl of 6 to 18 carbon atoms.

12. The organic light-emitting compound of claim 1, wherein the compound is any one selected from the following [Compound 1] to [Compound 157]:

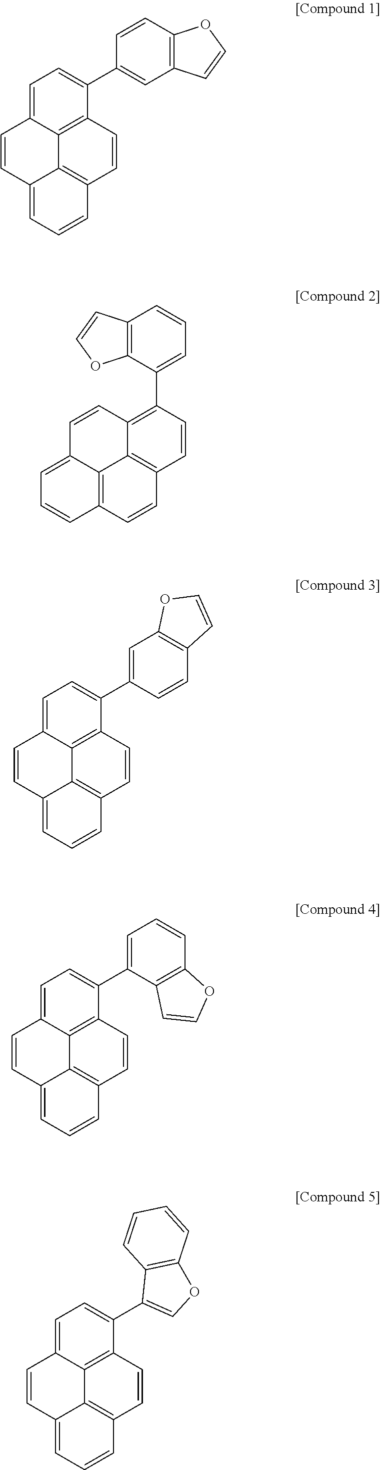 [SMILES: c1cc2ccc3ccc(-c4ccc5occc5c4)c4ccc(c1)c2c34 ; c1cc(-c2ccc3ccc4cccc5ccc2c3c45)c2occc2c1 ; c1cc2ccc3ccc(-c4ccc5ccoc5c4)c4ccc(c1)c2c34 ; c1cc2ccc3ccc(-c4cccc5occc45)c4ccc(c1)c2c34 ; c1cc2ccc3ccc(-c4coc5ccccc45)c4ccc(c1)c2c34]

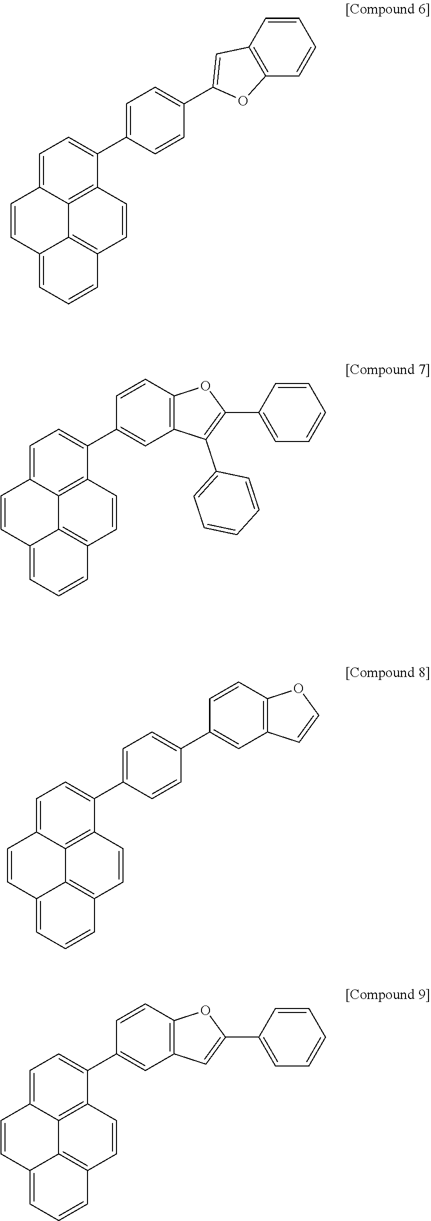 [SMILES: c1ccc2oc(-c3ccc(-c4ccc5ccc6cccc7ccc4c5c67)cc3)cc2c1 ; c1ccc(-c2oc3ccc(-c4ccc5ccc6cccc7ccc4c5c67)cc3c2-c2ccccc2)cc1 ; c1cc2ccc3ccc(-c4ccc(-c5ccc6occc6c5)cc4)c4ccc(c1)c2c34 ; c1ccc(-c2cc3cc(-c4ccc5ccc6cccc7ccc4c5c67)ccc3o2)cc1]

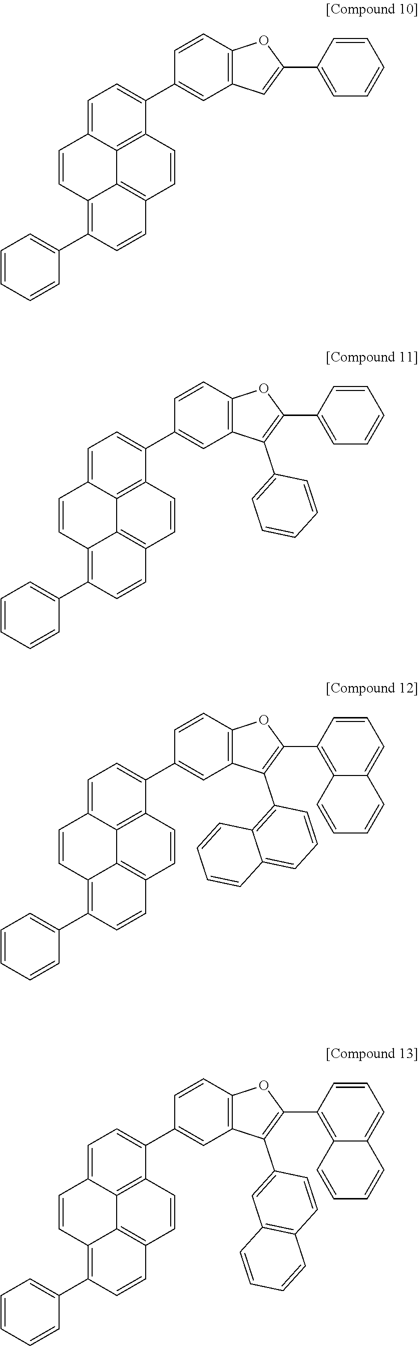 [SMILES: c1ccc(-c2cc3cc(-c4ccc5ccc6c(-c7ccccc7)ccc7ccc4c5c76)ccc3o2)cc1 ; c1ccc(-c2oc3ccc(-c4ccc5ccc6c(-c7ccccc7)ccc7ccc4c5c76)cc3c2-c2ccccc2)cc1 ; c1ccc(-c2ccc3ccc4c(-c5ccc6oc(-c7cccc8ccccc78)c(-c7cccc8ccccc78)c6c5)ccc5ccc2c3c54)cc1 ; c1ccc(-c2ccc3ccc4c(-c5ccc6oc(-c7cccc8ccccc78)c(-c7ccc8ccccc8c7)c6c5)ccc5ccc2c3c54)cc1]

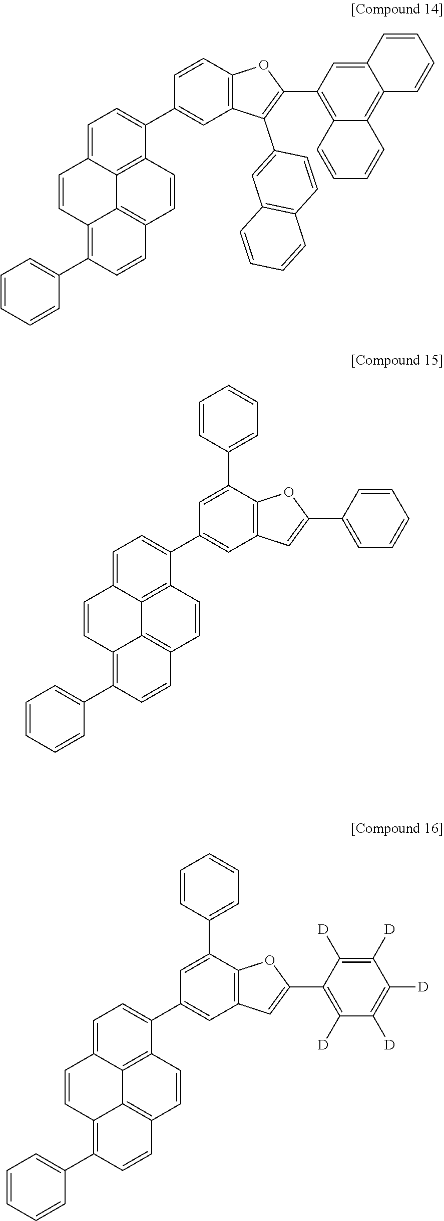 [SMILES: c1ccc(-c2ccc3ccc4c(-c5ccc6oc(-c7cc8ccccc8c8ccccc78)c(-c7ccc8ccccc8c7)c6c5)ccc5ccc2c3c54)cc1 ; c1ccc(-c2cc3cc(-c4ccc5ccc6c(-c7ccccc7)ccc7ccc4c5c76)cc(-c4ccccc4)c3o2)cc1 ; [2H]c1c([2H])c([2H])c(-c2cc3cc(-c4ccc5ccc6c(-c7ccccc7)ccc7ccc4c5c76)cc(-c4ccccc4)c3o2)c([2H])c1[2H]]

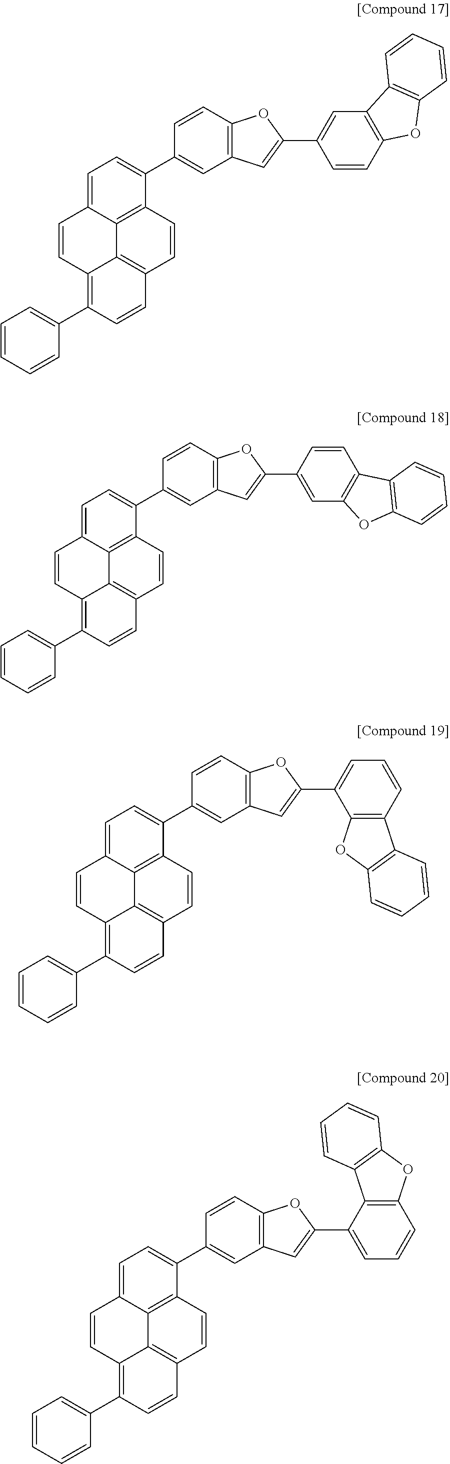 [SMILES: c1ccc(-c2ccc3ccc4c(-c5ccc6oc(-c7ccc8oc9ccccc9c8c7)cc6c5)ccc5ccc2c3c54)cc1 ; c1ccc(-c2ccc3ccc4c(-c5ccc6oc(-c7ccc8c(c7)oc7ccccc78)cc6c5)ccc5ccc2c3c54)cc1 ; c1ccc(-c2ccc3ccc4c(-c5ccc6oc(-c7cccc8c7oc7ccccc78)cc6c5)ccc5ccc2c3c54)cc1 ; c1ccc(-c2ccc3ccc4c(-c5ccc6oc(-c7cccc8oc9ccccc9c78)cc6c5)ccc5ccc2c3c54)cc1]

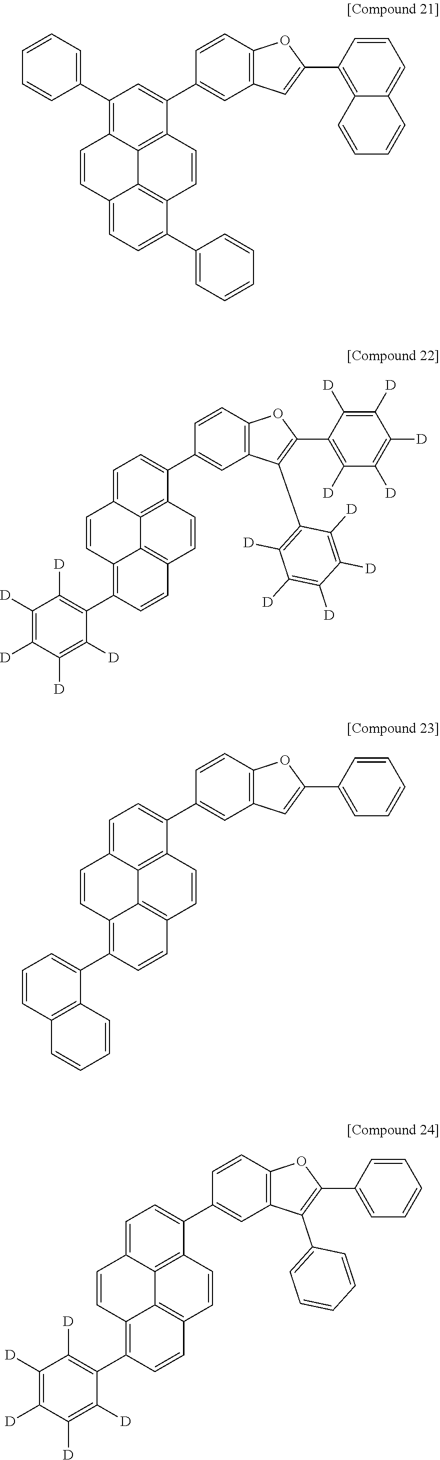 [SMILES: c1ccc(-c2ccc3ccc4c(-c5ccccc5)cc(-c5ccc6oc(-c7cccc8ccccc78)cc6c5)c5ccc2c3c45)cc1 ; [2H]c1c([2H])c([2H])c(-c2oc3ccc(-c4ccc5ccc6c(-c7c([2H])c([2H])c([2H])c([2H])c7[2H])ccc7ccc4c5c76)cc3c2-c2c([2H])c([2H])c([2H])c([2H])c2[2H])c([2H])c1[2H] ; c1ccc(-c2cc3cc(-c4ccc5ccc6c(-c7cccc8ccccc78)ccc7ccc4c5c76)ccc3o2)cc1 ; [2H]c1c([2H])c([2H])c(-c2ccc3ccc4c(-c5ccc6oc(-c7ccccc7)c(-c7ccccc7)c6c5)ccc5ccc2c3c54)c([2H])c1[2H]]

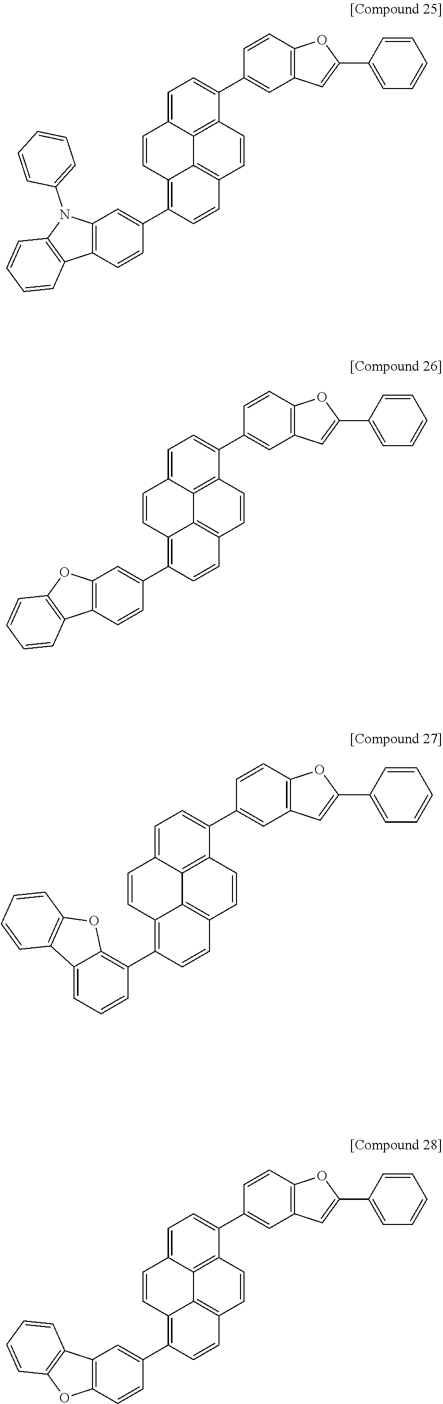 [SMILES: c1ccc(-c2cc3cc(-c4ccc5ccc6c(-c7ccc8c9ccccc9n(-c9ccccc9)c8c7)ccc7ccc4c5c76)ccc3o2)cc1 ; c1ccc(-c2cc3cc(-c4ccc5ccc6c(-c7ccc8c(c7)oc7ccccc78)ccc7ccc4c5c76)ccc3o2)cc1 ; c1ccc(-c2cc3cc(-c4ccc5ccc6c(-c7cccc8c7oc7ccccc78)ccc7ccc4c5c76)ccc3o2)cc1 ; c1ccc(-c2cc3cc(-c4ccc5ccc6c(-c7ccc8oc9ccccc9c8c7)ccc7ccc4c5c76)ccc3o2)cc1]

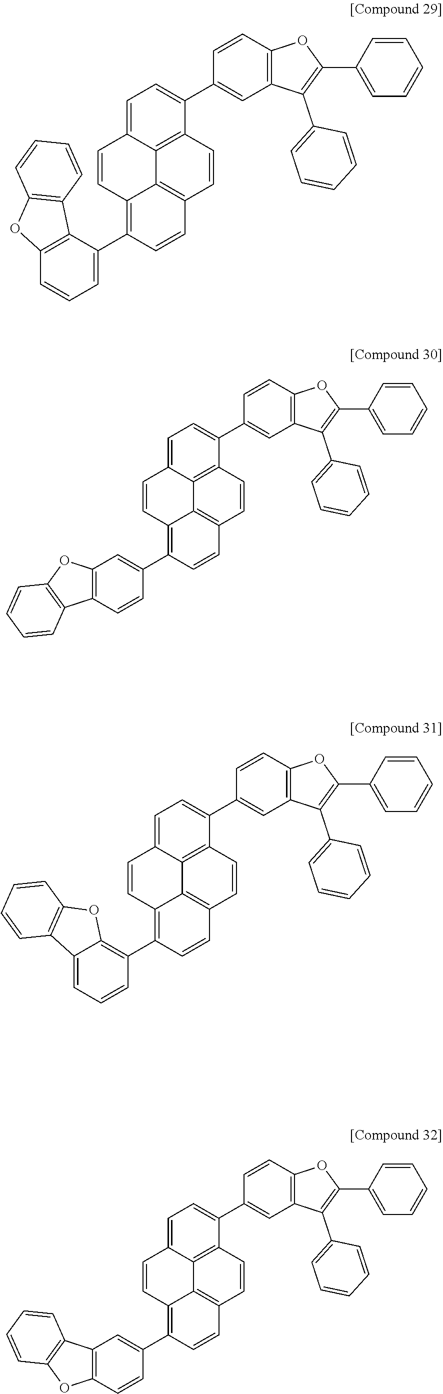 [SMILES: c1ccc(-c2oc3ccc(-c4ccc5ccc6c(-c7cccc8oc9ccccc9c78)ccc7ccc4c5c76)cc3c2-c2ccccc2)cc1 ; c1ccc(-c2oc3ccc(-c4ccc5ccc6c(-c7ccc8c(c7)oc7ccccc78)ccc7ccc4c5c76)cc3c2-c2ccccc2)cc1 ; c1ccc(-c2oc3ccc(-c4ccc5ccc6c(-c7cccc8c7oc7ccccc78)ccc7ccc4c5c76)cc3c2-c2ccccc2)cc1 ; c1ccc(-c2oc3ccc(-c4ccc5ccc6c(-c7ccc8oc9ccccc9c8c7)ccc7ccc4c5c76)cc3c2-c2ccccc2)cc1]

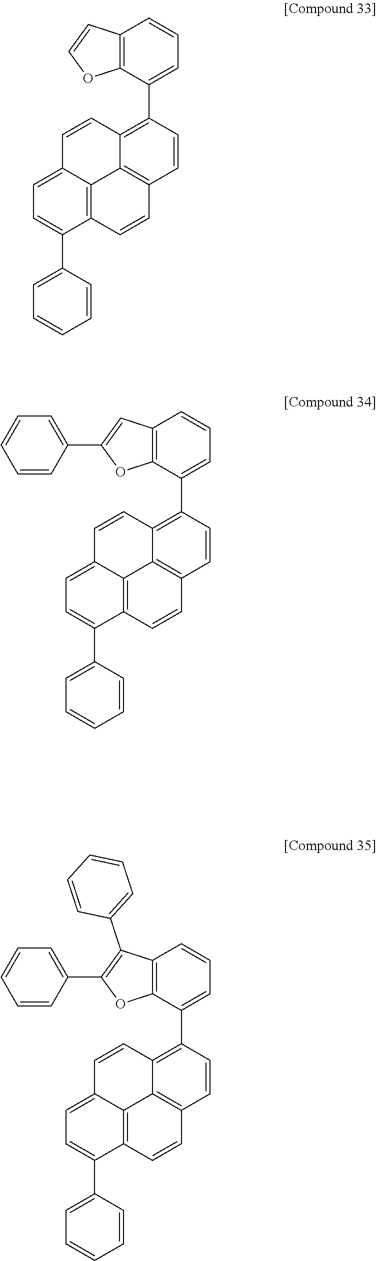 [SMILES: c1ccc(-c2ccc3ccc4c(-c5cccc6ccoc56)ccc5ccc2c3c54)cc1 ; c1ccc(-c2cc3cccc(-c4ccc5ccc6c(-c7ccccc7)ccc7ccc4c5c76)c3o2)cc1 ; c1ccc(-c2oc3c(-c4ccc5ccc6c(-c7ccccc7)ccc7ccc4c5c76)cccc3c2-c2ccccc2)cc1]

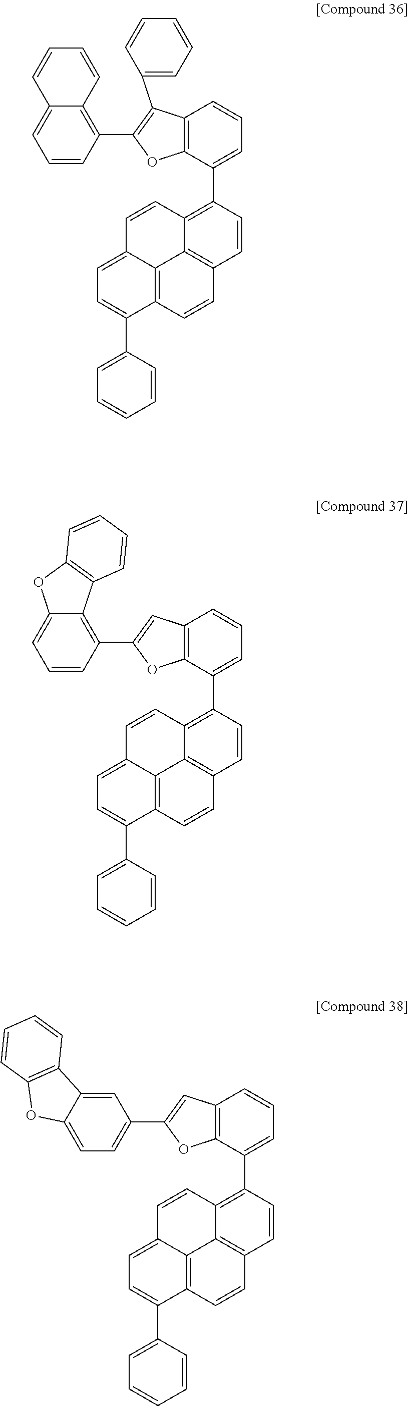 [SMILES: c1ccc(-c2c(-c3cccc4ccccc34)oc3c(-c4ccc5ccc6c(-c7ccccc7)ccc7ccc4c5c76)cccc23)cc1 ; c1ccc(-c2ccc3ccc4c(-c5cccc6cc(-c7cccc8oc9ccccc9c78)oc56)ccc5ccc2c3c54)cc1 ; c1ccc(-c2ccc3ccc4c(-c5cccc6cc(-c7ccc8oc9ccccc9c8c7)oc56)ccc5ccc2c3c54)cc1]

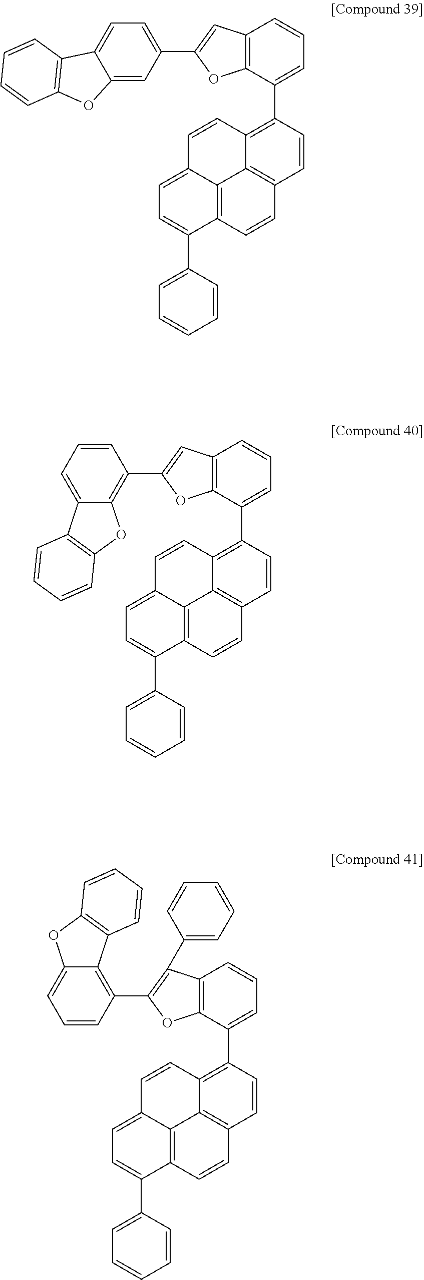 [SMILES: c1ccc(-c2ccc3ccc4c(-c5cccc6cc(-c7ccc8c(c7)oc7ccccc78)oc56)ccc5ccc2c3c54)cc1 ; c1ccc(-c2ccc3ccc4c(-c5cccc6cc(-c7cccc8c7oc7ccccc78)oc56)ccc5ccc2c3c54)cc1 ; c1ccc(-c2c(-c3cccc4oc5ccccc5c34)oc3c(-c4ccc5ccc6c(-c7ccccc7)ccc7ccc4c5c76)cccc23)cc1]

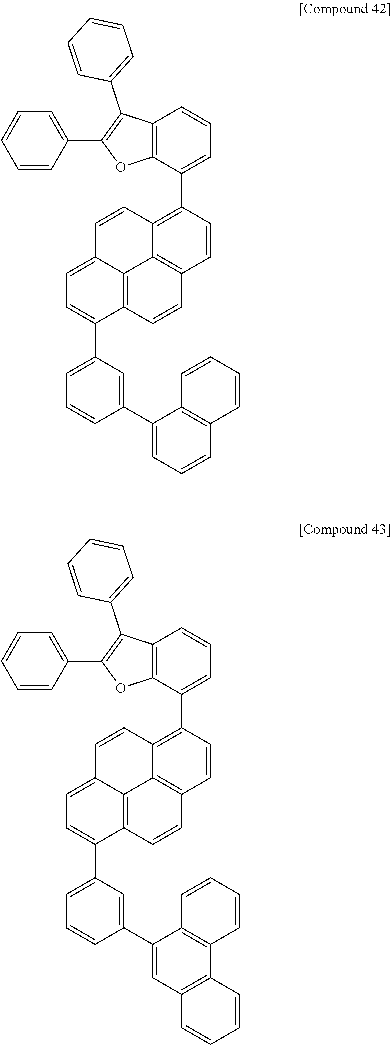 [SMILES: c1ccc(-c2oc3c(-c4ccc5ccc6c(-c7cccc(-c8cccc9ccccc89)c7)ccc7ccc4c5c76)cccc3c2-c2ccccc2)cc1 ; c1ccc(-c2oc3c(-c4ccc5ccc6c(-c7cccc(-c8cc9ccccc9c9ccccc89)c7)ccc7ccc4c5c76)cccc3c2-c2ccccc2)cc1]

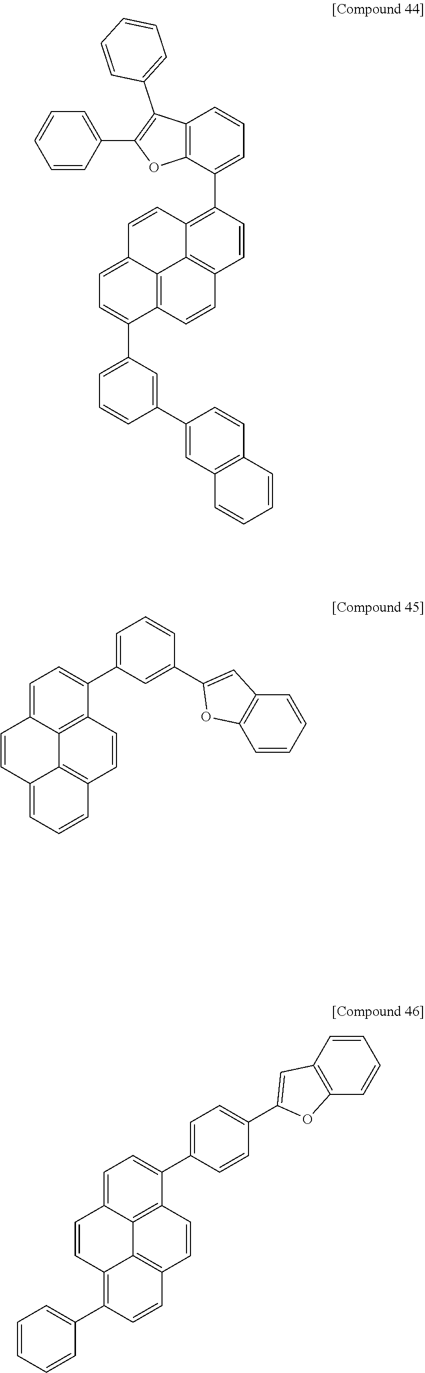 [SMILES: c1ccc(-c2oc3c(-c4ccc5ccc6c(-c7cccc(-c8ccc9ccccc9c8)c7)ccc7ccc4c5c76)cccc3c2-c2ccccc2)cc1 ; c1cc(-c2cc3ccccc3o2)cc(-c2ccc3ccc4cccc5ccc2c3c45)c1 ; c1ccc(-c2ccc3ccc4c(-c5ccc(-c6cc7ccccc7o6)cc5)ccc5ccc2c3c54)cc1]

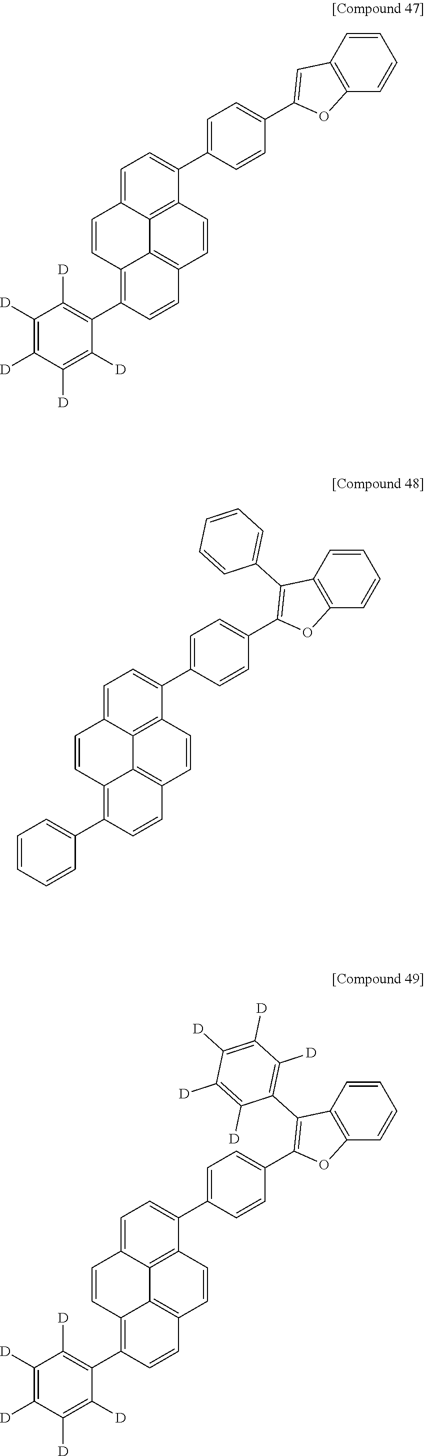 [SMILES: [2H]c1c([2H])c([2H])c(-c2ccc3ccc4c(-c5ccc(-c6cc7ccccc7o6)cc5)ccc5ccc2c3c54)c([2H])c1[2H] ; c1ccc(-c2c(-c3ccc(-c4ccc5ccc6c(-c7ccccc7)ccc7ccc4c5c76)cc3)oc3ccccc23)cc1 ; [2H]c1c([2H])c([2H])c(-c2c(-c3ccc(-c4ccc5ccc6c(-c7c([2H])c([2H])c([2H])c([2H])c7[2H])ccc7ccc4c5c76)cc3)oc3ccccc23)c([2H])c1[2H]]

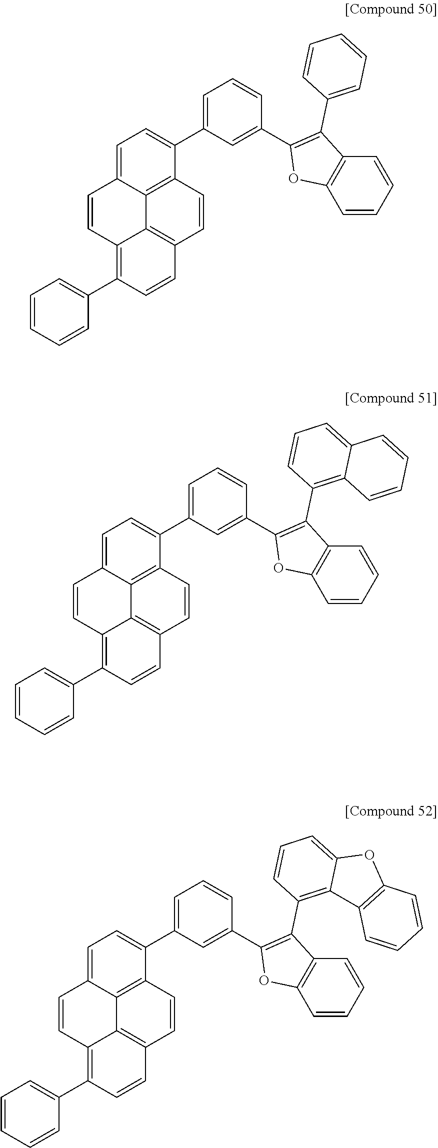 [SMILES: c1ccc(-c2c(-c3cccc(-c4ccc5ccc6c(-c7ccccc7)ccc7ccc4c5c76)c3)oc3ccccc23)cc1 ; c1ccc(-c2ccc3ccc4c(-c5cccc(-c6oc7ccccc7c6-c6cccc7ccccc67)c5)ccc5ccc2c3c54)cc1 ; c1ccc(-c2ccc3ccc4c(-c5cccc(-c6oc7ccccc7c6-c6cccc7oc8ccccc8c67)c5)ccc5ccc2c3c54)cc1]

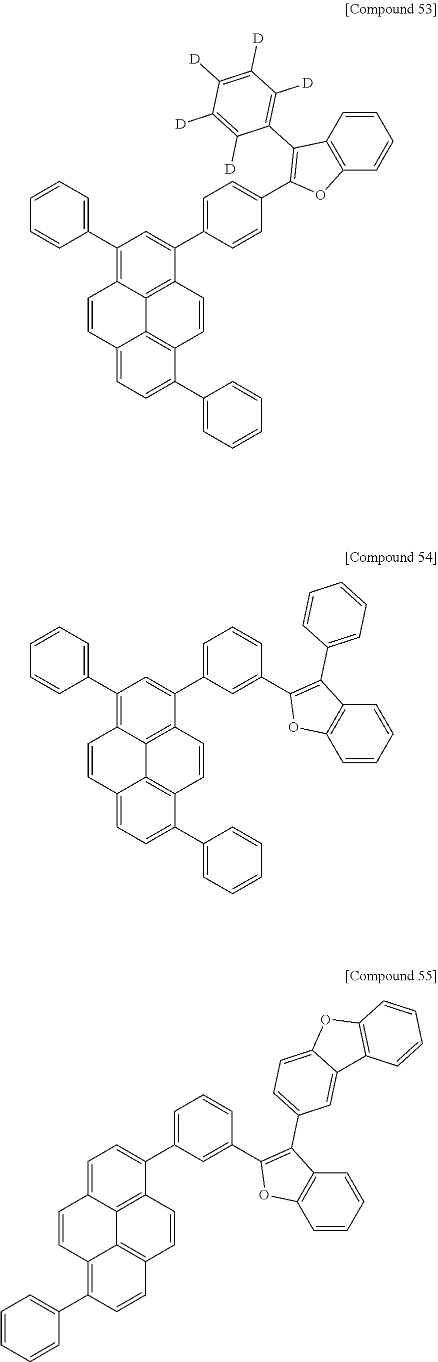 [SMILES: [2H]c1c([2H])c([2H])c(-c2c(-c3ccc(-c4cc(-c5ccccc5)c5ccc6ccc(-c7ccccc7)c7ccc4c5c67)cc3)oc3ccccc23)c([2H])c1[2H] ; c1ccc(-c2c(-c3cccc(-c4cc(-c5ccccc5)c5ccc6ccc(-c7ccccc7)c7ccc4c5c67)c3)oc3ccccc23)cc1 ; c1ccc(-c2ccc3ccc4c(-c5cccc(-c6oc7ccccc7c6-c6ccc7oc8ccccc8c7c6)c5)ccc5ccc2c3c54)cc1]

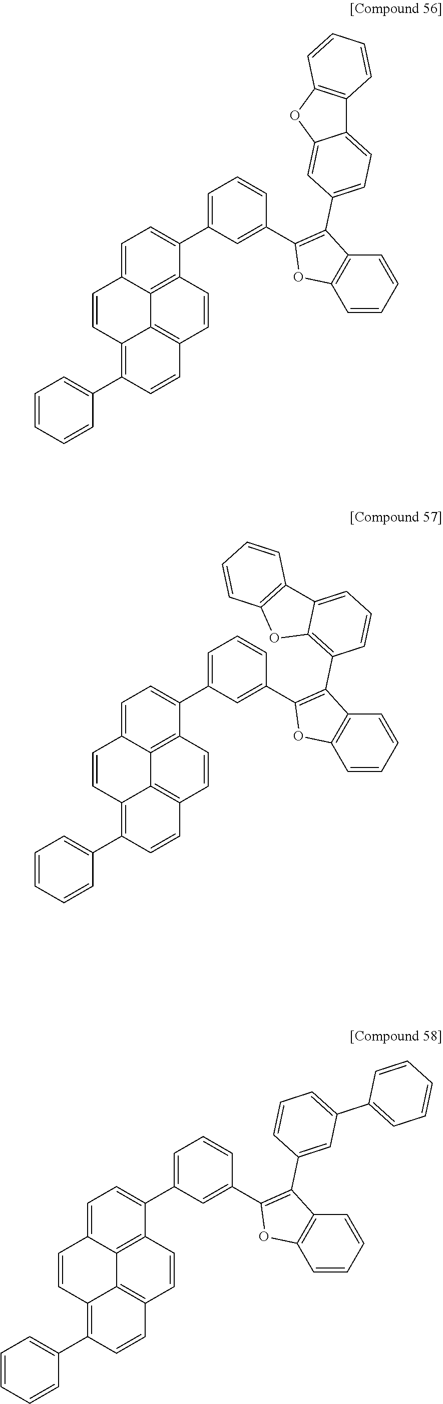 [SMILES: c1ccc(-c2ccc3ccc4c(-c5cccc(-c6oc7ccccc7c6-c6ccc7c(c6)oc6ccccc67)c5)ccc5ccc2c3c54)cc1 ; c1ccc(-c2ccc3ccc4c(-c5cccc(-c6oc7ccccc7c6-c6cccc7c6oc6ccccc67)c5)ccc5ccc2c3c54)cc1 ; c1ccc(-c2cccc(-c3c(-c4cccc(-c5ccc6ccc7c(-c8ccccc8)ccc8ccc5c6c87)c4)oc4ccccc34)c2)cc1]

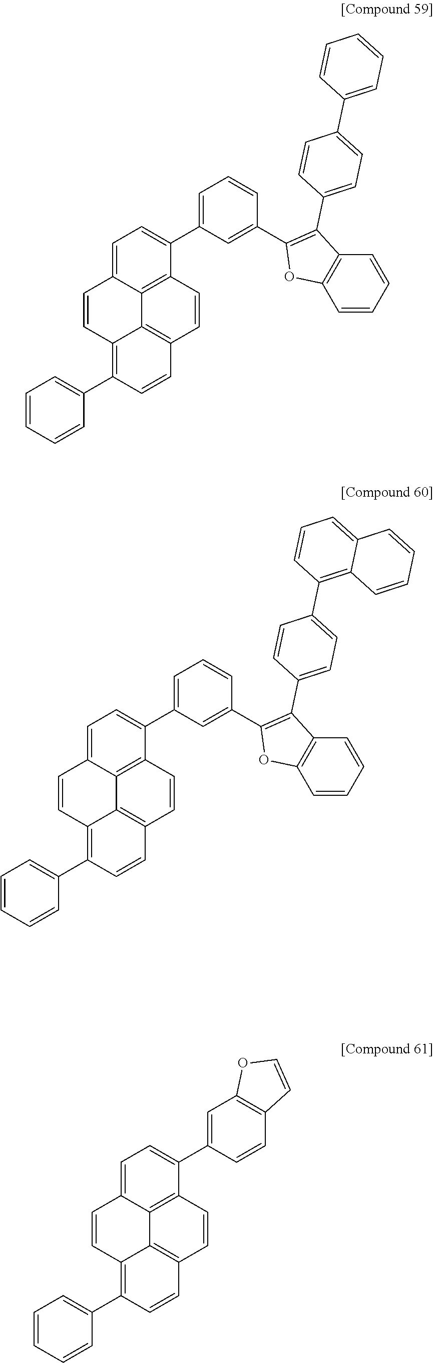 [SMILES: c1ccc(-c2ccc(-c3c(-c4cccc(-c5ccc6ccc7c(-c8ccccc8)ccc8ccc5c6c87)c4)oc4ccccc34)cc2)cc1 ; c1ccc(-c2ccc3ccc4c(-c5cccc(-c6oc7ccccc7c6-c6ccc(-c7cccc8ccccc78)cc6)c5)ccc5ccc2c3c54)cc1 ; c1ccc(-c2ccc3ccc4c(-c5ccc6ccoc6c5)ccc5ccc2c3c54)cc1]

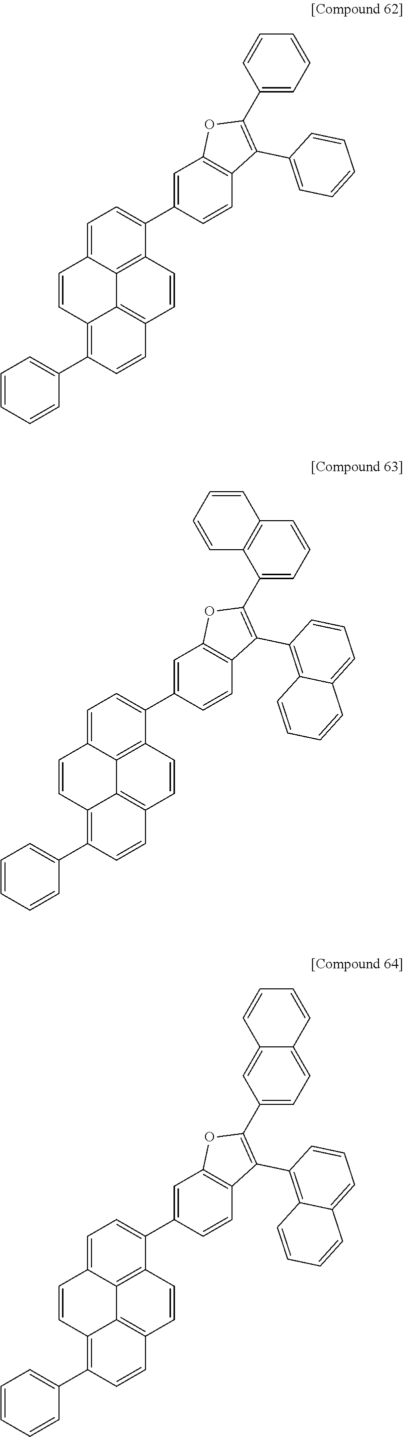 [SMILES: c1ccc(-c2oc3cc(-c4ccc5ccc6c(-c7ccccc7)ccc7ccc4c5c76)ccc3c2-c2ccccc2)cc1 ; c1ccc(-c2ccc3ccc4c(-c5ccc6c(-c7cccc8ccccc78)c(-c7cccc8ccccc78)oc6c5)ccc5ccc2c3c54)cc1 ; c1ccc(-c2ccc3ccc4c(-c5ccc6c(-c7cccc8ccccc78)c(-c7ccc8ccccc8c7)oc6c5)ccc5ccc2c3c54)cc1]

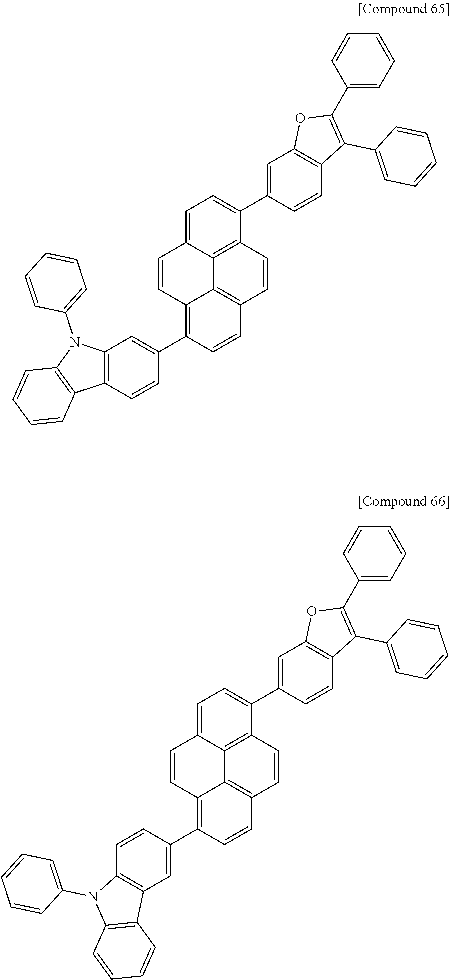 [SMILES: c1ccc(-c2oc3cc(-c4ccc5ccc6c(-c7ccc8c9ccccc9n(-c9ccccc9)c8c7)ccc7ccc4c5c76)ccc3c2-c2ccccc2)cc1 ; c1ccc(-c2oc3cc(-c4ccc5ccc6c(-c7ccc8c(c7)c7ccccc7n8-c7ccccc7)ccc7ccc4c5c76)ccc3c2-c2ccccc2)cc1]

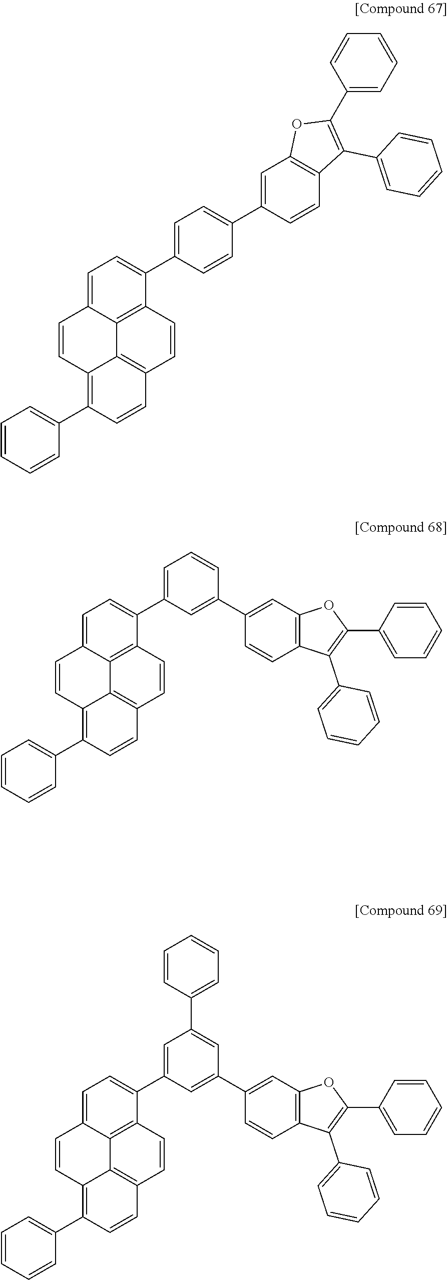 [SMILES: c1ccc(-c2oc3cc(-c4ccc(-c5ccc6ccc7c(-c8ccccc8)ccc8ccc5c6c87)cc4)ccc3c2-c2ccccc2)cc1 ; c1ccc(-c2oc3cc(-c4cccc(-c5ccc6ccc7c(-c8ccccc8)ccc8ccc5c6c87)c4)ccc3c2-c2ccccc2)cc1 ; c1ccc(-c2cc(-c3ccc4c(-c5ccccc5)c(-c5ccccc5)oc4c3)cc(-c3ccc4ccc5c(-c6ccccc6)ccc6ccc3c4c65)c2)cc1]

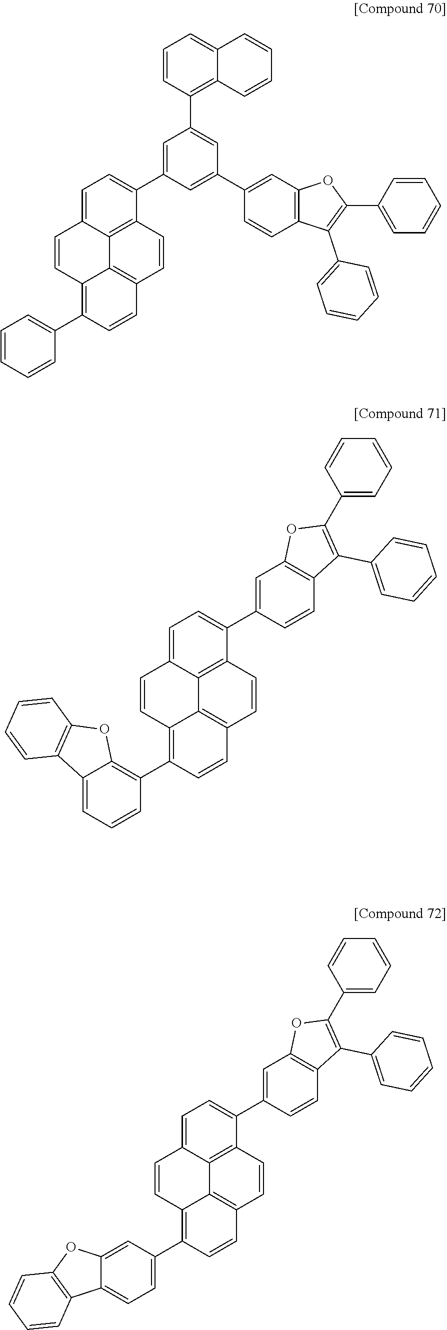 [SMILES: c1ccc(-c2oc3cc(-c4cc(-c5cccc6ccccc56)cc(-c5ccc6ccc7c(-c8ccccc8)ccc8ccc5c6c87)c4)ccc3c2-c2ccccc2)cc1 ; c1ccc(-c2oc3cc(-c4ccc5ccc6c(-c7cccc8c7oc7ccccc78)ccc7ccc4c5c76)ccc3c2-c2ccccc2)cc1 ; c1ccc(-c2oc3cc(-c4ccc5ccc6c(-c7ccc8c(c7)oc7ccccc78)ccc7ccc4c5c76)ccc3c2-c2ccccc2)cc1]

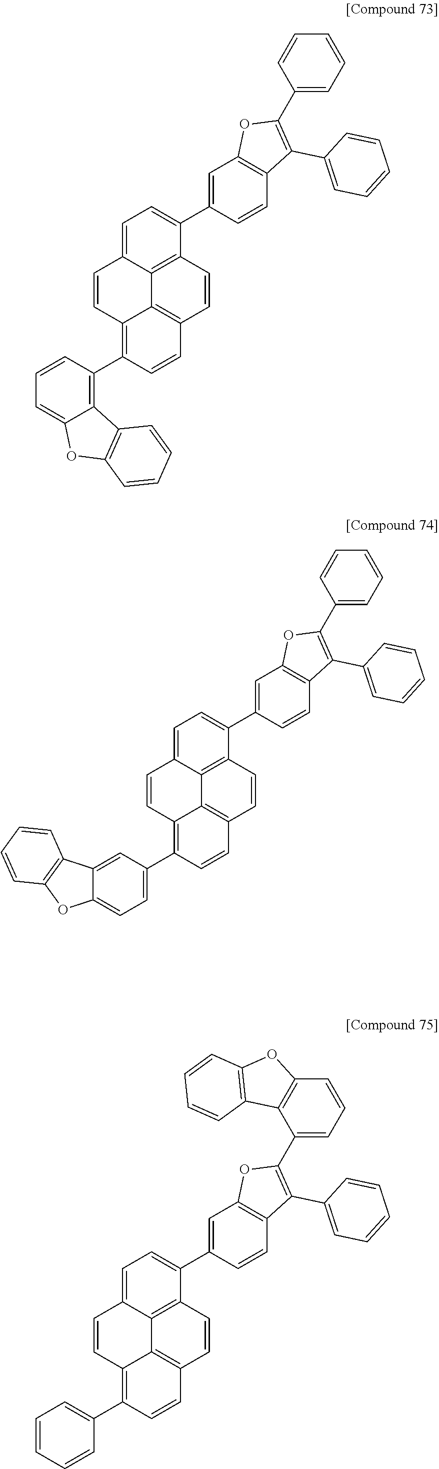 [SMILES: c1ccc(-c2oc3cc(-c4ccc5ccc6c(-c7cccc8oc9ccccc9c78)ccc7ccc4c5c76)ccc3c2-c2ccccc2)cc1 ; c1ccc(-c2oc3cc(-c4ccc5ccc6c(-c7ccc8oc9ccccc9c8c7)ccc7ccc4c5c76)ccc3c2-c2ccccc2)cc1 ; c1ccc(-c2c(-c3cccc4oc5ccccc5c34)oc3cc(-c4ccc5ccc6c(-c7ccccc7)ccc7ccc4c5c76)ccc23)cc1]

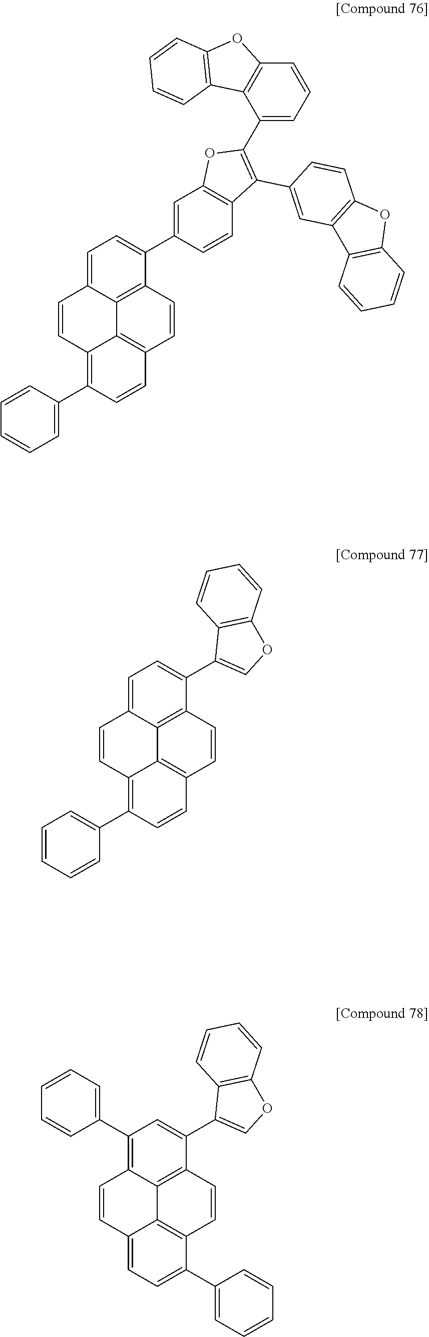 [SMILES: c1ccc(-c2ccc3ccc4c(-c5ccc6c(-c7ccc8oc9ccccc9c8c7)c(-c7cccc8oc9ccccc9c78)oc6c5)ccc5ccc2c3c54)cc1 ; c1ccc(-c2ccc3ccc4c(-c5coc6ccccc56)ccc5ccc2c3c54)cc1 ; c1ccc(-c2ccc3ccc4c(-c5ccccc5)cc(-c5coc6ccccc56)c5ccc2c3c45)cc1]

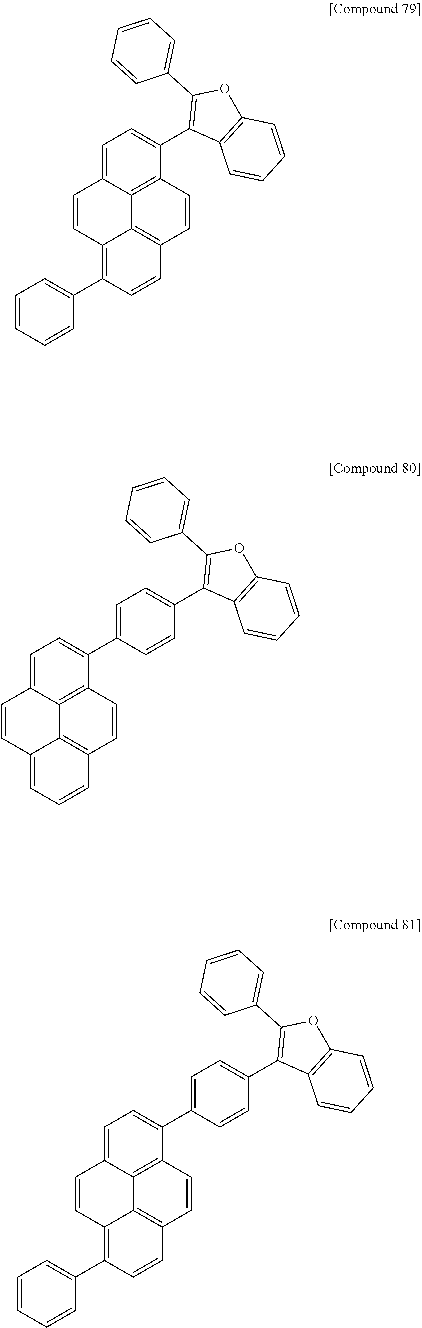 [SMILES: c1ccc(-c2oc3ccccc3c2-c2ccc3ccc4c(-c5ccccc5)ccc5ccc2c3c54)cc1 ; c1ccc(-c2oc3ccccc3c2-c2ccc(-c3ccc4ccc5cccc6ccc3c4c56)cc2)cc1 ; c1ccc(-c2oc3ccccc3c2-c2ccc(-c3ccc4ccc5c(-c6ccccc6)ccc6ccc3c4c65)cc2)cc1]

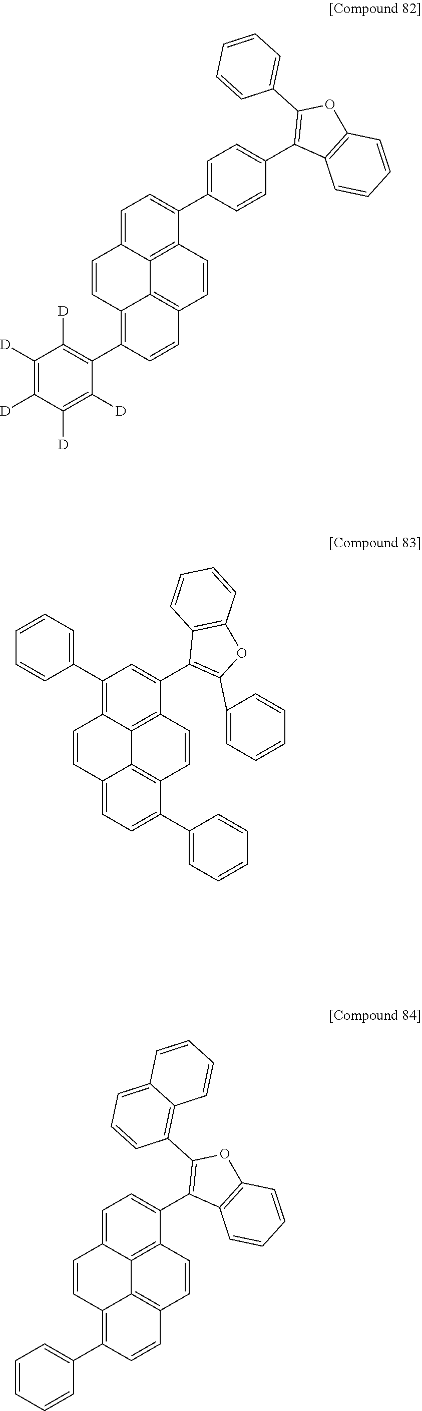 [SMILES: [2H]c1c([2H])c([2H])c(-c2ccc3ccc4c(-c5ccc(-c6c(-c7ccccc7)oc7ccccc67)cc5)ccc5ccc2c3c54)c([2H])c1[2H] ; c1ccc(-c2oc3ccccc3c2-c2cc(-c3ccccc3)c3ccc4ccc(-c5ccccc5)c5ccc2c3c45)cc1 ; c1ccc(-c2ccc3ccc4c(-c5c(-c6cccc7ccccc67)oc6ccccc56)ccc5ccc2c3c54)cc1]

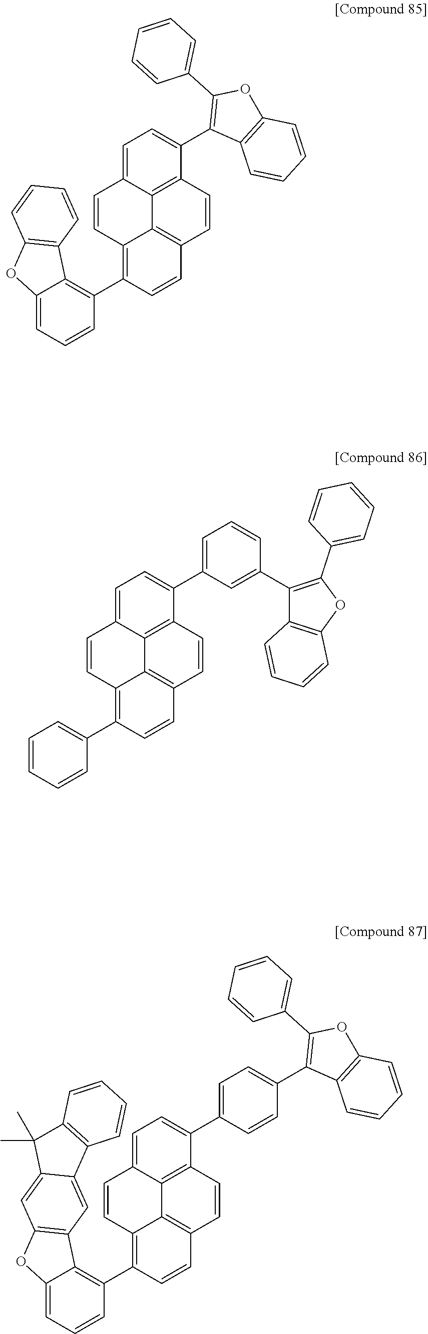 [SMILES: c1ccc(-c2oc3ccccc3c2-c2ccc3ccc4c(-c5cccc6oc7ccccc7c56)ccc5ccc2c3c54)cc1 ; c1ccc(-c2oc3ccccc3c2-c2cccc(-c3ccc4ccc5c(-c6ccccc6)ccc6ccc3c4c65)c2)cc1 ; CC1(C)c2ccccc2-c2cc3c(cc21)oc1cccc(-c2ccc4ccc5c(-c6ccc(-c7c(-c8ccccc8)oc8ccccc78)cc6)ccc6ccc2c4c65)c13]

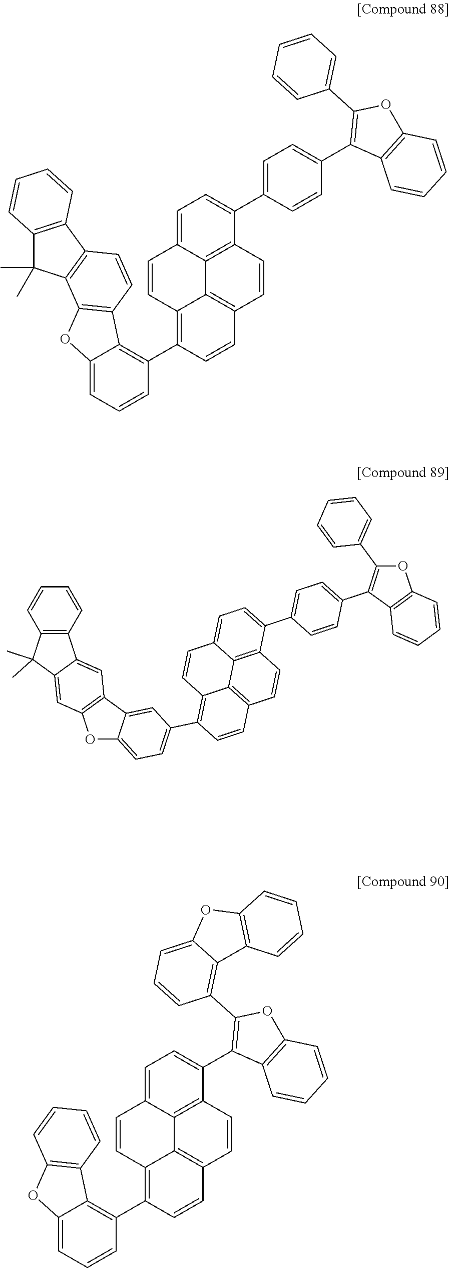 [SMILES: CC1(C)c2ccccc2-c2ccc3c(oc4cccc(-c5ccc6ccc7c(-c8ccc(-c9c(-c%10ccccc%10)oc%10ccccc9%10)cc8)ccc8ccc5c6c87)c43)c21 ; CC1(C)c2ccccc2-c2cc3c(cc21)oc1ccc(-c2ccc4ccc5c(-c6ccc(-c7c(-c8ccccc8)oc8ccccc78)cc6)ccc6ccc2c4c65)cc13 ; c1ccc2c(-c3ccc4ccc5c(-c6cccc7oc8ccccc8c67)ccc6ccc3c4c65)c(-c3cccc4oc5ccccc5c34)oc2c1]

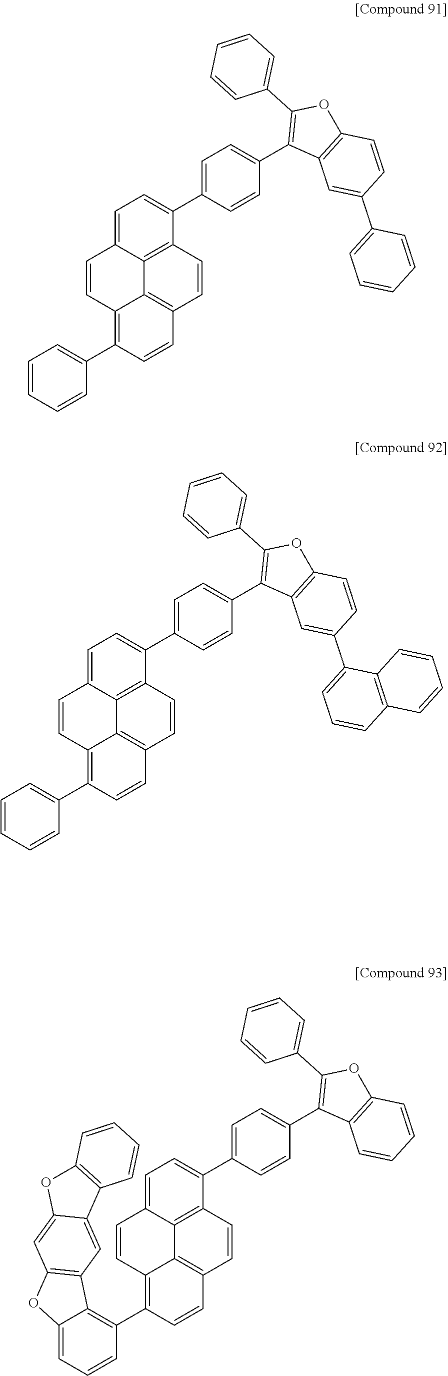 [SMILES: c1ccc(-c2ccc3oc(-c4ccccc4)c(-c4ccc(-c5ccc6ccc7c(-c8ccccc8)ccc8ccc5c6c87)cc4)c3c2)cc1 ; c1ccc(-c2oc3ccc(-c4cccc5ccccc45)cc3c2-c2ccc(-c3ccc4ccc5c(-c6ccccc6)ccc6ccc3c4c65)cc2)cc1 ; c1ccc(-c2oc3ccccc3c2-c2ccc(-c3ccc4ccc5c(-c6cccc7oc8cc9oc%10ccccc%10c9cc8c67)ccc6ccc3c4c65)cc2)cc1]

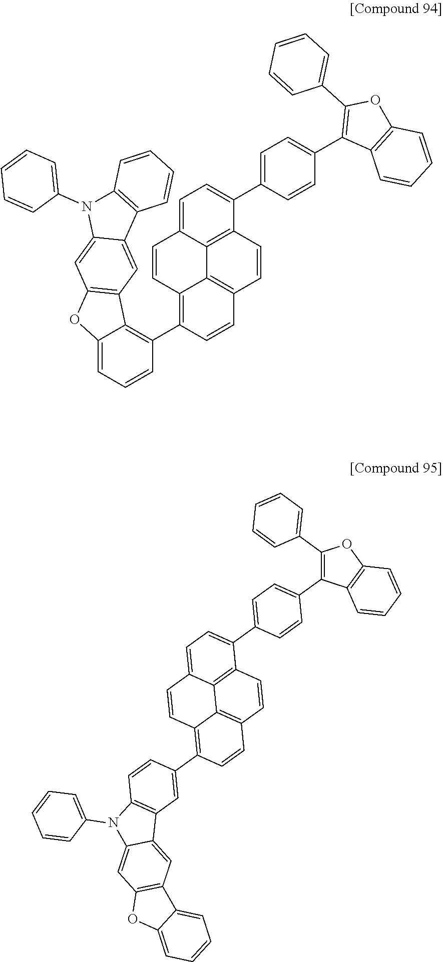 [SMILES: c1ccc(-c2oc3ccccc3c2-c2ccc(-c3ccc4ccc5c(-c6cccc7oc8cc9c(cc8c67)c6ccccc6n9-c6ccccc6)ccc6ccc3c4c65)cc2)cc1 ; c1ccc(-c2oc3ccccc3c2-c2ccc(-c3ccc4ccc5c(-c6ccc7c(c6)c6cc8c(cc6n7-c6ccccc6)oc6ccccc68)ccc6ccc3c4c65)cc2)cc1]

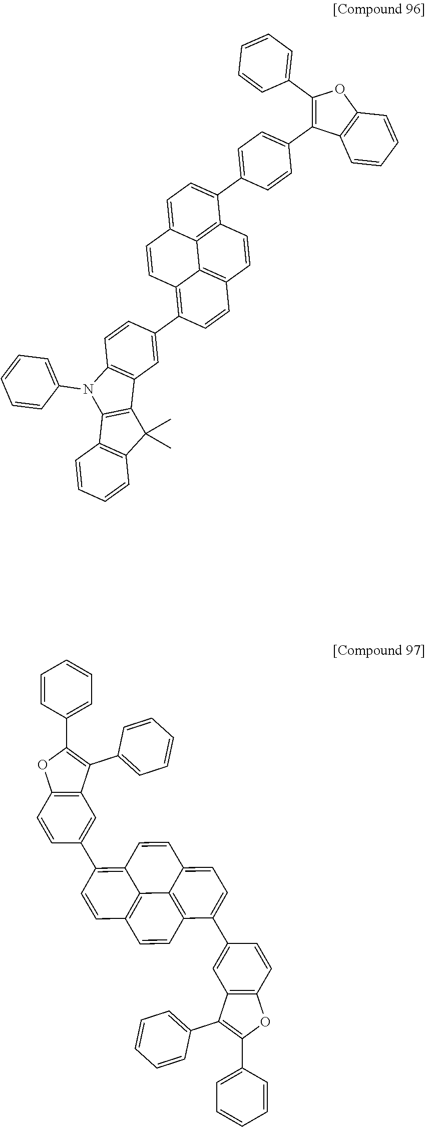 [SMILES: CC1(C)c2ccccc2-c2c1c1cc(-c3ccc4ccc5c(-c6ccc(-c7c(-c8ccccc8)oc8ccccc78)cc6)ccc6ccc3c4c65)ccc1n2-c1ccccc1 ; c1ccc(-c2oc3ccc(-c4ccc5ccc6c(-c7ccc8oc(-c9ccccc9)c(-c9ccccc9)c8c7)ccc7ccc4c5c76)cc3c2-c2ccccc2)cc1]

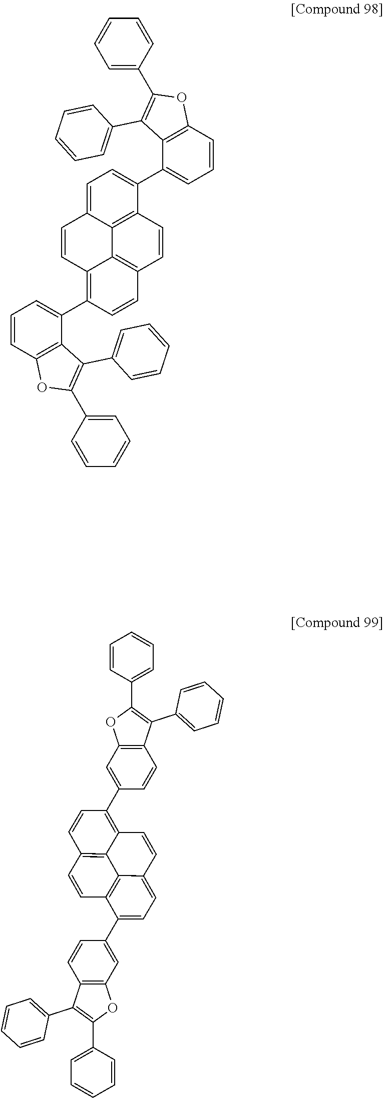 [SMILES: c1ccc(-c2oc3cccc(-c4ccc5ccc6c(-c7cccc8oc(-c9ccccc9)c(-c9ccccc9)c78)ccc7ccc4c5c76)c3c2-c2ccccc2)cc1 ; c1ccc(-c2oc3cc(-c4ccc5ccc6c(-c7ccc8c(-c9ccccc9)c(-c9ccccc9)oc8c7)ccc7ccc4c5c76)ccc3c2-c2ccccc2)cc1]

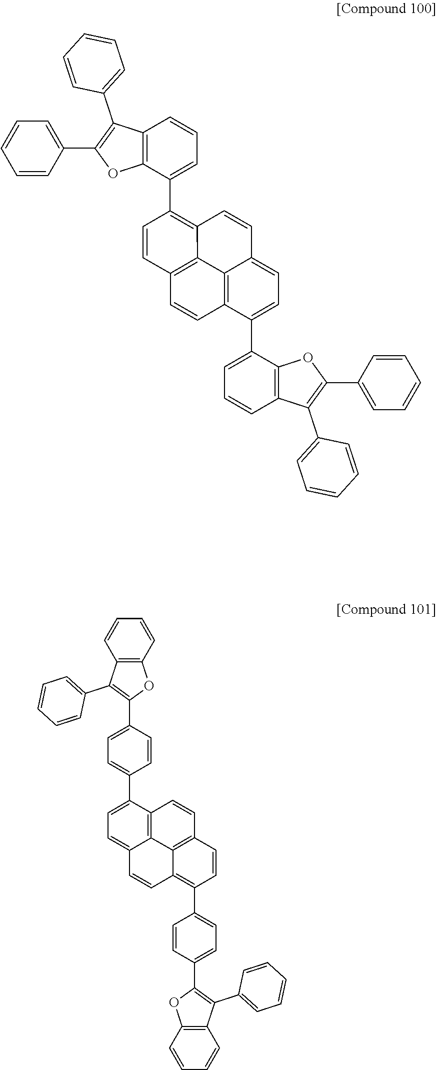 [SMILES: c1ccc(-c2oc3c(-c4ccc5ccc6c(-c7cccc8c(-c9ccccc9)c(-c9ccccc9)oc78)ccc7ccc4c5c76)cccc3c2-c2ccccc2)cc1 ; c1ccc(-c2c(-c3ccc(-c4ccc5ccc6c(-c7ccc(-c8oc9ccccc9c8-c8ccccc8)cc7)ccc7ccc4c5c76)cc3)oc3ccccc23)cc1]

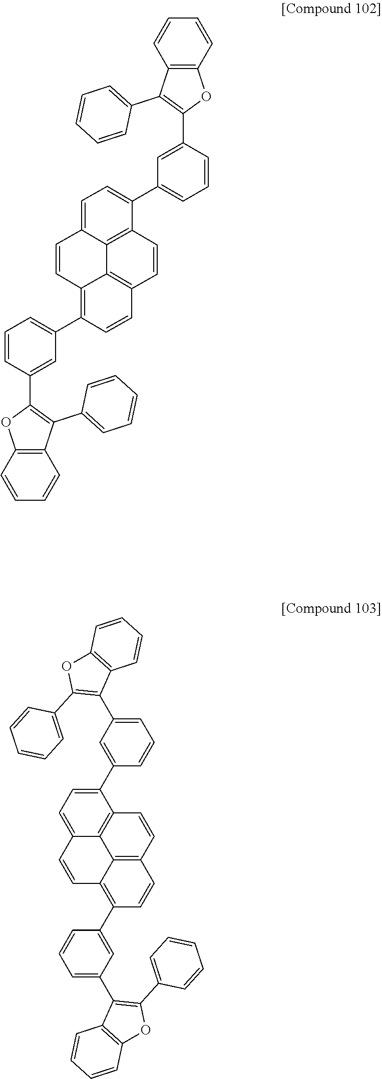 [SMILES: c1ccc(-c2c(-c3cccc(-c4ccc5ccc6c(-c7cccc(-c8oc9ccccc9c8-c8ccccc8)c7)ccc7ccc4c5c76)c3)oc3ccccc23)cc1 ; c1ccc(-c2oc3ccccc3c2-c2cccc(-c3ccc4ccc5c(-c6cccc(-c7c(-c8ccccc8)oc8ccccc78)c6)ccc6ccc3c4c65)c2)cc1]

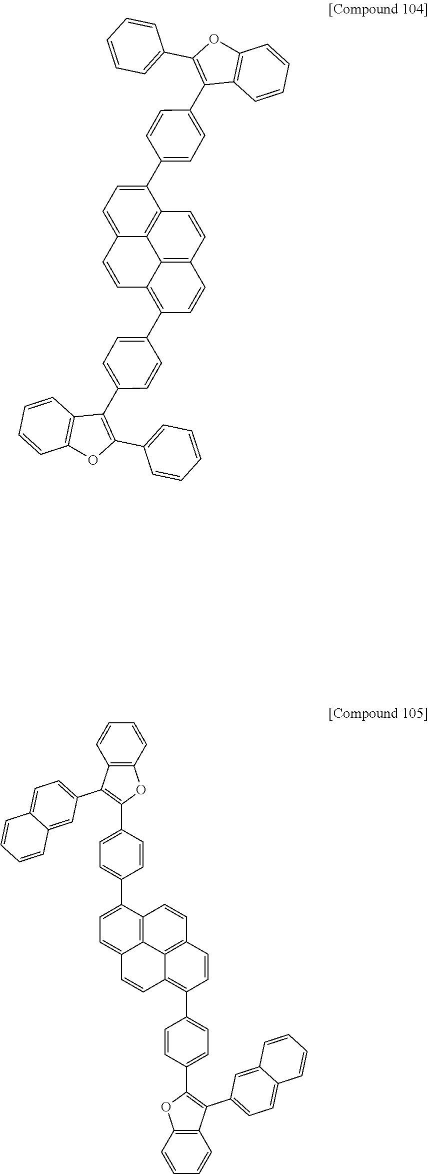 [SMILES: c1ccc(-c2oc3ccccc3c2-c2ccc(-c3ccc4ccc5c(-c6ccc(-c7c(-c8ccccc8)oc8ccccc78)cc6)ccc6ccc3c4c65)cc2)cc1 ; c1ccc2cc(-c3c(-c4ccc(-c5ccc6ccc7c(-c8ccc(-c9oc%10ccccc%10c9-c9ccc%10ccccc%10c9)cc8)ccc8ccc5c6c87)cc4)oc4ccccc34)ccc2c1]

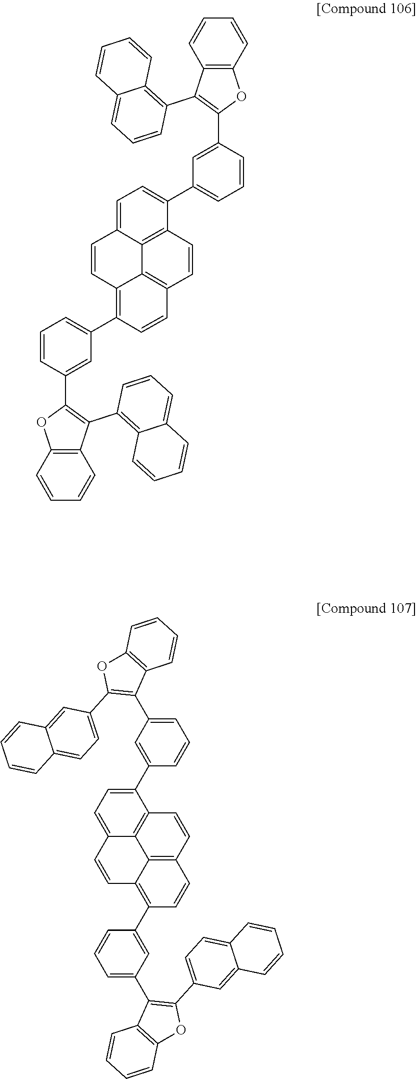 [SMILES: c1cc(-c2oc3ccccc3c2-c2cccc3ccccc23)cc(-c2ccc3ccc4c(-c5cccc(-c6oc7ccccc7c6-c6cccc7ccccc67)c5)ccc5ccc2c3c54)c1 ; c1cc(-c2c(-c3ccc4ccccc4c3)oc3ccccc23)cc(-c2ccc3ccc4c(-c5cccc(-c6c(-c7ccc8ccccc8c7)oc7ccccc67)c5)ccc5ccc2c3c54)c1]

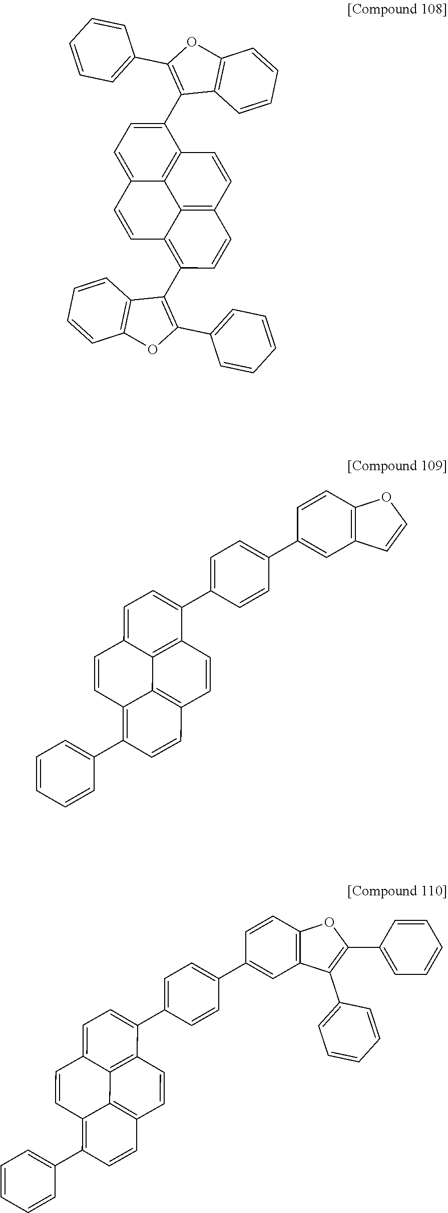 [SMILES: c1ccc(-c2oc3ccccc3c2-c2ccc3ccc4c(-c5c(-c6ccccc6)oc6ccccc56)ccc5ccc2c3c54)cc1 ; c1ccc(-c2ccc3ccc4c(-c5ccc(-c6ccc7occc7c6)cc5)ccc5ccc2c3c54)cc1 ; c1ccc(-c2oc3ccc(-c4ccc(-c5ccc6ccc7c(-c8ccccc8)ccc8ccc5c6c87)cc4)cc3c2-c2ccccc2)cc1]

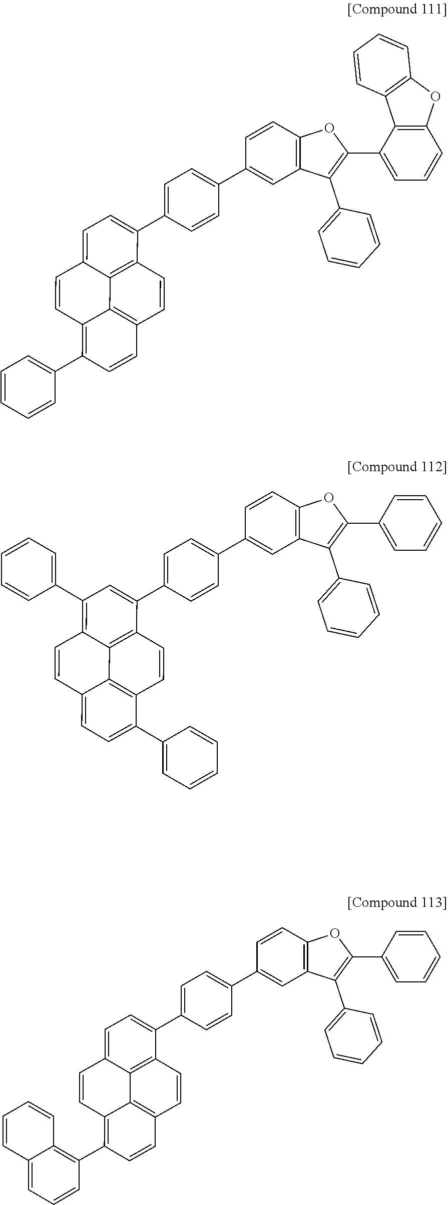 [SMILES: c1ccc(-c2c(-c3cccc4oc5ccccc5c34)oc3ccc(-c4ccc(-c5ccc6ccc7c(-c8ccccc8)ccc8ccc5c6c87)cc4)cc23)cc1 ; c1ccc(-c2oc3ccc(-c4ccc(-c5cc(-c6ccccc6)c6ccc7ccc(-c8ccccc8)c8ccc5c6c78)cc4)cc3c2-c2ccccc2)cc1 ; c1ccc(-c2oc3ccc(-c4ccc(-c5ccc6ccc7c(-c8cccc9ccccc89)ccc8ccc5c6c87)cc4)cc3c2-c2ccccc2)cc1]

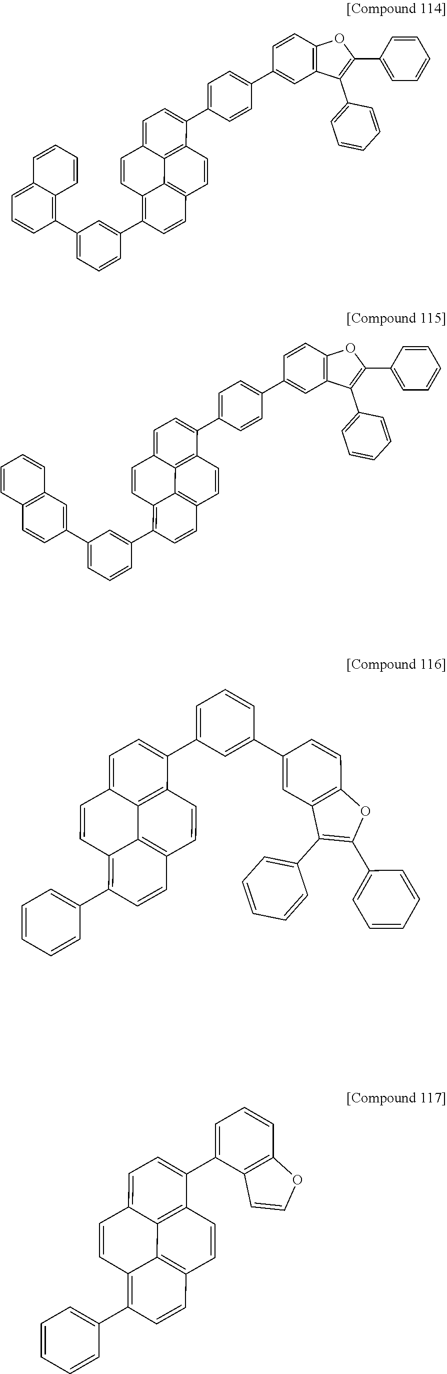 [SMILES: c1ccc(-c2oc3ccc(-c4ccc(-c5ccc6ccc7c(-c8cccc(-c9cccc%10ccccc9%10)c8)ccc8ccc5c6c87)cc4)cc3c2-c2ccccc2)cc1 ; c1ccc(-c2oc3ccc(-c4ccc(-c5ccc6ccc7c(-c8cccc(-c9ccc%10ccccc%10c9)c8)ccc8ccc5c6c87)cc4)cc3c2-c2ccccc2)cc1 ; c1ccc(-c2oc3ccc(-c4cccc(-c5ccc6ccc7c(-c8ccccc8)ccc8ccc5c6c87)c4)cc3c2-c2ccccc2)cc1 ; c1ccc(-c2ccc3ccc4c(-c5cccc6occc56)ccc5ccc2c3c54)cc1]

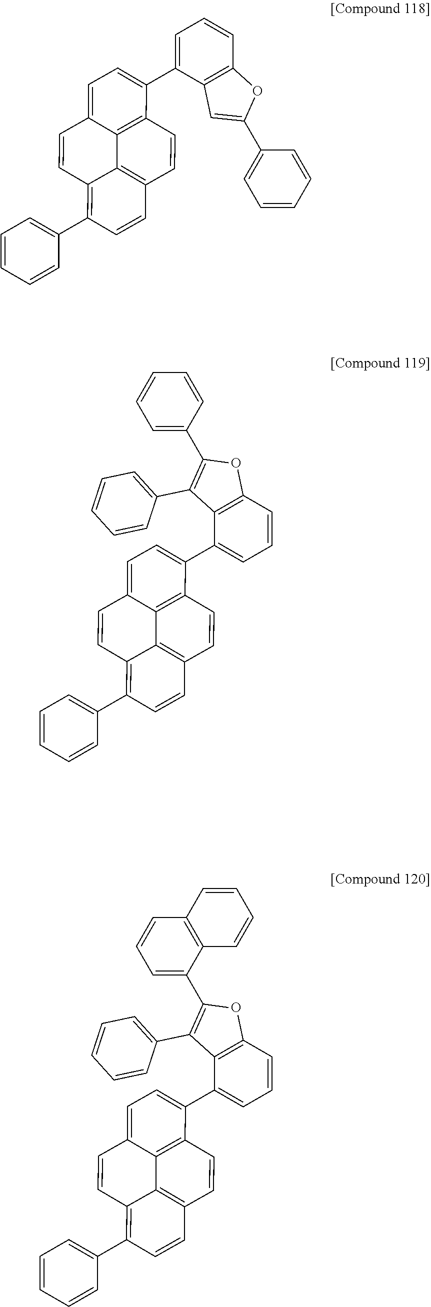 [SMILES: c1ccc(-c2cc3c(-c4ccc5ccc6c(-c7ccccc7)ccc7ccc4c5c76)cccc3o2)cc1 ; c1ccc(-c2oc3cccc(-c4ccc5ccc6c(-c7ccccc7)ccc7ccc4c5c76)c3c2-c2ccccc2)cc1 ; c1ccc(-c2ccc3ccc4c(-c5cccc6oc(-c7cccc8ccccc78)c(-c7ccccc7)c56)ccc5ccc2c3c54)cc1]

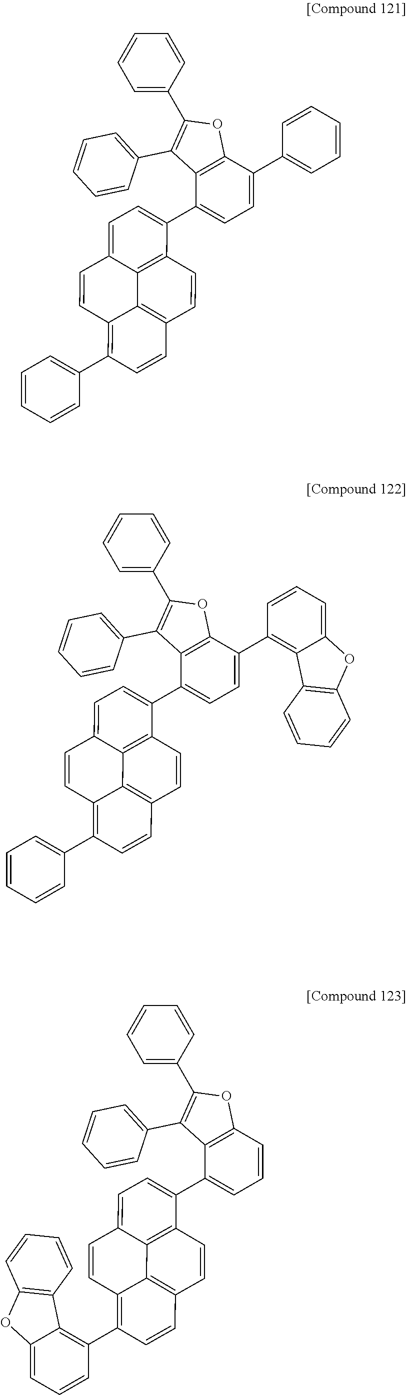 [SMILES: c1ccc(-c2oc3c(-c4ccccc4)ccc(-c4ccc5ccc6c(-c7ccccc7)ccc7ccc4c5c76)c3c2-c2ccccc2)cc1 ; c1ccc(-c2oc3c(-c4cccc5oc6ccccc6c45)ccc(-c4ccc5ccc6c(-c7ccccc7)ccc7ccc4c5c76)c3c2-c2ccccc2)cc1 ; c1ccc(-c2oc3cccc(-c4ccc5ccc6c(-c7cccc8oc9ccccc9c78)ccc7ccc4c5c76)c3c2-c2ccccc2)cc1]

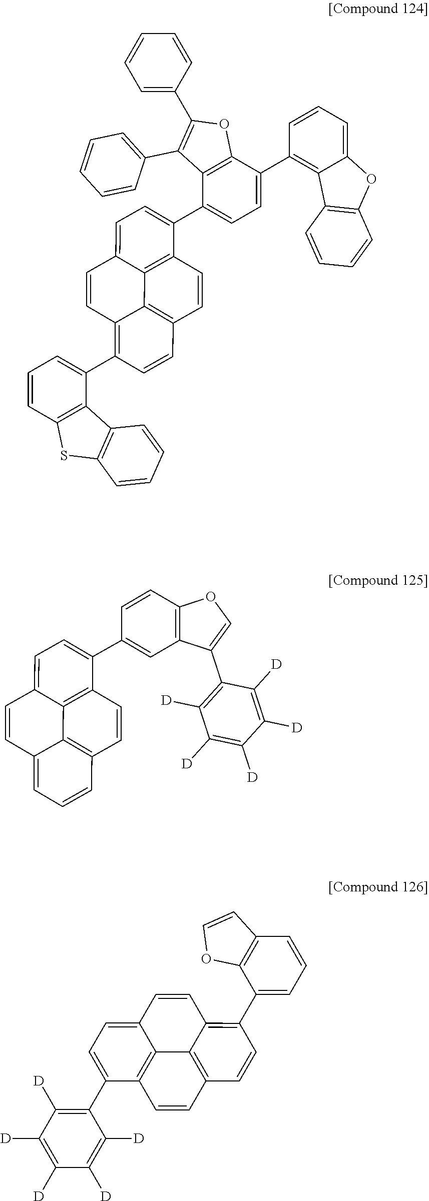 [SMILES: c1ccc(-c2oc3c(-c4cccc5oc6ccccc6c45)ccc(-c4ccc5ccc6c(-c7cccc8sc9ccccc9c78)ccc7ccc4c5c76)c3c2-c2ccccc2)cc1 ; [2H]c1c([2H])c([2H])c(-c2coc3ccc(-c4ccc5ccc6cccc7ccc4c5c67)cc23)c([2H])c1[2H] ; [2H]c1c([2H])c([2H])c(-c2ccc3ccc4c(-c5cccc6ccoc56)ccc5ccc2c3c54)c([2H])c1[2H]]

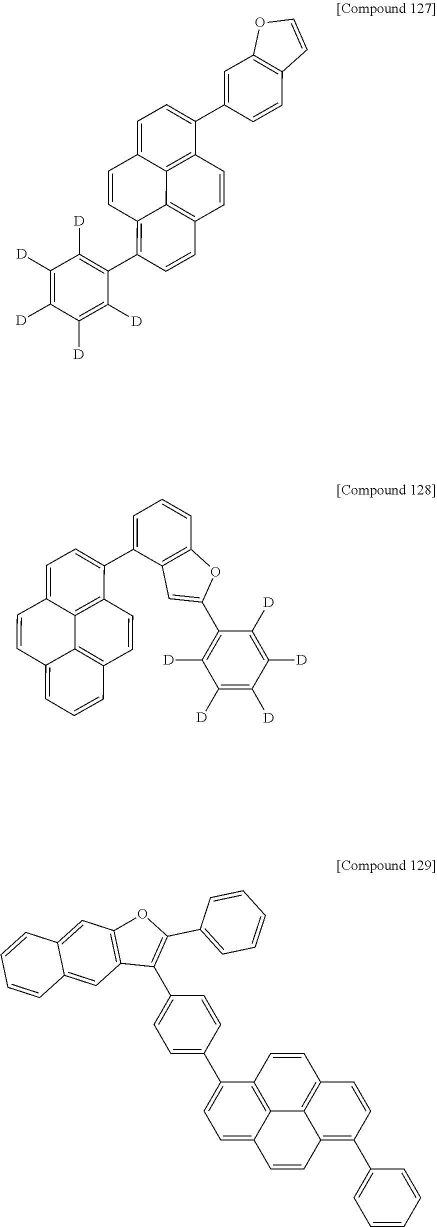 [SMILES: [2H]c1c([2H])c([2H])c(-c2ccc3ccc4c(-c5ccc6ccoc6c5)ccc5ccc2c3c54)c([2H])c1[2H] ; [2H]c1c([2H])c([2H])c(-c2cc3c(-c4ccc5ccc6cccc7ccc4c5c67)cccc3o2)c([2H])c1[2H] ; c1ccc(-c2oc3cc4ccccc4cc3c2-c2ccc(-c3ccc4ccc5c(-c6ccccc6)ccc6ccc3c4c65)cc2)cc1]

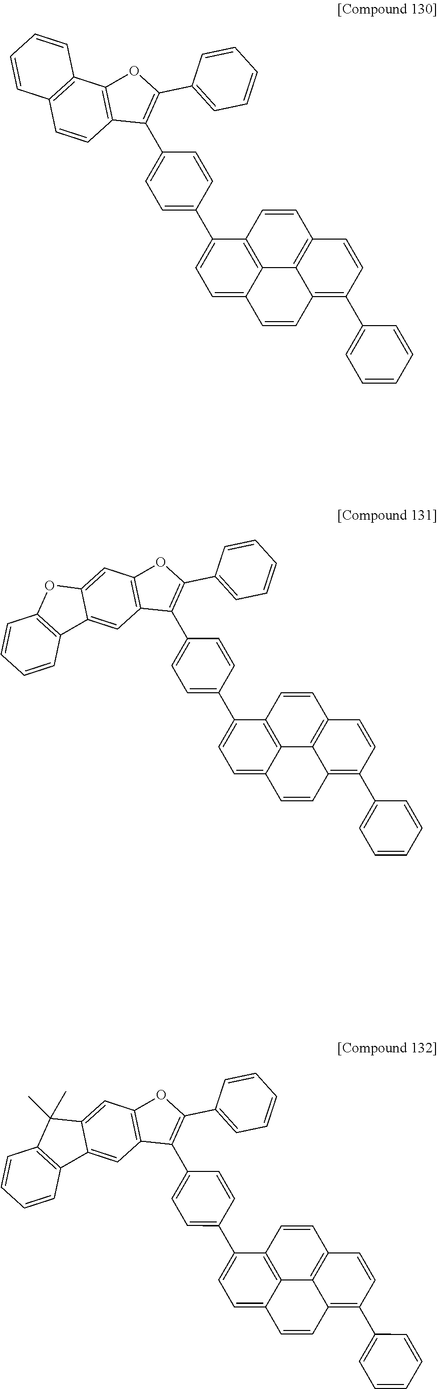 [SMILES: c1ccc(-c2oc3c(ccc4ccccc43)c2-c2ccc(-c3ccc4ccc5c(-c6ccccc6)ccc6ccc3c4c65)cc2)cc1 ; c1ccc(-c2oc3cc4oc5ccccc5c4cc3c2-c2ccc(-c3ccc4ccc5c(-c6ccccc6)ccc6ccc3c4c65)cc2)cc1 ; CC1(C)c2ccccc2-c2cc3c(-c4ccc(-c5ccc6ccc7c(-c8ccccc8)ccc8ccc5c6c87)cc4)c(-c4ccccc4)oc3cc21]

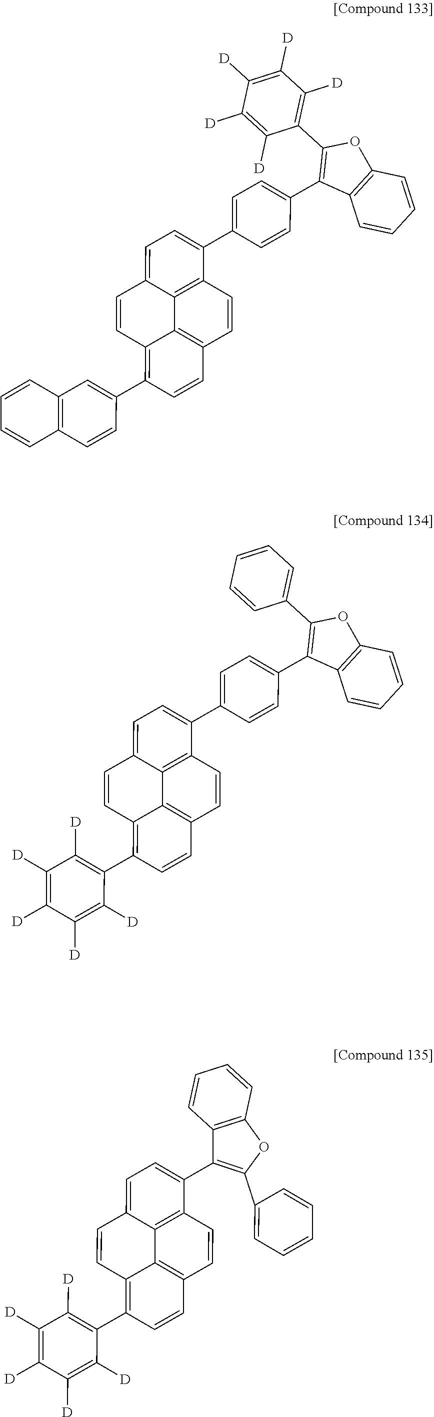 [SMILES: [2H]c1c([2H])c([2H])c(-c2oc3ccccc3c2-c2ccc(-c3ccc4ccc5c(-c6ccc7ccccc7c6)ccc6ccc3c4c65)cc2)c([2H])c1[2H] ; [2H]c1c([2H])c([2H])c(-c2ccc3ccc4c(-c5ccc(-c6c(-c7ccccc7)oc7ccccc67)cc5)ccc5ccc2c3c54)c([2H])c1[2H] ; [2H]c1c([2H])c([2H])c(-c2ccc3ccc4c(-c5c(-c6ccccc6)oc6ccccc56)ccc5ccc2c3c54)c([2H])c1[2H]]

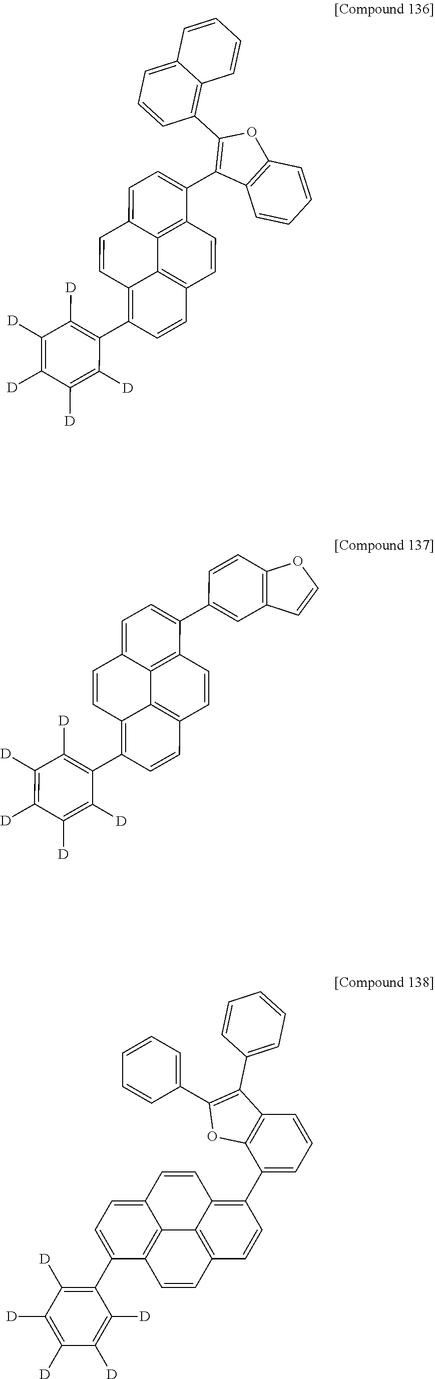 [SMILES: [2H]c1c([2H])c([2H])c(-c2ccc3ccc4c(-c5c(-c6cccc7ccccc67)oc6ccccc56)ccc5ccc2c3c54)c([2H])c1[2H] ; [2H]c1c([2H])c([2H])c(-c2ccc3ccc4c(-c5ccc6occc6c5)ccc5ccc2c3c54)c([2H])c1[2H] ; [2H]c1c([2H])c([2H])c(-c2ccc3ccc4c(-c5cccc6c(-c7ccccc7)c(-c7ccccc7)oc56)ccc5ccc2c3c54)c([2H])c1[2H]]

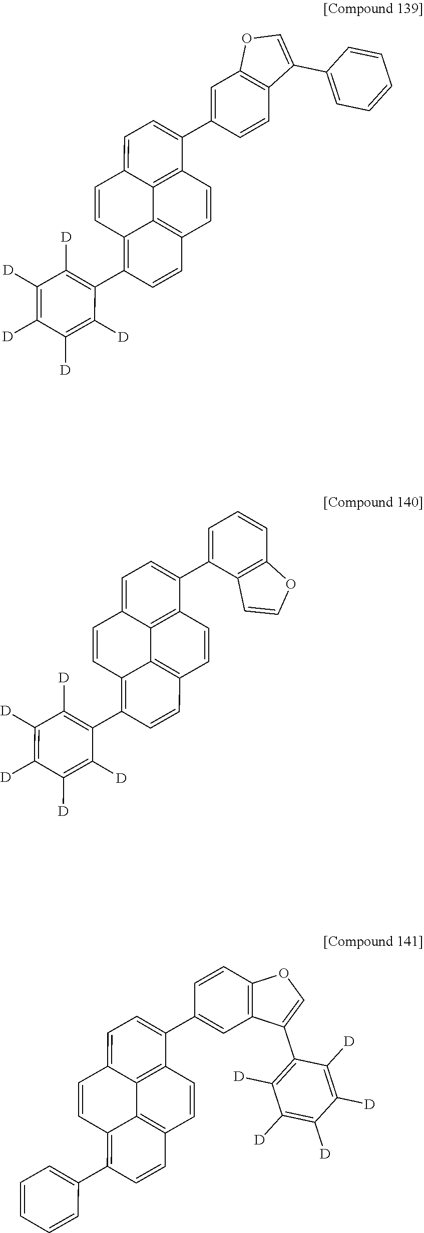 [SMILES: [2H]c1c([2H])c([2H])c(-c2ccc3ccc4c(-c5ccc6c(-c7ccccc7)coc6c5)ccc5ccc2c3c54)c([2H])c1[2H] ; [2H]c1c([2H])c([2H])c(-c2ccc3ccc4c(-c5cccc6occc56)ccc5ccc2c3c54)c([2H])c1[2H] ; [2H]c1c([2H])c([2H])c(-c2coc3ccc(-c4ccc5ccc6c(-c7ccccc7)ccc7ccc4c5c76)cc23)c([2H])c1[2H]]

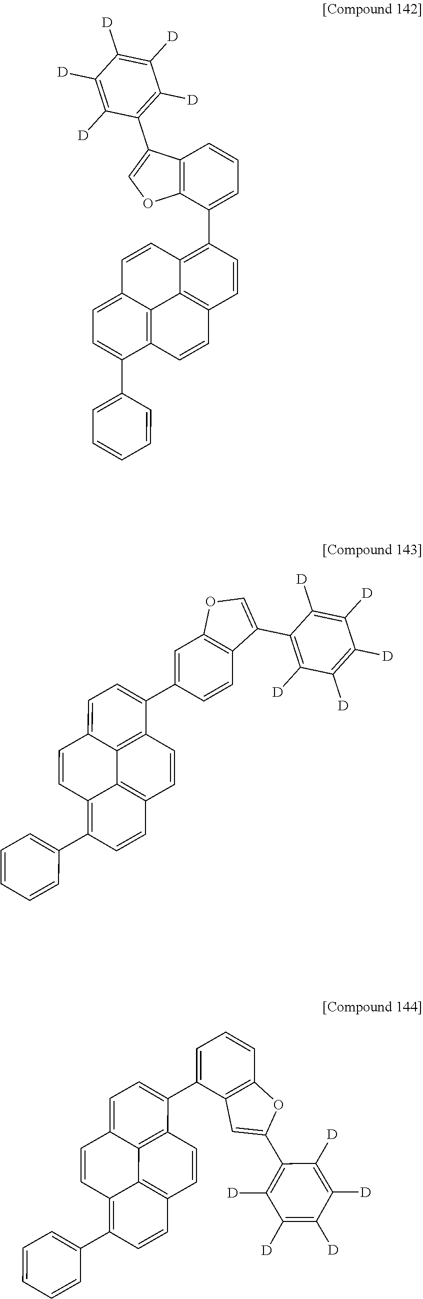 [SMILES: [2H]c1c([2H])c([2H])c(-c2coc3c(-c4ccc5ccc6c(-c7ccccc7)ccc7ccc4c5c76)cccc23)c([2H])c1[2H] ; [2H]c1c([2H])c([2H])c(-c2coc3cc(-c4ccc5ccc6c(-c7ccccc7)ccc7ccc4c5c76)ccc23)c([2H])c1[2H] ; [2H]c1c([2H])c([2H])c(-c2cc3c(-c4ccc5ccc6c(-c7ccccc7)ccc7ccc4c5c76)cccc3o2)c([2H])c1[2H]]

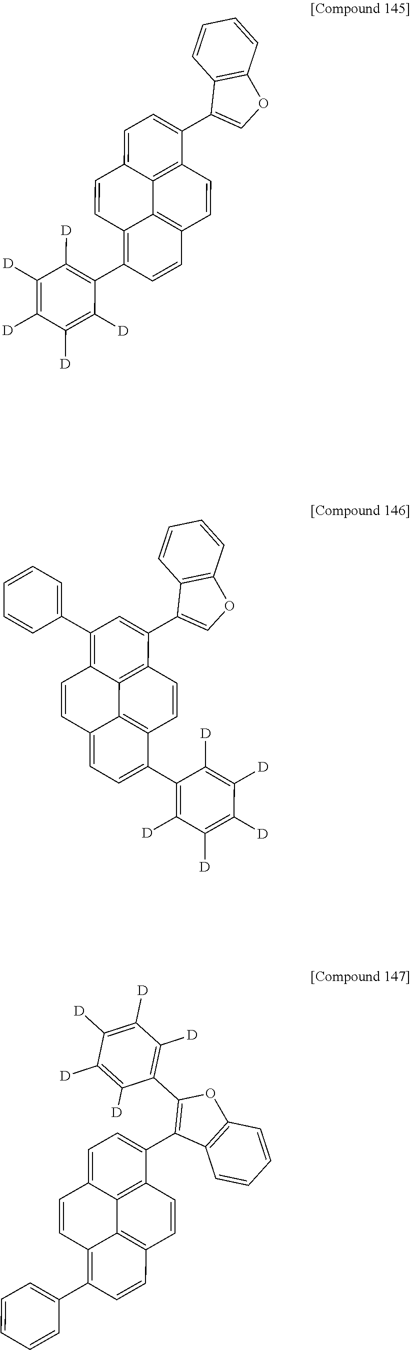 [SMILES: [2H]c1c([2H])c([2H])c(-c2ccc3ccc4c(-c5coc6ccccc56)ccc5ccc2c3c54)c([2H])c1[2H] ; [2H]c1c([2H])c([2H])c(-c2ccc3ccc4c(-c5ccccc5)cc(-c5coc6ccccc56)c5ccc2c3c45)c([2H])c1[2H] ; [2H]c1c([2H])c([2H])c(-c2oc3ccccc3c2-c2ccc3ccc4c(-c5ccccc5)ccc5ccc2c3c54)c([2H])c1[2H]]

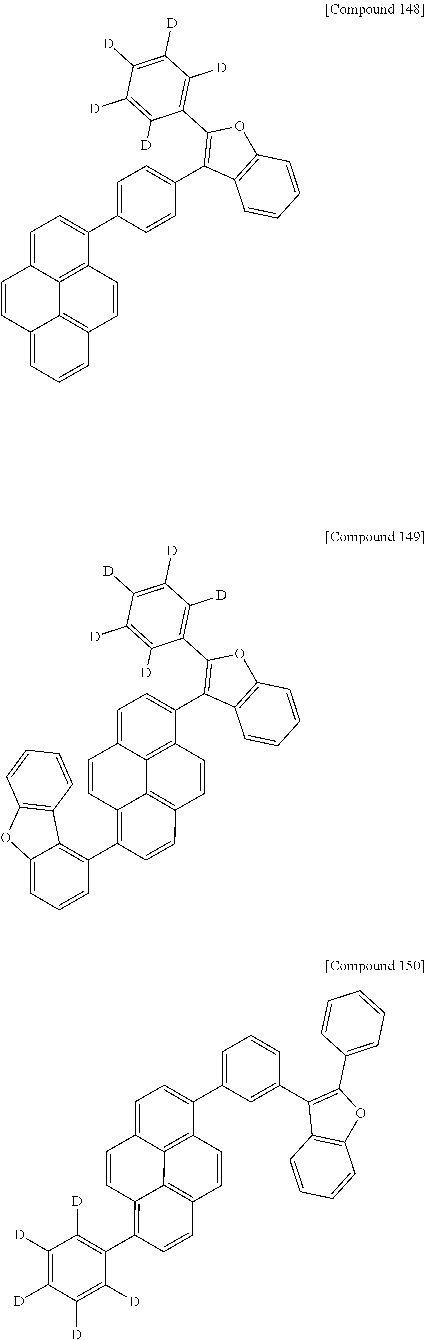 [SMILES: [2H]c1c([2H])c([2H])c(-c2oc3ccccc3c2-c2ccc(-c3ccc4ccc5cccc6ccc3c4c56)cc2)c([2H])c1[2H] ; [2H]c1c([2H])c([2H])c(-c2oc3ccccc3c2-c2ccc3ccc4c(-c5cccc6oc7ccccc7c56)ccc5ccc2c3c54)c([2H])c1[2H] ; [2H]c1c([2H])c([2H])c(-c2ccc3ccc4c(-c5cccc(-c6c(-c7ccccc7)oc7ccccc67)c5)ccc5ccc2c3c54)c([2H])c1[2H]]

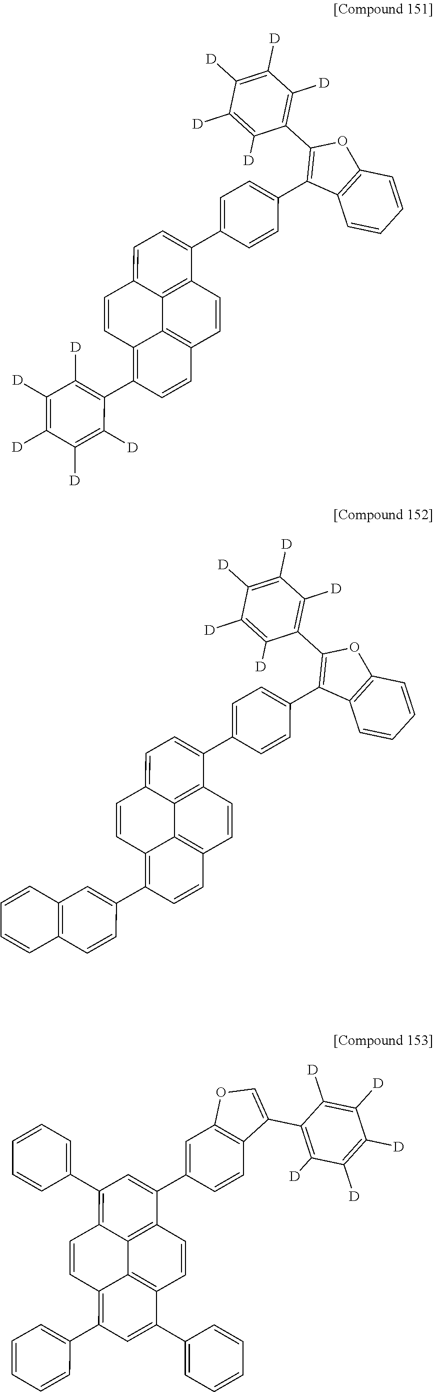 [SMILES: [2H]c1c([2H])c([2H])c(-c2oc3ccccc3c2-c2ccc(-c3ccc4ccc5c(-c6c([2H])c([2H])c([2H])c([2H])c6[2H])ccc6ccc3c4c65)cc2)c([2H])c1[2H] ; [2H]c1c([2H])c([2H])c(-c2oc3ccccc3c2-c2ccc(-c3ccc4ccc5c(-c6ccc7ccccc7c6)ccc6ccc3c4c65)cc2)c([2H])c1[2H] ; [2H]c1c([2H])c([2H])c(-c2coc3cc(-c4cc(-c5ccccc5)c5ccc6c(-c7ccccc7)cc(-c7ccccc7)c7ccc4c5c67)ccc23)c([2H])c1[2H]]

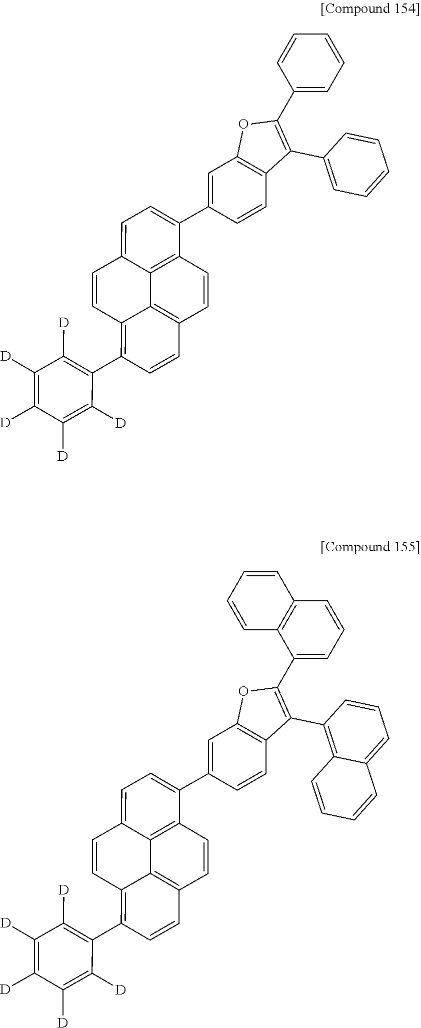 [SMILES: [2H]c1c([2H])c([2H])c(-c2ccc3ccc4c(-c5ccc6c(-c7ccccc7)c(-c7ccccc7)oc6c5)ccc5ccc2c3c54)c([2H])c1[2H] ; [2H]c1c([2H])c([2H])c(-c2ccc3ccc4c(-c5ccc6c(-c7cccc8ccccc78)c(-c7cccc8ccccc78)oc6c5)ccc5ccc2c3c54)c([2H])c1[2H]]

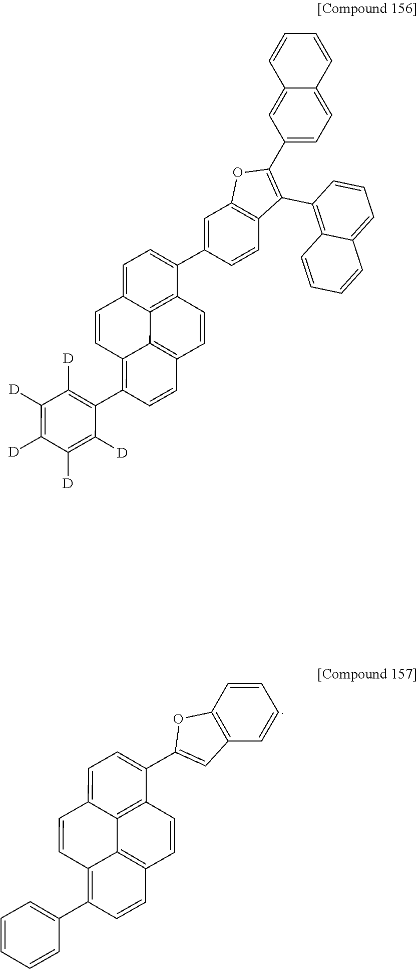 [SMILES: [2H]c1c([2H])c([2H])c(-c2ccc3ccc4c(-c5ccc6c(-c7cccc8ccccc78)c(-c7ccc8ccccc8c7)oc6c5)ccc5ccc2c3c54)c([2H])c1[2H] ; c1ccc(-c2ccc3ccc4c(-c5cc6ccccc6o5)ccc5ccc2c3c54)cc1]

13. An organic light-emitting diode, comprising:
a first electrode:
a second electrode facing the first electrode; and
a light-emitting layer disposed between the first electrode and the second electrode,
wherein the light-emitting layer comprises the compound of claim 1.

14. The organic light-emitting diode of claim 13, wherein the organic layer comprises at least one of a hole injection layer, a hole transport layer, a functional layer capable of both hole injection and hole transport, a light-emitting layer, an electron transport layer, and an electron injection layer.

15. The organic light-emitting diode of claim 14, wherein the organic layer disposed between the first electrode and the second electrode comprises a light-emitting layer composed of a host and a dopant, with the compound serving as a host in the light-emitting layer.

16. The organic light-emitting diode of claim 15, wherein the dopant is at least one selected from the compounds represented by the following Chemical Formulas D1 to Chemical Formula D10: 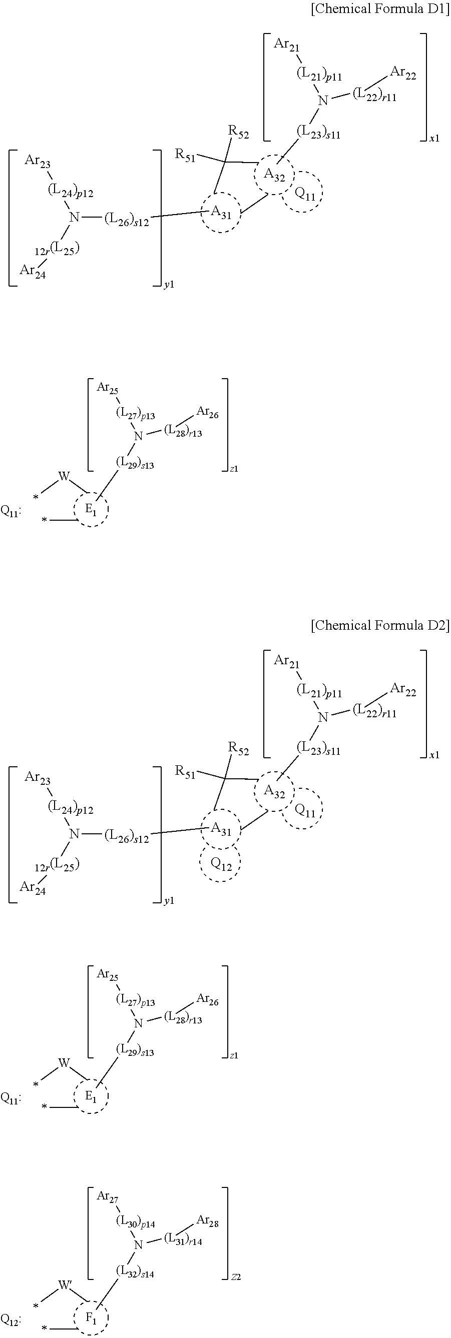
wherein, A₃₁, A₃₂, E₁, and F₁, which are same or different, are each independently a substituted or unsubstituted aromatic hydrocarbon ring of 6 to 50 carbon atoms, or a substituted or unsubstituted heteroaromatic ring of 2 to 40 carbon atoms;
two adjacent carbon atoms of the aromatic ring A₃₁ and two adjacent carbon atoms of the aromatic ring A₃₂ form a 5-membered fused ring together with a carbon atom to which substituents R₅₁ and R₅₂ are bonded;
linkers L₂₁ to L₃₂, which are same or different, are each independently selected from among a single bond, a substituted or unsubstituted alkylene of 1 to 60 carbon atoms, a substituted or unsubstituted alkenylene of 2 to 60 carbon atoms, a substituted or unsubstituted alkynylene of 2 to 60 carbon atoms, a substituted or unsubstituted cycloalkylene of 3 to 60 carbon atoms, a substituted or unsubstituted heterocycloalkylene of 2 to 60 carbon atoms, a substituted or unsubstituted arylene of 6 to 60 carbon atoms, and a substituted or unsubstituted heteroarylene of 2 to 60 carbon atoms,
W and W′, which are same or different, are each independently any one selected from among N—R₅₃, CR₅₄R₅₅, SiR₅₆R₅₇, GeR₅₈R₅₉, O, S, and Se;
R₅₁ to R₅₉ and Ar₂₁ to Ar₂₈, which are same or different, are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a substituted or unsubstituted alkylgermyl of 1 to 30 carbon atoms, a substituted or unsubstituted arylgermyl of 1 to 30 carbon atoms, a cyano, a nitro, and a halogen,
wherein R₅₁ and R₅₂ together can form a mono- or polycyclic aliphatic or aromatic ring that can be a heterocyclic ring bearing a heteroatom selected from among N, O, P, Si, S, Ge, Se, and Te as a ring member;
p11 to p14, r11 to r14, and s11 to s14 are each independently an integer of 1 to 3, wherein when any of them is 2 or greater, the corresponding linkers L₂₁ to L₃₂ can be same or different,
x1 is 1, and y1, z1, and z2, which are same or different, are each independently an integer of 0 to 1; and
Ar₂₁ can form a ring with Ar₂₂, Ar₂₃ can form a ring with Ar₂₄, Ar₂₅ can form a ring with Ar₂₆, and Ar₂₇ can form a ring with Ar₂₈;
two adjacent carbon atoms of the A₃₂ ring moiety of Chemical Formula D1 can occupy respective positions * of Structural Formula Q₁₁ to form a fused ring, and
two adjacent carbon atoms of the A₃₁ ring moiety of Chemical Formula D2 can occupy respective positions * of Structural Formula Q₁₂ to form a fused ring, and two adjacent carbon atoms of the A₃₂ ring moiety of Chemical Formula D2 can occupy respective positions * of Structural Formula Q₁₁ to form a fused ring, 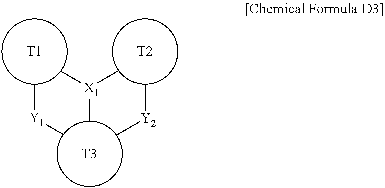
wherein,
X₁ is any one selected from among B, P, and P═O;
T1 to T3, which are same or different, are each independently a substituted or unsubstituted aromatic hydrocarbon ring of 6 to 50 carbon atoms, or a substituted or unsubstituted heteroaromatic ring of 2 to 40 carbon atoms;
Y₁ is any one selected from among N—R₆₁, CR₆₂R₆₃, O, S, and SiR₆₄R₆₅; and
Y₂ is any one selected from among N—R₆₆, CR₆₇R₆₈, O, S, and SiR₆₉R₇₀;
wherein R₆₁ to R₇₀, which are same or different, are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen, and wherein at least one of R₆₁ to R₇₀ can be connected to at least one of T1 to T3 to form an additional mono- or polycyclic aliphatic or aromatic ring. 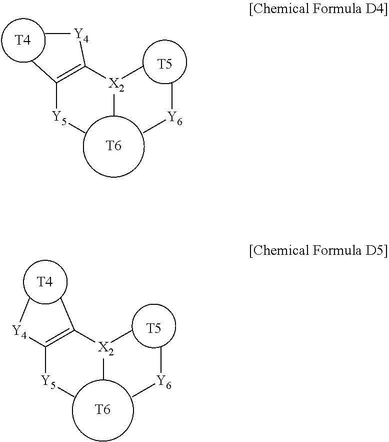
wherein,
X₂ is any one selected from among B, P, and P═O;
T4 to T6 are as defined for T1 to T3 in Chemical Formula D3;
Y₄ is any one selected from among N—R₆₁, CR₆₂R₆₃, O, S, and SiR₆₄R₆₅;
Y₅ is any one selected from among N—R₆₆, CR₆₇R₆₈, O, S, and SiR₆₉R₇₀; and
Y₆ is any one selected from among N—R₇₁, CR₇₂R₇₃, O, S, and SiR₇₄R₇₅,
R₆₁ to R₇₅ being as defined for R₆₁ to R₇₀ in Chemical Formula D3, 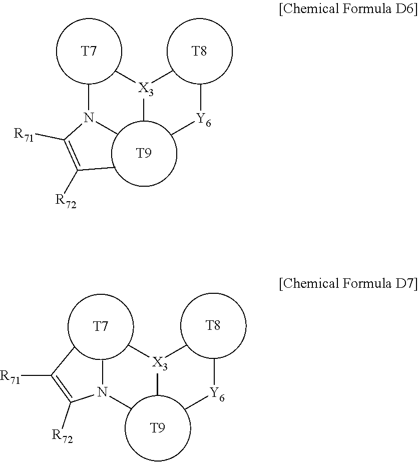
wherein, X₃ is any one selected from among B, P, and P═O;
T₇ to T₉ are defined as for T₁ to T₃ in Chemical Formula D3; and
Y₆ is any one selected from among N—R₆₁, CR₆₂R₆₃, O, S, and SiR₆₄R₆₅,
R₆₁ to R₆₅ and R₇₁ to R₇₂ being each as defined R₆₁ to R₇₀ in Chemical Formula D3,
wherein R₇₁ and R₇₂ can be connected to each other to form an additional mono- or polycyclic aliphatic or aromatic ring, or can be connected to the T7 ring moiety or T9 ring moiety to form an additional mono- or polycyclic aliphatic or aromatic ring, 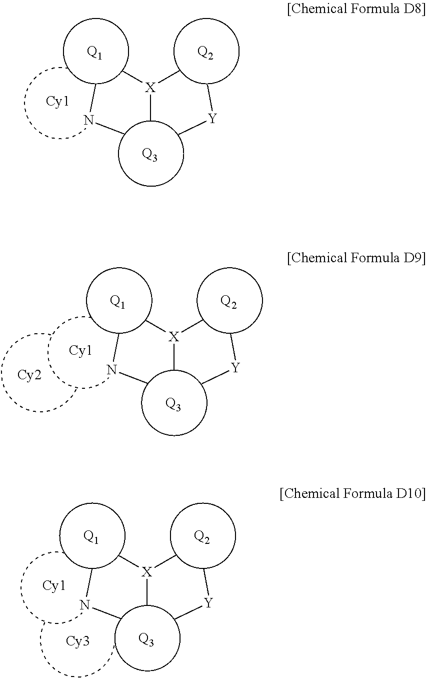
wherein,
X is any one selected from among B, P, and P═O;
Q₁ to Q₃ are each as defined for T1 to T3 in Chemical Formula D3;
Y is any one selected from among N—R₃, CR₄R₅, O, S, and Se,
R₃ to R₅ being each as defined for R₆₁ to R₇₀ in Chemical Formula D3,
wherein R₃ to R₅ can each be connected to the Q₂ or Q₃ ring moiety to form an additional mono- or polycyclic aliphatic or aromatic ring, and
R₄ and R₅ can be connected to each other to form an additional mono- or polycyclic aliphatic or aromatic ring;
the ring formed by Cy1 is a substituted or unsubstituted alkylene of 1 to 10 carbon atoms, except for the nitrogen (N) atom, the aromatic carbon atom of Q₁ to which the nitrogen (N) atom is connected, and the aromatic carbon atom of Q₁ to which Cy1 is to bond;
“Cy2” in Chemical Formula D9 can form a saturated hydrocarbon ring added to Cy1 wherein the ring formed by Cy2 is a substituted or unsubstituted alkylene of 1 to 10 carbon atoms, except for the carbon atoms included in Cy1; and
the ring formed by Cy3 in Chemical Formula D10 is a substituted or unsubstituted alkylene of 1 to 10 carbon atoms, except for the aromatic carbon atom of Q₃ to which Cy3 is to bond, the aromatic carbon atom of Q₃ to which the nitrogen (N) atom is connected, the nitrogen (N) atom, and the carbon atom of Cy1 to which the nitrogen (N) atom is connected,
wherein the term “substituted” in the expression “substituted or unsubstituted” used for compounds of Chemical Formulas D1 to D10 means having at least one substituent selected from the group consisting of a deuterium atom, a cyano, a halogen, a hydroxy, a nitro, an alkyl of 1 to 24 carbon atoms, a hydrogenated alkyl of 1 to 24 carbon atoms, an alkenyl of 2 to 24 carbon atoms, an alkynyl of 2 to 24 carbon atoms, a cycloalkyl of 3 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

17. The organic light-emitting diode of claim 14, wherein at least one selected from among the layers is deposited using a deposition process or a solution process.

18. The organic light-emitting diode of claim 13, wherein the organic light-emitting diode is used for a device selected from among a flat panel display device, a flexible display device, a monochrome or grayscale flat illumination device, a monochrome or grayscale flexible illumination device, a vehicle display device, and a display device for virtual or augmented reality.
